(19) United States
(12) Patent Application Publication
(10) Pub. No.: US 2007/0082347 A1
(43) Pub. Date: Apr. 12, 2007

(54) Gene variants and use thereof

(75) Inventors: Jerry Lanchbury, Salt Lake City, UT (US); Alexander Gutin, Sandy, UT (US); Andrey Zharkikh, Salt Lake City, UT (US); Julia Reid, Salt Lake City, UT (US); Kirsten Timms, Salt Lake City, UT (US); Susanne Wagner, Salt Lake City, UT (US); Ann-Marie Woodland, Roy, UT (US)

Correspondence Address: MYRIAD GENETICS INC.;INTELLECUTAL PROPERTY DEPARTMENT, 320 WAKARA WAY, SALT LAKE CITY, UT, 84108, US

(73) Assignee: Myriad Genetics, Incorporated, Salt Lake City, UT (US)

(21) Appl. No.: 11/450,909

(22) Filed: Jun. 8, 2006

Related U.S. Application Data
(60) Provisional application No. 60/688,459, filed on Jun. 8, 2005.
(60) Provisional application No. 60/688,592, filed on Jun. 8, 2005.
(60) Provisional application No. 60/698,179, filed on Jul. 11, 2005.
(60) Provisional application No. 60/698,211, filed on Jul. 11, 2005.
(60) Provisional application No. 60/741,173, filed on Nov. 30, 2005.
(60) Provisional application No. 60/741,274, filed on Nov. 30, 2005.
(60) Provisional application No. 60/741,350, filed on Nov. 30, 2005.
(60) Provisional application No. 60/741,351, filed on Nov. 30, 2005.

(51) Int. Cl. C12Q 1/68; C07H 21/04; C12M 3/00

(52) U.S. Cl. 435/6; 536/23.2; 435/287.2; 257/E51.02; 977/924

(57) ABSTRACT

Variants in TLK1, WARS2, ARTS2, MSR, AKAP9, DNAJD1, GOLPH4, RABEP1, TAP2, NARG2, DDX58, CD39, FKBP1a, SRI, XRRA1, IRF5 and AMFR genes are disclosed which are useful as biomarkers for predicting the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene expression level and the biological functions associated thereof.

### CROSS-REFERENCE TO RELATED APPLICATIONS

This application claims priority under 35 U.S.C. § 119(e) to U.S. provisional application Ser. No. 60/698,211, filed Jul. 11, 2005; U.S. provisional application Ser. No. 60/741,350, filed Nov. 30, 2005; U.S. provisional application Ser. No. 60/688,592, filed Jun. 8, 2005; U.S. provisional application Ser. No. 60/741,274, filed Nov. 30, 2005; U.S. provisional application Ser. No. 60/688,459, filed Jun. 8, 2005; U.S. provisional application Ser. No. 60/741,173, filed Nov. 30, 2005; U.S. provisional application Ser. No. 60/741,351, filed Nov. 30, 2005; and U.S. provisional application Ser. No. 60/698,179, filed Jul. 11, 2005, all of which are hereby incorporated by reference in their entirety.

### REFERENCE TO A “SEQUENCE LISTING,” A TABLE, OR A COMPUTER PROGRAM LISTING APPENDIX SUBMITTED ON A COMPACT DISC

A Sequence Listing containing all relevant nucleotide and/or amino acid sequences has been submitted on 3 compact discs (CDs). The CD labeled “CRF” contains one file entitled “3107-01-1U 2006-06-08 SEQ-LIST-TXT.” Tables 1-81 are also contained in the accompanying CDs, labeled “Copy 1” and “Copy 2,” each containing two files entitled “3107-01-1U 2006-06-08 SEQ-LIST-TXT” and “3107-01-1U 2006-06-08 Tables TH.” The file “3107-01-1U 2006-06-08 SEQ-LIST-TXT” was created and written onto CD on Jun. 8, 2006, and is 854 kilobytes in size. The file “3107-01-1U 2006-06-08 Tables TH” was created and written onto CD on Jun. 8, 2006, and is 2.1 megabytes in size. The information contained on these CDs is hereby incorporated by reference in its entirety.

### FIELD OF THE INVENTION

This invention generally relates to pharmacogenetics, particularly to the identification of genetic variants that are associated with gene expression, and methods of using the identified variants.

### BACKGROUND OF THE INVENTION

Genetic polymorphic variations such as single-nucleotide polymorphisms (SNPs) are valuable tools for deciphering mechanisms of biological functions and understanding the underlying basis of human diseases. See generally, Cooper et al. in The Metabolic and Molecular Bases of Inherited Diseases, 1:259-291 (1995), Scriver et al., eds., McGraw-Hill, New York. SNPs are small variations in genomes. They are among the most common forms of human genetic variations. A large number of monogenic human diseases are associated with genetic polymorphic variations such as SNPs in the so-called susceptibility genes. For example, polymorphic variations in the coagulation factor gene F5 have been linked directly to deep-vein thrombosis. See Bertina et al., Nature, 369:64-67 (1994). SNPs in the Apolipoprotein E gene correlate with the risk of Alzheimer's disease. See U.S. Pat. No. 5,773,220.

Genetic polymorphic variations are also associated with varying response to drugs and natural environmental agents. See generally, McCarthy et al., Nat. Biotechnol., 18:505-508 (2000); Nebert, Am. J. Hum. Genet. 60:265-271 (1997); and Puga et al., Crit. Rev. Toxicol. 27(2):199-222 (1997). Pharmacogenomic studies have found a large number of SNPs associated with differing drug response. For example, genetic polymorphic variations in the 5-lipoxygenase gene, which codes for an anti-asthma drug target, have been linked to variations in drug response. See Drazen et al., Nat. Genet. 22:168-170 (1999). In addition, genetic variants in the drug-metabolizing enzyme thiopurine methyltransferase correlate with adverse drug reactions. See Krynetski et al., Pharm. Res., 16:342-349 (1999).

Since proteins are intimately involved in essential biological functions and drug metabolism, the apparent nexus between genetic polymorphic variations and human diseases and drug responses is not at all surprising since any gene sequence change may potentially affect gene expression and protein function. For example, SNPs in exons may lead to different protein sequences exhibiting altered protein activities (e.g., sickle cell anemia). SNPs in exons, and thus mRNAs, may also affect the splicing, processing, transport, translation, or stability of the mRNAs that contain them. See e.g., Cooper et al., in The Metabolic and Molecular Bases of Inherited Diseases, 1:259-291 (1995), Scriver et al., eds., McGraw-Hill, New York. SNPs in exons may also alter mRNA secondary or tertiary structures, i.e., mRNA folding, and thus affect post-transcriptional gene regulation. See Shen et al., Proc. Natl. Acad. Sci. USA, 96:7871-7876 (1999).

Polymorphic variations in non-coding regions have also been linked to diseases and other phenotypic effects. For example, SNPs in introns can affect mRNA splicing and thus alter gene expression. See e.g., Otterness et al., J. Clin. Invest., 101(5):1036-44 (1998); Hayashi et al., Growth Horm. IGF Res., 9:434-437 (1999); Tsai et al., Biochem. Mol. Med., 61:9-15 (1997); Yu et al., Atherosclerosis, 146:125-31 (1999); Nemer et al., Blood, 89:4608-16 (1997); States et al., Mutat. Res., 363:171-7 (1996). Genetic variations in intronic sequences may also influence gene transcription or interactions between gene transcription products and other cellular machines. Likewise, polymorphic variations in transcriptional regulatory regions of a gene may alter transcriptional patterns of the gene. See McGuigan et al., Osteoporos. Int., 11:338-43 (2000); Arnaud et al., Arterioscler. Thromb. Vasc. Biol., 20:892-898 (2000).

Very often, a genetic polymorphic variant alone does not cause any detectable effect on gene expression or gene function. However, it may act in concert with other known or unknown polymorphic variants in the gene and cause cumulative or synergistic effect sufficient to alter gene expression pattern or the properties of the protein encoded by the gene. Even if a particular genetic polymorphic variant does not contribute to any changes in gene expression or protein function, it may be near or linked to one or more other genetic variants that directly cause phenotypic defects. Therefore, by identifying such genetic variants, one could reasonably predict the phenotypic effect in an individual having such genetic variants. In addition, one can also identify haplotypes, that is, combinations of genetic variants in a particular gene or chromosome present in an individual. Haplotypes represent patterns of genetic variations and are important tools for genetic analysis and diagnosis.

Indeed, genetic polymorphic variations such as SNPs and haplotypes containing SNPs are invaluable genetic markers for a variety of applications. For example, genetic polymorphic variations are useful in genetic analysis for studying polymorphic allele segregation and polymorphism origins. In addition, genetic polymorphisms can be used as markers in population studies, and in forensic medicine. More importantly, SNPs can be particularly useful in genetic diagnoses for identifying individuals predisposed to certain diseases. See e.g., U.S. Pat. Nos. 5,994,080, 5,942,390, 5,773,220, and 5,736,323. Further, SNPs can also be valuable tools for predicting an individual's response to drug treatment or other exogenous interventions.

Thus, there is need in the art to identify additional SNPs in the human genome, particularly those associated with defined phenotypes.

### SUMMARY OF THE INVENTION

The present invention is based on the discovery of a number of genetic polymorphic variations, particularly SNPs and haplotypes, in the human autocrine motility factor receptor gene (“AMFR”), human tousled-like kinase 1 gene (“TLK1”), human mitochondrial tryptophanyl-tRNA synthetase gene (“WARS2”), human adipocyte-derived leucine aminopeptidase gene (“ARTS2”), human methionine synthase reductase gene (“MSR”), human A-kinase anchor protein 9 gene (“AKAP9”), human Homosapiens DnaJ (Hsp40) homolog, subfamily D, member 1 gene (“DNAJD1”), human golgi phosphoprotein gene (“GOLPH4”), human RAB GTPase binding effector protein 1 gene (“RABEP1”), human transporter associated with antigen processing gene (“TAP2”), human NMDA receptor regulated gene 2 gene (“NARG2”), human DEAD-box polypeptide 58 gene (“DDX58”), human CD39 antigen gene (“CD39”), human FK506-binding protein 1a gene (“FKBP1a”), human sorcin gene (“SRI”), human X-ray resistance associated protein gene (“XRRA1”), human interferon regulatory factor gene (“IRF”) and human autocrine motility factor receptor gene (“AMFR”). The SNPs and/or haplotypes are summarized in Tables 1-35. It has also been surprisingly discovered that the mRNA expression levels of these genes are inherited in a Mendelian manner in humans. Furthermore, the SNPs and/or haplotypes are associated with the heritable mRNA levels of the gene transcripts. Thus, the SNPs and haplotypes are useful in predicting mRNA levels of the genes in human cells and tissues, and thus can be useful in predicting the gene expression and the biological functions associated therewith.

For example, over-expression of AMFR induces a transformed phenotype and produces tumors in nude mice. Also, expression levels of AMFR in tumor cells correlate with the cells' potential to metastasize. Thus, the SNPs and haplotypes of the present invention in AMFR, which are associated with inheritable AMFR mRNA expression levels, are useful biomarkers for the prognosis of cancer in patients.

In addition, TLK1 overexpression increases resistance to DNA damage caused by ionizing radiation. Expression of a dominant-negative kinase TLK1 mutant results in chromosome missegregation and aneuploidy, indicating the role of TLK1 in preserving genetic integrity. Thus, the SNPs and haplotypes of the present invention in TLK1, which are associated with inheritable TLK1 mRNA expression levels, are useful biomarkers for the prediction of response to radiation treatment in patients.

Dysregulation of WARS2 expression has been associated with diseases such as cancer, neurodegenerative and cardiovascular disease. Thus, SNPs and haplotypes of the present invention in TLK1, of the present invention are useful as biomarkers for the prediction and detection of such diseases, and for determining cancer prognosis in a patient.

ARTS1 expression levels have been linked to tumor suppression, blood pressure regulation, immune response and autoimmune disease. Thus, SNPs associated with alterations in ARTS1 mRNA expression levels are useful as biomarkers to predict or detect susceptibility to such diseases in patients, and to predict patient response to the treatment of such diseases.

MSR is a critical enzyme in methionine metabolism, reducing methionine synthetase cofactor cobalamin to its active state. As such, MSR levels are central to efficient methionine metabolism. Methionine is also necessary for cancer cell proliferation in vitro. Accordingly, altering levels of MSR affects the level of methionine thereby altering the rate of cancer cell growth. Thus, the SNPs and haplotypes of the present invention, which are associated with MSR mRNA expression levels, are useful as biomarkers for diseases such as atherosclerosis, thrombosis, methylmalonicaciduria and cancer.

AKAP9 expression has been linked to neurodegenerative disorders, cardiovascular disease, cancer and depression in humans. The SNPs of the present invention associated with altered levels of AKAP9 mRNA expression are therefore useful to predict or detect susceptibility to neurodegenerative disorders, cardiovascular disease, cancer and depression.

DNAJD1 expression levels have been associated with resistance to chemotherapeutics in ovarian cancer, and polymorphisms predicting DNAJD1 levels have utility as predictive markers for therapeutic responses to apoptosis-inducing agents in cancer therapy. Thus, the SNPs of the present invention in the DNAJD1 gene, which are associated with altered DNAJD1 expression levels, are useful biomarkers for predicting the effectiveness of cancer treatments in patients.

Under-expression of GOLPH4 results in protein accumulation in early/recycling endosomes of those proteins that use the bypass pathway. Under-expression of GOLPH4 also inhibits invasion of cells by toxins. Therefore, GOLPH4 plays an important role in the movement of proteins and toxins from endosomes to the Golgi apparatus via the bypass pathway. Thus, the SNPs of the present invention in the GOLPH4 gene, which are associated with altered GOLPH4 expression levels, are useful biomarkers for determining the susceptibility of patients to toxins and pathogen invasion.

RABEP1 levels have been shown to influence endosomal trafficking associated with neurodegenerative diseases. Altered RABEP1 expression has also been associated with tumor formation and growth. Thus, SNPs associated with altered RABEP1 mRNA levels are useful as biomarkers to predict and detect susceptibility to cancer and neurodegenerative disease in an individual, as well as to predict tumor progression.

TAP2 mRNA expression levels correlate with expression of cell surface proteins involved in autoimmune function, immune response and cancer cell metastasis. Thus, SNPs associated with altered TAP2 mRNA expression levels are useful in the prediction and detection of susceptibility to autoimmune disease, viral infection and cancer development in an individual. The SNPs are also useful in determining the potential for progression of viral infection and cancer cell metastasis.

NARG2 expression levels increase in the absence of NMDA receptor 1 demonstrating the influence of NARG2 level on neuronal cell differentiation. Thus, the SNPs of the present invention showing an association with NARG2 mRNA expression levels are useful as a means of predicting/detecting susceptibility and/or progression of neurological disease in an individual.

DDX58 levels correlate with COX2 levels, which have been shown to have an association with cancer, mainly tumor formation and growth. DDX58 expression levels are also associated with the activity of natural killer cells and cytotoxicity in response to viral infection. Thus, SNPs associated with DDX58 mRNA levels are useful as biomarkers to predict and detect susceptibility to cancer and viral infection.

CD39 levels correlate with susceptibility to vascular injury and coronary syndromes. CD39 expression levels are also associated with rates of hemostasis and thromobosis. Further, levels of CD39 have been associated with the inflammatory response. Thus, SNPs associated with CD39 mRNA levels are useful as biomarkers to predict and detect susceptibility to vascular injury, coronary disease, transplant rejection and inflammatory response.

FKBP1a levels correlate with susceptibility to vascular injury and coronary syndromes. FKBP1a expression levels are also associated with rates of hemostasis and thromobosis. Further, levels of FKBP1a have been associated with the inflammatory response. Thus, SNPs associated with FKBP1a mRNA levels are useful as biomarkers to predict and detect susceptibility to vascular injury, coronary disease, transplant rejection and inflammatory response.

Expression levels of SRI have been associated with the prognosis and remission rate of acute myeloid lymphoma cancer. SRI expression levels are also associated with resistance to chemotherapeutics in vitro and in vivo. The expression of SRI has also been shown to be associated with increased cardiac contractility and recovery of cardiomyopathy. Thus, SNP associated with SRI mRNA levels are useful as biomarkers to predict prognosis and remission rates of cancer as well as patient response to treatment with chemotherapeutics. The SRI SNP of the present invention is also useful as a biomarker in detecting ability to recover from cardiovascular disease.

XRRA1 levels correlate with cell sensitivity to ionizing radiation. Thus, SNPs associated with XRRA1 mRNA expression levels are useful as biomarkers to predict and detect sensitivity to ionizing radiation.

IRF5 mRNA expression levels have been associated with immune response to viral infection in humans. Thus, SNPs of the present invention are useful as biomarkers to predict and detect susceptibility to and progression of viral infection in an individual.

Accordingly, in a first aspect of the present invention, an isolated TLK1, WARS2, ARTS2, MSR, AKAP9, DNAJD1, GOLPH4, RABEP1, TAP2, NARG2, DDX58, CD39, FKBP1a, SRI, XRRA1, IRF5 and AMFR nucleic acid variant is provided containing at least one of the newly discovered genetic variants, as summarized in Tables 1-35. The present invention also encompasses an isolated oligonucleotide comprising a contiguous span of at least 18, preferably from 18 to 50 nucleotides of the sequence of one of the above nucleic acid variants, wherein the contiguous span encompasses and contains a nucleotide variant selected from those in Tables 1-35.

DNA microchips are also provided comprising one or more isolated nucleic acid variants and/or one or more isolated oligonucleotides according to the present invention.

In accordance with another aspect of the invention, an isolated TLK1, WARS2, ARTS2, MSR, AKAP9, DNAJD1, GOLPH4, RABEP1, TAP2, NARG2, DDX58, CD39, FKBP1a, SRI, XRRA1, IRF5 and AMFR protein variant, or a fragment thereof, is provided comprising one or more amino acid variants selected from those in Tables 1-35.

The present invention also provides an isolated antibody specifically immunoreactive with a protein variant of the present invention.

In accordance with yet another aspect of the present invention, a method is provided for genotyping an individual by determining whether the individual has a nucleotide variant or an amino acid variant provided in accordance with the present invention. In addition, the present invention also provides a method for predicting in an individual the gene expression level or mRNA level of the TLK1, WARS2, ARTS2, MSR, AKAP9, DNAJD1, GOLPH4, RABEP1, TAP2, NARG2, DDX58, CD39, FKBP1a, SRI, XRRA1, IRF5 and AMFR genes. The method comprises the step of detecting in the individual the presence or absence of a nucleotide variant, or an amino acid variant, provided according to the present invention, which is associated with an inheritable mRNA expression level.

In accordance with another aspect of the invention, a detection kit is also provided for genotyping the nucleotide variant in an individual. In a specific embodiment, the kit is used in predicting the level of gene expression in an individual. The kit may include, in a carrier or confined compartment, any nucleic acid probes or primers, or antibodies useful for detecting the nucleotide variants or amino acid variants of the present invention as described herein. The kit can also include other reagents such as DNA polymerase, buffers, nucleotides and others that can be used in the method of detecting the variants according to this invention. In addition, the kit preferably also contains instructions for its use.

The foregoing and other advantages and features of the invention, and the manner in which the same are accomplished, will become more readily apparent upon consideration of the following detailed description of the invention taken in conjunction with the accompanying examples and drawings, which illustrate preferred and exemplary embodiments.

### BRIEF DESCRIPTION OF THE DRAWINGS

FIGS. 1-17 show the relative mRNA expression levels of the variant genotypes for particular SNPs in the TLK1, WARS2, ARTS2, MSR, AKAP9, DNAJD1, GOLPH4, RABEP1, TAP2, NARG2, DDX58, CD39, FKBP1a, SRI, XRRA1, IRF5 and AMFR genes. The relative expression is based on signal intensities from the RNA expression chips in a logarithmic fashion. For instance, a difference of one unit represents a two-fold difference in mRNA expression levels, whereas a difference of three represents an eight-fold increase in expression;

FIG. 1 is a plot illustrating the association of the TLK1 genotype at EX7@+190 with the TLK1 mRNA level. The figure shows the relative expression levels of the different genotypes C/C, T/C and T/T;

FIG. 2 is a plot illustrating the association of the WARS2 genotype at EX6@2152 with the WARS2 mRNA level. The figure shows the relative expression levels of the different genotypes G/G, A/G and A/A;

FIG. 3 is a plot illustrating the association of the ARTS1 genotype at EX19@328 with the ARTS1 mRNA level. The figure shows the relative expression levels of the different genotypes C/C, C/T and T/T;

FIG. 4 is a plot illustrating the association of the MSR genotype at EX7@146 with the MSR mRNA level. The figure shows the relative expression levels of the different genotypes A/G and A/A;

FIG. 5 is a plot illustrating the association of the AKAP9 genotype at EX10@186 with the AKAP9 mRNA level. The figure shows the relative expression levels of the different genotypes G/G, A/G and A/A;

FIG. 6 is a plot illustrating the association of the DNAJD1 genotype at EX1@368 with the corresponding DNAJD1 mRNA level. The figure shows the relative expression levels of the different genotypes T/T, T/C and C/C;

FIG. 7 is a plot illustrating the association of the GOLPH4 genotype at EX15@−85 with the GOLPH4 mRNA level. The figure shows the relative expression levels of the genotypes G/G and C/G;

FIG. 8 is a plot illustrating the association of the RABEP1 genotype at EX17@15 with the RABEP1 mRNA level. The figure shows the relative expression levels of the genotypes A/A, A/G and G/G;

FIG. 9 is a plot illustrating the association of the TAP2 genotype at EX12@61 with the TAP2 mRNA level. The figure shows the relative expression levels of the genotypes A/A, A/G and G/G;

FIG. 10 is a plot illustrating the association of the NARG2 genotype at EX12@48 with the NARG2 mRNA level. The figure shows the relative expression levels of the genotypes C/C, C/T and T/T;

FIG. 11 is a plot illustrating the association of the DDX58 genotype at EX14@+78 with the DDX58 mRNA level. The figure shows the relative expression levels of the genotypes C/C, C/T and T/T;

FIG. 12 is a plot illustrating the association of the CD39 genotype at 97,374,982 with the CD39 mRNA level. The figure shows the relative expression levels of the different genotypes G/G, A/G and A/A;

FIG. 13 is a plot illustrating the association of the CD39 genotype at 97,374,982 with the FKBP1a mRNA level. The figure shows the relative expression levels of the genotypes G/G, A/G and A/A;

FIG. 14 is a plot illustrating the association of the SRI genotype at EX9@351 with the SRI mRNA level. The figure shows the relative expression levels of the genotypes C/C, C/T and T/T;

FIG. 15 is a plot illustrating the association of the XRRA1 genotype at EX2@26 with the XRRA1 mRNA level. The figure shows the relative expression levels of the different genotypes C/C, C/G and G/G;

FIG. 16 is a plot illustrating the association of the IRF5 genotype at EX19@9328 with the IRF5 mRNA level. The figure shows the relative expression levels of the genotypes A/A, A/G and G/G; and

FIG. 17 is a plot illustrating the association of the AMFR genotype at EX14@483 with the AMFR mRNA level. The figure shows the relative expression levels of the genotypes G/G, G/A and A/A.

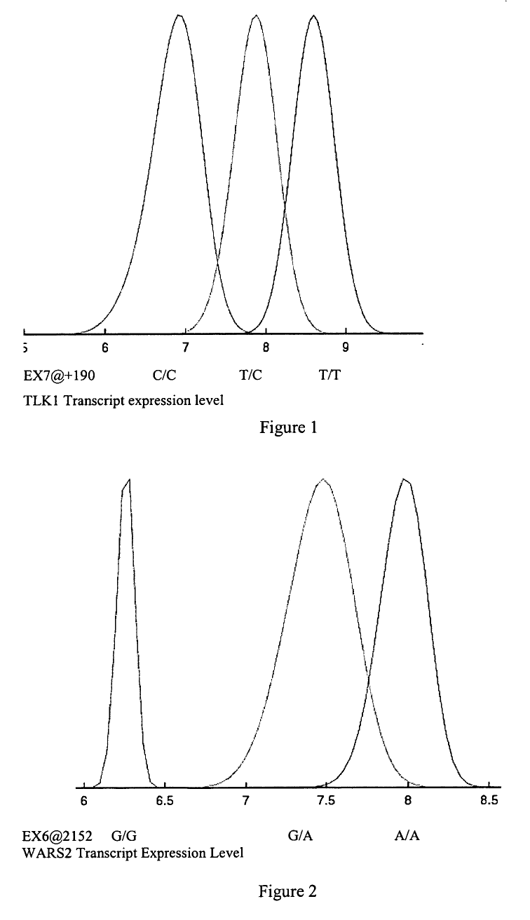

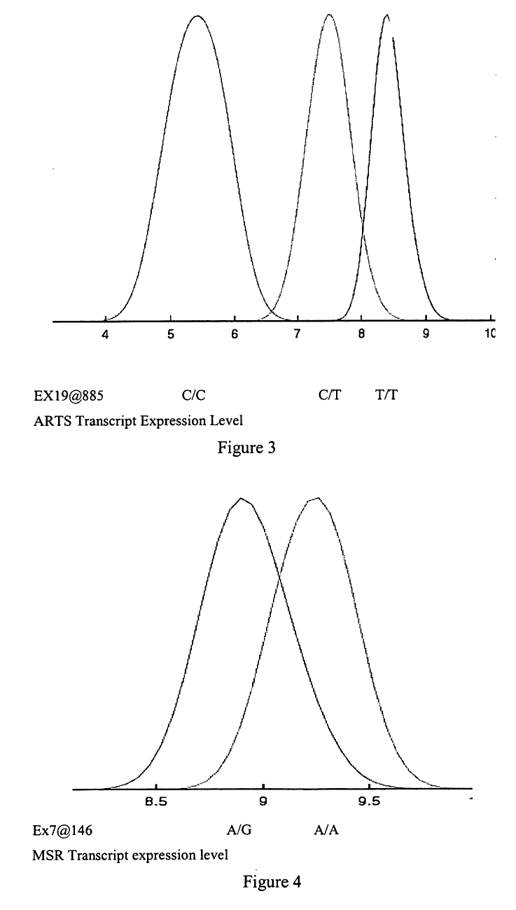

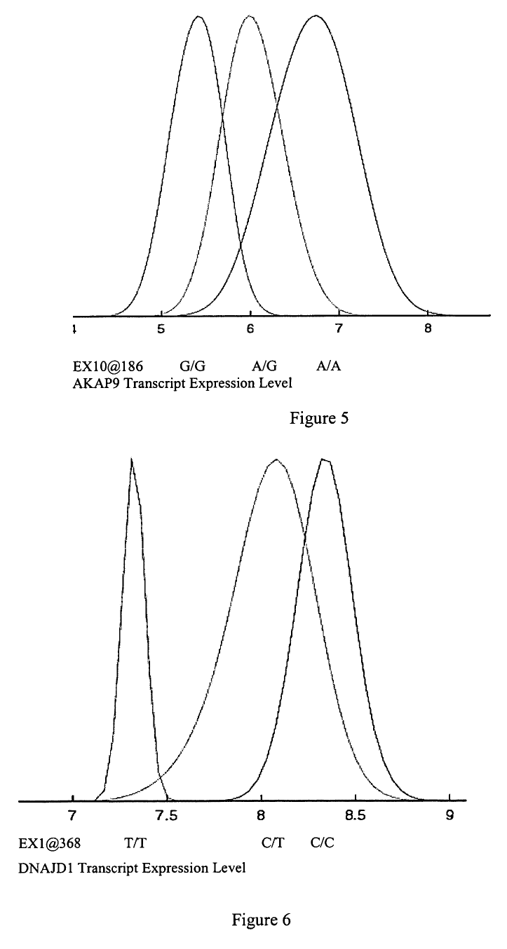

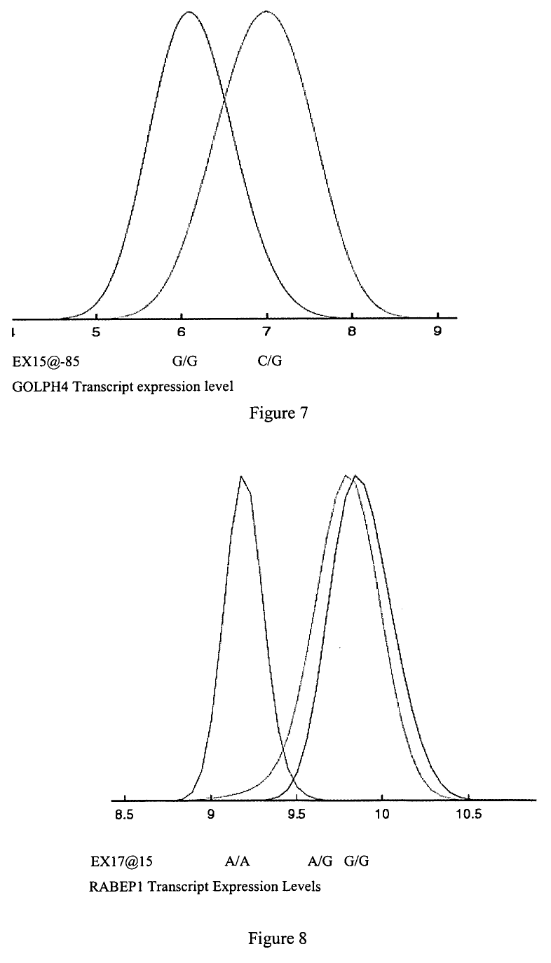

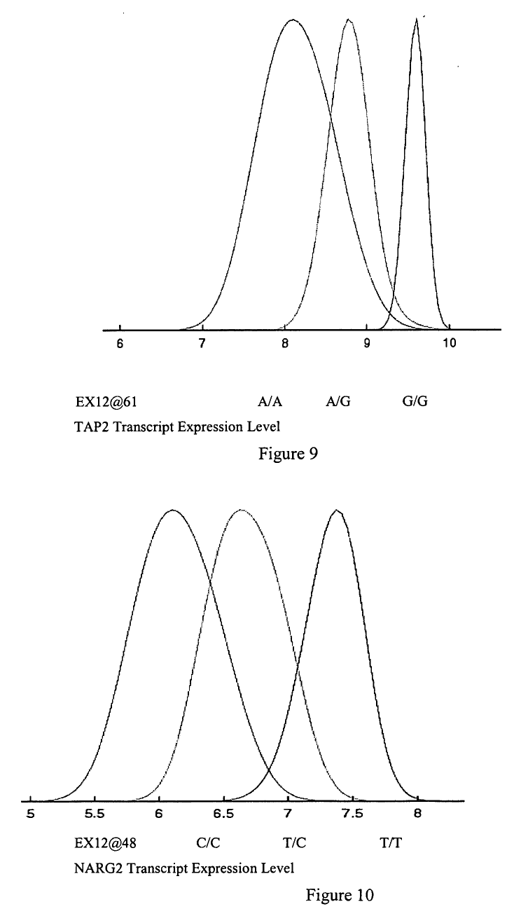

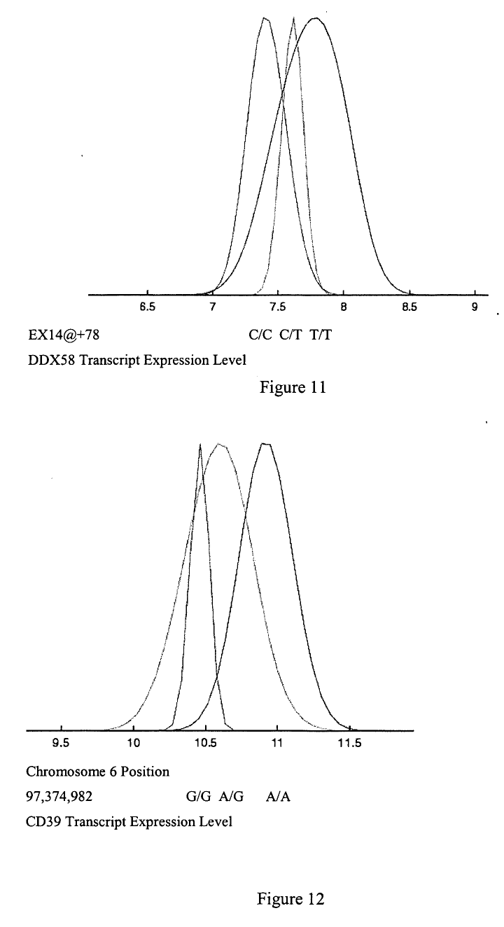

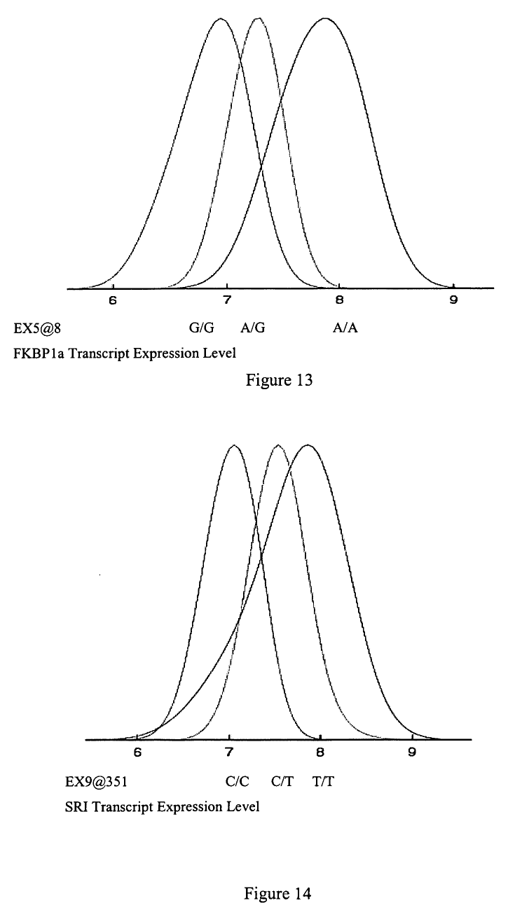

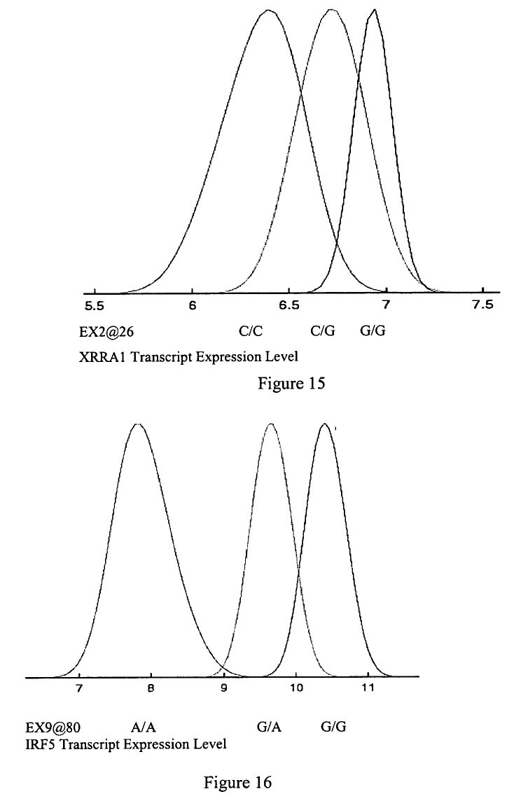

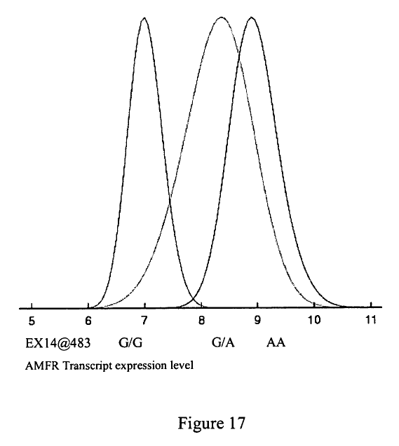

### DETAILED DESCRIPTION OF THE INVENTION

### 1. Definitions

The terms “genetic variant” and “nucleotide variant” are used herein interchangeably to refer to changes or alterations to, or variations in, the reference human TLK1, WARS2, ARTS2, MSR, AKAP9, DNAJD1, GOLPH4, RABEP1, TAP2, NARG2, DDX58, CD39, FKBP1a, SRI, XRRA1, IRF5 and AMFR gene or cDNA sequence at a particular locus, including, but not limited to, nucleotide base deletions, insertions, inversions, and substitutions in the coding and noncoding regions. Deletions may be of a single nucleotide base, a portion or a region of the nucleotide sequence of the gene, or of the entire gene sequence. Insertions may be of one or more nucleotide bases. The “genetic variant” or “nucleotide variant” may occur in transcriptional regulatory regions, untranslated regions of mRNA, exons, introns, or exon/intron junctions. The “genetic variant” or “nucleotide variant” may or may not result in stop codons, frame shifts, deletions of amino acids, altered gene transcript splice forms or altered amino acid sequence.

The term “allele” or “gene allele” is used herein to refer generally to a naturally occurring gene having a reference sequence or a gene containing a specific nucleotide variant.

As used herein, “haplotype” is a combination of genetic (nucleotide) variants in a region of an mRNA or a genomic DNA on a chromosome found in an individual. Thus, a haplotype includes a number of genetically linked polymorphic variants that are typically inherited together as a unit.

As used herein, the term “amino acid variant” is used to refer to an amino acid change to, or an amino acid variant of, a reference human TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR amino acid sequence resulting from a “genetic variant” or “nucleotide variant” of the human gene encoding TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR. The term “amino acid variant” is intended to encompass not only single amino acid substitutions, but also amino acid deletions, insertions, and other significant changes of amino acid sequence in TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR.

The term “genotype” as used herein means the nucleotide characters at a particular nucleotide variant marker (or locus) in either one allele or both alleles of a gene (or a particular intergenic chromosome region). With respect to a particular nucleotide position of a gene of interest, the nucleotide(s) at that locus or equivalent thereof in one or both alleles form the genotype of the gene at that locus. A genotype can be homozygous, heterozygous or hemizygous. Accordingly, “genotyping” means determining the genotype, that is, the nucleotide(s) at a particular chromosome locus. Genotyping can also be done by determining the amino acid variant at a particular position of a protein which can be used to deduce the corresponding nucleotide variant(s).

The term “locus” refers to a specific position or site in a gene sequence, chromosome, or protein. Thus, there may be one or more contiguous nucleotides in a particular gene or chromosomal locus, or one or more amino acids at a particular locus in a polypeptide. Moreover, “locus” may also be used to refer to a particular position in a gene or chromosome where one or more nucleotides have been deleted, inserted, or inverted.

As used herein, the terms “polypeptide,” “protein,” and “peptide” are used interchangeably to refer to an amino acid chain in which the amino acid residues are linked by covalent peptide bonds. The amino acid chain can be of any length of at least two amino acids, including full-length proteins. Unless otherwise specified, the terms “polypeptide,” “protein,” and “peptide” also encompass various modified forms thereof, including but not limited to glycosylated forms, phosphorylated forms, etc.

The terms “primer”, “probe,” and “oligonucleotide” are used herein interchangeably to refer to a relatively short nucleic acid fragment or sequence. They can be DNA, RNA, or a hybrid thereof, or chemically modified analogs or derivatives thereof. Typically, they are single-stranded. However, they can also be double-stranded having two complementing strands that can be separated by denaturation. Normally, they have a length of from about 8 nucleotides to about 200 nucleotides, preferably from about 12 nucleotides to about 100 nucleotides, and more preferably about 18 to about 50 nucleotides. They can be labeled with detectable markers or modified in any conventional manners for various molecular biological applications.

The term “isolated” when used in reference to nucleic acids (e.g., genomic DNAs, cDNAs, mRNAs, or fragments thereof) is intended to mean that a nucleic acid molecule is present in a form that is substantially separated from other naturally occurring nucleic acids that are normally associated with the molecule. Specifically, since a naturally existing chromosome (or a viral equivalent thereof) includes a long nucleic acid sequence, an “isolated nucleic acid” as used herein means a nucleic acid molecule having only a portion of the nucleic acid sequence in the chromosome but not one or more other portions present on the same chromosome. More specifically, an “isolated nucleic acid” typically includes no more than 25 kb naturally occurring nucleic acid sequences which immediately flank the nucleic acid in the naturally existing chromosome (or a viral equivalent thereof). However, it is noted that an “isolated nucleic acid” as used herein is distinct from a clone in a conventional library such as genomic DNA library and cDNA library in that the clone in a library is still in admixture with almost all the other nucleic acids of a chromosome or cell. Thus, an “isolated nucleic acid” as used herein also should be substantially separated from other naturally occurring nucleic acids that are on a different chromosome of the same organism. Specifically, an “isolated nucleic acid” means a composition in which the specified nucleic acid molecule is significantly enriched so as to constitute at least 10% of the total nucleic acids in the composition.

An “isolated nucleic acid” can be a hybrid nucleic acid having the specified nucleic acid molecule covalently linked to one or more nucleic acid molecules that are not the nucleic acids naturally flanking the specified nucleic acid. For example, an isolated nucleic acid can be in a vector. In addition, the specified nucleic acid may have a nucleotide sequence that is identical to a naturally occurring nucleic acid or a modified form or mutein thereof having one or more mutations such as nucleotide substitution, deletion, insertion, inversion, and the like.

An isolated nucleic acid can be prepared from a recombinant host cell (in which the nucleic acids have been recombinantly amplified and/or expressed), or can be a chemically synthesized nucleic acid having a naturally occurring nucleotide sequence or an artificially modified form thereof.

The term “isolated polypeptide” as used herein is defined as a polypeptide molecule that is present in a form other than that found in nature. Thus, an isolated polypeptide can be a non-naturally occurring polypeptide. For example, an “isolated polypeptide” can be a “hybrid polypeptide.” An “isolated polypeptide” can also be a polypeptide derived from a naturally occurring polypeptide by additions or deletions or substitutions of amino acids. An isolated polypeptide can also be a “purified polypeptide” which is used herein to mean a composition or preparation in which the specified polypeptide molecule is significantly enriched so as to constitute at least 10% of the total protein content in the composition. A “purified polypeptide” can be obtained from natural or recombinant host cells by standard purification techniques, or by chemically synthesis, as will be apparent to skilled artisans.

The terms “hybrid protein,” “hybrid polypeptide,” “hybrid peptide,” “fusion protein,” “fusion polypeptide,” and “fusion peptide” are used herein interchangeably to mean a non-naturally occurring polypeptide or isolated polypeptide having a specified polypeptide molecule covalently linked to one or more other polypeptide molecules that do not link to the specified polypeptide in nature. Thus, a “hybrid protein” may be two naturally occurring proteins or fragments thereof linked together by a covalent linkage. A “hybrid protein” may also be a protein formed by covalently linking two artificial polypeptides together. Typically but not necessarily, the two or more polypeptide molecules are linked or “fused” together by a peptide bond forming a single non-branched polypeptide chain.

The term “high stringency hybridization conditions,” when used in connection with nucleic acid hybridization, means hybridization conducted overnight at 42° C. in a solution containing 50% formamide, 5×SSC (750 mM NaCl, 75 mM sodium citrate), 50 mM sodium phosphate, pH 7.6, 5× Denhardt's solution, 10% dextran sulfate, and 20 microgram/ml denatured and sheared salmon sperm DNA, with hybridization filters washed in 0.1×SSC at about 65° C. The term “moderate stringent hybridization conditions,” when used in connection with nucleic acid hybridization, means hybridization conducted overnight at 37° C. in a solution containing 50% formamide, 5×SSC (750 mM NaCl, 75 mM sodium citrate), 50 mM sodium phosphate, pH 7.6, 5× Denhardt's solution, 10% dextran sulfate, and 20 microgram/ml denatured and sheared salmon sperm DNA, with hybridization filters washed in 1×SSC at about 50° C. It is noted that many other hybridization methods, solutions and temperatures can be used to achieve comparable stringent hybridization conditions as will be apparent to skilled artisans.

For the purpose of comparing two different nucleic acid or polypeptide sequences, one sequence (test sequence) may be described to be a specific “percentage identical to” another sequence (comparison or reference sequence) in the present disclosure. In this respect, the percentage identity is determined by the algorithm of Karlin and Altschul, Proc. Natl. Acad. Sci. USA, 90:5873-5877 (1993), which is incorporated into various BLAST programs. Specifically, the percentage identity is determined by the “BLAST 2 Sequences” tool, which is available through the National Center for Biotechnology Information's (NCBI's) website. See Tatusova and Madden, FEMS Microbiol. Lett., 174(2):247-250 (1999). For pairwise DNA-DNA comparison, the BLASTN 2.1.2 program is used with default parameters (Match: 1; Mismatch: −2; Open gap: 5 penalties; extension gap: 2 penalties; gap x_dropoff: 50; expect: 10; and word size: 11, with filter). For pairwise protein-protein sequence comparison, the BLASTP 2.1.2 program is employed using default parameters (Matrix: BLOSUM62; gap open: 11; gap extension: 1; x_dropoff: 15; expect: 10.0; and wordsize: 3, with filter). Percent identity of two sequences is calculated by aligning a test sequence with a comparison sequence using BLAST 2.1.2, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence. When BLAST 2.1.2 is used to compare two sequences, it aligns the sequences and yields the percent identity over defined, aligned regions. If the two sequences are aligned across their entire length, the percent identity yielded by the BLAST 2.1.1 is the percent identity of the two sequences. If BLAST 2.1.2 does not align the two sequences over their entire length, then the number of identical amino acids or nucleotides in the unaligned regions of the test sequence and comparison sequence is considered to be zero and the percent identity is calculated by adding the number of identical amino acids or nucleotides in the aligned regions and dividing that number by the length of the comparison sequence.

As used herein the term “linkage disequilibrium,” or “LD,” means that there is interdependence between alleles at loci closely positioned within a genome. More precisely, LD means that the probability to find allele A at locus 1 depends on whether allele B is present at locus 2. Complete LD means that alleles A and B are always found together. Pitchard and Przeworski, Am. J. Hum. Genet., 69:1-4 (2001) teaches a widely used measure of LD:
r²=(P(AB)−P(A)P(B))²/P(A)P(a)P(B)P(b),

where A and a are two alleles at locus 1, B and b are two alleles at locus 2, and P(X) is the probability of X

If LD is absent, then P(AB)=P(A)P(B), and, therefore, r²=0. In contrast, in the case of complete disequilibrium, P(AB)=P(A)=P(B), and, therefore, r²=1. In the case of partial LD, r² is between 0 and 1, and high values of r² correspond to strong LD. If allele A of locus 1 is associated with a disease, and there is a strong LD between locus 1 and locus 2, so that P(AB)>P(A)P(B), then allele B is associated with the disease too. To define strong LD, a threshold of r²>0.8 is usually used. See Carlson et al, Nat. Genet., 33(4):518-21 (2003). This threshold has been applied in identifying additional SNPs that are in LD with the disease, disorder or phenotype-associated SNP of the instant invention.

Thus, when a second nucleotide variant is said herein to be in linkage disequilibrium, or LD, with a first nucleotide variant, it is meant that a second variant is closely dependent upon a first variant, with a r² value of at least 0.8, as calculated by the formula above. Thus, the term “LD variants” as used herein means variants that are in linkage disequilibrium with an r² value of at least 0.8.

The term “reference sequence” refers to a polynucleotide or polypeptide sequence known in the art, including those disclosed in publicly accessible databases, e.g., Entrez or GenBank, or a newly identified gene sequence, used simply as a reference with respect to the nucleotide variants provided in the present invention. The nucleotide or amino acid sequence in a reference sequence is contrasted to the alleles disclosed in the present invention having newly discovered nucleotide or amino acid variants. In the instant disclosure, for TLK1 genomic DNA, the sequence provided by GenBank Accession No. AC007739 (PRI 7-Oct.-2000) or AC009953 (PRI 30-Sep.-2000) or AC010092 (PRI 08-Nov.-2000) is used as a reference sequence, while the nucleotide and amino acid sequences provided by GenBank Accession No. AB004885 (PRI 5-Feb.-1999) (see SEQ ID NOs:1 and 2) are used as the reference sequences for TLK1 cDNA and protein, respectively.

For WARS2 genomic DNA, the sequence provided by GenBank Accession No. AL139420 (PRI 18-May-2005), AL359823 (PRI 19-May-2005) and AL590288 (PRI 18-May-2005) are used as a reference sequence, while the nucleotide and amino acid sequences provided by GenBank Accession No. NM_{—}015836 (PRI 23-Apr.-2005) (see SEQ ID NO:18 and 19) and NM_{—}201263 (PRI 5-Jun.-2005) (see SEQ ID NOs:20 and 21) are used as the reference sequences for WARS2 cDNA and protein, respectively.

For ARTS2 genomic DNA, the sequence provided by GenBank Accession No. AC009073 (PRI 5-Jul.-2000) is used as a reference sequence, while the nucleotide and amino acid sequences provided by GenBank Accession No. NM_{—}016442 (PRI 23-Apr.-2005) (see SEQ ID NOs:30 and 31) and AF183569 (PRI 29-Dec.-1999) (see SEQ ID NOs:32 and 33) are used as the reference sequences for ARTS1 cDNA and protein, respectively.

For MSR genomic DNA, the sequence provided by GenBank Accession No. AC025174 (PRI 28-Mar.-2002) is used as a reference sequence, while the nucleotide and amino acid sequences provided by GenBank Accession No. NM_{—}002454 (PRI 22-Apr.-2005) (see SEQ ID NOs:66 and 67) and NM_{—}024010 (PRI 22-Apr.-2005) (see SEQ ID NOs:68 and 69) are used as the reference sequences for MSR cDNA and protein, respectively.

For AKAP9 genomic DNA, the sequence provided by GenBank Accession No. AC003086 (PRI 21-Dec.-1999) is used as a reference sequence, while the nucleotide and amino acid sequences provided by GenBank Accession No. NM_{—}005751 (PRI 5-Jun.-2005) (see SEQ ID NOs:90 and 91) are used as the reference sequences for AKAP9 cDNA and protein, respectively.

For DNAJD1 genomic DNA, the sequence provided by GenBank Accession No. AL445217 (PRI 18-May-2005) is used as a reference sequence, while the nucleotide and amino acid sequences provided by GenBank Accession No. NM_{—}013238 (PRI 24-May-2005) (see SEQ ID NOs:149 and 150) are used as the reference sequences for DNAJD1 cDNA and protein, respectively.

For GOLPH4 genomic DNA, the sequence provided by GenBank Accession No. AC117467 (PRI 1-Aug.-2002) and GenBank Accession No. AC069243 (PRI 28-SEP-2002) are used as a reference sequence, while the nucleotide and amino acid sequences provided by GenBank Accession No. NM_{—}014498 (PRI 22-Apr.-2005) (see SEQ ID NOs:156 and 157) are used as the reference sequences for GOLPH4 cDNA and protein, respectively.

For RABEP1 genomic DNA, the sequence provided by GenBank Accession No. NM_{—}004703 (PRI 8-Jun.-2005) is used as a reference sequence, while the nucleotide and amino acid sequences provided by GenBank Accession No. NM_{—}004703 (PRI 8-Jun.-2005) (see SEQ ID NOs:170 and 171) are used as the reference sequences for RABEP1 cDNA and protein, respectively.

For TAP2 genomic DNA, the sequence provided by GenBank Accession No. AL671681 (PRI 18-May-2005) is used as a reference sequence, while the nucleotide and amino acid sequences provided by GenBank Accession No. NM_{—}000544 (PRI 10-Jun.-2005) (see SEQ ID NO:202 and 203) and NM_{—}018833 (PRI 10-Jun.-2005) (see SEQ ID NO:204 and 205) are used as the reference sequences for TAP2 cDNA and protein, respectively.

For NARG2 genomic DNA, the sequence provided by GenBank Accession No. AC087385 (PRI 29-Jul.-2002) is used as a reference sequence, while the nucleotide and amino acid sequences provided by GenBank Accession No. NM_{—}024611 (PRI 8-Jun.-2005) (see SEQ ID NOs:230 and 231) are used as the reference sequences for NARG2 cDNA and protein, respectively.

For DDX58 genomic DNA, the sequences provided by GenBank Accession Nos. AL161783 (PRI 18-May-2005) and AL353671 (PRI 18-May-2005) are used as a reference sequence, while the nucleotide and amino acid sequences provided by GenBank Accession No. NM_{—}014314 (PRI 2-Apr.-2006) (see SEQ ID NOs:274 and 275) are used as the reference sequences for DDX58 cDNA and protein, respectively.

For CD39 genomic DNA, the sequences provided by GenBank Accession Nos. AL356632 (PRI 18-May-2005) and AL365273 (PRI 18-May-2005) are used as a reference sequence, while the nucleotide and amino acid sequence provided by GenBank Accession No. NM_{—}001776 (PRI 15-Oct.-2006) (see SEQ ID NOs:243 and 244) are used as the reference sequences for CD39 cDNA and protein, respectively.

For FKBP1a genomic DNA, the sequences provided by GenBank Accession Nos. AL136531 (PRI 18-May-2005) and AL109658 (PRI 18-May-2005) are used as a reference sequence, while the nucleotide and amino acid sequences provided by GenBank Accession No. NM_{—}000801 (PRI 6-Nov.-2005) (see SEQ ID NO:249 and 250) is used as the reference sequences for FKBP1a cDNA and protein, respectively.

For SRI genomic DNA, the sequences provided by GenBank Accession Nos. AC003991 (PRI 4-Feb.-2000) and AC005075 (PRI 2-Oct.-2000) are used as a reference sequences, while the nucleotide and amino acid sequences provided by GenBank Accession Nos. NM_{—}003130 (PRI 15-Jan.-2006) (see SEQ ID NO:253 and 254) are used as the reference sequences for SRI cDNA and protein, respectively.

For XRRA1 genomic DNA, the sequences provided by GenBank Accession Nos. AP000560 (PRI 15-Mar.-2003) and AP001992 (PRI 15-Mar.-2003) are used as a reference sequences, while the nucleotide and amino acid sequences provided by GenBank Accession Nos. XM_{—}374912 (PRI 19-Feb.-2004) (see SEQ ID NO:257 and 258) are used as the reference sequences for XRRA1 cDNA and protein, respectively.

For IRF5 genomic DNA, the sequences provided by GenBank Accession Nos. AC025594 (PRI 28-Nov.-2000) are used as a reference sequences, while the nucleotide and amino acid sequences provided by GenBank Accession Nos. NM_{—}002200 (PRI 18-Oct.-2005) (see SEQ ID NO:280) are used as the reference sequences for IRF5 cDNA and protein, respectively.

For AMFR genomic DNA, the sequence provided by GenBank Accession No. AC092140 (PRI 3-Jan.-2004) is used as a reference sequence, while the nucleotide sequences provided by GenBank Accession No. NM_{—}001144 (PRI 27-Oct.-2004) (see SEQ ID NO:1) and NM_{—}138958 (PRI 27-Oct.-2004) (SEQ ID NO:3) are used as the reference sequences for AMFR cDNA and protein, respectively.

As used herein, the term “TLK1 nucleic acid” means a nucleic acid molecule the nucleotide sequence of which is uniquely found in a TLK1 gene. That is, a “TLK1 nucleic acid” is either a TLK1 genomic DNA or mRNA/cDNA, having a naturally existing nucleotide sequence encoding a naturally existing TLK1 protein (wild-type or mutant form). The sequence of an example of a naturally existing TLK1 nucleic acid is found in GenBank Accession No. AB004885 (PRI 5-Feb.-1999) (see SEQ ID NO:1). Other examples include nucleic acids provided by GenBank Accession Nos. AK091975 (PRI 30-Jan.-2004) (see SEQ ID NO:14), AK090779 (PRI 30-Jan.-2004) (see SEQ ID NO:15) and NM_{—}012290 (PRI 23-Apr.-2005) (see SEQ ID NO:16).

As used herein, the term “TLK1 protein” means a polypeptide molecule the amino acid sequence of which is found uniquely in a TLK1 protein. That is, “TLK1 protein” is a naturally existing TLK1 protein (wild-type or mutant form). The sequence of a wild-type form of a TLK1 protein is provided by GenBank Accession No. AB004885 (PRI 5-Feb.-1999) (see SEQ ID NO:2). Other examples include amino acid sequences listed in GenBank Accession Nos. AK091975 (PRI 30-Jan.-2004), AK090779 (PRI 30-Jan.-2004) and NM_{—}012290 (PRI 23-Apr.-2005) (see SEQ ID NO:13).

As used herein, the term “WARS2 nucleic acid” means a nucleic acid molecule the nucleotide sequence of which is uniquely found in a WARS2 gene. That is, a “WARS2 nucleic acid” is either a WARS2 genomic DNA or mRNA/cDNA, having a naturally existing nucleotide sequence encoding a naturally existing WARS2 protein (wild-type or mutant form). The sequence of an example of a naturally existing WARS2 nucleic acid is found in GenBank Accession No. NM_{—}015836 (PRI 23-Apr.-2005) (see SEQ ID NO:18) and NM_{—}201263 (PRI 5-Jun.-2005) (SEQ ID NO:20).

As used herein, the term “WARS2 protein” means a polypeptide molecule the amino acid sequence of which is found uniquely in a WARS2 protein. That is, “WARS2 protein” is a naturally existing WARS2 protein (wild-type or mutant form). The sequence of a wild-type form of a WARS2 protein is found in GenBank Accession No. NM_{—}015836 (PRI 23-Apr.-2005) (see SEQ ID NO:19) and NM_{—}201263 (PRI 5-Jun.-2005) (SEQ ID NO:21).

As used herein, the term “ARTS1 nucleic acid” means a nucleic acid molecule the nucleotide sequence of which is uniquely found in an ARTS1 gene. That is, a “ARTS1 nucleic acid” is either an ARTS1 genomic DNA or mRNA/cDNA, having a naturally existing nucleotide sequence encoding a naturally existing ARTS1 protein (wild-type or mutant form). The sequences of examples of naturally existing ARTS1 nucleic acid are found in GenBank Accession No. NM_{—}016442 (PRI 23-Apr.-2005) (see SEQ ID NO:30) and AF183569 (PRI 29-Dec.-1999) (SEQ ID NO:32).

As used herein, the term “ARTS1 protein” means a polypeptide molecule the amino acid sequence of which is found uniquely in an ARTS1 protein. That is, “ARTS1 protein” is a naturally existing ARTS1 protein (wild-type or mutant form). The sequence of a wild-type form of an ARTS1 protein is found in GenBank Accession No. NM_{—}016442 (PRI 23-Apr.-2005) (see SEQ ID NO:31) and AF183569 (PRI 29-Dec.-1999) SEQ ID NO:33).

As used herein, the term “MSR nucleic acid” means a nucleic acid molecule the nucleotide sequence of which is uniquely found in a MSR gene. That is, a “MSR nucleic acid” is either an MSR genomic DNA or mRNA/cDNA, having a naturally existing nucleotide sequence encoding a naturally existing MSR protein (wild-type or mutant form). The sequence of an example of a naturally existing MSR nucleic acid is found in GenBank Accession No. NM_{—}002454 (PRI 22-Apr.-2005) (see SEQ ID NO:66) and NM_{—}024010 (PRI 22-Apr.-2005) (see SEQ ID NO:68).

As used herein, the term “MSR protein” means a polypeptide molecule the amino acid sequence of which is found uniquely in an MSR protein. That is, “MSR protein” is a naturally existing MSR protein (wild-type or mutant form). The sequence of a wild-type form of a MSR protein is found in GenBank Accession No. NM_{—}002454 (PRI 22-Apr.-2005) (see SEQ ID NO:67) and NM_{—}024010 (PRI 22-Apr.-2005) (see SEQ ID NO:69).

As used herein, the term “AKAP9 nucleic acid” means a nucleic acid molecule the nucleotide sequence of which is uniquely found in an AKAP9 gene. That is, an “AKAP9 nucleic acid” is either an AKAP9 genomic DNA or mRNA/cDNA, having a naturally existing nucleotide sequence encoding a naturally existing AKAP9 protein (wild-type or mutant form). The sequence of an example of a naturally existing AKAP9 nucleic acid is found in GenBank Accession No. NM_{—}005751 (PRI 8-Jun.-2005) (see SEQ ID NO:90), NM_{—}147171 (PRI 8-Jun.-2005) (see SEQ ID NO:92), NM_{—}147185 (PRI 8-Jun.-2005) (see SEQ ID NO:93), NM_{—}147166 (PRI 8-Jun.-2005) (see SEQ ID NO:94) and AK000270 (PRI 13-SEP-2003) (see SEQ ID NO:95).

As used herein, the term “AKAP9 protein” means a polypeptide molecule the amino acid sequence of which is found uniquely in an AKAP9 protein. That is, “AKAP9 protein” is a naturally existing AKAP9 protein (wild-type or mutant form). The sequence of a wild-type form of an AKAP9 protein is found in GenBank Accession No. NM_{—}005751 (PRI 8-Jun.-2005) (see SEQ ID NO:91).

As used herein, the term “DNAJD1 nucleic acid” means a nucleic acid molecule the nucleotide sequence of which is uniquely found in a DNAJD1 gene. That is, a “DNAJD1 nucleic acid” is either a DNAJD1 genomic DNA or mRNA/cDNA, having a naturally existing nucleotide sequence encoding a naturally existing DNAJD1 protein (wild-type or mutant form). The sequence of an example of a naturally existing DNAJD1 nucleic acid is found in GenBank Accession No. NM_{—}013238 (PRI 24-May-2005) (see SEQ ID NO:149).

As used herein, the term “DNAJD1 protein” means a polypeptide molecule the amino acid sequence of which is found uniquely in a DNAJD1 protein. That is, “DNAJD1 protein” is a naturally existing DNAJD1 protein (wild-type or mutant form). The sequence of a wild-type form of a DNAJD1 protein is found in GenBank Accession No. NM_{—}013238 (PRI 24-May-2005) (see SEQ ID NO:150).

As used herein, the term “GOLPH4 nucleic acid” means a nucleic acid molecule the nucleotide sequence of which is uniquely found in an GOLPH4 gene. That is, a “GOLPH4 nucleic acid” is either a GOLPH4 genomic DNA or mRNA/cDNA, having a naturally existing nucleotide sequence encoding a naturally existing GOLPH4 protein (wild-type or mutant form). The sequence of an example of a naturally existing GOLPH4 nucleic acid is found in GenBank Accession No. NM_{—}014498 (PRI 22-Apr.-2005) (see SEQ ID NO:156).

As used herein, the term “GOLPH4 protein” means a polypeptide molecule the amino acid sequence of which is found uniquely in an GOLPH4 protein. That is, “GOLPH4 protein” is a naturally existing GOLPH4 protein (wild-type or mutant form). The sequence of a wild-type form of a GOLPH4 protein is found in GenBank Accession No. NM_{—}014498 (PRI 22-Apr.-2005) (see SEQ ID NO:157).

As used herein, the term “RABEP1 nucleic acid” means a nucleic acid molecule the nucleotide sequence of which is uniquely found in an RABEP1 gene. That is, a “RABEP1 nucleic acid” is either an RABEP1 genomic DNA or mRNA/cDNA, having a naturally existing nucleotide sequence encoding a naturally existing RABEP1 protein (wild-type or mutant form). The sequence of an example of a naturally existing RABEP1 nucleic acid is found in GenBank Accession No. NM_{—}004703 (PRI 8-Jun.-2005) (see SEQ ID NO:170).

As used herein, the term “RABEP1 protein” means a polypeptide molecule the amino acid sequence of which is found uniquely in an RABEP1 protein. That is, “RABEP1 protein” is a naturally existing RABEP1 protein (wild-type or mutant form). The sequence of a wild-type form of a RABEP1 protein is found in GenBank Accession No. NM_{—}004703 (PRI 8-Jun.-2005) (see SEQ ID NO:171).

As used herein, the term “TAP2 nucleic acid” means a nucleic acid molecule the nucleotide sequence of which is uniquely found in a TAP2 gene. That is, a “TAP2 nucleic acid” is either a TAP2 genomic DNA or mRNA/cDNA, having a naturally existing nucleotide sequence encoding a naturally existing TAP2 protein (wild-type or mutant form). The sequence of an example of a naturally existing TAP2 nucleic acid is found in GenBank Accession No. NM_{—}000544 (PRI 10-Jun.-2005) (see SEQ ID NO:202) and NM_{—}018833 (PRI 10-Jun.-2005) (see SEQ ID NO:204).

As used herein, the term “TAP2 protein” means a polypeptide molecule the amino acid sequence of which is found uniquely in a TAP2 protein. That is, “TAP2 protein” is a naturally existing TAP2 protein (wild-type or mutant form). The sequence of a wild-type form of a TAP2 protein is found in GenBank Accession No. NM_{—}000544 (PRI 10-Jun.-2005) (see SEQ ID NO:203) and NM_{—}018833 (PRI 10-Jun.-2005) (see SEQ ID NO:205).

As used herein, the term “NARG2 nucleic acid” means a nucleic acid molecule the nucleotide sequence of which is uniquely found in an NARG2 gene. That is, a “NARG2 nucleic acid” is either an NARG2 genomic DNA or mRNA/cDNA, having a naturally existing nucleotide sequence encoding a naturally existing NARG2 protein (wild-type or mutant form). The sequence of an example of a naturally existing NARG2 nucleic acid is found in GenBank Accession No. NM_{—}024611 (PRI 8-Jun.-2005) (see SEQ ID NO:230).

As used herein, the term “NARG2 protein” means a polypeptide molecule the amino acid sequence of which is found uniquely in an NARG2 protein. That is, “NARG2 protein” is a naturally existing NARG2 protein (wild-type or mutant form). The sequence of a wild-type form of a NARG2 protein is found in GenBank Accession No. NM_{—}024611 (PRI 8-Jun.-2005) (see SEQ ID NO:231).

As used herein, the term “DDX58 nucleic acid” means a nucleic acid molecule the nucleotide sequence of which is uniquely found in a DDX58 gene. That is, a “DDX58 nucleic acid” is either a DDX58 genomic DNA or mRNA/cDNA, having a naturally existing nucleotide sequence encoding a naturally existing DDX58 protein (wild-type or mutant form). The sequence of an example of a naturally existing DDX58 nucleic acid is found in GenBank Accession No. NM_{—}014314 (PRI 2-Apr.-2006) (see SEQ ID NO:274).

As used herein, the term “DDX58 protein” means a polypeptide molecule the amino acid sequence of which is found uniquely in a DDX58 protein. That is, “DDX58 protein” is a naturally existing DDX58 protein (wild-type or mutant form). The sequence of a wild-type form of a DDX58 protein is found in GenBank Accession No. NM_{—}014314 (PRI 2-Apr.-2006) (see SEQ ID NO:275).

As used herein, the term “CD39 nucleic acid” means a nucleic acid molecule the nucleotide sequence of which is uniquely found in a CD39 gene. That is, a “CD39 nucleic acid” is either a CD39 genomic DNA or mRNA/cDNA, having a naturally existing nucleotide sequence encoding a naturally existing CD39 protein (wild-type or mutant form). The sequence of an example of a naturally existing CD39 nucleic acid is found in GenBank Accession Nos. NM_{—}001776 (PRI 15-Jan.-2006) (see SEQ ID NO:243).

As used herein, the term “CD39 protein” means a polypeptide molecule the amino acid sequence of which is found uniquely in a CD39 protein. That is, “CD39 protein” is a naturally existing CD39 protein (wild-type or mutant form). The sequence of a wild-type form of a CD39 protein is found in GenBank Accession No. NM_{—}001776 (PRI 15-Jan.-2006) (see SEQ ID NO:244).

As used herein, the term “FKBP1a nucleic acid” means a nucleic acid molecule the nucleotide sequence of which is uniquely found in a FKBP1a gene. That is, a “FKBP1a nucleic acid” is either a FKBP1a genomic DNA or mRNA/cDNA, having a naturally existing nucleotide sequence encoding a naturally existing FKBP1a protein (wild-type or mutant form). The sequence of an example of a naturally existing FKBP1a nucleic acid is found in GenBank Accession No. NM_{—}000801 (PRI 6-Nov.-2005) (see SEQ ID NO:249).

As used herein, the term “FKBP1a protein” means a polypeptide molecule the amino acid sequence of which is found uniquely in an FKBP1a protein. That is, “FKBP1a protein” is a naturally existing FKBP1a protein (wild-type or mutant form). The sequence of a wild-type form of an FKBP1a protein is found in GenBank Accession Nos. NM_{—}000801 (PRI 6-Nov.-2005) (see SEQ ID NO:250).

As used herein, the term “SRI nucleic acid” means a nucleic acid molecule the nucleotide sequence of which is uniquely found in an SRI gene. That is, an “SRI nucleic acid” is either an SRI genomic DNA or mRNA/cDNA, having a naturally existing nucleotide sequence encoding a naturally existing SRI protein (wild-type or mutant form). The sequences of exemplary naturally existing SRI nucleic acid are found in GenBank Accession No. NM_{—}003130 (PRI 15-Jan.-2006) (SEQ ID NO:253).

As used herein, the term “SRI protein” means a polypeptide molecule the amino acid sequence of which is found uniquely in an SRI protein. That is, an “SRI protein” is a naturally existing SRI protein (wild-type or mutant form). The sequence of a wild-type form of an SRI protein is found in GenBank Accession No. NM_{—}003130 (PRI 15-Jan.-2006) (SEQ ID NO:254).

As used herein, the term “XRRA1 nucleic acid” means a nucleic acid molecule the nucleotide sequence of which is uniquely found in an XRRA1 gene. That is, an “XRRA1 nucleic acid” is either an XRRA1 genomic DNA or mRNA/cDNA, having a naturally existing nucleotide sequence encoding a naturally existing XRRA1 protein (wild-type or mutant form). The sequences of exemplary naturally existing XRRA1 nucleic acid are found in GenBank Accession No. XM_{—}374912 (PRI 19-Feb.-2004) (SEQ ID NO:257).

As used herein, the term “XRRA1 protein” means a polypeptide molecule the amino acid sequence of which is found uniquely in an XRRA1 protein. That is, an “XRRA1 protein” is a naturally existing XRRA1 protein (wild-type or mutant form). The sequence of a wild-type form of an XRRA1 protein is found in GenBank Accession No. XM_{—}374912 (PRI 19-Feb.-2004) (SEQ ID NO:258).

As used herein, the term “IRF5 nucleic acid” means a nucleic acid molecule the nucleotide sequence of which is uniquely found in an IRF5 gene. That is, an “IRF5 nucleic acid” is either an IRF5 genomic DNA or mRNA/cDNA, having a naturally existing nucleotide sequence encoding a naturally existing IRF5 protein (wild-type or mutant form). The sequences of exemplary naturally existing IRF5 nucleic acid are found in GenBank Accession No. NM_{—}002200 (PRI 18-Oct.-2005) (see SEQ ID NO:280).

As used herein, the term “IRF5 protein” means a polypeptide molecule the amino acid sequence of which is found uniquely in an IRF5 protein. That is, an “IRF5 protein” is a naturally existing IRF5 protein (wild-type or mutant form). The sequence of a wild-type form of an IRF5 protein is found in GenBank Accession No. NM_{—}002200 (PRI 18-Oct.-2005) (see SEQ ID NO:281).

As used herein, the term “AMFR nucleic acid” means a nucleic acid molecule the nucleotide sequence of which is uniquely found in an AMFR gene. That is, an “AMFR nucleic acid” is either an AMFR genomic DNA or mRNA/cDNA, having a naturally existing nucleotide sequence encoding a naturally existing AMFR protein (wild-type or mutant form). The sequences of exemplary naturally existing AMFR nucleic acid are found in GenBank Accession No. NM_{—}001144 (PRI 27-Oct.-2004) (see SEQ ID NO:291) and NM_{—}138958 (PRI 27-Oct.-2004) (SEQ ID NO:293).

As used herein, the term “AMFR protein” means a polypeptide molecule the amino acid sequence of which is found uniquely in an AMFR protein. That is, an “AMFR protein” is a naturally existing AMFR protein (wild-type or mutant form). The sequence of a wild-type form of an AMFR protein is found in GenBank Accession No. NM_{—}001144 (PRI 27-Oct.-2004) (see SEQ ID NO:292) and NM_{—}138958 (PRI 27-Oct.-2004) (SEQ ID NO:294).

### 2. Nucleotide and Amino Acid Variants

Thus, in accordance with the present invention, genetic variants, i.e., single nucleotide polymorphisms (SNPs) and/or haplotypes have been discovered in the TLK1, WARS2, ARTS2, MSR, AKAP9, DNAJD1, GOLPH4, RABEP1, TAP2, NARG2, DDX58, CD39, FKBP1a, SRI, XRRA1, IRF5 and AMFR genes. The identified SNPs and/or haplotypes are summarized in Tables 1-35. Exemplary TLK1, WARS2, ARTS2, MSR, AKAP9, DNAJD1, GOLPH4, RABEP1, TAP2, NARG2, DDX58, CD39, FKBP1a, SRI, XRRA1, IRF5 and AMFR gene sequences spanning the SNPs in the tables are provided in the sequence listing.

The nucleotide positions are assigned by aligning the variant allele sequences to the above-identified cDNA reference sequence and genomic reference sequence. Specifically, the nucleotide position of a SNP is indicated relative to the nearest exon. As a general example, EX6@51 means the SNP is located within exon 6 of a gene at the 51^{st} nucleotide position counting in a 5′ to 3′ direction from the 5′ end of exon 6 with the 5′ end nucleotide of exon 6 being the 1^{st} position. EX7@+14 means the SNP is located within the intron immediately 3′ to exon 7 of a gene at the 14^{th} nucleotide position counting in a 5′ to 3′ direction from the 5′ end of that intron with the 5′ end nucleotide of that intron being the 1^{st} nucleotide position. EX13@−27 means the SNP is located within the intron immediately 5′ to exon 13 of the gene at the 27^{th} nucleotide position counting in a 3′ to 5′ direction from the 3′ end nucleotide of the intron with the 3′ end nucleotide of that intron being the 1^{st} nucleotide position. Likewise, EX1@−761 means that the SNP is located at the 761^{st} nucleotide position upstream from the 5′ end nucleotide of exon 1, with the first intron/regulatory nucleotide immediately 5′ to exon 1 being the 1^{st} nucleotide position.

The amino acid substitutions caused by the nucleotide variants (SNPs) are also identified according to conventional practice. For example, A160V means the amino acid variant at position 160 is V in contrast to A in the reference sequence identified above with the N-terminus amino acid being at position 1.

In some cases, the nucleotide positions and nucleotide variant identities are provided by reference to corresponding reference cluster ID (rs#) assigned in the public dbSNP database accessible at the NCBI website, or by chromosome locations.

Thus, the SNPs identified according to the present invention are associated with “expression phenotypes” in humans. That is, it has been discovered that the baseline expression level of the genes in Tables 1-35 in human cells is an inheritable “quantitative trait” with genetic determinants. Furthermore, it has been surprisingly discovered that the SNPs and haplotypes in accordance with the present invention are associated with the “quantitative trait”, i.e., the mRNA level of the genes in human cells. Thus, the SNPs and haplotypes are particularly useful in predicting the gene expression in an individual.

In response to DNA damage cells initiate a series of processes that prevent replication of damaged DNA and maintain integrity of the genome. The DNA damage checkpoint induces cell cycle arrest, allowing for repair mechanisms to occur before transmission of damaged chromosomes. DNA damage can be caused by a variety of agents including ultraviolet radiation (UV), mutagenic chemicals, radiation, free radicals and teratogens. Cancer and heart disease are among the diseases associated with damage to DNA. TLK1 has been identified as a regulator of the DNA damage checkpoint system. TLK1 is the mammalian homolog of the plant Tousled gene which regulates flower development. TLK1 is 84% similar to Tousled on an amino acid level and shares its kinase activity. TLK1 is a 81.9 kD nuclear localized kinase having 5-domains including an N-terminus nuclear localization signal, a protein kinase ATP-binding motif, a nucleotide binding motif, and a single catalytic domain near the C-terminus. See Sillje et al., EMBO. J., 18(20):5691-702 (1999). TLK1 phosphorylates ASF1, a chromatin assembly factor, implicating it in the regulation of chromatin remodeling. Mammalian TLKs have been implicated in regulation of chromatin remodeling through the observation that they phosphorylate the chromatin assembly factor ASF1. ASF1 synergizes with another chromatin assembly factor, CAF1, in replication- and repair-coupled chromatin remodeling. See Sillje et al., Curr Biol., 11(13):1068-73 (2001).

Underlining the importance of TLK1 in the preservation of genomic integrity, the normally active TLK1 is inhibited in the event of DNA damage such as a double-strand break (DSB). Inhibition of TLK depends on the action of the DNA damage checkpoint system including the kinases ATM (ataxia telangiectasia mutated) and ATR (ataxia and Rad3 related). ATM is a key regulator in response to ionizing radiation (IR), while ATR induces cell cycle arrest in response to a wide range of agents producing DNA double strand breaks. Both kinases relay the checkpoint signal through phosphorylation of checkpoint kinases: Chk2 (also known as Rad53) and Chk1. Inhibition of TLK1 in response to IR depends on ATM and Chk1, as well as the sensor protein NBS1. Expression of a dominant-negative kinase mutant of TLK1 results in chromosome missegregation and aneuploidy, underlining the importance of TLK1 in preserving genomic integrity. Experiments using mouse cell lines have shown that TLK1 overexpression increases cell resistance to DNA damage caused by ionizing radiation. See Sunalava-Dossabhoy et al., BMC Cell Biol., 4(1):16 (2003). Thus, expression levels of TLK1 affect the ability to control DNA damage and can be used to determine resistance to the DNA damage and susceptibility to diseases resulting from such carcinogens. Further, common cancer treatments that function by inducing DNA damage will be affected by TLK1 levels.

Histone H3, a modifier of chromatin condensation, is present in the cytosol at the highest levels during active DNA replication. See Senshu et al., Eur J. Biochem., 146(2):261-6 (1985). Phosphorylation of histone H3 influences transcription, chromosome condensation, DNA repair and apoptosis. TLK1 has been shown to phosphorylate histone H3 in vitro and in vivo. See Sunavala-Dossabhoy et al., BMC Cell Biol., 4(1): 16 (2003). Downregulation of histone H3 mRNA levels occurs in parallel with the inhibition of DNA synthesis during S-phase upon DNA damage. See Zhao, J., Cell Cycle., 3(6):695-7 (2004). Levels of TLK1 are elevated 9.4 times in patients with stage I to II breast cancer. See Norton et al., J Surg Res., 116(1):98-103 (2004). This suggests that TLK1 is linked to the S-phase DNA damage checkpoint and DNA replication in the cell. Thus, increased H3 histone phosphorylation as a result of TLK1 overexpression causes chromosome instability and, thus, may play a role in carcinogenesis. Accordingly, polymorphisms influencing expression levels of TLK1 would be useful in predicting cancer progression and response to radiation treatment as well as susceptibility to DNA damage by ionizing radiation. See Li et al., Oncogene., 20(6):726-38 (2001).

An isoform of TLK1, named SNAK for SNARE kinase, was shown to phosphorylate SNAP-23, a component of the SNARE complex. SNAP-23 is the ubiquitiously expressed homolog of the neuron-specific SNAP-25 and is essential for the regulation of exocytosis in several cell types. For example, fusion of GLUT4 containing vesicles with the plasma membrane involves the target membrane SNAREs syntaxin 4 and SNAP-23 and the vesicle-associated SNARE VAMP2. SNAP-23 is palmitoylated post-translationally and this acylated form readily associates with membranes. Phosphorylation increases the stability of SNAP-23 and promotes the formation of SNARE complexes. Expression levels of TLK1 could therefore influence the kinetics of SNARE assembly and contribute to SNARE associated metabolic disorders. See Cabaniols et al., Mol. Biol. Cell, 10(12):4033-4041 (1999).

WARS2 is the mitochondrial form of tryptophanyl-tRNA synthetase, the enzyme that links the nucleotide triplets in the genetic code to form amino acid units by catalyzing the loading of the tryptophan-specific tRNA with the amino acid tryptophan. WARS2 is a 360-amino acid 40.1 kDa α₂ dimer protein of the class IaaRSs. See Jorgensen et al., J. Biol. Chem., 275(22):16820-16826 (2002). Nearly 100 disease-correlated mutations in the mitochondrial genome are known to be located in mitochondrial tRNA genes. Mitochondrial dysfunction is a mechanism in the progression of neurodegenerative disorders such as Friedeich's ataxia, Huntington's disease, Alzheimer's disease amyotrophic lateral sclerosis (ALS) and Parkinson's disease. Although no specific disorders have been associated with WARS2 mutation, variations in the expression levels of WARS2 are likely to contribute to disorders associated with mutations in the corresponding mitochondrial tRNA gene. Thus, polymorphisms correlating with expression levels of WARS2 are useful to predict of detect susceptibility to neurodegenerative disease, such as Friedreich's ataxia, Huntington's disease, Alzheimer's disease, ALS and Parkinson's disease, in an individual.

Polymorphisms in mitochondrial DNA have also been linked to mitochondria dysfunction associated with cardiovascular disease. Such diseases include dilated and hypertrophic cardiomyopathy, cardiac conduction defects and sudden death, ischemic and alcoholic cardiomyopathy, and myocarditis. These abnormalities result in dysfunction in oxidative phosphorylation and fatty acid beta-oxidation. See Marin-Garcia, J. and Goldenthal, M. J., J. Card. Fail., 8(5):347-61 (2002). Thus, SNPs correlating with WARS2 mRNA expression levels are useful as a means of predicting or detecting susceptibility to cardiovascular disease in an individual.

An N-terminally truncated WARS2 fragment, T2-TrpRS, is a potent antagonist of vascular endothelial growth-factor induced angiogenesis. Particularly, T2-TrpRS regulates extracellular signal activated protein kinase, Akt, and EC NO synthase activation pathways involved in angiogenesis, cytoskeletal reorganization, and shear stress-responsive gene expression. Angiogenesis stimulating gene expression was shown to be linked to tumor progression in nude mice. See Yoneda et al., J. Natl. Cancer Inst., 90:447-454 (1998). Expression of T2-TrpRS blocks vascular endothelial growth factor-stimulated angiogenesis. Higher concentration of T2-TrpRS further decreased activity of VEGF-induced angiogenesis. See Otani et al., Proc. Natl. Acad. Sci. USA., 99(1):173083 (2002). Proteins demonstrating a similar angiogenesis inhibiting effect are currently used in the treatment of cancer to prevent the spread of cancer cells by blood vessel growth. See Langer, et al., Proc. Natl. Acad. Sci., 77(7):4331-5 (1980). As T2-TrpRS is a naturally occurring protein, it provides an advantage as a potentially nonimmunogenic compound for the treatment of cancer. See Otani et al., Proc. Natl. Acad. Sci. USA., 99(1):173083 (2002).

IFN-γ has been shown to be involved in an anti-tumorigenic signaling pathway making it useful in the prognosis and therapy of cancer. See Blanck, G., Arch. Immunol. Ther. Exp. 50(3):151-8 (2002). T2-TrpRS shows high expression in the presence of IFN-γ. T2-TrpRS is cleaved by PMN elastase protein, which is expressed in human colorectal cancer, breast cancer and non-small cell lung cancers (NSCLCs). The resulting truncated protein, TrpTS, acts as an angiostatic inhibitor slowing tumor growth. See Wakasugi et al., Proc. Natl. Acad. Sci. USA., 99(1):173-177 (2002). Accordingly, the SNPs associated with WARS2 expression levels can be used to predict or detect cancer susceptibility, to predict cancer prognosis in an individual.

ARTS1 is a 120 kD type II integral membrane aminopeptidase critical in the metabolism of proteins involved in processes such as blood pressure regulation, hypertension pathogenesis and immune response. ARTS1 creates bioactive peptides through hydrolysis of various inactive peptides within the ER. ARTS1 contains an N-terminal signal peptide, 5 potential N-glycosylation sites, a potential membrane-spanning domain, and a zinc-binding motif. See Hattori et al., J. Biochem., 125:931-938 (1999).

Coimmunoprecipitation assays have shown that ARTS1 binds TNFR1 to form a complex that promotes TNFR1 ecto-domain shedding. TNFR1 is a tumor necrosis factor that, in its cleaved state, is a multifunctional cytokine involved in the regulation of processes such as inflammatory response, immunoregulation, cytotoxicity, antiviral actions and transcriptional regulation of genes. See Vilcek and Lee, J. Biol. Chem., 266:7313-7316 (1991). ARTS1 levels directly correlate with TNFR1 shedding. Overexpression of ARTS1 mRNA results in increased TNFR shedding and diminished membrane-associated TNFR1. Conversely, the expression of anti-sense ARTS1 mRNA leads to decreased TNFR1 shedding and membrane associated ARTS1 as well as increased membrane associated TNFR1. See Cui et al., J Clin Invest., 110(4):515-26 (2002). TNFR1 shedding deficiencies have been associated with autoimmune diseases such as endotoxic shock, TNF-dependent arthritis, and encephalomyelitis. See Xanthoulea et al., J. Exp. Med. 200(3):367-76 (2004). In view of the above, SNPs associated with ARTS1 mRNA levels could be useful in the prediction and detection of susceptibility to autoimmune and inflammatory disease in humans.

Activation of vascular endothelial growth factor (VEGF) initiates cell proliferation and is a hallmark of tumor progression. Angiotensin II exposure induces VEGF activity in several cell types. Overexpression of ARTS1 in human endometrial carcinoma cells inhibited angiotensin II stimulated VEGF expression, suggesting that elevated ARTS1 levels reduce the amount of functional angiotensin II and suppress the proliferative effect of VEGF on tumor cells. See Wantanabe et al., Clin. Cancer Res., 9(17):6497-503 (2003). Thus, SNPs associated with ARTS1 mRNA expression levels are useful as a means of predicting or detecting cancer susceptibility, predicting cancer progression in an individual as well as and predicting patient response to cancer treatment.

Expression of ARTS1 in Chinese hamster ovary cells showed that ARTS1 hydrolyzes angiotensin II, a protein involved in the regulation of blood pressure. At high levels, angiotensin induces hypertension, cardiac hypertrophy, myocardial damage and coronary heart disease. See Caulfield et al., N. Engl. J. Med., 330(23):1629-33 (1994) and Finkenberg et al., J. Hypertens., 23(2):375-80 (2005). Treatment of patients with angiotensin-enzyme converting inhibitor A-I was shown to reduce death in ischemia patients. See Zuliana et al., J. Gerontol. A. Biol. Sci. Med. Sci., 60(4):463-5 (2005). Thus, SNPs associated with ARTS1 mRNA expression are useful to predict and detect susceptibility to heart disease in an individual.

Cell surface antigens detect and eliminate foreign cells such as those virally infected or tumorigenic. Before transport to the cell surface, antigen precursor proteins are synthesized in the cells endoplasmic reticulum (ER). Unprocessed precursor peptides bind on the surface of major histocompatibity complex (MHC) where ARTS1 has been shown to trim extra terminal residues from precursor proteins thereby creating 8-11 residue peptide fragments. These fragments are then transported to the cell surface facilitating cell recognition and elimination of virally infected cells. In other words, ARTS1 hydrolyzes proteins creating functional antigenic precursors necessary for immune system cell recognition. ARTS1 overexpression stimulates processing and presentation of antigenic precursors in the ER demonstrating the importance of ARTS1 levels in immune response. Further, agents that block precursor trimming in the ER have been shown to inhibit ARTS1 function. See Cui, et al., J. Clin. Invest., 110(4):515-526 (2002). Accordingly, ARTS1 levels could be used to predict immune response in an individual, especially that associated with viral infection.

MSR is an essential enzyme in folate/cobalamin metabolism. MSR functions by recycling cobalamin, a cofactor necessary for activation of the enzyme methionine synthetase (MS), which catalyzes the remethylation of homocysteine to methionine. See Wilson et al., Human Molecular Genetics, 8(11):2009-2016 (1999). As cobalamin is used, it becomes oxidized rendering it inactive. See Banerjee, R., Chem Biol., 4, 175-186 (1997). MSR catalyzes regeneration of cobalamin to its active state where it can be used as a cofactor activating MS. Active MS is necessary for catalyzing the final step in methionine synthesis. MSR polymorphisms S175 was shown to be a genetic determinant of plasma homocysteine levels. This polymorphism has been linked to premature coronary artery disease, Down's syndrome and neural tube defects. See Gaughan et al., Atherosclerosis, 157(2): 451-6 (2001) and Olteanu et al., Biochemistry, 43(7):1988-97 (2004) and Bosco et al., Am. J. Med. Genet. A., 121(3):219-24 (2003). Similarly, MSR mutations resulting in cobalamin deficiency have been shown to cause homocystinuria, hyperhomocysteinemia and hypomethioninemia. See Watkins et al., Am. J. Hum. Genet., 71(1):143-153 (2002).

Homocysteine is produced during the metabolism of methionine and can be remethylated to methionine through the action of MS. Elevated levels of homocysteine (hyperhomocysteinemia) have been linked to cardiovascular disease, atherosclerosis, recurrent arterial and venous thrombosis and premature coronary artery disease. See Cattaneo, M., Thromb Haemost., 81(2):165-76 (1999). MSR deficiency causes hyperhomocysteinemia, hypomethioninemia and megaloblastic aneimia showing the effect of MSR levels on homocysteine metabolism. See Wilson, et al., Hum. Mol. Genet., 8(11):2009-2016 (1999). Studies have also shown an association of hyperhomocysteinaemia with neural tube defects such as spina bifida. See Mills et al., Lancet, 345(8943):149-51 (1995) and D'Angelo et al., Haematologica, 82(2):211-9 (1997). Children with Down's syndrome have significantly lower plasma levels of homocysteine. See Pogribna et al., Am. J. Hum. Genet., 69(1):88-95 (2001). Wild-type minigene expression of MSR in patients having cb1E type homocystinuria resulted in a four-fold increase in MSR-facilitated methionine synthesis. This demonstrates the association of MSR levels with the conversion of homocysteine to methionine. See Zavadakova et al., Hum. Mutat., 25(3):239-47 (2005). Accordingly, SNPs associated with MSR expression levels are useful as markers for determining or predicting relative plasma homocysteine, methionine and cobalamin levels, and for diagnosing and/or predicting diseases and disorders associated with aberrant plasma homocysteine, methionine and cobalamin levels. In addition, the SNPs are also useful as markers for detecting or predicting a predisposition to hyperhomocysteinemia, cardiovascular disease, atherosclerosis, recurrent arterial and venous thrombosis and premature coronary artery disease, neural tube defects and Down's syndrome. The SNPs may also be useful in determining prognosis of hyperhomocysteinemia, cardiovascular disease, atherosclerosis, recurrent arterial and venous thrombosis and premature coronary artery disease, neural tube defects and Down's syndrome in an individual.

MSR facilitated methionine metabolism has also been associated with cancer cell proliferation. A number of cancer cell types are methionine dependent. In other words, the cells are unable to grow on a medium where methionine has been replaced with homocysteine precursor. The doubling time of non-small lung cell cancer lines was shown to have a significant association with levels of methionine synthetase. See Zhang et al., Cancer Res., 65(4): 1554-60 (2005). Since the SNPs and haplotypes described here are associated with the expression level of MSR, they can be used to predict cancer susceptibility and prognosis in patients.

The AKAP9 gene is one of a family of scaffolding proteins that are critical for the subcellular distribution and catalytic activity of the signaling mediator protein kinase A. AKAP9 protein has been shown to regulate ion channel activity, synaptic transmission and cell motility. Also known as AKAP350, AKAP450 and CGNAP, the 350-450 kDa protein has been shown to be localized to the centrosomes and the Golgi apparatus. See Steadman et al., J. Biol. Chem., 277(33):30165-76 (2002).

The Golgi apparatus functions in neurons by processing polypeptides important in fast axoplasmic transport. AKAP9 is expressed and interacts with CIP4 at the Golgi apparatus. Disruption of the AKAP9-CIP4 interaction through expression of the CIP4 binding domain in AKAP9 or silencing AKAP9 expression by RNA interference causes structural changes in the Golgi apparatus. See Larocca et al., Mol. Biol. Cell, 15(6):2771-81 (2004). Such structural changes in Golgi apparatus have a role in the pathogenesis of AD, ALS and neurodegeneration caused by aging. Particularly, fragmentation of the Golgi apparatus in motor neurons has been associated with Alzheimer's disease (AD), amyotrophic lateral sclerosis (ALS) and aging. See Steiber et al., Am. J. Pathol., 148(2):355-60 (1996). Defects in the structure of the Golgi apparatus are found in the ballooned neurons commonly found in those diagnosed with AD, dementia, Creutzfeldt-Jakob disease and Pick's disease. See Aoki et al., Acta. Neuropathol. (Berl)., 106(5):436-40 (2003). Thus, SNPs associated with mRNA expression levels are useful to detect or predict susceptibility in an individual to AD, ALS, neurodegeneration caused by aging, dementia, Creutzfeldt-Jakob disease and Pick's disease.

Irregularities in centromsomes have been linked to cancer development due to their central role in chromosome segregation, irregularities. See Kong et al., Drug News Perspect., 17(3):195-200 (2004). Over-expression of AKAP9 resulted in an increase in the number of centrosomes in Chinese hamster ovary cells. See Nishimura et al., Gene Cells, 10(1):75-86 (2005). Experiments have shown that an increased number of centrosomes can be correlated with tumor progression. Like many known tumor supressors, AKAP9 localized to the centrosomes demonstrating that it likely regulates chromosome duplication and function. See Fisk et al., Curr. Opin. Cell Biol., 14(6):700-5 (2002). The increase in centrosome number in response to AKAP9 expression suggests an association with the risk of developing cancer and with tumor progression. Thus, SNPs associated with AKAP9 mRNA expression are useful as a means of detecting or predicting an individual's susceptibility to cancer and tumor progression.

Cyclic AMP-dependent kinase signaling abnormalities have been associated with depression and symptoms of depression. See Shelton et al. Int. J. Neuropsychopharmacol. 3:187-192 (1999) reported that reduced PKA in fibroblasts was associated with melancholic major depression. Dwivedi et al. Biol. Psychiatry 56(1):30-40 (2004) reported that PKA levels were altered in the brains of learned helpless rats. Perera et al. CNS Spectrums 6(7):565-572 (2001) discusses the potential roles of PKA in depression and antidepressants. Thus, AKAP9 mediated signaling may be involved in the etiology of depression by modulating PKA signaling.

One splice variant of AKAP9 has 3,908 amino acids, and there are at least five other different AKAP9 isoforms produced by alternative splicing. An alternative splice variant of AKAP9 consisting of 1,642 residues is found at neuronal and neuromuscular synapses. It specifically interacts with the N-methyl-D-aspartate (NMDA) receptor (NR1) in brain, and may function to attach NR1 to the postsynaptic cytoskeleton. It also binds to PKA and to type I protein phosphatase (PP1), leading to the conclusion that it is an AKAP that functions to bring together NR1 and its regulatory enzymes, thus regulating NR1 channel activity (Lin et al. J. Neuroscience 18:2012-2027 (1998)). Increases in expression levels of NMDA receptor subunits and associated intracellular proteins have been observed in patients with schizophrenia, bipolar disorder and major depression (see, e.g., Nudmamud-Thanoi et al. Neurosci. Lett. 30:173-7 (2004); Clinton et al. Neuropsychopharmacology 29(7):1353-62 (2004); and Heresco-Levy et al. Eur. Neuropsychopharmacol., 8(2):141-52 (1998)). This link between AKAP9 and the depression phenotype demonstrates that SNPs associated with AKAP9 mRNA expression are useful to predict or detect susceptibility to depression in an individual.

In the cardiac myocyte several AKAP proteins are involved in regulating β1 adrenergic receptor-induced, cAMP-dependent signaling mediated by PKA. Repolarization of the cardiac action potential at the plasma membrane occurs via ion flow through potassium channels during the QT interval of the cardiogram. Voltage-gated potassium channel 1 (KCNQ1) encodes a subunit of the potassium channel required for the delayed rectifier K+ current (Iₖ). This subunit is mutated in heritable forms of the long QT syndrome, a disease characterized by prolonged QT intervals and associated with arrhythmias. The slow component of the Iₖ current (Iₖₛ) is regulated by PKA. The yotiao splice form of AKAP9 has been found complexed to KCNQ1. Regulation of KCNQ1 by PKA and protein phosphatase PP1 requires interaction of KCNQ1 with yotiao through a leucine zipper motif. Congenital long QT syndrome is characterized by ventricular fibrillation with prolonged QT intervals and associated with increased risk of sudden death. One mutation found in patients with long QT syndrome is a single amino acid change in the leucine zipper that serves as interaction surface with yotiao. The mutation abolishes KCNQ1 binding of yotiao and renders the channel insensitive to cAMP signaling. Recent evidence indicates that yotiao not only provides recruitment sites for KCNQ1 regulators, but also serves as a signaling sensor of the phosphorylation state of the channel subunit. See Saucerman et al., Circ Res., 95(12):1216-24 (2004). Thus, SNPs associated with AKAP9 mRNA expression are useful to detect or predict susceptibility to heart disease and associated disorders, especially arrhythmia, long QT syndrome, ventricular fibrillation, cardiac arrest and sudden death.

DNAJD1 is a member of a highly conserved family of heat shock proteins (“Hsp”) containing four distinct domains including a 70-amino acid residue referred to as the DNAJ domain. This domain is necessary in forming an interaction between DJAJD1 and interacting proteins. See Kelley, W. L., Trends Biochem. Sci., 23:222-227 (1998). Specifically, DNAJD1 is a member of the Hsp40 heat shock protein family, a set of chaperone proteins, which participates in protein folding and regulation of diverse cellular processes including protein transport, cell cycle and stress responses. DNAJD1 functions in concert with Hsp70 family members to assist in protein folding and prevention of protein misfolding. See Hendrick, J. P. and Hartl, F. U., FASEB J., 9, 1559-1569 (1995) and Hartl, F. U., Nature, 381, 571-579 (1996). For example, DNAJD1 and DNAJA2 participate in mitochondrial protein import and DNAJA4 is involved in heat stress response.

DNAJD1 was cloned by differential display PCR from ovarian epithelial cells and named methylation-controlled J protein, MCJ. Analysis by RT-PCR has shown that two-thirds of primary ovarian tumors have either a complete loss or decreased expression levels of MCJ. See Shridhar et al., Cancer Research, 61:4258-65 (2001). DNA methylation has been shown to inactivate tumor suppressor genes and is increasingly being viewed as a tumor risk marker in non-cancerous tissues. See Malfoy, B., J. Cell Sci., 113(Pt 22):3887-8 (2002) and Muller et al., Ann. N.Y. Acad. Sci., 1022:44-9 (2004). Loss of heterozygosity and PCR studies confirmed that MCJ expression is suppressed by methylation, which can be reduced by methyltransfer inhibitors. These results suggest a role of MCJ as a tumor suppressor. See Starthdee et al., Carcinogenesis, 25(5):693-701 (2004). MCJ overexpression performed through colony-forming assays led to increased sensitivity to the anti-tumor drugs pacilitaxel, topotecan and cisplatin. Moreover, loss of MCJ expression has been correlated with resistance to these types of drugs. See Shridhar et al., Cancer Research, 61:4258-65 (May 2001). As such, SNPs associated with expression levels of DNAJD1 can be used as markers to predict or detect cancer in a patient as well as to predict the effectiveness of anti-tumor agents in the treatment of cancer.

Ataxin-1 aggregation and astrocyte injury have been implicated in neurodegeneration. See Forman et al., J Neurosci., 25(14):3539-50 (2005) and Cummings et al., Philos. Trans. R. Soc. Lond. B. Biol. Sci., 354(1386):1079-81 (1999). Aggregation of insoluble proteins such as ataxin-1 has been associated with neurodegenerative diseases such as Huntington's disease, Alzheimer's disease, Parkinson's disease, the prion disorders, dentatorubral-pallidoluysian atrophy (DRPLA) and spinocerebellar ataxia type 1 and 3 (SCA1 and SCA3). DNAJD1 overexpression decreased ataxin-1 aggregation in HeLa cells. See Cummings et al., Nat. Genet., 19(2):148-152 (1998). Another potential cause of neurodegenerative disease is astrocyte injury occurring as a result of CNS trauma and ischemia. Astrocytes are glial cells which respond upon injury to the brain. Injury to these cells is associated with neurodegenerative diseases such as Alzheimer's as well as psychiatric disorders such as schizophrenia and depression. Overexpression of DNAJD1 resulted in a significant reduction of astrocyte injury. See Qiao et al., J. Cereb. Blood Flow Metab., 23(10):1113-6. This suggests a potential mechanism by which SNPs associated with DNAJD1 levels are useful as markers to predict or detect patient susceptibility to neurodegenerative disease such as neurodegeneration, Alzheimer's disease, Parkinson's disease, Huntington's disease, prion disorders, DRPLA, SCA1, SCA3, schizophrenia and depression as well as to predict the progression of or ability to recover from such diseases.

Ischemia results from oxygen deprivation in tissues usually due to lack of blood flow. There is evidence that DNAJD1 may provide protection against injury caused by ischemia such as brain, myocardial and intestinal injury. See Qiao et al., J. Cereb. Blood Flow Metab., 23(10):1113-6. As such, SNPs associated with mRNA levels of DNAJD1 can be useful to predict or detect ischemia and ischemic-type injury, especially cardiomyopathy, coronary disease, coronary artery disease, heart attack, stroke, and intestinal ischemia. They are also useful as a means of prognosis and progression of such injury.

GOLPH4 is a ubiquitously expressed, type II membrane protein that resides in the cis-Golgi. This human protein contains a short, cytoplasmic tail of twelve amino acids and a large lumenal domain of 664 residues. See Natarajan et al., Mol. Biol. Cell, 15(11): 4798-806 (2004); Linstedt et al., Mol. Biol. Cell, 8(6): 1073-87 (1997). The outer region of the lumenal domain is very acidic because it is enriched in acidic amino acids. See Natarajan et al., Mol. Biol. Cell, 15(11): 4798-806 (2004). The region of GOLPH4 closest to the membrane contains elements that facilitate its pH sensitive targeting. See Natarajan et al., Mol. Biol. Cell, 15(11): 4798-806 (2004); Bachert et al., Mol. Biol. Cell, 12(10):3152-60 (2002). When treated with an agent that prevents acidification, GOLPH4 moves to endosomes. Upon removal of such an agent, GOLPH4 redistributes to the cis-Golgi. See Puri et al., Traffic 3(9):641-53 (2002). GOLPH4 is also known as GPP130 because it is 130-kDa in size. Linstedt et al., Mol. Biol. Cell, 8(6): 1073-87 (1997).

GOLPH4 seems to play an important role in the movement of proteins and toxins from endosomes to the Golgi via the bypass pathway. Studies on Shigella toxin B subunit trafficking revealed that the bypass pathway bypasses the conventional route, the late endosome/pre-lysosome pathway. The bypass pathway transports proteins or toxins directly from early endosomes to the Golgi and it is branched out of the plasma membrane receptor re-cycling route. Early sorting may be advantageous in that it may reduce the amount of degradation incurred by proteins or toxins that cycle between the Golgi and plasma membrane. In the study, GOLPH4 silencing by RNAi disrupted the normal movement of proteins and toxins. Two proteins dependant on the bypass pathway accumulated in the early/recycling endosomes. Shiga toxin B movement was also inhibited. Yet, proteins known to travel via the late endosome pathway continued to travel to the Golgi. See Natarajan et al., Mol. Biol. Cell, 15(11): 4798-806 (2004).

Thus, GOLPH4 affects the functionality of a normal retrieval route for proteins and Shiga B toxin to the Golgi. It may also determine the viability of invasion by other toxins, since bacterial and plant toxins are taken up by cells through endocytosis and then exert their effect in the cytoplasm. See Natarajan et al., Mol. Biol. Cell, 15(11): 4798-806 (2004). Polymorphisms that correlate with expression levels of GOLPH4 may be used to predict human susceptibility to diseases that occur as a result of the non-movement of proteins to the Golgi due to low levels or an absence of GOLPH4. Absence of or low levels of expression of GOLPH4 may also predict conditions caused by premature direction of components in the endocytosis pathway towards the endosomal degradation pathway. Finally, polymorphisms that correlate with normal or increased expression levels of GOLPH4 may predict human susceptibility to bacterial toxins.

RABEP1 is a 100-kD protein that regulates endosome trafficking by providing a tether between transport vesicles and target membranes. The RABEP1 protein interacts with Rab5, a regulator of endocytic vesicle trafficking. As with other Ras protein family members, Rab5 recruits RABEP1 to the early endosome. Trafficking pathways involving RABEP1 include homotypic fusion of endosomes, transport from the trans-Golgi network to the endosome and recycling of membranes from the early endsome to the Golgi. Overexpression of RABEP1 was shown to cause morphological alterations to the early endosome similar to those caused by the overexpression of interacting protein Rab5. Further, immunodepletion of RABEP1 causes a significant decline of early endosome fusion regulated by Rab5. The central role of RABEP1 is to provide a tether between vesicles and target membranes prior to vesicle fusion allowing SNAREs and other membrane and vesicle proteins to establish contact and determine vesicle-target membrane specificity. RABEP1 mediated “docking” of vesicles is a rate-limiting step in vesicle fusion. Trafficking pathways dependent on rabpatin-5 include homotypic fusion of endosomes, transport from the trans-Golgi network (TGN) to the endosome and recycling of membranes from the early endosome to the Golgi complex. See Stenmark et al., Cell, 83(3):423-32 (1995).

Rabaptin-5 binds both the small GTPase Rab5 as well as the Rab5 GTP exchange factor (GEF) Rabex-5. Rabex-5 promotes the exchange of GDP for GTP on Rab5 thereby catalyzing the formation of active Rab5-GTP on membranes and increasing the affinity of Rab5 for Rabaptin-5. These interactions are thought to promote endosome fusion. See Mattera et al., EMBO, 22(1):78-88 (2003). Inhibition of vesicle production was shown to occur upon depletion of RABEP1/Rabex-5 complex. This further suggests the role of RABEP1 in vesicle recycling. See Pagano et al., Mol. Biol. Cell, 15(11):4990-5000 (2004).

The N-terminus of Rabaptin-5 contains a binding site for the small GTPase Rab4. The GTPase Rab4 is a marker for early endosomes and regulates recycling of membrane to the Golgi complex. An adjacent GAE binding site interacts with γ1-adaptin, a subunit of the adaptor complex 1 (API). The AP-1 complex mediates transport from the endosome to the Golgi. Adaptor protein complexes are hetero-tetramers that integrate cargo selection with the formation of clathrin-coated vesicles. By binding both Rab4 and γ1-adaptin, Rabaptin-5 regulates endosome-to-Golgi transport. See Deneka et al., EMBO, 22(11):2645-2657 (2003). Defects in endosomal trafficking have been associated with neurodegenerative diseases such as Alzheimer's disease and Niemann-Pick type C disease.

Specifically, defects in synaptic vesicle recycling such as those associated with RABEP1 may be involved in synapse loss common in Alzheimer's disease. See Yao et al., Neurosci. Lett., 252(1):33-36 (1998). Accumulation and formation of beta-amyloid plaques in brain tissue is a hallmark of Alzheimer's disease pathology. Recently, high levels of RABEP1 interacting protein Rab5 were demonstrated to be highly enriched with beta-amyloid precursor protein APP. See Ikin et al., J. Biol. Chem., 271(50):31783-6 (1996).

Receptor tyrosine kinases (RTK) are transmembrane proteins that transduce extracellular signals to the cytoplasm and initiate a variety of cell responses through phosphorylation cascades. An extracellular domain receives activating signals through specific ligands inducing receptor multimerization. The intracellular domain of RTKs contains a tyrosine kinase activity which, upon ligand binding, autophosphorylates the receptor dimer. The phosphorylated form of the receptor recruits accessory proteins that propagate the signal through subsequenct phosphorylation/dephosphorylation events resulting in activation of growth-promoting or growth-inhibiting genes. RTK signaling is abrogated by internalization of the receptor through endocytosis.

Upregulation of RTKs is a frequent event in tumor formation and metastasis. Transforming mutations lead to permanent or increased activation of the receptor by gene fusion events resulting in overexpression, mutations causing ligand-independent activation or mutations activating the kinase domain. Disruptions in receptor endocytosis and recycling are another process that can affect the level of effective receptor activity on the cell surface. Rab5 has been shown to contribute to EGFR recycling and changes in Rab5 expression levels modulate the amount of EGFR present on the cell membrane. See Dinneen et al., Exp. Cell Res., 294(2):509-22 (2004). Expression levels of Rabaptin-5 could equally modify the availability of EGFR and other receptor kinases thereby affecting cancer cell growth and differentiation.

TAP has an ATP-binding cassette and translocates peptides from the cytosol to the lumen of the endoplasmic reticuclum (ER) where antigen presenting MHC class I molecules bind. ATP is hydrolyzed by TAP to facilitate the peptide transport process. The TAP complex is composed of two polypeptide subunits, TAP1 and TAP2. These proteins are similar in structure, each containing a transmembrane domain and a nucleotide binding domain. See Chen et al., J. Biol. Chem., 279(44):46073-46081 (2004). The TAP2 subunit was shown to form the pore of the TAP complex demonstrating its essential role in mediating peptide loading. TAP2 is also required for recruitment of tapasin polypeptide, another necessary component in TAP-mediated protein translocation. See Koch et al., J. Biol. Chem., 27(11):10142-10147 (2004).

Surface expression of class I major histocompatibility complex (MHC class I) is critical in the ability of the immune system to recognize and eliminate mutated and infected cells. MHC class I proteins synthesized in the ER are expressed on cell surfaces. Foreign peptides resulting from malignancy (e.g. cancer cells) or viral infection are presented on the MHC class I protein triggering immune response. Viral pathogens have evolved by developing methods of evading immune system detection and response. For example, herpes-simplex virus (HSV) escapes immune recognition through downregulation of MHC class I surface expression. See Lankat-Buttgereit, B. and Tampe, R., Physiol. Rev., 82(1):187-204 (2002). The demonstrated ability of HSV protein ICP47 inhibits the TAP complex thereby preventing production and presentation of MHC class I antigens. See Ahn et al., EMBO J., 15(13):3247-3255 (1996) and Kyritsis et al., J. Biol. Chem., 276(51):48031-9 (2001). Thus, a decline in TAP2 levels leading to decreased MHC class I presentation would result in impaired immune response and increased susceptibility to viral infection in an individual.

Cancer cells lack the ability to present cell surface antigens. Several cancers, such as melanomas, exhibit of decreased levels of MHC class I surface presenting proteins. See Sherman et al., Crit. Rev. Immunol., 18(1):47-54 (1998). Diminished TAP2 expression levels are present in breast cancer and human non-small cell lung cancer cells. See Alimonti et al., Nature Biotechnol., 18(1):515-520 (2000) and Seliger et al., Immunol. Today, 18(1):292-299 (1997). TAP complex downregulation has also been shown to cause HLA class I antigen loss. See Chen et al., Nat. Genet., 13(1):210-213 (1996). Decreased TAP2 expression would lead to a decline in MHC class I and HLA class I molecules necessary for cancer cell recognition and elimination. Thus, TAP2 expression levels are indicative of cancer cell presence as well as potential to metastasize.

Occasionally, the immune system attacks endogenous self-proteins which it mistakes as foreign pathogens. This process, known as autoimmunity, can result in diseases such as Wegener's granulomatosis, multiple sclerosis, type 1 diabetes mellitus and rheumatoid arthritis. One example of such an autoimmune response is the recognition and destruction of cells lacking HLA class I proteins by natural killer (NK) cells. Impaired HLA class I expression increases susceptibility to autoimmune disease caused by NK cell mediated cytotoxicity. HLA class I proteins are synthesized from proteins imported into the ER lumen by the TAP complex and show diminished expression in individuals with TAP2 deficiency. See Vitale et al., Blood, 99(5):1723-1729 (2002). Underexpression of TAP2 leads to autoimmunity caused by lack of HLA class I proteins.

NARG2 is expressed in fetal tissue, but is significantly down-regulated in adults with the most substantial expression levels found in the kidney, testes, liver and brain. Down-regulation of NARG2 was shown to be disrupted in the absence of NMDA receptor in NMDAR1 knockout mice. Intermediate levels of NARG2 were present in NMDAR1+/− mutants. Further, P19 culture cells treated with retinoic acid to induce neuronal differentiation caused a decline in NARG2 expression levels concurrent with increased NMDAR1 expression levels. See Sugiura et al., Eur. J. Biochem., 271(23-24):4629-37 (2004). These studies demonstrate a correlation between NARG2 expression levels and neuronal differentiation, mainly the transition of neuronal precursor cells to neurons.

The inability of the mammalian CNS to regenerate after damage has been implicated in a number of neurodegenerative diseases such as amyotrophic lateral sclerosis (ALS), Parkinson's disease, Alzheimer's disease and other types of brain and spinal cord injury. See Chen et al., Proc. Natl. Acad. Sci. USA., 101(46):16357-62 (2004). Stimulation by agents that induce neuronal differentiation creates a source of regenerative cells with a therapeutic effect in the treatment of such neurodegenerative diseases. See Richarson et al., Brain Res., 1032(1-2): 11-22 (2005). Thus, the SNPs of the present invention are useful to predict or detect susceptibility to neurodegenerative diseases in an individual as well as to predict progression of these diseases.

NMDA receptors have been studied extensively. NMDA receptors are known mediate synaptic transmission and neural plasticity in the mammalian central nervous system. (See, Monaghan Annu Rev Pharmacol Toxicol, 29:365-402 (1989); Collingridge Pharmacol Rev, 41:143-210 (1989); McBain Physiol Rev, 74:723-60 (1994)). NMDA receptors are differentially expressed during development (Sheng Nature, 368:144-7 (1994)). NMDA receptors are involved in a variety of fundamental biological processes including brain development by stabilizing converging synapses (Scheetz Faseb J, 8:745-52 (1994)), stimulating cerebellar granule cell migration (Hitoshi et al., Science, 260:95-97 (1993); Farrant Nature, 368:335-9 (1994); Rossi Neuropharmacology, 32:1239-48 (1993)) and development (Burgoyne J Neurocytol, 22:689-95 (1993)), inducing long term depression (Battistin Eur J Neurosci, 6:1750-5 (1994); Komatsu Neuroreport, 4:907-10 (1993); Tsumoto Jpn J. Physiol., 40:573-93 (1990)) and apoptosis (Finiels J Neurochem, 65:1027-34 (1995); Ankarcrona FEBS Lett, 394:321-4 (1996)). NMDA receptors are also known contribute to excitatory cell death in a number of adult pathological conditions (Greenamyre Neurobiol Aging, 10:593-602 (1989); Meldrum Trends Pharmacol Sci, 11, (1990) 379-87; Clark, S, “The NMDA receptor in epilepsy”, 2 edn., Oxford University Press, Oxford, 1994, 395-427 pp.; Doble, A., Therapie, 50:319-37 (1995)).

Excitatory amino acid receptors, including NMDA receptors, are known to be involved in neurodegenerative diseases, and specific NMDA antagonists are being used in clinical research (Lipton Trends Neurosci, 16:527-32 (1993)) for the potential treatment of stroke, CNS trauma (Faden Trends Pharmacol Sci, 13:29-35 (1992)), epilepsy (Thomas J Am Geriatr Soc, 43:1279-89 (1995); Perucca Pharmacol Res, 28:89-106 (1993)), pain (Elliott Neuropsychopharmacology, 13:347-56 (1995)), Huntington's disease (Purdon J Psychiatry Neurosci, 19:359-67 (1994)), AIDS dementia (Lipton Dev Neurosci, 16:145-51 (1994); Lipton Ann N Y Acad Sci, 747:205-24 (1994)), and Alzheimer's disease (Barry Arch Phys Med Rehabil, 72:1095-101 (1991)) and Parkinson's disease (Ossowska N Neural Transm Park Dis Dement Sect, 8:39-71 (1994)) (Rogawski Trends Pharmacol Sci, 14:325-31 (1993)). In vivo treatment with some of these agents manifest PCP-like psychotomimetic effects. Hence, research has been underway to discover and develop more therapeutically useful and less toxic drugs (Willetts Trends Pharmacol Sci, 11:423-8 (1990)). One less-toxic NMDA antagonist candidate is Ro-01-6794/706 or dextrorphan (Ann N Y Acad Sci, 765 249-61, 298 (1995)). Dextromethorphan and it's metabloite dextrorphan are widely used over the counter as antitussives (Irwin Drugs, 46:80-91 (1993)) which are NMDA channel blockers (Fekany Eur J Pharmacol, 151:151-4 (1988); Choi J Pharmacol Exp Ther, 242:713-20 (1987)) that may be a clinically useful neuroprotectant (Steinberg Neurosci Lett 133:225-8 (1991)). Therapeutically tolerated doses of roughly 30 mg (q.i.d.) orally are used for the over the counter antitussive action, and to 90 mg (q.i.d.) orally for clinical treatment of brain ischemia (Albers Clin. Neuropharmacol., 15:509-14 (1992)). Side effects at high doses of dextromethorphan and dextrorphan included drowsiness, nausea, and decreased coordination. Toxic high doses of dextromethorphan and dextrorphan have been described (Wolfe Am J Emerg Med, 13:174-6 (1995); Hinsberger J Psychiatry Neurosci, 19:375-7 (1994)); Loscher Eur J Pharmacol, 238:191-200 (1993)).

Numerous potentially clinically useful NMDA antagonists have been studied (Jane “Agonists and competitive antagonists: structure-activity and molecular modeling studies”, 2 edn., Oxford University Press, Oxford, 1994, 31-104 pp; Andaloro Society for Neuroscience Abstracts, 604 (1996); Bigge Biochem Pharmacol, 45:1547-61 (1993); Ornstein, P., “The development of novel competitive N-methyl-D-aspartate antagonists as useful therapeutic agents: Discovery of LY274614 and LY233536”, Raven Press, New York, 1991, 415-423 pp), and some are even orally available, including some derivatives EAB-515 (Li J Med Chem, 38 1955-65 (1995); Lowe Neurochem Int, 25:583-600 (1994)), memantine (Parsons Neuropharmacology, 34:1239-58 (1995); Kornhuber J Neural Transm Suppl, 43:91-104 (1994); Wenk Eur J Pharmacol, 293 267-70 (1995)), and ketamine (Parsons Neuropharmacology, 34:1239-58 (1995); Sagratella Pharmacol Res, 32:1-13 (1995); Porter J Neurochem, 64:614-23 (1995)). Some of these NMDA antagonists are approved for use, several others are in clinical trials for the treatment of neurodegenerative disease, epilepsy, stroke, and other diseases.

References which disclose other NMDA receptor blockers as well as assays for identifying an agent that acts as such a blocker and toxicity studies for pharmacologic profiles are disclosed in the foregoing and following articles which are all hereby incorporated by reference in their entirety. (See also Jia-He Li, et al., J Med Chem 38:1955-1965 (1995); Steinberg et al., Neurosci Lett, 133:225-8 (1991); Meldrum et al., Trends Pharmacol Sci, 11:379-87 (1990); Willetts et al., Trends Pharmacol Sci, 11:423-8 (1990); Faden et al., Trends Pharmacol Sci, 13:29-35 (1992); Rogawski, Trends Pharmacol Sci, 14:325-31 (1993); Albers et al, Clinical Neuropharm, 15:509-514 (1992); Wolfe et al., Am J Emerg Med, 13:174-6 (1995); Bigge, Biochem Pharmacol, 45:1547-61 (1993)). Examples of known NMDA receptor antagonists include memantine, adamantane, amantadine, an adamantane derivative, dextromethorphan, dextrorphan, dizocilpine, ibogaine, ketamine, remacemide, and phencyclidine. The SNPs in NARG2 useful in predicting NARG2 gene expression can also be used in predicting the gene expression of NMDAR1, because of the inverse correlation between NARG2 expression and NMDAR1 expression.

RNA helicases of the DEAD box family are present in almost all organisms and function in a variety of RNA metabolism related processes. RNA metabolism involves a dynamic rearrangement of RNA and proteins during transcription, pre-mRNA splicing, translation initiation, RNA transport and RNA degradation. Single-stranded RNA is also prone to form partial intra- and intermolecular interactions, which might interfere with or regulate some of the above processes. DEAD box RNA helicases unwind RNA double strands and dissociate RNA-protein complexes. The ATPase component of the DEAD box motif provides the energy required for unwinding RNA duplexes, rearranging RNA secondary structure or regrouping RNA-protein interactions. See Imaizumi, et al., Biochem. Biophys. Res. Commun., 292(1):274-9 (2002).

Innate immune response is an organism's first-line defense against pathogens in advance of the subsequent process of adaptive immunity. One essential component of innate immune response are cytokines of the type I interferon family which are induced by bacterial molecules such as lipopolysaccharide (LPS) and CpG DNA, and viral infection. Signaling by pathogen associated molecular patterns (PAMPs) is received by members of the Toll-like receptor (TLRs) family. Extracellular double-stranded RNA, LPS, viral single-stranded RNA and CpG DNA are recognized by TLR3, TLR4, TLR8 and TLR9, respectively. Ligand binding at the extracellular, leucine-rich repeats induces recruitment of adaptor molecules such as MyD88, IRAK, TRAF6 and Trif to the cytoplasmic domain. This initiates a signaling cascade that ends with the activation of transcription factors, among them IRF3 and NFkB, and the activation of a pro-inflammatory transcriptional program. See Yoneyama, et al., Nature Immunology, 5(7):730-737 (2004).

During infection RNA viruses enter the cell through membrane fusion, delivering their RNA genome in form of a RNP particle to the cytoplasm and bypassing both the extracellular TLR3 as well as TLR3 molecules in the endocytotic pathways. The cytoplasmic sensor for double-stranded RNA is the RNA helicase DDX58, also known as RIG-1. See Li, et al., J. Biol. Chem., 280(17):16739-47 (2005). The protein not only contains a RNA helicase motif, but also two N-terminal death-like domains, which are related to the protein-protein interaction domains DED and CARD. These domains promote homotypic interactions and serve as platforms for the assembly of signaling complexes. It has recently been shown that RIG-1 regulates dsRNA induced signaling and is essential for induction of IRF3 after viral infection. The RNA helicase domain is required for response to viral infection and appears to negatively regulate the CARD domain in the absence of double-stranded RNA. The CARD-like domain of RIG-1 constitutively activates IRF3 and NFkB when expressed in mouse L929 cells. Abrogation of RIG-1 by RNAi impairs activation of IRF3 in response to viral infection and over-expression of RIG-1 reduces viral yield. RIG-1/DDX58 thus is a crucial component of the innate immune response and levels of RIG-1 could predict individual variation in immune response to viruses. See Yoneyama, et al., Nature Immunology, 5(7):730-737 (2004). Overexpression of RIG-1 has also been shown to increase ISGI5 levels resulting in an increase in natural killer cells and cytotoxicity. See Cui, et al., Biochem. Cell Bio., 82(3):401-5 (2004). Furthermore, silencing of RIGI expression impaired response to Sendai virus in hepatocytes. See Li, et al., J. Biochem., 280(17):16739-47 (2005).

COX2 overexpression has been linked to carcinogenesis and tumorigenesis. Specifically, COX2 expression is sufficient to induce mammary gland tumorigenesis. See Lui, et al., J. Biol. Chem. 276: 18563-18569 (2001). Overexpression of COX2 was also shown to be an early, central event in carcinogenesis in Apc(delta-716) knockout mice. See Oshima, et al., Cell 87: 803-809 (1996). RIGI overexpression lead to increased expression of COX2 mRNA in bladder cancer cells and further induces COX2 activity in endothelial cells. This demonstrates the potential role of RIGI expression levels in the role of cancer development and tumor progression. See Imaizumi, et al., Biochem. Biophys. Res. Commun., 292(1):274-9 (2002).

Vascular injury, shear stress, hypoxic conditions and inflammatory mediators induce the release of adenosine nucleotides into the local intracellular vasculature. ATP and ADP are also present in extracellular fluid due to plasma membrane permeability and exocytotic vesicles. In blood ATP and ADP regulate platelet aggregation through binding to purinergic P2 receptors on the platelet surface. Free ADP is a potent activator of platelet aggregation, while ATP acts as a competitive antagonist. ATP also acts as an anti-thrombotic through activation of two inhibitors of platelet aggregation, prostacyclin (PGI2) and nitric oxide (NO). Both PGI2 and NO improve blood flow by relaxing smooth muscles and promoting vasodilation as well as inhibiting local effects of ADP on platelet activation. The breakdown product of AMP, adenosine, is also an anti-thrombotic with actions on platelet aggregation that oppose those of ADP. See Burnstock, G., and Williams, M., J. Pharmacol. Exp. Ther., 295(3):862-9 (2000).

Extracellular ATP and ADP are hydrolyzed by ecto-nucleoside triphosphate diphosphohydrolases (ENTPDases), nucleotide pyrophosphatase/phospho-diesterase I family members and alkaline phosphatases. Members of all three families are present in plasma. The main ecto-nucleosidase in vascular endothelial cells responsible for the hydrolysis of ADP is CD39, which recognizes all forms of nucleoside triphosphates that occur physiologically. It is a transmembrane protein with two TM domains, one located N-terminally and a second one at the C-terminus. This topology creates two intracellular domains at the N- and C-termini and a large extracellular loop located between the TM domains. CD39 appears to be constitutively palmitoylated at Cys13 and this fatty acid modification promotes targeting to calveolae, plasmamembrane regions enriched in signaling molecules including purinergic receptors. According to data from CD39 deficient mice CD39 is the main ecto-nucleosidase at the inner vascular surface. CD39 converts ATP to ADP and qADP to AMP. AMP is catabolized by ecto-5′-nucleotidase to adenosine. By promoting the degradation of ADP to adenosine CD39 is a critical regulatory molecule for maintaining blood flow. See Koziak et al., J. Biol. Chem., 275(3):2057-62 (2000).

CD39 knockout mice exhibited longer bleeding times and defects in platelet aggregation despite little changes in platelet numbers, plasma ATP or ADP concentrations. Instead disrupted thromboregulation was due to desensitization of purinergic receptors that could be reconstituted by exposure to exogenous ATPDases. CD39−/− endothelial cells failed to abrogate platelet aggregation after stimulation with ADP. Increased susceptibility of CD39 negative mice to vascular injury was suggested by increased deposition of fibrin in most vasculatures, including pulmonary, cardiac, renal, cerebral and splenic. CD39-null mice showed impaired chemotactic response of macrophages and monocytes and absence of new vessel growth. See Mizumoto et al., Nature Medicine, 8(4):358-365 (2002).

Transgenic expression of CD39 in mice also results in increased bleeding times and disruption of platelet aggregation. However, CD39 transgenic mice are protected against systemic thrombosis when challenged with collagen or ADP. See Enjyoji et al., Nature Medicine, 5(9):1010-1017 (1999). Increased expression of CD39 has also been related to plaque stability and reduced thrombus formation in angina pectoris patients suggesting a role in prevention of acute coronary syndromes. See Hatakeyama et al., Am. J. Cardiol., 95(5):632-5 (2005).

Acute rejection of allograft transplants has been reduced through the application of immuno-suppressants. A critical feature is inflammatory response that triggers platelet deposition and small vessel thrombosis. CD39 expression levels appear to correlate with rejection risk. Xenografts from CD39 deficient mice had higher rejection rates while grafts from CD39 transgenic mice showed increased survival times. See Imai et al., Mol. Med., 5(11): 743-52 (1999).

Langerhans cells (LC) are dendritic cells within the epidermis that contribute to T cell stimulation and inflammation by presenting antigens for T cell responses. In CD39 deficient cells ecto-nucleosidase activity is absent in Langerhans cells indicating that ENTPD1 is the main ecto-nucleosidase in LC cells. In CD39−/− mice inflammatory responses to skin irritants were increased. Pro-inflammatory mediators ATP and ADP released by keratinocytes contribute to the activation of T cells, which trigger a second set of ATP dependent signaling events between T cells and dendritic cells. The level of ATP appears to be critical in determining the severity of the response and the expression level of CD39, responsible for ATP hydrolysis, could reflect the inter-individual variability in inflammatory response to irritants and immunogens. See Mizumoto et al., Nature Medicine, 8(4):358-365 (2002).

FKBP1a is the major binding protein for macrolide immunosuppressant drugs including FK506 and rapamycin. The FK506/FKBP1a complex binds to and inhibits the Ca2+ dependent phosphatase calcineurin. Calcineurin is a central regulator of T cell activation by dephosphorylating the transcription factor NFAT which regulates expression of several cytokines. The rapamycin/FKBP1a complex acts through a different pathway by inhibiting the serine-threonine kinase mTOR resulting in cell cycle arrest in G1 phase. Both actions are the basis for the use of FK506 type immunosuppressants in transplantation surgery. The FKBP gene family has been shown to be associated with antitumor activities. Particularly, FKBP1a gene expression has been shown to have antitumor effects through binding rapamycin, thereby arresting cells in G1. It was also noted that antitumorogenic effects could be related to the stimulation of T cell function by FKBP1a. The cell cycle inhibitory function of the rapamycin/FKBP1a interaction has led to applications of rapamycin as an anti-proliferative agent in cancer therapy. See Fong et al., PNAS, 100(24):14253-14258 (2003).

FKBP1a expression levels have been shown to increase in the case of nerve damage. Particularly, FKBP1a is upregulated subsequent to nerve crush injury. See Sezen et al., Int. J. Impot. Res., 14(6):506-12 (2002). Regenerating neurons identified by retrograde labelling were found to have upregulated FKBP1a mRNA levels. See Mason et al., Exp. Neurol., 181 (2):181-9 (2003).

Calcium binding proteins of the penta-EF family (PEF) contain a conserved helix-loop-helix signature that coordinates an Ca⁽²⁺⁾ ion in a pentagonal bipyramidal configuration using the oxygen atoms of an invariant glutamate or aspartate for ligand binding. PEF proteins include sorcin, grancalcin, peflin, calpain and ALG-2. PEF family members contain five repetitive EF-hand motifs, dimerize through unpaired C-terminal EFs and can also form heterodimers. An N-terminal hydrophobic domain promotes Ca⁽²⁺⁾ dependent translocation from a soluble Ca⁽²⁺⁾ free form to a Ca⁽²⁺⁾ bound membrane-attached version. See Hansen, et al., FEBS Lett., 545(2-3_{—}:151-4 (2003) and Farrell, et al., Biol. Res., 37(4):609-12 (2004). Sorcin was originally identified as a gene amplified in drug-resistant cancer cells. Its chromosomal location is close to that of ABCB1 (MDR1), a gene frequently found amplified in response to chemotherapy. Widely expressed in many tissues sorcin co-localizes with NMDA receptors in brain and is found near T-tubules in heart muscle. Several interactors for sorcin have been identified, including annexin VII and presenilin 2, both suggesting a role for sorcin in regulation of calcium homeostasis. See Meyers, et al., J. Biol. Chem., 278(31):28865-71 (2003).

Best described is its involvement in the regulation of excitation-contraction coupling in the heart muscle. Contraction of the heart muscle depends on coordinated intracellular calcium cycling. It is initiated by the influx of a small Ca⁽²⁺⁾ current through the voltage-activated LTCC channel (L type Ca⁽²⁺⁾ channel) during membrane depolarization. This triggers a much larger release of calcium from the sarcoplasmatic reticulum (SR) through the calcium release channel of the SR, the ryanodine receptor RYR2. Calcium binds to tropomyosin C in the myofilaments stimulating contraction. Dissociation of calcium from tropomyosin begins relaxation and free calcium is returned through efflux pumps like the Ca⁽²⁺⁾ ATPase in the SR and the Na⁺-CA⁽²⁺⁾ exchanger. Each of these transporters is regulated by phosphorylation, either directly or via phosphorylation of accessory proteins. See Marx, et al., Cell, 101(4):365-76 (2000).

The RYR2 homo-tetramer forms a complex with FKBP12.6, calmodulin, protein kinase A, protein phosphatase 1 and protein phosphatase 2A. Binding of FKBP12.6 stabilizes the closed form of the channel. Hyperphosphorylation of RYR2 as observed in heart failure, results in dissociation of FKBP12.6 from RyR2, destabilization of the closed state and Ca⁽²⁺⁾ leaks during diastole. Since FKBP12.6 promotes coupling between RYR2 receptors, loss of FKBP12.6 also disrupts the simultaneous systolic opening and diastolic closing of RYR2s. Both the reduced Ca⁽²⁺⁾ concentration in the SR and the uncoupling of RYR2 function contribute to cardiac arrhythmia.

The Ca⁽²⁺⁾ ATPase SERCA2 is regulated by the accessory protein phospholamban (PLN). PLN in its unphosphorylated state inhibits SERCA2 activity. Calcium release via RYR2 activates CAMKII, which phosphorylates PLN and relieves SERCA2 inhibition. This promotes cardiac contractility by recharging the SR calcium stores for the next cycle of release. In failing hearts a chronically activated Gαq-coupled receptor system activates protein kinase C which phosphorylates inhibitor-1, thus abrogating down-regulation of protein phosphatase 1 (PP1). PP1 induced hypophosphorylation of PLN diminishes the activity of SERCA2 and prevents proper calcium recycling. Dysfunction in the calcium cycling process triggers compensatory mechanisms in the heart, including hypertrophy, remodeling and apoptosis. The critical role of calcium in regulating cardiac contractility has made calcium-binding proteins a new interest for drug-based interference in heart failure.

Sorcin has been found complexed to three different cardiac calcium channels: the LTCC channel, responsible for the initial inward Ca⁽²⁺⁾ current, the RYR2 receptor which controls release of the Ca⁽²⁺⁾ stores from the SR and SERCA2 which actively returns Ca⁽²⁺⁾ to the SR. The calcium bound form of sorcin inhibits receptor activity. Phosphorylation of sorcin by PKA results in loss of receptor inhibition. Transgenic mice over-expressing sorcin in heart muscle cells lack obvious signs of cardiomyopathy, but show defects in cardiac contractility. Sorcin protein preferentially localizes to the Z-lines that contain high concentrations of both the LTCC and RYR2 channels. This co-localization is disrupted in myocytes from a rat model of heart failure. Binding of sorcin to RYR2 decreases specifically the inward Ca⁽²⁺⁾ current triggered activity of RYR2, thereby diminishing the excitation-contraction coupling. Overexpression of sorcin in rat cardiomyocytes also affects the Ca⁽²⁺⁾ uptake and Ca⁽²⁺⁾ load in the sarcoplasmic reticulum, indicating activation of the Ca⁽²⁺⁾ ATPase. This activity is reduced in failing hearts, which show increased PKA dependent phosphorylation of sorcin. Since sorcin is regulated by phosphorylation through the same set of enzymes as several other components of the cardiac calcium cycle (PKA, PP1), it does functionally resemble the two previous described accessory proteins. See Matsumoto, et al., Basic Res. Cardiol., (2005) and Meyers, et al., J. Biol. Chem., 278(31):28865-71 (2003). Consistent with this, sorcin overexpression has been associated with an increase in cardiac contractility and the rescue of abnormal contractile function in the diabetic heart. See Suarez, et al., Am. J. Physiol. Heart Circ. Physiol., 286(1):H68-75 (2004). Recently, Sorcin overexpression was demonstrated to improve cardiac contractility in vivo in transfected rat hearts. See Frank, et al., J. Mol. Cell. Cardiol., 38(4):607-15 (2005). The level of sorcin expression could thus affect susceptibility to heart failure and contribute to variability in response to medication targeting cardiac contractility.

Sorcin expression levels were measured in leukemic blast cells of patients with acute myloid leukemia (AML). Poor patient prognosis was associated with sorcin overexpression and remission rates were shown to be higher in patients with low sorcin expression than those with higher expression. See Tan, et al., Leuk. Res., 27(2): 125-31 (2003). Sorcin expression has also been shown to have an effect on cell resistance to chemotherapeutics. Specifically, sorcin overexpression via gene transfection in K562 cells resulted in increased resistance to chemotherapeutics including doxorubicin, etoposide, homoharringtonine and vincristine. Sorcin expression was also inhibited in these cells resulting in a reversal of drug resistance. See Zhou, et al., Luek. Res., (2005).

Exposure to environmental radiation is a normal hazard for all cells and enhanced radiation is used as a major modus of cancer treatment. Genetic factors that modulate sensitivity or resistance to radiation have considerable relevance for the development of diagnostics for susceptibility to cancer and treatment selection in cancer therapy. Irradiation of cells induces multiple cellular responses to radiation stress, including sensing mechanisms for DNA damage, signaling pathways, DNA repair systems and, if necessary, apoptosis. See Snyder, et al., Cancer Metastasis Rev., 23(3-4):259-268 (2004).

Genome-wide expression profiling has identified sets of genes that are differentially regulated upon exposure to ionizing radiation. As expected they include genes involved in cell cycle progression, cell survival, DNA repair and growth control. XRRA1 was cloned from a human colorectal tumor cell line, HCT116. Several clones of HCT116 with different responses to radiation were isolated. XRRA1 was down-regulated in an untreated cell clone resistant to radiation. The 559aa long protein is highly conserved among vertebrates. It contains several leucine-rich repeats (LLRs), a feature that has been implicated in protein-protein interactions. The repeat pattern observed in this protein is consistent with a motif found in proteins acting as Ran GTPase activating factors (RanGAPs). XRRA1 is expressed in many tissues with higher levels observed in testis, prostate and ovary. The XRRA1 protein was localized both in the nucleus and the cytoplasm.

The expression pattern of XRRA1 in HCT116 clones in response to irradiation showed differences depending on radiation sensitivity. A resistant clone had lower basal levels, but strongly induced XRRA1 within minutes upon radiation treatment. Levels then decreased slowly. A clone with increased radiation sensitivity had higher basal levels, but failed to induce XRRA1 upon irradiation. Instead levels dropped significantly and only recovered 24 hours post treatment. See Mesak, et al., BMC Genomics, 4(1):32 (2003). This differential pattern suggests that XRRA1 is involved in the response to radiation and levels of XRRA1 may be indicative of resistance and/or sensitivity to radiation.

IRF5 is a 504 amino acid interferon regulatory factor with a critical role in inducing expression of antiviral proteins in response to viral infection. IFNA is produced by cells in response to viral infection. Defects in IFNA production were found in 30 children with recurrent respiratory infection. See Isaacs et al., Lancet II, 950-952 (1981). Overexpression of IRF5 induces IFNA gene expression, underlining the importance of IRF5 levels in immune system response. Viral infection induces phosphorylation of IRF5 and increases binding to the virus regulatory element. See Barnes et al., J. Biol. Chem., 276(26):23382-90 (2001).

Toll-like receptors (“TLRs”) can detect most foreign microbes and are thus essential for innate recognition of viral pathogens in mammals. See Beutler, B., Nature, 430(6996):257-63 (2004). Reduced TLR induction was observed in IRF5 deficient mice demonstrating another association of IRF5 with immune response. See Takaoka et al., Nature, 434(7030):243-9 (2005). In view of the above, IRF5 mRNA expression levels are useful to predict and/or detect an individual's susceptibility to viral infection as well as the likelihood of post-infection viral progression.

AMFR is a receptor for autocrine motility factor, which is a cytokine secreted by tumor cells to promote tumor motility and metastasis. AMFR is a 78-KD cell surface glycoprotein (gp78) that transduces signals from the mitogenic cytokine AMF regulating cell motility in cell based assays and tumor metastasis in vivo. Aside from its seven transmembrane domains, AMFR contains a RING-H2 motif and a leucine zipper. See Shimizu et al., FEBS Lett., 456:295-300 (1999). Over-expression of AMFR induces a transformed phenotype and produces tumor in nude mice. See Onishi et al., Clin. Exp. Metastasis, 20(1):51-58 (2003). Signaling through AMF/AMFR induces vascular endothelial growth factor receptor FLT1 thereby stimulating cell growth through tyrosine phosphorylation pathways. See Funasaka et al., Int. J. Cancer, 101:217-23 (2002). Expression levels of AMFR in melanoma cell lines correlates with their potential to metastasize. See Timar et al., Clin. Exp. Metastasis, 19(3):225-232 (2002). Analysis of primary human skin melanoma tumors identified three types: weak, heterogenous and strong expression. Expression levels appeared to correlate with growth phenotype, strong expression being found in tumors with more pronounced vertical growth indicating a more invasive phenotype. See Timar et al., Clin. Exp. Metastasis, 19(3):225-232 (2002). About 40% of non-small cell lung cancers express AMFR, with expression being associated with type mainly in adenocarcinoma. Survival was significantly worse in patients with AMFR expression. The AMFR expression was also associated with VEGF expression, both indicating worse prognosis. See Kara et al., Ann. Thorac. Surg., 71:944-8 (2001). Approx. 30% of thymomas showed expression of AMFR, again expression being associated with worse outcome. See Ohta et al., Int. J. Oncol., 17:259-64 (2000). Since the SNPs and haplotypes described here are associated with the expression level of AMFR, they can be used to predict cancer susceptibility and prognosis in patients. They may also be useful in predicting patients' response to treatment with VEGF-targeting drugs.

### 3. Isolated Nucleic Acids

Accordingly, the present invention provides an isolated TLK1 nucleic acid containing at least one of the newly discovered nucleotide variants as summarized in Tables 1, 2 and 3. The term “TLK1 nucleic acid” is as defined above and means a naturally existing nucleic acid coding for a wild-type or variant or mutant TLK1. The term “TLK1 nucleic acid” is inclusive and may be in the form of either double-stranded or single-stranded nucleic acids, and a single strand can be either of the two complementing strands. The isolated TLK1 nucleic acid can be naturally existing genomic DNA, mRNA or cDNA. Sequences of several naturally existing TLK1 cDNAs and proteins are provided in SEQ ID NOs: 1, 2 and 14-17.

In yet another embodiment, the isolated TLK1 nucleic acid has a nucleotide sequence encoding TLK1 protein having an amino acid sequence according to SEQ ID NO:2 but contains one or more amino acid variants of Tables 1-3 (e.g., EX11@51G>A). Isolated TLK1 nucleic acids having a nucleotide sequence that is the complement of the sequence are also encompassed by the present invention.

In yet another embodiment, the isolated TLK1 nucleic acid has a nucleotide sequence encoding a TLK1 protein having an amino acid sequence that is at least 95%, preferably at least 97% and more preferably at least 99% identical to SEQ ID NO:2 but contains one or more amino acid variants of Tables 1-3 (e.g., EX11@51G>A), or the complement thereof.

In another embodiment, the present invention provides an isolated nucleic acid, naturally occurring or artificial, having a nucleotide sequence encoding a TLK1 protein having an amino acid sequence according to SEQ ID NO:2 but containing one or more amino acid variants of Tables 1-3 (e.g., EX11@51G>A). Isolated nucleic acids having a nucleotide sequence that is the complement of the sequence are also encompassed by the present invention.

In addition, isolated nucleic acids are also provided which have a nucleotide sequence encoding a protein having an amino acid sequence that is at least 95%, preferably at least 97% and more preferably at least 99% identical to SEQ ID NO:2 but containing one or more amino acid variants of Tables 1-3 (e.g., EX11@51G>A), or the complement thereof.

Also encompassed are isolated TLK1 nucleic acids obtainable by:

(a) providing a human genomic library;

(b) screening the genomic library using a probe having a nucleotide sequence according to any one of SEQ ID NOs: 1, 3-10; and

(c) producing a genomic DNA comprising a contiguous span of at least 30 nucleotides of any one of SEQ ID NOs:1, 3-10, wherein the genomic DNA thus produced contains one or more of the SNPs of the present invention in Tables 1-3, such as EX7@+63A, EX7@+190C, EX11@51A and EX25@855G.

The present invention also includes isolated TLK1 nucleic acids obtainable by:

(i) providing a cDNA library using human mRNA from a human tissue, e.g., blood;

(ii) screening the cDNA library using a probe having a nucleotide sequence according to any one of SEQ ID NOs: 1, 3-10; and

(iii) producing a cDNA DNA comprising a contiguous span of at least 30 nucleotides of any one of SEQ ID NOs:1, 3-10, wherein the cDNA thus produced contains one or more of the SNPs of the present invention in Tables 1-3, such as EX7@+63A, EX7@+190C, EX11@51A and EX25@855G.

The present invention also encompasses an isolated nucleic acid comprising the nucleotide sequence of a region of a TLK1 genomic DNA or cDNA or mRNA, wherein the region contains one or more nucleotide variants as provided in Tables 1-3 (e.g., EX7@+63A, EX7@+190C, EX11@51A and EX25@855G), or one or more nucleotide variants that will give rise to one or more amino acid variants of Table 2, or the complement thereof. Such regions can be isolated and analyzed to efficiently detect the nucleotide variants of the present invention. Also, such regions can also be isolated and used as probes or primers in detection of the nucleotide variants of the present invention and other uses as will be clear from the descriptions below.

Thus, in one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of a TLK1 nucleic acid, the contiguous span containing one or more nucleotide variants of Tables 1-3 (e.g., EX7@+63A, EX7@+190C, EX11@51A and EX25@855G), or the complement thereof. In specific embodiments, the isolated nucleic acid are oligonucleotides having a contiguous span of from about 17, 18, 19, 20, 21, 22, 23 or 25 to about 30, 40 or 50, preferably from about 21 to about 30 nucleotide residues, of any TLK1 nucleic acid, said contiguous span containing one or more nucleotide variants of Tables 1-3 (e.g., EX7@+63A, EX7@+190C, EX11@51A and EX25@855G).

In one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of any one of SEQ ID NOs:3-10, containing one or more nucleotide variants of Tables 1-3 (e.g., EX7@+63A, EX7@+190C, EX11@51A and EX25@855G), or the complement thereof. In specific embodiments, the isolated nucleic acid comprises a nucleotide sequence according to any one of SEQ ID NOs:3-10. In preferred embodiments, the isolated nucleic acid are oligonucleotides having a contiguous span of from about 17, 18, 19, 20, 21, 22, 23 or 25 to about 30, 40 or 50, preferably from about 21 to about 30 nucleotide residues, of any one of SEQ ID NOs: 3-10 and containing one or more nucleotide variants of Tables 1-3 (e.g., EX7@+63A, EX7@+190C, EX11@51A and EX25@855G). The complements of the isolated nucleic acids are also encompassed by the present invention.

Thus, for example, an isolated nucleic acid of the present invention can have a contiguous span of at least 18, 19, 20, 21, 22, 23, 24, 25, 26, 27, 28, 29, or 30 or 40 or 50 nucleotide residues of SEQ ID NO:3 containing the nucleotide variant EX7@+63A (nucleotide residue No. 51 in SEQ ID NO:3), or a contiguous span of at least 18, 19, 20, 21, 22, 23, 24, 25, 26, 27, 28, 29, or 30 or 40 or 50 nucleotide residues of SEQ ID NO:4 containing the nucleotide variant EX7@+190C (nucleotide residue No. 51 in SEQ ID NO:4), or a contiguous span of at least 18, 19, 20, 21, 22, 23, 24, 25, 26, 27, 28, 29, or 30 or 40 or 50 nucleotide residues of SEQ ID NO:5 containing the nucleotide variant EX11@51A (nucleotide residue No. 51 in SEQ ID NO:5), or a contiguous span of at least 18, 19, 20, 21, 22, 23, 24, 25, 26, 27, 28, 29, or 30 or 40 or 50 nucleotide residues of SEQ ID NO:6 containing the nucleotide variant EX25@855G (nucleotide residue Nos. 51 in SEQ ID NO:6), or the complements thereof.

The present invention further provides an isolated WARS2 nucleic acid containing at least one of the newly discovered nucleotide variants as summarized in Table 4, e.g., EX1@−963G, EX1@−103T, EX6@780G, EX6@842T and EX6@2152G. The term “WARS2 nucleic acid” is as defined above and means a naturally existing nucleic acid coding for a wild-type or variant or mutant WARS2. The term “WARS2 nucleic acid” is inclusive and may be in the form of either double-stranded or single-stranded nucleic acids, and a single strand can be either of the two complementing strands. The isolated WARS2 nucleic acid can be naturally existing genomic DNA, mRNA or cDNA. In one embodiment, the isolated WARS2 nucleic acid has a nucleotide sequence encoding WARS2 protein having an amino acid sequence according to SEQ ID NO:19 but contains one or more amino acid variants. Isolated WARS2 nucleic acids having a nucleotide sequence that is the complement of the sequence are also encompassed by the present invention.

In yet another embodiment, the isolated WARS2 nucleic acid has a nucleotide sequence encoding a WARS2 protein having an amino acid sequence that is at least 95%, preferably at least 97% and more preferably at least 99% identical to SEQ ID NO:19 but contains one or more amino acid variants, or the complement thereof.

The present invention also provides an isolated nucleic acid, naturally occurring or artificial, having a nucleotide sequence that is at least 95%, preferably at least 97% and more preferably at least 99% identical to SEQ ID NO:18 except for containing one or more nucleotide variants of Table 4 (e.g., EX1@−963G, EX1@−103T, EX6@780G, EX6@842T and EX6@2152G), or the complement thereof.

In another embodiment, the present invention provides an isolated nucleic acid, naturally occurring or artificial, having a nucleotide sequence encoding a WARS2 protein having an amino acid sequence according to SEQ ID NO:19 but containing one or more amino acid variants. Isolated nucleic acids having a nucleotide sequence that is the complement of the sequence are also encompassed by the present invention.

In addition, isolated nucleic acids are also provided which have a nucleotide sequence encoding a protein having an amino acid sequence that is at least 95%, preferably at least 97% and more preferably at least 99% identical to SEQ ID NO:19 but containing one or more amino acid variants, or the complement thereof.

Also encompassed are isolated WARS2 nucleic acids obtainable by:

(a) providing a human genomic library;

(b) screening the genomic library using a probe having a nucleotide sequence according to any one of SEQ ID NOs:18, 20, 22-26; and

(c) producing a genomic DNA comprising a contiguous span of at least 30 nucleotides of any one of SEQ ID NOs:18, 20, 22-26, wherein the genomic DNA thus produced contains one or more of the SNPs of the present invention in Table 4, such as EX1@−963G, EX1@−103T, EX6@780G, EX6@842T and EX6@2152G.

The present invention also includes isolated WARS2 nucleic acids obtainable by:

(i) providing a cDNA library using human mRNA from a human tissue, e.g., blood;

(ii) screening the cDNA library using a probe having a nucleotide sequence according to any one of SEQ ID NOs: 18, 20, 22-26; and

(iii) producing a cDNA DNA comprising a contiguous span of at least 30 nucleotides of any one of SEQ ID NOs: 18, 20, 22-26, wherein the cDNA thus produced contains one or more of the SNPs of the present invention in Table 4, such as EX1@−963G, EX1@−103T, EX6@780G, EX6@842T and EX6@2152G.

The present invention also encompasses an isolated nucleic acid comprising the nucleotide sequence of a region of a WARS2 genomic DNA or cDNA or mRNA, wherein the region contains one or more nucleotide variants as provided in 4 (e.g., EX1@−963G, EX1@−103T, EX6@780G, EX6@842T and EX6@2152G), or one or more nucleotide variants that will give rise to one or more amino acid variants, or the complement thereof. Such regions can be isolated and analyzed to efficiently detect the nucleotide variants of the present invention. Also, such regions can also be isolated and used as probes or primers in detection of the nucleotide variants of the present invention and other uses as will be clear from the descriptions below.

Thus, in one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of a WARS2 nucleic acid, the contiguous span containing one or more nucleotide variants of Table 4 (e.g., EX1@−963G, EX1@−103T, EX6@780G, EX6@842T and EX6@2152G), or the complement thereof. In specific embodiments, the isolated nucleic acid are oligonucleotides having a contiguous span of from about 17, 18, 19, 20, 21, 22, 23 or 25 to about 30, 40 or 50, preferably from about 21 to about 30 nucleotide residues, of any WARS2 nucleic acid, said contiguous span containing one or more nucleotide variants of Table 4 (e.g., EX1@−963G, EX1@−103T, EX6@780G, EX6@842T and EX6@2152G).

In one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of any one of SEQ ID NOs:22-29, containing one or more nucleotide variants of Table 4 (e.g., EX1@−963G, EX1@−103T, EX6@780G, EX6@842T and EX6@2152G), or the complement thereof. In specific embodiments, the isolated nucleic acid comprises a nucleotide sequence according to any one of SEQ ID NOs: 22-29. In preferred embodiments, the isolated nucleic acid are oligonucleotides having a contiguous span of from about 17, 18, 19, 20, 21, 22, 23 or 25 to about 30, 40 or 50, preferably from about 21 to about 30 nucleotide residues, of any one of SEQ ID NOs: 22-29 and containing one or more nucleotide variants of Table 4 (e.g., EX1@−963G, EX1@−103T, EX6@780G, EX6@842T and EX6@2152G). The complements of the isolated nucleic acids are also encompassed by the present invention.

Thus, for example, an isolated nucleic acid of the present invention can have a contiguous span of at least 18, 19, 20, 21, 22, 23, 24, 25, 26, 27, 28, 29, or 30 or 40 or 50 nucleotide residues of SEQ ID NO:22 containing the nucleotide variant EX1@−963G (nucleotide residue No. 51 in SEQ ID NO:22), or a contiguous span of at least 18, 19, 20, 21, 22, 23, 24, 25, 26, 27, 28, 29, or 30 or 40 or 50 nucleotide residues of SEQ ID NO:23 containing the nucleotide variant EX1@−103T (nucleotide residue No. 51 in SEQ ID NO: 23), or a contiguous span of at least 18, 19, 20, 21, 22, 23, 24, 25, 26, 27, 28, 29, or 30 or 40 or 50 nucleotide residues of SEQ ID NO:24 containing the nucleotide variant EX6@780G (nucleotide residue No. 51 in SEQ ID NO:24), or a contiguous span of at least 18, 19, 20, 21, 22, 23, 24, 25, 26, 27, 28, 29, or 30 or 40 or 50 nucleotide residues of SEQ ID NO:25 containing the nucleotide variant EX6@842T (nucleotide residue Nos. 51 in SEQ ID NO:25), or a contiguous span of at least 18, 19, 20, 21, 22, 23, 24, 25, 26, 27, 28, 29, or 30 or 40 or 50 nucleotide residues of SEQ ID NO:26 containing the nucleotide variant EX6@2152G (nucleotide residue Nos. 51 in SEQ ID NO:26), or the complements thereof.

The present invention further provides an isolated ARTS1 nucleic acid containing at least one of the newly discovered nucleotide variants as summarized in Tables 5-10, or one or more nucleotide variants that will result in the amino acid variants provided in Tables 5-11, e.g., EX1@−1125T, EX2@397C, EX20@1085G, EX15@74A, EX19@885T, EX20@2105C, EX20@719C and EX20@1038A. The term “ARTS1 nucleic acid” is as defined above and means a naturally existing nucleic acid coding for a wild-type or variant or mutant ARTS1. The term “ARTS1 nucleic acid” is inclusive and may be in the form of either double-stranded or single-stranded nucleic acids, and a single strand can be either of the two complementing strands. The isolated ARTS1 nucleic acid can be naturally existing genomic DNA, mRNA or cDNA. In one embodiment, the isolated ARTS1 nucleic acid has a nucleotide sequence according to SEQ ID NO:30 but containing one or more exonic nucleotide variants of Tables 5-11 (e.g., EX2@397C and EX15@74A), or the complement thereof.

In another embodiment, the isolated ARTS1 nucleic acid has a nucleotide sequence that is at least 95%, preferably at least 97% and more preferably at least 99% identical to SEQ ID NO:30 but contains one or more exonic nucleotide variants of Tables 5-11 (e.g., EX2@397C and EX15@74A), or one or more nucleotide variants that will result in one or more amino acid variants of Tables 5-11, or the complement thereof.

In yet another embodiment, the isolated ARTS1 nucleic acid has a nucleotide sequence encoding ARTS1 protein having an amino acid sequence according to SEQ ID NO:31 but contains one or more amino acid variants of Tables 5-11 (e.g., EX2@397C and EX15@74A). Isolated ARTS1 nucleic acids having a nucleotide sequence that is the complement of the sequence are also encompassed by the present invention.

In yet another embodiment, the isolated ARTS1 nucleic acid has a nucleotide sequence encoding a ARTS1 protein having an amino acid sequence that is at least 95%, preferably at least 97% and more preferably at least 99% identical to SEQ ID NO:31 but contains one or more amino acid variants of Tables 5-11 (e.g., EX2@397C and EX15@74A), or the complement thereof.

The present invention also provides an isolated nucleic acid, naturally occurring or artificial, having a nucleotide sequence that is at least 95%, preferably at least 97% and more preferably at least 99% identical to SEQ ID NO:30 except for containing one or more nucleotide variants of Tables 5-11 (e.g., EX2@397C and EX15@74A), or the complement thereof.

In another embodiment, the present invention provides an isolated nucleic acid, naturally occurring or artificial, having a nucleotide sequence encoding a ARTS1 protein having an amino acid sequence according to SEQ ID NO:31 but containing one or more amino acid variants of Tables 5-11 (e.g., EX2@397C and EX15@74A). Isolated nucleic acids having a nucleotide sequence that is the complement of the sequence are also encompassed by the present invention.

In addition, isolated nucleic acids are also provided which have a nucleotide sequence encoding a protein having an amino acid sequence that is at least 95%, preferably at least 97% and more preferably at least 99% identical to SEQ ID NO:31 but containing one or more amino acid variants of Tables 5-11 (e.g., EX2@397C and EX15@74A), or the complement thereof.

Also encompassed are isolated ARTS1 nucleic acids obtainable by:

(a) providing a human genomic library;

(b) screening the genomic library using a probe having a nucleotide sequence according to any one of SEQ ID NOs:30, 32, 34-35;

(c) producing a genomic DNA comprising a contiguous span of at least 30 nucleotides of any one of SEQ ID NOs: 30, 32, 34-35, wherein the genomic DNA thus produced contains one or more of the SNPs of the present invention in Tables 5-11, such as EX1@−1125T, EX2@397C, EX20@1085G, EX15@74A, EX19@885T, EX20@2105C, EX20@719C and EX20@1038A.

The present invention also includes isolated ARTS1 nucleic acids obtainable by:

(i) providing a cDNA library using human mRNA from a human tissue, e.g., blood;

(ii) screening the cDNA library using a probe having a nucleotide sequence according to any one of SEQ ID Nos: 30, 32, 34-35; and

(iii) producing a cDNA DNA comprising a contiguous span of at least 30 nucleotides of any one of SEQ ID NOs: 30, 32, 34-35, wherein the cDNA thus produced contains one or more of the SNPs of the present invention in Tables 5-11, such as EX2@397C and EX15@74A.

The present invention also encompasses an isolated nucleic acid comprising the nucleotide sequence of a region of a ARTS1 genomic DNA or cDNA or mRNA, wherein the region contains one or more nucleotide variants as provided in Tables 5-11 (e.g., EX1@−1125T, EX2@397C, EX20@1085G, EX15@74A, EX19@885T, EX20@2105C, EX20@719C and EX20@1038A), or one or more nucleotide variants that will give rise to one or more amino acid variants of Tables 5-11, or the complement thereof. Such regions can be isolated and analyzed to efficiently detect the nucleotide variants of the present invention. Also, such regions can also be isolated and used as probes or primers in detection of the nucleotide variants of the present invention and other uses as will be clear from the descriptions below.

Thus, in one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of a ARTS1 nucleic acid, the contiguous span containing one or more nucleotide variants of Tables 5-11 (e.g., EX1@−1125T, EX2@397C, EX20@1085G, EX15@74A, EX19@885T, EX20@2105C, EX20@719C and EX20@1038A), or the complement thereof. In specific embodiments, the isolated nucleic acid are oligonucleotides having a contiguous span of from about 17, 18, 19, 20, 21, 22, 23 or 25 to about 30, 40 or 50, preferably from about 21 to about 30 nucleotide residues, of any ARTS1 nucleic acid, said contiguous span containing one or more nucleotide variants of Tables 5-11 (e.g., EX1@−1125T, EX2@397C, EX20@1085G, EX15@74A, EX19@885T, EX20@2105C, EX20@719C and EX20@1038A).

In one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of any one of SEQ ID NOs: 26, 28, 30-51, containing one or more nucleotide variants of Tables 5-11 (e.g., EX1@−1125T, EX2@397C, EX20@1085G, EX15@74A, EX19@885T, EX20@2105C, EX20@719C and EX20@1038A), or the complement thereof. In specific embodiments, the isolated nucleic acid comprises a nucleotide sequence according to any one of SEQ ID NOs: 30, 32, 34-35. In preferred embodiments, the isolated nucleic acid are oligonucleotides having a contiguous span of from about 17, 18, 19, 20, 21, 22, 23 or 25 to about 30, 40 or 50, preferably from about 21 to about 30 nucleotide residues, of any one of SEQ ID NOs: 30, 32, 34-35 and containing one or more nucleotide variants of Tables 5-11 (e.g., EX1@−1125T, EX2@397C, EX20@1085G, EX15@74A, EX19@885T, EX20@2105C, EX20@719C and EX20@1038A). The complements of the isolated nucleic acids are also encompassed by the present invention.

Thus, for example, an isolated nucleic acid of the present invention can have a contiguous span of at least 18, 19, 20, 21, 22, 23, 24, 25, 26, 27, 28, 29, or 30 or 40 or 50 nucleotide residues of SEQ IUD NO:34 containing the nucleotide variant EX1@−1125T (nucleotide residue No. 51 in SEQ ID NO:34), or a contiguous span of at least 18, 19, 20, 21, 22, 23, 24, 25, 26, 27, 28, 29, or 30 or 40 or 50 nucleotide residues of SEQ ID NO:35 containing the nucleotide variant EX2@397C (nucleotide residue No. 51 in SEQ ID NO:35), or a contiguous span of at least 18, 19, 20, 21, 22, 23, 24, 25, 26, 27, 28, 29, or 30 or 40 or 50 nucleotide residues of SEQ ID NO:52 containing the nucleotide variant, EX20@1085G (nucleotide residue No. 51 in SEQ ID NO:52), or a contiguous span of at least 18, 19, 20, 21, 22, 23, 24, 25, 26, 27, 28, 29, or 30 or 40 or 50 nucleotide residues of SEQ ID NO:45 containing the nucleotide variant EX15@74A (nucleotide residue Nos. 51, 52 and 53 in SEQ ID NO:45), or a contiguous span of at least 18, 19, 20, 21, 22, 23, 24, 25, 26, 27, 28, 29, or 30 or 40 or 50 nucleotide residues of SEQ ID NO:49 containing the nucleotide variant EX19@885T (nucleotide residue No. 51 in SEQ ID NO:49), or a contiguous span of at least 18, 19, 20, 21, 22, 23, 24, 25, 26, 27, 28, 29, or 30 or 40 or 50 nucleotide residues of SEQ ID NO:53 containing the nucleotide variant EX20@2105C (nucleotide residue No. 51 in SEQ ID NO:53), or a contiguous span of at least 18, 19, 20, 21, 22, 23, 24, 25, 26, 27, 28, 29, or 30 or 40 or 50 nucleotide residues of SEQ ID NO:50 containing the nucleotide variant EX20@719C (nucleotide residue No. 51 in SEQ ID NO:50), or a contiguous span of at least 18, 19, 20, 21, 22, 23, 24, 25, 26, 27, 28, 29, or 30 or 40 or 50 nucleotide residues of SEQ ID NO:51 containing the nucleotide variant EX20@1038A (nucleotide residue No. 51 in SEQ ID NO:51), or the complements thereof.

The present invention further provides an isolated MSR nucleic acid containing at least one of the newly discovered nucleotide variants as summarized in Table 12, or one or more nucleotide variants that will result in the amino acid variants provided in Table 12, e.g., EX1@−674G, EX1@19C, EX1@+129m, EX5@123T, EX10@+83G, EX11@+54C and EX14@14T. The term “MSR nucleic acid” is as defined above and means a naturally existing nucleic acid coding for a wild-type or variant or mutant MSR. The term “MSR nucleic acid” is inclusive and may be in the form of either double-stranded or single-stranded nucleic acids, and a single strand can be either of the two complementing strands. The isolated MSR nucleic acid can be naturally existing genomic DNA, mRNA or cDNA. In one embodiment, the isolated MSR nucleic acid has a nucleotide sequence according to SEQ ID NO:66 but containing one or more exonic nucleotide variants of Table 12 (e.g., EX5@123T and EX14@14T), or the complement thereof.

In another embodiment, the isolated MSR nucleic acid has a nucleotide sequence that is at least 95%, preferably at least 97% and more preferably at least 99% identical to SEQ ID NO:64 but contains one or more exonic nucleotide variants of Table 12 (e.g., EX5@123T and EX14@14T), or one or more nucleotide variants that will result in one or more amino acid variants of Table 12, or the complement thereof.

In yet another embodiment, the isolated MSR nucleic acid has a nucleotide sequence encoding MSR protein having an amino acid sequence according to SEQ ID NO:67 but contains one or more amino acid variants of Table 12 (e.g., EX14@14T). Isolated MSR nucleic acids having a nucleotide sequence that is the complement of the sequence are also encompassed by the present invention.

In yet another embodiment, the isolated MSR nucleic acid has a nucleotide sequence encoding a MSR protein having an amino acid sequence that is at least 95%, preferably at least 97% and more preferably at least 99% identical to SEQ ID NO:65 but contains one or more amino acid variants of Table 12 (e.g., EX14@14T), or the complement thereof.

The present invention also provides an isolated nucleic acid, naturally occurring or artificial, having a nucleotide sequence that is at least 95%, preferably at least 97% and more preferably at least 99% identical to SEQ ID NO:66 except for containing one or more nucleotide variants of Table 12 (e.g., EX5@123T and EX14@14T), or the complement thereof.

In another embodiment, the present invention provides an isolated nucleic acid, naturally occurring or artificial, having a nucleotide sequence encoding a MSR protein having an amino acid sequence according to SEQ ID NO:67 but containing one or more amino acid variants of Table 12 (e.g., EX14@14T). Isolated nucleic acids having a nucleotide sequence that is the complement of the sequence are also encompassed by the present invention.

In addition, isolated nucleic acids are also provided which have a nucleotide sequence encoding a protein having an amino acid sequence that is at least 95%, preferably at least 97% and more preferably at least 99% identical to SEQ ID NO:67 but containing one or more amino acid variants of Table 12 (e.g., EX14@14T), or the complement thereof.

Also encompassed are isolated MSR nucleic acids obtainable by:

(a) providing a human genomic library;

(b) screening the genomic library using a probe having a nucleotide sequence according to any one of SEQ ID NOs:66, 68, 70-81; and

(c) producing a genomic DNA comprising a contiguous span of at least 30 nucleotides of any one of SEQ ID NOs: 66, 68, 70-81, wherein the genomic DNA thus produced contains one or more of the SNPs of the present invention in Table 12, such as EX1@−674G, EX1@19C, EX1@+129m, EX5@123T, EX10@+83G, EX11@+54C and EX14@14T.

The present invention also includes isolated MSR nucleic acids obtainable by:

(i) providing a cDNA library using human mRNA from a human tissue, e.g., blood;

(ii) screening the cDNA library using a probe having a nucleotide sequence according to any one of SEQ ID NOs: 66, 68, 70-81; and

(iii) producing a cDNA DNA comprising a contiguous span of at least 30 nucleotides of any one of SEQ ID NOs: 66, 68, 70-81, wherein the cDNA thus produced contains one or more of the SNPs of the present invention in Table 12, such as EX5@123T and EX14@14T.

The present invention also encompasses an isolated nucleic acid comprising the nucleotide sequence of a region of a MSR genomic DNA or cDNA or mRNA, wherein the region contains one or more nucleotide variants as provided in Table 12 (e.g., EX1@−674G, EX1@19C, EX1@+129m, EX5@123T, EX10@+83G, EX11@+54C and EX14@14T), or one or more nucleotide variants that will give rise to one or more amino acid variants of Table 12, or the complement thereof. Such regions can be isolated and analyzed to efficiently detect the nucleotide variants of the present invention. Also, such regions can also be isolated and used as probes or primers in detection of the nucleotide variants of the present invention and other uses as will be clear from the descriptions below.

Thus, in one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of a MSR nucleic acid, the contiguous span containing one or more nucleotide variants of Table 12 (e.g., EX1@−674G, EX1@19C, EX1@+129m, EX5@123T, EX10@+83G, EX11@+54C and EX14@14T), or the complement thereof. In specific embodiments, the isolated nucleic acid are oligonucleotides having a contiguous span of from about 17, 18, 19, 20, 21, 22, 23 or 25 to about 30, 40 or 50, preferably from about 21 to about 30 nucleotide residues, of any MSR nucleic acid, said contiguous span containing one or more nucleotide variants of Table 12 (e.g., EX1@−674G, EX1@19C, EX1@+129m, EX5@123T, EX10@+83G, EX11@+54C and EX14@14T).

In one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of any one of SEQ ID NOs:70-81, containing one or more nucleotide variants of Table 12 (e.g., EX1@−674G, EX1@19C, EX1@+129m, EX5@123T, EX10@+83G, EX11@+54C and EX14@14T), or the complement thereof. In specific embodiments, the isolated nucleic acid comprises a nucleotide sequence according to any one of SEQ ID NOs:70-81. In preferred embodiments, the isolated nucleic acid are oligonucleotides having a contiguous span of from about 17, 18, 19, 20, 21, 22, 23 or 25 to about 30, 40 or 50, preferably from about 21 to about 30 nucleotide residues, of any one of SEQ ID NOs:70-81 and containing one or more nucleotide variants of Table 12 (e.g., EX1@−674G, EX1@19C, EX1@+129m, EX5@123T, EX10@+83G, EX11@+54C and EX14@14T). The complements of the isolated nucleic acids are also encompassed by the present invention.

For example, an isolated nucleic acid of the present invention can have a contiguous span of at least 18, 19, 20, 21, 22, 23, 24, 25, 26, 27, 28, 29, or 30 or 40 or 50 nucleotide residues of SEQ ID NO:70 containing the nucleotide variant EX1@−674G (nucleotide residue No. 51 in SEQ ID NO:70), or a contiguous span of at least 18, 19, 20, 21, 22, 23, 24, 25, 26, 27, 28, 29, or 30 or 40 or 50 nucleotide residues of SEQ ID NO:71 containing the nucleotide variant EX1@19C (nucleotide residue No. 51 in SEQ ID NO:71), or a contiguous span of at least 18, 19, 20, 21, 22, 23, 24, 25, 26, 27, 28, 29, or 30 or 40 or 50 nucleotide residues of SEQ ID NO:72 containing the nucleotide variant EX1@+129m (nucleotide residue No. 51 in SEQ ID NO:72), or a contiguous span of at least 18, 19, 20, 21, 22, 23, 24, 25, 26, 27, 28, 29, or 30 or 40 or 50 nucleotide residues of SEQ ID NO:73 containing the nucleotide variant EX5@123T (nucleotide residue Nos. 51 in SEQ ID NO:73), or a contiguous span of at least 18, 19, 20, 21, 22, 23, 24, 25, 26, 27, 28, 29, or 30 or 40 or 50 nucleotide residues of SEQ ID NO:76 containing the nucleotide variant EX10@+83G (nucleotide residue Nos. 51 in SEQ ID NO:76), or a contiguous span of at least 18, 19, 20, 21, 22, 23, 24, 25, 26, 27, 28, 29, or 30 or 40 or 50 nucleotide residues of SEQ ID NO:77 containing the nucleotide variant EX11@+54C (nucleotide residue Nos. 51 in SEQ ID NO:77), or a contiguous span of at least 18, 19, 20, 21, 22, 23, 24, 25, 26, 27, 28, 29, or 30 or 40 or 50 nucleotide residues of SEQ ID NO:78 containing the nucleotide variant EX14@14T (nucleotide residue Nos. 51 in SEQ ID NO:78), or the complements thereof.

The present invention further provides an isolated AKAP9 nucleic acid containing at least one of the newly discovered nucleotide variants as summarized in Table 13, or one or more nucleotide variants that will result in the amino acid variants provided in Table 13, e.g., EX10@186G, EX16@−59G, EX39@121T, EX40@470A, EX40@1055G and EX19@1101C. The term “AKAP9 nucleic acid” is as defined above and means a naturally existing nucleic acid coding for a wild-type or variant or mutant AKAP9. The term “AKAP9 nucleic acid” is inclusive and may be in the form of either double-stranded or single-stranded nucleic acids, and a single strand can be either of the two complementing strands. The isolated AKAP9 nucleic acid can be naturally existing genomic DNA, mRNA or cDNA. In one embodiment, the isolated AKAP9 nucleic acid has a nucleotide sequence according to SEQ ID NO:90 but containing one or more exonic nucleotide variants of Table 13 (e.g., EX10@186G and EX39@121T), or the complement thereof.

In another embodiment, the isolated AKAP9 nucleic acid has a nucleotide sequence that is at least 95%, preferably at least 97% and more preferably at least 99% identical to SEQ ID NO:90 but contains one or more exonic nucleotide variants of Tables 13 and 14 (e.g., EX10@186G and EX39@121T), or one or more nucleotide variants that will result in one or more amino acid variants of Tables 13 and/or 14, or the complement thereof.

In yet another embodiment, the isolated AKAP9 nucleic acid has a nucleotide sequence encoding AKAP9 protein having an amino acid sequence according to SEQ ID NO:91 but contains one or more amino acid variants of Table 13 and/or 14 (e.g., EX9@459T and EX35@215G). Isolated AKAP9 nucleic acids having a nucleotide sequence that is the complement of the sequence are also encompassed by the present invention.

In yet another embodiment, the isolated AKAP9 nucleic acid has a nucleotide sequence encoding a AKAP9 protein having an amino acid sequence that is at least 95%, preferably at least 97% and more preferably at least 99% identical to SEQ ID NO:91 but contains one or more amino acid variants of Table 13 and/or 14 (e.g., EX9@459T and EX35@215G), or the complement thereof.

The present invention also provides an isolated nucleic acid, naturally occurring or artificial, having a nucleotide sequence that is at least 95%, preferably at least 97% and more preferably at least 99% identical to SEQ ID NO:90 except for containing one or more nucleotide variants of Tables 13 and/or 14 (e.g., EX10@186G and EX39@121T), or the complement thereof.

In another embodiment, the present invention provides an isolated nucleic acid, naturally occurring or artificial, having a nucleotide sequence encoding a AKAP9 protein having an amino acid sequence according to SEQ ID NO:91 but containing one or more amino acid variants of Table 13 and/or 14 (e.g., EX9@459T and EX35@215G). Isolated nucleic acids having a nucleotide sequence that is the complement of the sequence are also encompassed by the present invention.

In addition, isolated nucleic acids are also provided which have a nucleotide sequence encoding a protein having an amino acid sequence that is at least 95%, preferably at least 97% and more preferably at least 99% identical to SEQ ID NO:91 but containing one or more amino acid variants of Table 13 and/or 14 (e.g., EX9@459T and EX35@215G), or the complement thereof.

Also encompassed are isolated AKAP9 nucleic acids obtainable by:

(a) providing a human genomic library;

(b) screening the genomic library using a probe having a nucleotide sequence according to any one of SEQ ID NOs:90, 92-94, 96-120; and

(c) producing a genomic DNA comprising a contiguous span of at least 30 nucleotides of any one of SEQ ID NOs: 90, 92-94, 96-120, wherein the genomic DNA thus produced contains one or more of the SNPs of the present invention in Tables 13 and/or 14, such as EX10@186G, EX16@−59G, EX39@121T, EX40@470A, EX40@1055G and EX19@1011C.

The present invention also includes AKAP9 nucleic acids obtainable by:

(i) providing a cDNA library using human mRNA from a human tissue, e.g., blood;

(ii) screening the cDNA library using a probe having a nucleotide sequence according to any one of SEQ ID NOs:90, 96-120; and

(iii) producing a cDNA DNA comprising a contiguous span of at least 30 nucleotides of any one of SEQ ID NOs: 90, 96-120, wherein the cDNA thus produced contains one or more of the SNPs of the present invention in Tables 13 and/or 14, such as EX10@186G and EX39@121T.

The present invention also encompasses an isolated nucleic acid comprising the nucleotide sequence of a region of a AKAP9 genomic DNA or cDNA or mRNA, wherein the region contains one or more nucleotide variants as provided in Tables 13 and 14 (e.g., EX10@186G, EX16@−59G, EX39@121T, EX40@470A, EX40@1055G and EX19@1011C), or one or more nucleotide variants that will give rise to one or more amino acid variants of Tables 13 and/or 14, or the complement thereof. Such regions can be isolated and analyzed to efficiently detect the nucleotide variants of the present invention. Also, such regions can also be isolated and used as probes or primers in detection of the nucleotide variants of the present invention and other uses as will be clear from the descriptions below.

Thus, in one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of a AKAP9 nucleic acid, the contiguous span containing one or more nucleotide variants of Tables 13 and/or 14 (e.g., EX10186G, EX16@−59G, EX39@121T, EX40@470A, EX40@1055G and EX19@1011C), or the complement thereof. In specific embodiments, the isolated nucleic acids are oligonucleotides having a contiguous span of from about 17, 18, 19, 20, 21, 22, 23 or 25 to about 30, 40 or 50, preferably from about 21 to about 30 nucleotide residues, of any AKAP9 nucleic acid, said contiguous span containing one or more nucleotide variants of Tables 13 and/or 14 (e.g., EX10@186G, EX16@−59G, EX39@121T, EX40@470A, EX40@1055G and EX19@1011C).

In one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of any one of SEQ ID NOs:96-120, containing one or more nucleotide variants of Tables 13 and 14 (e.g., EX10@186G, EX16@−59G, EX39@121T, EX40@470A, EX40@1055G and EX19@1011C), or the complement thereof. In specific embodiments, the isolated nucleic acid comprises a nucleotide sequence according to any one of SEQ ID NOs: 96-120. In preferred embodiments, the isolated nucleic acids are oligonucleotides having a contiguous span of from about 17, 18, 19, 20, 21, 22, 23 or 25 to about 30, 40 or 50, preferably from about 21 to about 30 nucleotide residues, of any one of SEQ ID NOs: 92-116 and containing one or more nucleotide variants of Tables 13 and/or 14 (e.g., EX10@186G, EX16@−59G, EX39@121T, EX40@470A, EX40@1055G and EX19@1011C). The complements of the isolated nucleic acids are also encompassed by the present invention.

Thus, for example, an isolated nucleic acid of the present invention can have a contiguous span of at least 18, 19, 20, 21, 22, 23, 24, 25, 26, 27, 28, 29, or 30 or 40 or 50 nucleotide residues of SEQ ID NO:100 containing the nucleotide variant EX10@186G (nucleotide residue No. 51 in SEQ ID NO:100), or a contiguous span of at least 18, 19, 20, 21, 22, 23, 24, 25, 26, 27, 28, 29, or 30 or 40 or 50 nucleotide residues of SEQ ID NO:102 containing the nucleotide variant EX1616-59G (nucleotide residue No. 51 in SEQ ID NO:102), or a contiguous span of at least 18, 19, 20, 21, 22, 23, 24, 25, 26, 27, 28, 29, or 30 or 40 or 50 nucleotide residues of SEQ ID NO:100 containing the nucleotide variant EX39@121T (nucleotide residue No. 110 in SEQ ID NO:106), or a contiguous span of at least 18, 19, 20, 21, 22, 23, 24, 25, 26, 27, 28, 29, or 30 or 40 or 50 nucleotide residues of SEQ ID NO:111 containing the nucleotide variant EX40@470A (nucleotide residue Nos. 51 in SEQ ID NO:1111), or a contiguous span of at least 18, 19, 20, 21, 22, 23, 24, 25, 26, 27, 28, 29, or 30 or 40 or 50 nucleotide residues of SEQ ID NO:113 containing the nucleotide variant EX40@1055G (nucleotide residue Nos. 51, 52 and 53 in SEQ ID NO:113), or a contiguous span of at least 18, 19, 20, 21, 22, 23, 24, 25, 26, 27, 28, 29, or 30 or 40 or 50 nucleotide residues of SEQ ID NO:117 containing the nucleotide variant EX19@1011 (nucleotide residue Nos. 51, 52 and 53 in SEQ ID NO:117), or the complements thereof.

The present invention further provides an isolated DNAJD1 nucleic acid containing at least one of the newly discovered nucleotide variants as summarized in Tables 15 and 16, or one or more nucleotide variants that will result in the amino acid variants provided in Tables 15 and/or 16, e.g., EX1@527G. The term “DNAJD1 nucleic acid” is as defined above and means a naturally existing nucleic acid coding for a wild-type or variant or mutant DNAJD1. The term “DNAJD1 nucleic acid” is inclusive and may be in the form of either double-stranded or single-stranded nucleic acids, and a single strand can be either of the two complementing strands. The isolated DNAJD1 nucleic acid can be naturally existing genomic DNA, mRNA or cDNA. In one embodiment, the isolated DNAJD1 nucleic acid has a nucleotide sequence according to SEQ ID NO:149 but containing one or more exonic nucleotide variants of Tables 15 and/or 16 (e.g., EX1@527G, and EX1@368T), or the complement thereof.

In another embodiment, the isolated DNAJD1 nucleic acid has a nucleotide sequence that is at least 95%, preferably at least 97% and more preferably at least 99% identical to SEQ ID NO:149 but contains one or more exonic nucleotide variants of Tables 15 and/or 16 (e.g., EX1@527G and EX1@368T), or one or more nucleotide variants that will result in one or more amino acid variants of Tables 15 and/or 16, or the complement thereof.

In yet another embodiment, the isolated DNAJD1 nucleic acid has a nucleotide sequence encoding DNAJD1 protein having an amino acid sequence according to SEQ ID NO:150 but contains one or more amino acid variants of Tables 15 and/or 16 (e.g., EX1@527G). Isolated DNAJD1 nucleic acids having a nucleotide sequence that is the complement of the sequence are also encompassed by the present invention.

In yet another embodiment, the isolated DNAJD1 nucleic acid has a nucleotide sequence encoding a DNAJD1 protein having an amino acid sequence that is at least 95%, preferably at least 97% and more preferably at least 99% identical to SEQ ID NO:150 but contains one or more amino acid variants of Tables 15 and/or 16 (e.g., EX1@527G), or the complement thereof.

The present invention also provides an isolated nucleic acid, naturally occurring or artificial, having a nucleotide sequence that is at least 95%, preferably at least 97% and more preferably at least 99% identical to SEQ ID NO:149 except for containing one or more nucleotide variants of Tables 15 and/or 16 (e.g., EX1@527G), or the complement thereof.

In another embodiment, the present invention provides an isolated nucleic acid, naturally occurring or artificial, having a nucleotide sequence encoding a DNAJD1 protein having an amino acid sequence according to SEQ ID NO:150 but containing one or more amino acid variants of Tables 15 and/or 16 (e.g., EX1@527G). Isolated nucleic acids having a nucleotide sequence that is the complement of the sequence are also encompassed by the present invention.

In addition, isolated nucleic acids are also provided which have a nucleotide sequence encoding a protein having an amino acid sequence that is at least 95%, preferably at least 97% and more preferably at least 99% identical to SEQ ID NO:150 but containing the amino acid variant of Tables 15 and/or 16 (e.g., EX1@527G), or the complement thereof.

Also encompassed are isolated DNAJD1 nucleic acids obtainable by:

(a) providing a human genomic library;

(b) screening the genomic library using a probe having a nucleotide sequence according to any one of SEQ ID NOs:149, 151-153; and

(c) producing a genomic DNA comprising a contiguous span of at least 30 nucleotides of any one of SEQ ID NOs:149, 151-153, wherein the genomic DNA thus produced contains one or more of the SNPs of the present invention in Tables 15 and/or 16, such as EX1@527G, EX1@368T and EX5@+72m.

The present invention also includes isolated DNAJD1 nucleic acids obtainable by:

(i) providing a cDNA library using human mRNA from a human tissue, e.g., blood;

(ii) screening the cDNA library using a probe having a nucleotide sequence according to any one of SEQ ID NOs: 149, 151-153; and

(iii) producing a cDNA DNA comprising a contiguous span of at least 30 nucleotides of any one of SEQ ID NOs: 149, 151-153, wherein the cDNA thus produced contains one or more of the SNPs of the present invention in Tables 15 and/or 16, such as EX1@527G, EX1@368T and EX5@+72m.

The present invention also encompasses an isolated nucleic acid comprising the nucleotide sequence of a region of a DNAJD1 genomic DNA or cDNA or mRNA, wherein the region contains one or more nucleotide variants as provided in Tables 15 and/or 16 (e.g., EX1@527G, EX1@368T and EX5@+72m), or one or more nucleotide variants that will give rise to one or more amino acid variants of Tables 15 and/or 16, or the complement thereof. Such regions can be isolated and analyzed to efficiently detect the nucleotide variants of the present invention. Also, such regions can also be isolated and used as probes or primers in detection of the nucleotide variants of the present invention and other uses as will be clear from the descriptions below.

Thus, in one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of a DNAJD1 nucleic acid, the contiguous span containing one or more nucleotide variants of Tables 15 and/or 16 (e.g., EX1@527G, EX1@368T and EX5@+72m), or the complement thereof. In specific embodiments, the isolated nucleic acid are oligonucleotides having a contiguous span of from about 17, 18, 19, 20, 21, 22, 23 or 25 to about 30, 40 or 50, preferably from about 21 to about 30 nucleotide residues, of any DNAJD1 nucleic acid, said contiguous span containing one or more nucleotide variants of Tables 15 and/or 16 (e.g., EX1@527G, EX1@368T and EX5@+72m).

In one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of any one of SEQ ID NOs:147-151, containing one or more nucleotide variants of Tables 15 and/or 16 (e.g., EX1@527G, EX1@368T and EX5@+72m), or the complement thereof. In specific embodiments, the isolated nucleic acid comprises a nucleotide sequence according to any one of SEQ ID NOs:151-153. In preferred embodiments, the isolated nucleic acid are oligonucleotides having a contiguous span of from about 17, 18, 19, 20, 21, 22, 23 or 25 to about 30, 40 or 50, preferably from about 21 to about 30 nucleotide residues, of any one of SEQ ID NOs: 151-153 and containing one or more nucleotide variants of Tables 15 and/or 16 (e.g., EX1@527G, EX1@368T and EX5@+72m). The complements of the isolated nucleic acids are also encompassed by the present invention.

Thus, for example, an isolated nucleic acid of the present invention can have a contiguous span of at least 18, 19, 20, 21, 22, 23, 24, 25, 26, 27, 28, 29, or 30 or 40 or 50 nucleotide residues of SEQ ID NO:151 containing the nucleotide variant EX1@368T (nucleotide residue No. 51 in SEQ ID NO:151), or a contiguous span of at least 18, 19, 20, 21, 22, 23, 24, 25, 26, 27, 28, 29, or 30 or 40 or 50 nucleotide residues of SEQ ID NO:152 containing the nucleotide variant EX1@527G (nucleotide residue No. 51 in SEQ ID NO:152), or a contiguous span of at least 18, 19, 20, 21, 22, 23, 24, 25, 26, 27, 28, 29, or 30 or 40 or 50 nucleotide residues of SEQ ID NO:153 containing the nucleotide variant EX1@+72m (nucleotide residue No. 51-53 in SEQ ID NO:153), or the complements thereof.

Accordingly, the present invention provides an isolated GOLPH4 nucleic acid containing at least one of the newly discovered nucleotide variants as summarized in Table 17, or one or more nucleotide variants that will result in the amino acid variants provided in Table 17, e.g., EX15@−85G, EX15@+86G, EX16@323A, EX16@737G. The term “GOLPH4 nucleic acid” is as defined above and means a naturally existing nucleic acid coding for a wild-type or variant or mutant GOLPH4. The term “GOLPH4 nucleic acid” is inclusive and may be in the form of either double-stranded or single-stranded nucleic acids, and a single strand can be either of the two complementing strands. The isolated GOLPH4 nucleic acid can be naturally existing genomic DNA, mRNA or cDNA. In one embodiment, the isolated GOLPH4 nucleic acid has a nucleotide sequence according to SEQ ID NO:153 but containing one or more exonic nucleotide variants of Tables 17 and/or 18 (e.g., EX15@−85G, EX15@+86G, EX16@323A, EX16@737G) or the complement thereof.

In yet another embodiment, the isolated GOLPH4 nucleic acid has a nucleotide sequence encoding GOLPH4 protein having an amino acid sequence according to SEQ ID NO:157 but contains one or more amino acid variants of Tables 17 and/or 18. Isolated GOLPH4 nucleic acids having a nucleotide sequence that is the complement of the sequence are also encompassed by the present invention.

The present invention also provides an isolated nucleic acid, naturally occurring or artificial, having a nucleotide sequence that is at least 95%, preferably at least 97% and more preferably at least 99% identical to SEQ ID NO:156 except for containing one or more nucleotide variants of Tables 17 and/or 18 (e.g. EX15@−85G, EX15@+86G, EX16@323A, EX16@737G), or the complement thereof.

Also encompassed are isolated GOLPH4 nucleic acids obtainable by:

(a) providing a human genomic library;

(b) screening the genomic library using a probe having a nucleotide sequence according to anyone of SEQ ID NOs:152, 154-159; and

(c) producing a genomic DNA comprising a contiguous span of at least 30 nucleotides of any one of SEQ ID NOs:152, 154-159, wherein the genomic DNA thus produced contains one or more of the SNPs of the present invention in Tables 17 and/or 18, such as EX15@−85G, EX15@+86G, EX16@323A, EX16@737G.

The present invention also includes isolated GOLPH4 nucleic acids obtainable by:

(i) providing a cDNA library using human mRNA from a human tissue, e.g., blood;

(ii) screening the cDNA library using a probe having a nucleotide sequence according to any one of SEQ ID NOs: 152, 154-159; and

(iii) producing a cDNA DNA comprising a contiguous span of at least 30 nucleotides of any one of SEQ ID NOs: 152, 154-159, wherein the cDNA thus produced contains one or more of the SNPs of the present invention in Tables 17 and/or 18, such as EX15@−85G, EX15@+86G, EX16@323A, EX16@737G.

The present invention also encompasses an isolated nucleic acid comprising the nucleotide sequence of a region of a GOLPH4 genomic DNA or cDNA or mRNA, wherein the region contains one or more nucleotide variants as provided in Tables 17 and/or 18 (e.g., EX15@−85G, EX15@+86G, EX16@323A, EX16@737G), or one or more nucleotide variants that will give rise to one or more amino acid variants of Tables 17 and/or 18, or the complement thereof. Such regions can be isolated and analyzed to efficiently detect the nucleotide variants of the present invention. Also, such regions can also be isolated and used as probes or primers in detection of the nucleotide variants of the present invention and other uses as will be clear from the descriptions below.

Thus, in one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of a GOLPH4 nucleic acid, the contiguous span containing one or more nucleotide variants of Tables 17 and/or 18 (e.g. EX15@−85G, EX15@+86G, EX16@323A, EX16@737G), or the complement thereof. In specific embodiments, the isolated nucleic acid are oligonucleotides having a contiguous span of from about 17, 18, 19, 20, 21, 22, 23 or 25 to about 30, 40 or 50, preferably from about 21 to about 30 nucleotide residues, of any GOLPH4 nucleic acid, said contiguous span containing one or more nucleotide variants of Tables 17 and/or 18 (e.g. EX15@−85G, EX15@+86G, EX16@323A, EX16@737G).

In one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of any one of SEQ ID NOs:158-167, containing one or more nucleotide variants of Tables 17 and/or 18 (e.g., EX15@−85G, EX15@+86G, EX16@323A, EX16@737G), or the complement thereof. In specific embodiments, the isolated nucleic acid comprises a nucleotide sequence according to any one of SEQ ID NOs: 158-167. In preferred embodiments, the isolated nucleic acid are oligonucleotides having a contiguous span of from about 17, 18, 19, 20, 21, 22, 23 or 25 to about 30, 40 or 50, preferably from about 21 to about 30 nucleotide residues, of any one of SEQ ID NOs: 158-167 and containing one or more nucleotide variants of Tables 17 and/or 18 (e.g., EX15@−85G, EX15@+86G, EX16@323A, EX16@737G). The complements of the isolated nucleic acids are also encompassed by the present invention.

Thus, for example, an isolated nucleic acid of the present invention can have a contiguous span of at least 18, 19, 20, 21, 22, 23, 24, 25, 26, 27, 28, 29, or 30 or 40 or 50 nucleotide residues of SEQ ID NO:159 containing the nucleotide variant EX15@−85G (nucleotide residue No. 51 in SEQ ID NO:159), or a contiguous span of at least 18, 19, 20, 21, 22, 23, 24, 25, 26, 27, 28, 29, or 30 or 40 or 50 nucleotide residues of SEQ ID NO:160 containing the nucleotide variant EX15@+86G (nucleotide residue No. 51 in SEQ ID NO:160), or a contiguous span of at least 18, 19, 20, 21, 22, 23, 24, 25, 26, 27, 28, 29, or 30 or 40 or 50 nucleotide residues of SEQ ID NO:161 containing the nucleotide variant EX16@323A (nucleotide residue No. 51 in SEQ ID NO:161), or a contiguous span of at least 18, 19, 20, 21, 22, 23, 24, 25, 26, 27, 28, 29, or 30 or 40 or 50 nucleotide residues of SEQ ID NO:162 containing the nucleotide variant EX16@737G (nucleotide residue Nos. 51, 52 and 53 in SEQ ID NO:162), or the complements thereof.

The present invention further provides an isolated RABEP1 nucleic acid containing at least one of the newly discovered nucleotide variants as summarized in Tables 19-22, or one or more nucleotide variants that will result in the amino acid variants provided in Tables 19-23, e.g., EX1@−511T, EX18@646m, EX18@690m, EX18@903m, EX18@1689m and EX18@2373m. The term “RABEP1 nucleic acid” is as defined above and means a naturally existing nucleic acid coding for a wild-type or variant or mutant RABEP1. The term “RABEP1 nucleic acid” is inclusive and may be in the form of either double-stranded or single-stranded nucleic acids, and a single strand can be either of the two complementing strands. The isolated RABEP1 nucleic acid can be naturally existing genomic DNA, mRNA or cDNA. In one embodiment, the isolated RABEP1 nucleic acid has a nucleotide sequence according to SEQ ID NO:170 but containing one or more exonic nucleotide variants of Tables 19-23 (e.g., EX1@73C, EX14@30C, EX17@15G, EX17@36C and EX17@87A), or the complement thereof.

In another embodiment, the isolated RABEP1 nucleic acid has a nucleotide sequence that is at least 95%, preferably at least 97% and more preferably at least 99% identical to SEQ ID NO:162 but contains one or more exonic nucleotide variants of Tables 19-23 (e.g., EX1@73C, EX14@30C, EX17@15G, EX17@36C and EX17@87A), or one or more nucleotide variants that will result in one or more amino acid variants of Tables 19-23, or the complement thereof.

The present invention also provides an isolated nucleic acid, naturally occurring or artificial, having a nucleotide sequence that is at least 95%, preferably at least 97% and more preferably at least 99% identical to SEQ ID NO:170 except for containing one or more nucleotide variants of Tables 19-23 (e.g., EX1@73C, EX14@30C, EX17@15G, EX17@36C and EX17@87A), or the complement thereof.

Also encompassed are isolated RABEP1 nucleic acids obtainable by:

(a) providing a human genomic library;

(b) screening the genomic library using a probe having a nucleotide sequence according to any one of SEQ ID NOs:170, 172-196; and

(c) producing a genomic DNA comprising a contiguous span of at least 30 nucleotides of any one of SEQ ID NOs:170, 172-196, wherein the genomic DNA thus produced contains one or more of the SNPs of the present invention in Tables 19-23, such as EX1@−551T, EX18@646m, EX18@690m, EX18@903m, EX18@1689m and EX18@2373m.

The present invention also includes isolated RABEP1 nucleic acids obtainable by:

(i) providing a cDNA library using human mRNA from a human tissue, e.g., blood;

(ii) screening the cDNA library using a probe having a nucleotide sequence according to any one of SEQ ID NOs:170, 172-196; and

(iii) producing a cDNA DNA comprising a contiguous span of at least 30 nucleotides of any one of SEQ ID NOs:170, 172-196, wherein the cDNA thus produced contains one or more of the SNPs of the present invention in Tables 19-23, such as EX1@173C, EX14@30C, EX17@15G, EX17@36C and EX17@87A.

The present invention also encompasses an isolated nucleic acid comprising the nucleotide sequence of a region of a RABEP1 genomic DNA or cDNA or mRNA, wherein the region contains one or more nucleotide variants as provided in Tables 19-23 (e.g., EX1@−551T, EX18@646m, EX18@690m, EX18@903m, EX18@1689m and EX18@2373m), or one or more nucleotide variants that will give rise to one or more amino acid variants of Tables 19-23, or the complement thereof. Such regions can be isolated and analyzed to efficiently detect the nucleotide variants of the present invention. Also, such regions can also be isolated and used as probes or primers in detection of the nucleotide variants of the present invention and other uses as will be clear from the descriptions below.

Thus, in one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of a RABEP1 nucleic acid, the contiguous span containing one or more nucleotide variants of Tables 19-23 (e.g., EX1@−551T, EX18@646m, EX18@690m, EX18@903m, EX18@1689m and EX18@2373m), or the complement thereof. In specific embodiments, the isolated nucleic acid are oligonucleotides having a contiguous span of from about 17, 18, 19, 20, 21, 22, 23 or 25 to about 30, 40 or 50, preferably from about 21 to about 30 nucleotide residues, of any RABEP1 nucleic acid, said contiguous span containing one or more nucleotide variants of Tables 19-23 (e.g., EX1@−551T, EX18@646m, EX18@690m, EX18@903m, EX18@1689m and EX18@2373m).

In one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of any one of SEQ ID NOs:172-196, containing one or more nucleotide variants of Tables 19-23 (e.g., EX1@−551T, EX18@646m, EX18@690m, EX18@903m, EX18@1689m and EX18@2373m), or the complement thereof. In specific embodiments, the isolated nucleic acid comprises a nucleotide sequence according to any one of SEQ ID NOs:172-196. In preferred embodiments, the isolated nucleic acid are oligonucleotides having a contiguous span of from about 17, 18, 19, 20, 21, 22, 23 or 25 to about 30, 40 or 50, preferably from about 21 to about 30 nucleotide residues, of any one of SEQ ID NOs:172-196 and containing one or more nucleotide variants of Tables 19-23 (e.g., EX1@−551T, EX18@646m, EX18@690m, EX18@903m, EX18@1689m and EX18@2373m). The complements of the isolated nucleic acids are also encompassed by the present invention.

Thus, for example, an isolated nucleic acid of the present invention can have a contiguous span of at least 18, 19, 20, 21, 22, 23, 24, 25, 26, 27, 28, 29, or 30 or 40 or 50 nucleotide residues of SEQ ID NO:172 containing the nucleotide variant EX1@−551T (nucleotide residue No. 51 in SEQ ID NO:172), or a contiguous span of at least 18, 19, 20, 21, 22, 23, 24, 25, 26, 27, 28, 29, or 30 or 40 or 50 nucleotide residues of SEQ ID NO:180 containing the nucleotide variant EX18@646m (nucleotide residue Nos. 51-69 in SEQ ID NO:180), or a contiguous span of at least 18, 19, 20, 21, 22, 23, 24, 25, 26, 27, 28, 29, or 30 or 40 or 50 nucleotide residues of SEQ ID NO:181 containing the nucleotide variant EX18@690m (nucleotide residue Nos. 51-55 in SEQ ID NO:181), or a contiguous span of at least 18, 19, 20, 21, 22, 23, 24, 25, 26, 27, 28, 29, or 30 or 40 or 50 nucleotide residues of SEQ ID NO:182 containing the nucleotide variant EX18@903m (nucleotide residue No. 51 in SEQ ID NO:182), or a contiguous span of at least 18, 19, 20, 21, 22, 23, 24, 25, 26, 27, 28, 29, or 30 or 40 or 50 nucleotide residues of SEQ ID NO:185 containing the nucleotide variant EX18@1689m (nucleotide residue Nos. 51-55 in SEQ ID NO:185), or a contiguous span of at least 18, 19, 20, 21, 22, 23, 24, 25, 26, 27, 28, 29, or 30 or 40 or 50 nucleotide residues of SEQ ID NO:189 containing the nucleotide variant EX18@2373m (nucleotide residue No. 51 in SEQ ID NO:189), or the complements thereof.

The present invention further provides an isolated TAP2 nucleic acid containing at least one of the newly discovered nucleotide variants as summarized in Table 24, or one or more nucleotide variants that will result in the amino acid variants provided in Tables 24 and/or 25, e.g., EX12@356T, EX12@358m and EX12@1132m. The term “TAP2 nucleic acid” is as defined above and means a naturally existing nucleic acid coding for a wild-type or variant or mutant TAP2. The term “TAP2 nucleic acid” is inclusive and may be in the form of either double-stranded or single-stranded nucleic acids, and a single strand can be either of the two complementing strands. The isolated TAP2 nucleic acid can be naturally existing genomic DNA, mRNA or cDNA. In one embodiment, the isolated TAP2 nucleic acid has a nucleotide sequence according to SEQ ID NO:202 but containing one or more exonic nucleotide variants of Tables 24 and/or 25 (e.g., EX11@17G, EX12@61G, EX12@127C and EX12@159T), or the complement thereof.

In another embodiment, the isolated TAP2 nucleic acid has a nucleotide sequence that is at least 95%, preferably at least 97% and more preferably at least 99% identical to SEQ ID NO:202 but contains one or more exonic nucleotide variants of Tables 24 and/or 25 (e.g., EX11@17G, EX12@61G, EX12@127C and EX12@159T), or one or more nucleotide variants that will result in one or more amino acid variants of Tables 24 and/or 25, or the complement thereof.

In yet another embodiment, the isolated TAP2 nucleic acid has a nucleotide sequence encoding TAP2 protein having an amino acid sequence according to SEQ ID NO:203 but contains one or more amino acid variants of Tables 24 and/or 25 (e.g., EX12@19T and EX12@61G). Isolated TAP2 nucleic acids having a nucleotide sequence that is the complement of the sequence are also encompassed by the present invention.

In yet another embodiment, the isolated TAP2 nucleic acid has a nucleotide sequence encoding a TAP2 protein having an amino acid sequence that is at least 95%, preferably at least 97% and more preferably at least 99% identical to SEQ ID NO:203 but contains one or more amino acid variants of Tables 24 and/or 25 (e.g., EX12@19T and EX12@61G), or the complement thereof.

The present invention also provides an isolated nucleic acid, naturally occurring or artificial, having a nucleotide sequence that is at least 95%, preferably at least 97% and more preferably at least 99% identical to SEQ ID NO:202 except for containing one or more nucleotide variants of Tables 24 and/or 25 (e.g., EX11@17G, EX12@61G, EX12@127C and EX12@159T), or the complement thereof.

In another embodiment, the present invention provides an isolated nucleic acid, naturally occurring or artificial, having a nucleotide sequence encoding a TAP2 protein having an amino acid sequence according to SEQ ID NO:203 but containing one or more amino acid variants of Tables 24 and/or 25 (e.g., EX12@19T and EX12@61G). Isolated nucleic acids having a nucleotide sequence that is the complement of the sequence are also encompassed by the present invention.

In addition, isolated nucleic acids are also provided which have a nucleotide sequence encoding a protein having an amino acid sequence that is at least 95%, preferably at least 97% and more preferably at least 99% identical to SEQ ID NO:203 but containing one or more amino acid variants of Tables 24 and/or 25 (e.g., EX12@19T and EX12@61G), or the complement thereof.

Also encompassed are isolated TAP2 nucleic acids obtainable by:

(a) providing a human genomic library;

(b) screening the genomic library using a probe having a nucleotide sequence according to any one of SEQ ID NOs:202, 204, 206-227; and

(c) producing a genomic DNA comprising a contiguous span of at least 30 nucleotides of any one of SEQ ID NOs: 202, 204, 206-227, wherein the genomic DNA thus produced contains one or more of the SNPs of the present invention in Tables 24 and/or 25, such as EX12@356T, EX12@358m and EX12@1132m.

The present invention also includes isolated TAP2 nucleic acids obtainable by:

(i) providing a cDNA library using human mRNA from a human tissue, e.g., blood;

(ii) screening the cDNA library using a probe having a nucleotide sequence according to any one of SEQ ID Nos: 202, 204, 206-227; and

(iii) producing a cDNA DNA comprising a contiguous span of at least 30 nucleotides of any one of SEQ ID NOs: 202, 204, 206-227, wherein the cDNA thus produced contains one or more of the SNPs of the present invention in Tables 24 and/or 25, such as EX12@356T, EX12@358m and EX12@1132m.

The present invention also encompasses an isolated nucleic acid comprising the nucleotide sequence of a region of a TAP2 genomic DNA or cDNA or mRNA, wherein the region contains one or more nucleotide variants as provided in Tables 24 and/or 25 (e.g., EX12@356T, EX12@358m and EX12@1132m), or one or more nucleotide variants that will give rise to one or more amino acid variants of Tables 24 and/or 25, or the complement thereof. Such regions can be isolated and analyzed to efficiently detect the nucleotide variants of the present invention. Also, such regions can also be isolated and used as probes or primers in detection of the nucleotide variants of the present invention and other uses as will be clear from the descriptions below.

Thus, in one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of a TAP2 nucleic acid, the contiguous span containing one or more nucleotide variants of Tables 24 and/or 25 (e.g., EX12@356T, EX12@358m and EX12@1132m), or the complement thereof. In specific embodiments, the isolated nucleic acid are oligonucleotides having a contiguous span of from about 17, 18, 19, 20, 21, 22, 23 or 25 to about 30, 40 or 50, preferably from about 21 to about 30 nucleotide residues, of any TAP2 nucleic acid, said contiguous span containing one or more nucleotide variants of Tables 24 and/or 25 (e.g., EX12@356T, EX12@358m and EX12@1132m).

In one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of any one of SEQ ID NOs:206-227, containing one or more nucleotide variants of Tables 24 and/or 25 (e.g., EX12@356T, EX12@358m and EX12@1132m), or the complement thereof. In specific embodiments, the isolated nucleic acid comprises a nucleotide sequence according to any one of SEQ ID NOs: 206-227. In preferred embodiments, the isolated nucleic acid are oligonucleotides having a contiguous span of from about 17, 18, 19, 20, 21, 22, 23 or 25 to about 30, 40 or 50, preferably from about 21 to about 30 nucleotide residues, of any one of SEQ ID NOs: 206-227 and containing one or more nucleotide variants of Tables 24 and/or 25 (e.g., EX12@356T, EX12@358m and EX12@1132m). The complements of the isolated nucleic acids are also encompassed by the present invention.

Thus, for example, an isolated nucleic acid of the present invention can have a contiguous span of at least 18, 19, 20, 21, 22, 23, 24, 25, 26, 27, 28, 29, or 30 or 40 or 50 nucleotide residues of SEQ ID NO:212 containing the nucleotide variant EX12@356T (nucleotide residue No. 51 in SEQ ID NO:212), or a contiguous span of at least 18, 19, 20, 21, 22, 23, 24, 25, 26, 27, 28, 29, or 30 or 40 or 50 nucleotide residues of SEQ ID NO:217 containing the nucleotide variant, EX12@358m (nucleotide residue No. 51-60 in SEQ ID NO:217), or a contiguous span of at least 18, 19, 20, 21, 22, 23, 24, 25, 26, 27, 28, 29, or 30 or 40 or 50 nucleotide residues of SEQ ID NO:225 containing the nucleotide variant and EX12@1132m (nucleotide residue No. 51-226 in SEQ ID NO:225), or the complements thereof.

The present invention further provides an isolated NARG2 nucleic acid containing at least one of the newly discovered nucleotide variants as summarized in Table 26, or one or more nucleotide variants that will result in the amino acid variants provided in Table 26, e.g., EX14@+15C, EX16@1757m, EX16@2306G, EX16@2547G and EX16@4025m. The term “NARG2 nucleic acid” is as defined above and means a naturally existing nucleic acid coding for a wild-type or variant or mutant NARG2. The term “NARG2 nucleic acid” is inclusive and may be in the form of either double-stranded or single-stranded nucleic acids, and a single strand can be either of the two complementing strands. The isolated NARG2 nucleic acid can be naturally existing genomic DNA, mRNA or cDNA. In one embodiment, the isolated NARG2 nucleic acid has a nucleotide sequence according to SEQ ID NO:230 but containing one or more exonic nucleotide variants of Table 26 (e.g., EX12@48C), or the complement thereof.

In another embodiment, the isolated NARG2 nucleic acid has a nucleotide sequence that is at least 95%, preferably at least 97% and more preferably at least 99% identical to SEQ ID NO:230 but contains one or more exonic nucleotide variants of Table 26 (e.g., EX12@48C), or one or more nucleotide variants that will result in one or more amino acid variants of Table 26, or the complement thereof.

In yet another embodiment, the isolated NARG2 nucleic acid has a nucleotide sequence encoding NARG2 protein having an amino acid sequence according to SEQ ID NO:231 but contains one or more amino acid variants of Table 26 (e.g., EX12@48C). Isolated NARG2 nucleic acids having a nucleotide sequence that is the complement of the sequence are also encompassed by the present invention.

The present invention also provides an isolated nucleic acid, naturally occurring or artificial, having a nucleotide sequence that is at least 95%, preferably at least 97% and more preferably at least 99% identical to SEQ ID NO:230 except for containing one or more nucleotide variants of Table 26 (e.g., EX12@48C), or the complement thereof.

Also encompassed are isolated NARG2 nucleic acids obtainable by:

(a) providing a human genomic library;

(b) screening the genomic library using a probe having a nucleotide sequence according to any one of SEQ ID NOs:230, 232-238; and

(c) producing a genomic DNA comprising a contiguous span of at least 30 nucleotides of any one of SEQ ID NOs: 230, 232-238, wherein the genomic DNA thus produced contains one or more of the SNPs of the present invention in Table 26, such as EX14@+15C, EX16@1757m, EX16@2306G, EX16@2547G and EX16@4025m.

The present invention also includes isolated NARG2 nucleic acids obtainable by:

(i) providing a cDNA library using human mRNA from a human tissue, e.g., blood;

(ii) screening the cDNA library using a probe having a nucleotide sequence according to any one of SEQ ID NOs 230, 232-238; and

(iii) producing a cDNA DNA comprising a contiguous span of at least 30 nucleotides of any one of SEQ ID NOs: 230, 232-238, wherein the cDNA thus produced contains one or more of the SNPs of the present invention in Table 26, such as EX12@48C.

The present invention also encompasses an isolated nucleic acid comprising the nucleotide sequence of a region of a NARG2 genomic DNA or cDNA or mRNA, wherein the region contains one or more nucleotide variants as provided in Table 26 (e.g., EX14@+15C, EX16@1757m, EX16@2306G, EX16@2547G and EX16@4025m), or one or more nucleotide variants that will give rise to one or more amino acid variants of Table 26, or the complement thereof. Such regions can be isolated and analyzed to efficiently detect the nucleotide variants of the present invention. Also, such regions can also be isolated and used as probes or primers in detection of the nucleotide variants of the present invention and other uses as will be clear from the descriptions below.

Thus, in one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of a NARG2 nucleic acid, the contiguous span containing one or more nucleotide variants of Table 26 (e.g., EX14@+15C, EX16@1757m, EX16@2306G, EX16@2547G and EX16@4025m), or the complement thereof. In specific embodiments, the isolated nucleic acid are oligonucleotides having a contiguous span of from about 17, 18, 19, 20, 21, 22, 23 or 25 to about 30, 40 or 50, preferably from about 21 to about 30 nucleotide residues, of any NARG2 nucleic acid, said contiguous span containing one or more nucleotide variants of Table 26 (e.g., EX14@+15C, EX16@1757m, EX16@2306G, EX16@2547G and EX16@4025m).

In one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of any one of SEQ ID NOs:232-238, containing one or more nucleotide variants of Table 26 (e.g., EX14@+15C, EX16@1757m, EX16@2306G, EX16@2547G and EX16@4025m), or the complement thereof. In specific embodiments, the isolated nucleic acid comprises a nucleotide sequence according to any one of SEQ ID NOs: 232-238. In preferred embodiments, the isolated nucleic acid are oligonucleotides having a contiguous span of from about 17, 18, 19, 20, 21, 22, 23 or 25 to about 30, 40 or 50, preferably from about 21 to about 30 nucleotide residues, of any one of SEQ ID NOs: 232-238 and containing one or more nucleotide variants of Table 26 (e.g., EX14@+15C, EX16@1757m, EX16@2306G, EX16@2547G and EX16@4025m). The complements of the isolated nucleic acids are also encompassed by the present invention.

Thus, for example, an isolated nucleic acid of the present invention can have a contiguous span of at least 18, 19, 20, 21, 22, 23, 24, 25, 26, 27, 28, 29, or 30 or 40 or 50 nucleotide residues of SEQ ID NO:234 containing the nucleotide variant EX14@+15C (nucleotide residue No. 51 in SEQ ID NO:234), or a contiguous span of at least 18, 19, 20, 21, 22, 23, 24, 25, 26, 27, 28, 29, or 30 or 40 or 50 nucleotide residues of SEQ ID NO:235 containing the nucleotide variant EX16@1757m (nucleotide residue No. 51 in SEQ ID NO:235), or a contiguous span of at least 18, 19, 20, 21, 22, 23, 24, 25, 26, 27, 28, 29, or 30 or 40 or 50 nucleotide residues of SEQ ID NO:237 containing the nucleotide variant EX16@2547G (nucleotide residue No. 51 in SEQ ID NO:237), or a contiguous span of at least 18, 19, 20, 21, 22, 23, 24, 25, 26, 27, 28, 29, or 30 or 40 or 50 nucleotide residues of SEQ ID NO:236 containing the nucleotide variant EX16@2306G (nucleotide residue Nos. 51, 52 and 53 in SEQ ID NO:236), or a contiguous span of at least 18, 19, 20, 21, 22, 23, 24, 25, 26, 27, 28, 29, or 30 or 40 or 50 nucleotide residues of SEQ ID NO:238 containing the nucleotide variant EX16@4025m (nucleotide residue Nos. 51, 52, 53, 54 and 55 in SEQ ID NO:238), or the complements thereof.

The present invention further provides an isolated DDX58 nucleic acid containing at least one of the newly discovered nucleotide variants as summarized in Table 27, or one or more nucleotide variants that will result in the amino acid variants provided in Table 27. The term “DDX58 nucleic acid” is as defined above and means a naturally existing nucleic acid coding for a wild-type or variant or mutant DDX58. The term “DDX58 nucleic acid” is inclusive and may be in the form of either double-stranded or single-stranded nucleic acids, and a single strand can be either of the two complementing strands. The isolated DDX58 nucleic acid can be naturally existing genomic DNA, mRNA or cDNA. In one embodiment, the isolated DDX58 nucleic acid has a nucleotide sequence according to SEQ ID NO:274 but containing one or more exonic nucleotide variants of Table 27 (e.g., EX17@63), or the complement thereof.

In another embodiment, the isolated DDX58 nucleic acid has a nucleotide sequence that is at least 95%, preferably at least 97% and more preferably at least 99% identical to SEQ ID NO:274 but contains one or more exonic nucleotide variants of Table 27, or one or more nucleotide variants that will result in one or more amino acid variants of Table 27, or the complement thereof.

In yet another embodiment, the isolated DDX58 nucleic acid has a nucleotide sequence encoding DDX58 protein having an amino acid sequence according to SEQ ID NO:275 but contains one or more amino acid variants of Table 27. Isolated DDX58 nucleic acids having a nucleotide sequence that is the complement of the sequence are also encompassed by the present invention.

In yet another embodiment, the isolated DDX58 nucleic acid has a nucleotide sequence encoding a DDX58 protein having an amino acid sequence that is at least 95%, preferably at least 97% and more preferably at least 99% identical to SEQ ID NO:275 but contains one or more amino acid variants of Table 27, or the complement thereof.

The present invention also provides an isolated nucleic acid, naturally occurring or artificial, having a nucleotide sequence that is at least 95%, preferably at least 97% and more preferably at least 99% identical to SEQ ID NO:274 except for containing one or more nucleotide variants of Table 27, or the complement thereof.

In another embodiment, the present invention provides an isolated nucleic acid, naturally occurring or artificial, having a nucleotide sequence encoding a DDX58 protein having an amino acid sequence according to SEQ ID NO:275 but containing one or more amino acid variants of Table 27. Isolated nucleic acids having a nucleotide sequence that is the complement of the sequence are also encompassed by the present invention.

In addition, isolated nucleic acids are also provided which have a nucleotide sequence encoding a protein having an amino acid sequence that is at least 95%, preferably at least 97% and more preferably at least 99% identical to SEQ ID NO:275 but containing one or more amino acid variants of Table 27, or the complement thereof.

Also encompassed are isolated DDX58 nucleic acids obtainable by:

(a) providing a human genomic library;

(b) screening the genomic library using a probe having a nucleotide sequence according to any one of SEQ ID NOs: 274, 276, 277; and

(c) producing a genomic DNA comprising a contiguous span of at least 30 nucleotides of any one of SEQ ID NOs: 274, 276, 277, wherein the genomic DNA thus produced contains one or more of the SNPs of the present invention in Table 27.

The present invention also includes isolated DDX58 nucleic acids obtainable by:

(i) providing a cDNA library using human mRNA from a human tissue, e.g., blood;

(ii) screening the cDNA library using a probe having a nucleotide sequence according to any one of SEQ ID NOs: 274, 276, 277; and

(iii) producing a cDNA DNA comprising a contiguous span of at least 30 nucleotides of any one of SEQ ID NOs: 274, 276, 277, wherein the cDNA thus produced contains one or more of the variants of the present invention in Table 27.

The present invention also encompasses an isolated nucleic acid comprising the nucleotide sequence of a region of a DDX58 genomic DNA or cDNA or mRNA, wherein the region contains one or more nucleotide variants as provided in Table 27, or one or more nucleotide variants that will give rise to one or more amino acid variants of Tables 27, or the complement thereof. Such regions can be isolated and analyzed to efficiently detect the nucleotide variants of the present invention. Also, such regions can also be isolated and used as probes or primers in detection of the nucleotide variants of the present invention and other uses as will be clear from the descriptions below.

Thus, in one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of a DDX58 nucleic acid, the contiguous span containing one or more nucleotide variants selected from those in Table 27, or the complement thereof. In specific embodiments, the isolated nucleic acids are oligonucleotides having a contiguous span of from about 17, 18, 19, 20, 21, 22, 23 or 25 to about 30, 40 or 50, preferably from about 21 to about 30 nucleotide residues, of any DDX58 nucleic acid, said contiguous span containing one or more nucleotide variants of Table 27.

In one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of any one of SEQ ID NOs:276-277, containing one or more nucleotide variants of Tables 27, or the complement thereof. In specific embodiments, the isolated nucleic acid comprises a nucleotide sequence according to any one of SEQ ID NOs:276-277. In preferred embodiments, the isolated nucleic acids are oligonucleotides having a contiguous span of from about 17, 18, 19, 20, 21, 22, 23 or 25 to about 30, 40 or 50, preferably from about 21 to about 30 nucleotide residues, of any one of SEQ ID NOs:276-277 and containing one or more nucleotide variants of Tables 27. The complements of the isolated nucleic acids are also encompassed by the present invention.

Thus, for example, an isolated nucleic acid of the present invention can have a contiguous span of at least 18, 19, 20, 21, 22, 23, 24, 25, 26, 27, 28, 29, or 30 or 40 or 50 nucleotide residues of SEQ ID NO:276 containing the nucleotide variant EX14@+78 (nucleotide residue No. 51 in SEQ ID NO:276), or a contiguous span of at least 18, 19, 20, 21, 22, 23, 24, 25, 26, 27, 28, 29, or 30 or 40 or 50 nucleotide residues of SEQ ID NO:277 containing the nucleotide variant EX17@63 (nucleotide residue No. 51 in SEQ ID NO:277, or the complements thereof.

The present invention further provides an isolated CD39 nucleic acid containing at least one of the newly discovered nucleotide variants as summarized in Table 28, or one or more nucleotide variants that will result in the amino acid variants provided in Tables 28 and/or 29. The term “CD39 nucleic acid” is as defined above and means a naturally existing nucleic acid coding for a wild-type or variant or mutant CD39. The term “CD39 nucleic acid” is inclusive and may be in the form of either double-stranded or single-stranded nucleic acids, and a single strand can be either of the two complementing strands. The isolated CD39 nucleic acid can be naturally existing genomic DNA, mRNA or cDNA. In one embodiment, the isolated CD39 nucleic acid has a nucleotide sequence according to SEQ ID NO:243 but containing one or more exonic nucleotide variants of Table 28 and/or 29 (e.g., EX17@63), or the complement thereof.

In another embodiment, the isolated CD39 nucleic acid has a nucleotide sequence that is at least 95%, preferably at least 97% and more preferably at least 99% identical to SEQ ID NO:243, but contains one or more exonic nucleotide variants of Table 28 and/or Table 29, or one or more nucleotide variants that will result in one or more amino acid variants of Table 28 and/or Table 29, or the complement thereof.

In yet another embodiment, the isolated CD39 nucleic acid has a nucleotide sequence encoding CD39 protein having an amino acid sequence according to SEQ ID NO:244 but contains one or more amino acid variants of Table 29 and/or Table 29. Isolated CD39 nucleic acids having a nucleotide sequence that is the complement of the sequence are also encompassed by the present invention.

In yet another embodiment, the isolated CD39 nucleic acid has a nucleotide sequence encoding a CD39 protein having an amino acid sequence that is at least 95%, preferably at least 97% and more preferably at least 99% identical to SEQ ID NO:244 but contains one or more amino acid variants of Table 28 and/or Table 29, or the complement thereof.

The present invention also provides an isolated nucleic acid, naturally occurring or artificial, having a nucleotide sequence that is at least 95%, preferably at least 97% and more preferably at least 99% identical to SEQ ID NO:243 except for containing one or more nucleotide variants of Table 28 and/or Table 29, or the complement thereof.

In another embodiment, the present invention provides an isolated nucleic acid, naturally occurring or artificial, having a nucleotide sequence encoding a CD39 protein having an amino acid sequence according to SEQ ID NO:244 but containing one or more amino acid variants of Table 28 and/or Table 29. Isolated nucleic acids having a nucleotide sequence that is the complement of the sequence are also encompassed by the present invention.

In addition, isolated nucleic acids are also provided which have a nucleotide sequence encoding a protein having an amino acid sequence that is at least 95%, preferably at least 97% and more preferably at least 99% identical to SEQ ID NO:244 but containing one or more amino acid variants of Table 28 and/or Table 29, or the complement thereof.

Also encompassed are isolated CD39 nucleic acids obtainable by:

(a) providing a human genomic library;

(b) screening the genomic library using a probe having a nucleotide sequence according to any one of SEQ ID NOs: 244, 246, 247; and

(c) producing a genomic DNA comprising a contiguous span of at least 30 nucleotides of any one of SEQ ID NOs: 244, 246, 247, wherein the genomic DNA thus produced contains one or more of the SNPs of the present invention in Table 28 and/or Table 29.

The present invention also includes isolated CD39 nucleic acids obtainable by:

(i) providing a cDNA library using human mRNA from a human tissue, e.g., blood;

(ii) screening the cDNA library using a probe having a nucleotide sequence according to any one of SEQ ID NOs: 244, 246, 247; and

(iii) producing a cDNA DNA comprising a contiguous span of at least 30 nucleotides of any one of SEQ ID NOs: 244, 246, 247, wherein the cDNA thus produced contains one or more of the variants of the present invention in Table 28 and/or Table 29.

The present invention also encompasses an isolated nucleic acid comprising the nucleotide sequence of a region of a CD39 genomic DNA or cDNA or mRNA, wherein the region contains one or more nucleotide variants as provided in Tables 1 and 2, or one or more nucleotide variants that will give rise to one or more amino acid variants of Table 28 and/or Table 29, or the complement thereof. Such regions can be isolated and analyzed to efficiently detect the nucleotide variants of the present invention. Also, such regions can also be isolated and used as probes or primers in detection of the nucleotide variants of the present invention and other uses as will be clear from the descriptions below.

Thus, in one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of a CD39 nucleic acid, the contiguous span containing one or more nucleotide variants selected from those in Tables 1 and 2, or the complement thereof. In specific embodiments, the isolated nucleic acids are oligonucleotides having a contiguous span of from about 17, 18, 19, 20, 21, 22, 23 or 25 to about 30, 40 or 50, preferably from about 21 to about 30 nucleotide residues, of any CD39 nucleic acid, said contiguous span containing one or more nucleotide variants of Table 28 and/or Table 29.

In one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of any one of SEQ ID NOs:245-246, containing one or more nucleotide variants of Table 28 and/or Table 29, or the complement thereof. In specific embodiments, the isolated nucleic acid comprises a nucleotide sequence according to any one of SEQ ID NOs: 245-246. In preferred embodiments, the isolated nucleic acids are oligonucleotides having a contiguous span of from about 17, 18, 19, 20, 21, 22, 23 or 25 to about 30, 40 or 50, preferably from about 21 to about 30 nucleotide residues, of any one of SEQ ID NOs: 245-246 and containing one or more nucleotide variants of Table 28 and/or Table 29. The complements of the isolated nucleic acids are also encompassed by the present invention.

Thus, for example, an isolated nucleic acid of the present invention can have a contiguous span of at least 18, 19, 20, 21, 22, 23, 24, 25, 26, 27, 28, 29, or 30 or 40 or 50 nucleotide residues of SEQ ID NO:245 containing the nucleotide variant EX4@−10 (nucleotide residue No. 51 in SEQ ID NO:245), or a contiguous span of at least 18, 19, 20, 21, 22, 23, 24, 25, 26, 27, 28, 29, or 30 or 40 or 50 nucleotide residues of SEQ ID NO:246 containing the nucleotide variant EX10@3061 (nucleotide residue No. 51 in SEQ ID NO:246), or the complements thereof.

Accordingly, the present invention provides an isolated FKBP1a nucleic acid containing at least one of the newly discovered nucleotide variants as summarized in Table 30, or one or more nucleotide variants that will result in the amino acid variants provided in Table 30. The term “FKBP1a nucleic acid” is as defined above and means a naturally existing nucleic acid coding for a wild-type or variant or mutant FKBP1a. The term “FKBP1a nucleic acid” is inclusive and may be in the form of either double-stranded or single-stranded nucleic acids, and a single strand can be either of the two complementing strands. The isolated FKBP1a nucleic acid can be naturally existing genomic DNA, mRNA or cDNA. In one embodiment, the isolated FKBP1a nucleic acid has a nucleotide sequence according to SEQ ID NO:249 but containing one or more exonic nucleotide variants of Table 30 (e.g., EX5@8A), or the complement thereof.

In another embodiment, the isolated FKBP1a nucleic acid has a nucleotide sequence that is at least 95%, preferably at least 97% and more preferably at least 99% identical to SEQ ID NO:249 but contains one or more exonic nucleotide variants of Table 30, or one or more nucleotide variants that will result in one or more amino acid variants of Table 30, or the complement thereof.

In yet another embodiment, the isolated FKBP1a nucleic acid has a nucleotide sequence encoding FKBP1a protein having an amino acid sequence according to SEQ ID NO:250 but contains one or more amino acid variants of Table 30. Isolated FKBP1a nucleic acids having a nucleotide sequence that is the complement of the sequence are also encompassed by the present invention.

In yet another embodiment, the isolated FKBP1a nucleic acid has a nucleotide sequence encoding a FKBP1a protein having an amino acid sequence that is at least 95%, preferably at least 97% and more preferably at least 99% identical to SEQ ID NO:250 but contains one or more amino acid variants of Table 30, or the complement thereof.

The present invention also provides an isolated nucleic acid, naturally occurring or artificial, having a nucleotide sequence that is at least 95%, preferably at least 97% and more preferably at least 99% identical to SEQ ID NO:249 except for containing one or more nucleotide variants of Table 30, or the complement thereof.

In another embodiment, the present invention provides an isolated nucleic acid, naturally occurring or artificial, having a nucleotide sequence encoding a FKBP1a protein having an amino acid sequence according to SEQ ID NO:250 but containing one or more amino acid variants of Table 30. Isolated nucleic acids having a nucleotide sequence that is the complement of the sequence are also encompassed by the present invention.

In addition, isolated nucleic acids are also provided which have a nucleotide sequence encoding a protein having an amino acid sequence that is at least 95%, preferably at least 97% and more preferably at least 99% identical to SEQ ID NO:250 but containing one or more amino acid variants of Table 30, or the complement thereof.

Also encompassed are isolated FKBP1a nucleic acids obtainable by:

(a) providing a human genomic library;

(b) screening the genomic library using a probe having a nucleotide sequence according to any one of SEQ ID NOs:249, 251; and

(c) producing a genomic DNA comprising a contiguous span of at least 30 nucleotides of any one of SEQ ID NOs: 249, 251, wherein the genomic DNA thus produced contains one or more of the SNPs of the present invention in Table 30.

The present invention also includes isolated FKBP1a nucleic acids obtainable by:

(i) providing a cDNA library using human mRNA from a human tissue, e.g., blood;

(ii) screening the cDNA library using a probe having a nucleotide sequence according to any one of SEQ ID NOs:249, 251; and

(iii) producing a cDNA DNA comprising a contiguous span of at least 30 nucleotides of any one of SEQ ID NOs:249, 251, wherein the cDNA thus produced contains one or more of the variants of the present invention in Table 30.

The present invention also encompasses an isolated nucleic acid comprising the nucleotide sequence of a region of a FKBP1a genomic DNA or cDNA or mRNA, wherein the region contains one or more nucleotide variants as provided in Table 30, or one or more nucleotide variants that will give rise to one or more amino acid variants of Table 30, or the complement thereof. Such regions can be isolated and analyzed to efficiently detect the nucleotide variants of the present invention. Also, such regions can also be isolated and used as probes or primers in detection of the nucleotide variants of the present invention and other uses as will be clear from the descriptions below.

Thus, in one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of a FKBP1a nucleic acid, the contiguous span containing one or more nucleotide variants selected from those in Table 30, or the complement thereof. In specific embodiments, the isolated nucleic acids are oligonucleotides having a contiguous span of from about 17, 18, 19, 20, 21, 22, 23 or 25 to about 30, 40 or 50, preferably from about 21 to about 30 nucleotide residues, of any FKBP1a nucleic acid, said contiguous span containing one or more nucleotide variants of Table 30.

In one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of any one of SEQ ID NO:251, containing one or more nucleotide variants of Table 30, or the complement thereof. In specific embodiments, the isolated nucleic acid comprises a nucleotide sequence according to any one of SEQ ID NO:251. In preferred embodiments, the isolated nucleic acids are oligonucleotides having a contiguous span of from about 17, 18, 19, 20, 21, 22, 23 or 25 to about 30, 40 or 50, preferably from about 21 to about 30 nucleotide residues, of any one of SEQ ID NO:251 and containing one or more nucleotide variants of Table 30. The complements of the isolated nucleic acids are also encompassed by the present invention.

Thus, for example, an isolated nucleic acid of the present invention can have a contiguous span of at least 18, 19, 20, 21, 22, 23, 24, 25, 26, 27, 28, 29, or 30 or 40 or 50 nucleotide residues of SEQ ID NO:251 containing the nucleotide variant EX5@8A (nucleotide residue No. 51 in SEQ ID NO:251), or the complements thereof.

The present invention further provides an isolated SRI nucleic acid containing at least one of the newly discovered nucleotide variants as summarized in Tables 1 and 2, or one or more nucleotide variants that will result in the amino acid variants provided in Table 31. The term “SRI nucleic acid” is as defined above and means a naturally existing nucleic acid coding for a wild-type or variant or mutant SRI. The term “SRI nucleic acid” is inclusive and may be in the form of either double-stranded or single-stranded nucleic acids, and a single strand can be either of the two complementing strands. The isolated SRI nucleic acid can be naturally existing genomic DNA, mRNA or cDNA. In one embodiment, the isolated SRI nucleic acid has a nucleotide sequence according to SEQ ID NO:253 but containing one or more exonic nucleotide variants of Table 31 (e.g., EX9@351), or the complement thereof.

In another embodiment, the isolated SRI nucleic acid has a nucleotide sequence that is at least 95%, preferably at least 97% and more preferably at least 99% identical to SEQ ID NO:253 but contains one or more exonic nucleotide variants of Table 31, or one or more nucleotide variants that will result in one or more amino acid variants of Table 31, or the complement thereof.

In yet another embodiment, the isolated SRI nucleic acid has a nucleotide sequence encoding SRI protein having an amino acid sequence according to SEQ ID NO:254 but contains one or more amino acid variants of Table 31. Isolated SRI nucleic acids having a nucleotide sequence that is the complement of the sequence are also encompassed by the present invention.

In yet another embodiment, the isolated SRI nucleic acid has a nucleotide sequence encoding a SRI protein having an amino acid sequence that is at least 95%, preferably at least 97% and more preferably at least 99% identical to SEQ ID NO:254 but contains one or more amino acid variants of Table 31, or the complement thereof.

The present invention also provides an isolated nucleic acid, naturally occurring or artificial, having a nucleotide sequence that is at least 95%, preferably at least 97% and more preferably at least 99% identical to SEQ ID NO:253 except for containing one or more nucleotide variants of Table 31, or the complement thereof.

In another embodiment, the present invention provides an isolated nucleic acid, naturally occurring or artificial, having a nucleotide sequence encoding a SRI protein having an amino acid sequence according to SEQ ID NO:254 but containing one or more amino acid variants of Table 31. Isolated nucleic acids having a nucleotide sequence that is the complement of the sequence are also encompassed by the present invention.

In addition, isolated nucleic acids are also provided which have a nucleotide sequence encoding a protein having an amino acid sequence that is at least 95%, preferably at least 97% and more preferably at least 99% identical to SEQ ID NO:254 but containing one or more amino acid variants of Table 31, or the complement thereof.

Also encompassed are isolated SRI nucleic acids obtainable by:

(a) providing a human genomic library;

(b) screening the genomic library using a probe having a nucleotide sequence according to any one of SEQ ID NO:253, 255; and

(c) producing a genomic DNA comprising a contiguous span of at least 30 nucleotides of any one of SEQ ID NOs:253, 255, wherein the genomic DNA thus produced contains one or more of the SNPs of the present invention in Table 31.

The present invention also includes isolated SRI nucleic acids obtainable by:

(i) providing a cDNA library using human mRNA from a human tissue, e.g., blood;

(ii) screening the cDNA library using a probe having a nucleotide sequence according to any one of SEQ ID NO:253, 255; and

(iii) producing a cDNA DNA comprising a contiguous span of at least 30 nucleotides of any one of SEQ ID NO:253, 255, wherein the cDNA thus produced contains one or more of the variants of the present invention in Table 31.

The present invention also encompasses an isolated nucleic acid comprising the nucleotide sequence of a region of a SRI genomic DNA or cDNA or mRNA, wherein the region contains one or more nucleotide variants as provided in Table 31, or one or more nucleotide variants that will give rise to one or more amino acid variants of Table 31, or the complement thereof. Such regions can be isolated and analyzed to efficiently detect the nucleotide variants of the present invention. Also, such regions can also be isolated and used as probes or primers in detection of the nucleotide variants of the present invention and other uses as will be clear from the descriptions below.

Thus, in one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of a SRI nucleic acid, the contiguous span containing one or more nucleotide variants selected from those in Table 31, or the complement thereof. In specific embodiments, the isolated nucleic acids are oligonucleotides having a contiguous span of from about 17, 18, 19, 20, 21, 22, 23 or 25 to about 30, 40 or 50, preferably from about 21 to about 30 nucleotide residues, of any SRI nucleic Table 31.

In one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of SEQ ID NO: 255, containing one or more nucleotide variants of Table 31, or the complement thereof. In specific embodiments, the isolated nucleic acid comprises a nucleotide sequence according to SEQ ID NO:253, 255. In preferred embodiments, the isolated nucleic acids are oligonucleotides having a contiguous span of from about 17, 18, 19, 20, 21, 22, 23 or 25 to about 30, 40 or 50, preferably from about 21 to about 30 nucleotide residues, of SEQ ID NO:253, 255 and containing one or more nucleotide variants of Table 31. The complements of the isolated nucleic acids are also encompassed by the present invention.

Thus, for example, an isolated nucleic acid of the present invention can have a contiguous span of at least 18, 19, 20, 21, 22, 23, 24, 25, 26, 27, 28, 29, or 30 or 40 or 50 nucleotide residues of SEQ ID NO:255 containing the nucleotide variant EX9@351C (nucleotide residue No. 51 in SEQ ID NO:255), or the complements thereof.

Accordingly, the present invention provides an isolated XRRA1 nucleic acid containing at least one of the newly discovered nucleotide variants as summarized in Table 32, or one or more nucleotide variants that will result in the amino acid variants provided in Table 32. The term “XRRA1 nucleic acid” is as defined above and means a naturally existing nucleic acid coding for a wild-type or variant or mutant XRRA1. The term “XRRA1 nucleic acid” is inclusive and may be in the form of either double-stranded or single-stranded nucleic acids, and a single strand can be either of the two complementing strands. The isolated XRRA1 nucleic acid can be naturally existing genomic DNA, mRNA or cDNA. In one embodiment, the isolated XRRA1 nucleic acid has a nucleotide sequence according to SEQ ID NO:257 but containing one or more exonic nucleotide variants of Table 32 (e.g., EX2@26, EX11@51 and EX13@62), or the complement thereof.

In another embodiment, the isolated XRRA1 nucleic acid has a nucleotide sequence that is at least 95%, preferably at least 97% and more preferably at least 99% identical to SEQ ID NO:257 but contains one or more exonic nucleotide variants of Table 32, or one or more nucleotide variants that will result in one or more amino acid variants of Table 32, or the complement thereof.

In yet another embodiment, the isolated XRRA1 nucleic acid has a nucleotide sequence encoding XRRA1 protein having an amino acid sequence according to SEQ ID NO:258 but contains one or more amino acid variants of Table 32. Isolated XRRA1 nucleic acids having a nucleotide sequence that is the complement of the sequence are also encompassed by the present invention.

In yet another embodiment, the isolated XRRA1 nucleic acid has a nucleotide sequence encoding a XRRA1 protein having an amino acid sequence that is at least 95%, preferably at least 97% and more preferably at least 99% identical to SEQ ID NO:258 but contains one or more amino acid variants of Table 32, or the complement thereof.

The present invention also provides an isolated nucleic acid, naturally occurring or artificial, having a nucleotide sequence that is at least 95%, preferably at least 97% and more preferably at least 99% identical to SEQ ID NO:257 except for containing one or more nucleotide variants of Table 32, or the complement thereof.

In another embodiment, the present invention provides an isolated nucleic acid, naturally occurring or artificial, having a nucleotide sequence encoding a XRRA1 protein having an amino acid sequence according to SEQ ID NO:258 but containing one or more amino acid variants of Table 32. Isolated nucleic acids having a nucleotide sequence that is the complement of the sequence are also encompassed by the present invention.

In addition, isolated nucleic acids are also provided which have a nucleotide sequence encoding a protein having an amino acid sequence that is at least 95%, preferably at least 97% and more preferably at least 99% identical to SEQ ID NO:258 but containing one or more amino acid variants of Table 32, or the complement thereof.

Also encompassed are isolated XRRA1 nucleic acids obtainable by:

(a) providing a human genomic library;

(b) screening the genomic library using a probe having a nucleotide sequence according to any one of SEQ ID NOs:257, 259-263; and

(c) producing a genomic DNA comprising a contiguous span of at least 30 nucleotides of any one of SEQ ID NOs:257, 259-263, wherein the genomic DNA thus produced contains one or more of the SNPs of the present invention in Table 32.

The present invention also includes isolated XRRA1 nucleic acids obtainable by:

(i) providing a cDNA library using human mRNA from a human tissue, e.g., blood;

(ii) screening the cDNA library using a probe having a nucleotide sequence according to any one of SEQ ID NOs:257, 259-263; and

(iii) producing a cDNA DNA comprising a contiguous span of at least 30 nucleotides of any one of SEQ ID NOs:257, 259-263, wherein the cDNA thus produced contains one or more of the variants of the present invention in Table 32.

The present invention also encompasses an isolated nucleic acid comprising the nucleotide sequence of a region of a XRRA1 genomic DNA or cDNA or mRNA, wherein the region contains one or more nucleotide variants as provided in Table 32, or one or more nucleotide variants that will give rise to one or more amino acid variants of Table 32, or the complement thereof. Such regions can be isolated and analyzed to efficiently detect the nucleotide variants of the present invention. Also, such regions can also be isolated and used as probes or primers in detection of the nucleotide variants of the present invention and other uses as will be clear from the descriptions below.

Thus, in one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of a XRRA1 nucleic acid, the contiguous span containing one or more nucleotide variants selected from those in Table 32, or the complement thereof. In specific embodiments, the isolated nucleic acids are oligonucleotides having a contiguous span of from about 17, 18, 19, 20, 21, 22, 23 or 25 to about 30, 40 or 50, preferably from about 21 to about 30 nucleotide residues, of any XRRA1 nucleic acid, said contiguous span containing one or more nucleotide variants of Table 32.

In one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of any one of SEQ ID NOs:259-263, containing one or more nucleotide variants of Table 32, or the complement thereof. In specific embodiments, the isolated nucleic acid comprises a nucleotide sequence according to any one of SEQ ID NOs:259-263. In preferred embodiments, the isolated nucleic acids are oligonucleotides having a contiguous span of from about 17, 18, 19, 20, 21, 22, 23 or 25 to about 30, 40 or 50, preferably from about 21 to about 30 nucleotide residues, of any one of SEQ ID NOs:259-263 and containing one or more nucleotide variants of Table 32. The complements of the isolated nucleic acids are also encompassed by the present invention.

Thus, for example, an isolated nucleic acid of the present invention can have a contiguous span of at least 18, 19, 20, 21, 22, 23, 24, 25, 26, 27, 28, 29, or 30 or 40 or 50 nucleotide residues of SEQ ID NO:259 containing the nucleotide variant EX2@26C (nucleotide residue No. 51 in SEQ ID NO:259), or a contiguous span of at least 18, 19, 20, 21, 22, 23, 24, 25, 26, 27, 28, 29, or 30 or 40 or 50 nucleotide residues of SEQ ID NO:261 containing the nucleotide variant EX11@51C (nucleotide residue No. 51 in SEQ ID NO:261), or a contiguous span of at least 18, 19, 20, 21, 22, 23, 24, 25, 26, 27, 28, 29, or 30 or 40 or 50 nucleotide residues of SEQ ID NO:262 containing the nucleotide variant EX13@62C (nucleotide residue No. 51 in SEQ ID NO:262), or the complements thereof.

The present invention provides an isolated IRF5 nucleic acid containing at least one of the newly discovered nucleotide variants as summarized in Table 33, or one or more nucleotide variants that will result in the amino acid variants provided in Table 1, e.g., EX1@−82m and EX6@91m. The term “IRF5 nucleic acid” is as defined above and means a naturally existing nucleic acid coding for a wild-type or variant or mutant IRF5. The term “IRF5 nucleic acid” is inclusive and may be in the form of either double-stranded or single-stranded nucleic acids, and a single strand can be either of the two complementing strands. The isolated IRF5 nucleic acid can be naturally existing genomic DNA, mRNA or cDNA. In one embodiment, the isolated IRF5 nucleic acid has a nucleotide sequence according to SEQ ID NO:280 but containing one or more exonic nucleotide variants of Table 33 (e.g., EX6@91), or the complement thereof.

In another embodiment, the isolated IRF5 nucleic acid has a nucleotide sequence that is at least 95%, preferably at least 97% and more preferably at least 99% identical to SEQ ID NO:280 but contains one or more exonic nucleotide variants of Table 33 (e.g., EX6@91), or one or more nucleotide variants that will result in one or more amino acid variants of Table 33, or the complement thereof.

The present invention also provides an isolated nucleic acid, naturally occurring or artificial, having a nucleotide sequence that is at least 95%, preferably at least 97% and more preferably at least 99% identical to SEQ ID NO:280 except for containing one or more nucleotide variants of Table 33 (e.g., EX6@91), or the complement thereof.

Also encompassed are isolated IRF5 nucleic acids obtainable by:

(a) providing a human genomic library;

(b) screening the genomic library using a probe having a nucleotide sequence according to any one of SEQ ID NOs:280, 282-290; and

(c) producing a genomic DNA comprising a contiguous span of at least 30 nucleotides of any one of SEQ ID NOs:280, 282-290, wherein the genomic DNA thus produced contains one or more of the SNPs of the present invention in Table 33, such as EX1@−82m and EX6@91m.

The present invention also includes isolated IRF5 nucleic acids obtainable by:

(i) providing a cDNA library using human mRNA from a human tissue, e.g., blood;

(ii) screening the cDNA library using a probe having a nucleotide sequence according to any one of SEQ ID NOs:280, 282-290; and

(iii) producing a cDNA DNA comprising a contiguous span of at least 30 nucleotides of any one of SEQ ID NOs:280, 282-290, wherein the cDNA thus produced contains one or more of the SNPs of the present invention in Table 33, such as EX6@91.

The present invention also encompasses an isolated nucleic acid comprising the nucleotide sequence of a region of a IRF5 genomic DNA or cDNA or mRNA, wherein the region contains one or more nucleotide variants as provided in Table 33 (e.g., EX1@−82m and EX6@91m), or one or more nucleotide variants that will give rise to one or more amino acid variants of Table 33, or the complement thereof. Such regions can be isolated and analyzed to efficiently detect the nucleotide variants of the present invention. Also, such regions can also be isolated and used as probes or primers in detection of the nucleotide variants of the present invention and other uses as will be clear from the descriptions below.

Thus, in one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of a IRF5 nucleic acid, the contiguous span containing one or more nucleotide variants of Table 33 (e.g., EX1@−82m and EX6@91m), or the complement thereof. In specific embodiments, the isolated nucleic acid are oligonucleotides having a contiguous span of from about 17, 18, 19, 20, 21, 22, 23 or 25 to about 30, 40 or 50, preferably from about 21 to about 30 nucleotide residues, of any IRF5 nucleic acid, said contiguous span containing one or more nucleotide variants of Table 33 (e.g., EX1@−82m and EX6@91m).

In one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of any one of SEQ ID NOs:3-11, containing one or more nucleotide variants of Table 33 (e.g., EX1@−82m and EX6@91m), or the complement thereof. In specific embodiments, the isolated nucleic acid comprises a nucleotide sequence according to any one of SEQ ID NOs:282-290. In preferred embodiments, the isolated nucleic acid are oligonucleotides having a contiguous span of from about 17, 18, 19, 20, 21, 22, 23 or 25 to about 30, 40 or 50, preferably from about 21 to about 30 nucleotide residues, of any one of SEQ ID NOs: 282-290 and containing one or more nucleotide variants of Table 33 (e.g., EX1@−82m and EX6@91m). The complements of the isolated nucleic acids are also encompassed by the present invention.

Thus, for example, an isolated nucleic acid of the present invention can have a contiguous span of at least 18, 19, 20, 21, 22, 23, 24, 25, 26, 27, 28, 29, or 30 or 40 or 50 nucleotide residues of SEQ ID NO:284 containing the nucleotide variant EX1@−82m (nucleotide residue No. 51 in SEQ ID NO:284), or a contiguous span of at least 18, 19, 20, 21, 22, 23, 24, 25, 26, 27, 28, 29, or 30 or 40 or 50 nucleotide residues of SEQ ID NO:285 containing the nucleotide variant EX6@91m (nucleotide residue No. 51 in SEQ ID NO:285), or the complements thereof.

The present invention further provides an isolated AMFR nucleic acid containing at least one of the newly discovered nucleotide variants as summarized in Tables 34 and 35, or one or more nucleotide variants that will result in the amino acid variants provided in Tables 34 and 35. The term “AMFR nucleic acid” is as defined above and means a naturally existing nucleic acid coding for a wild-type or variant or mutant AMFR. The term “AMFR nucleic acid” is inclusive and may be in the form of either double-stranded or single-stranded nucleic acids, and a single strand can be either of the two complementing strands. The isolated AMFR nucleic acid can be naturally existing genomic DNA, mRNA or cDNA. In one embodiment, the isolated AMFR nucleic acid has a nucleotide sequence according to SEQ ID NO:291, but containing one or more exonic nucleotide variants of Tables 34 and/or 35, or the complement thereof. In a specific embodiment, the isolated AMFR nucleic acid has a nucleotide sequence according to SEQ ID NO:291 but containing one or more exonic nucleotide variants of Haplotype I in Table 34, or the complement thereof. In another specific embodiment, the isolated AMFR nucleic acid has a nucleotide sequence according to SEQ ID NO:291 except for containing one or more exonic nucleotide variants of Haplotype II in Table 35, or the complement thereof.

In another embodiment, the isolated AMFR nucleic acid has a nucleotide sequence that is at least 95%, preferably at least 97% and more preferably at least 99% identical to SEQ ID NO:291 but contains one or more exonic nucleotide variants of Table 1, or one or more nucleotide variants that will result in one or more amino acid variants of Tables 34 and/or 35, or the complement thereof.

In yet another embodiment, the isolated AMFR nucleic acid has a nucleotide sequence encoding AMFR protein having an amino acid sequence according to SEQ ID NO:292 but contains one or more amino acid variants of Tables 34 and/or 35. Isolated AMFR nucleic acids having a nucleotide sequence that is the complement of the sequence are also encompassed by the present invention.

In yet another embodiment, the isolated AMFR nucleic acid has a nucleotide sequence encoding a AMFR protein having an amino acid sequence that is at least 95%, preferably at least 97% and more preferably at least 99% identical to SEQ ID NO:292 but contains one or more amino acid variants of Tables 34 and/or 35, or the complement thereof.

The present invention also provides an isolated nucleic acid, naturally occurring or artificial, having a nucleotide sequence that is at least 95%, preferably at least 97% and more preferably at least 99% identical to SEQ ID NO:291 except for containing one or more nucleotide variants of Tables 34 and/or 35, or the complement thereof.

In another embodiment, the present invention provides an isolated nucleic acid, naturally occurring or artificial, having a nucleotide sequence encoding a AMFR protein having an amino acid sequence according to SEQ ID NO:292 but containing one or more amino acid variants of Tables 34 and/or 35. Isolated nucleic acids having a nucleotide sequence that is the complement of the sequence are also encompassed by the present invention.

In addition, isolated nucleic acids are also provided which have a nucleotide sequence encoding a protein having an amino acid sequence that is at least 95%, preferably at least 97% and more preferably at least 99% identical to SEQ ID NO:292 but containing one or more amino acid variants of Tables 34 and/or 35, or the complement thereof.

The present invention also encompasses an isolated nucleic acid comprising the nucleotide sequence of a region of a AMFR genomic DNA or cDNA or mRNA, wherein the region contains one or more nucleotide variants as provided in Tables 34 and/or 35 above, or one or more nucleotide variants that will give rise to one or more amino acid variants of Tables 34 and/or 35, or the complement thereof. Such regions can be isolated and analyzed to efficiently detect the nucleotide variants of the present invention. Also, such regions can also be isolated and used as probes or primers in detection of the nucleotide variants of the present invention and other uses as will be clear from the descriptions below.

Thus, in one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of an AMFR nucleic acid, the contiguous span containing one or more nucleotide variants in Tables 34 and/or 35, or the complement thereof. In specific embodiments, the isolated nucleic acid are oligonucleotides having a contiguous span of from about 17, 18, 19, 20, 21, 22, 23 or 25 to about 30, 40 or 50, preferably from about 21 to about 30 nucleotide residues, of any AMFR nucleic acid, said contiguous span containing one or more nucleotide variants of Tables 34 and/or 35.

In one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of any one of SEQ ID NOs:295-301, containing one or more nucleotide variants Tables 34-35, or the complement thereof. In specific embodiments, the isolated nucleic acid comprises a nucleotide sequence according to any one of SEQ ID NOs:295-301. In preferred embodiments, the isolated nucleic acid are oligonucleotides having a contiguous span of from about 17, 18, 19, 20, 21, 22, 23 or 25 to about 30, 40 or 50, preferably from about 21 to about 30 nucleotide residues, of any one of SEQ ID NOs:295-301 and containing one or more nucleotide variants of Table 1. The complements of the isolated nucleic acids are also encompassed by the present invention.

Thus, for example, an isolated nucleic acid of the present invention can have a contiguous span of at least 18, 19, 20, 21, 22, 23, 24, 25, 26, 27, 28, 29, or 30 or 40 or 50 nucleotide residues of SEQ ID NO:295 containing the nucleotide variant EX4@+14C (nucleotide residue No. 51 in SEQ ID NO:295), or a contiguous span of at least 18, 19, 20, 21, 22, 23, 24, 25, 26, 27, 28, 29, or 30 or 40 or 50 nucleotide residues of SEQ ID NO:296 containing the nucleotide variant EX12@+62G (nucleotide residue No. 51 in SEQ ID NO:296), or a contiguous span of at least 18, 19, 20, 21, 22, 23, 24, 25, 26, 27, 28, 29, or 30 or 40 or 50 nucleotide residues of SEQ ID NO:297 containing the nucleotide variant EX14@1359T (nucleotide residue No. 51 in SEQ ID NO:297), or a contiguous span of at least 18, 19, 20, 21, 22, 23, 24, 25, 26, 27, 28, 29, or 30 or 40 or 50 nucleotide residues of SEQ ID NO:298 containing the nucleotide variant EX14@483A (nucleotide residue No. 51, in SEQ ID NO:298), or the complements thereof.

In preferred embodiments, an isolated oligonucleotide of the present invention is specific to a TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR allele (“allele-specific”) containing one or more nucleotide variants as disclosed in the present invention. That is, the isolated oligonucleotide is capable of selectively hybridizing, under high stringency conditions generally recognized in the art, to a TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR genomic or cDNA or mRNA containing one or more nucleotide variants as disclosed in the present invention, but not to a TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene. Such oligonucleotides will be useful in a hybridization-based method for detecting the nucleotide variants of the present invention as described in details below. An ordinarily skilled artisan would recognize various stringent conditions which enable the oligonucleotides of the present invention to differentiate between a TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene having a reference sequence and a variant TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene of the present invention. For example, the hybridization can be conducted overnight in a solution containing 50% formamide, 5×SSC, pH7.6, 5× Denhardt's solution, 10% dextran sulfate, and 20 microgram/ml denatured, sheared salmon sperm DNA. The hybridization filters can be washed in 0.1×SSC at about 65° C. Alternatively, typical PCR conditions employed in the art with an annealing temperature of about 55° C. can also be used.

In the isolated TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR oligonucleotides containing a nucleotide variant according to the present invention, the nucleotide variant can be located in any position. In one embodiment, a nucleotide variant is at the 5′ or 3′ end of the oligonucleotides. In a more preferred embodiment, a TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR oligonucleotide contains only one nucleotide variant according to the present invention, which is located at the 3′ end of the oligonucleotide. In another embodiment, a nucleotide variant of the present invention is located within no greater than four (4), preferably no greater than three (3), and more preferably no greater than two (2) nucleotides of the center of the oligonucleotide of the present invention. In more preferred embodiment, a nucleotide variant is located at the center or within one (1) nucleotide of the center of the oligonucleotide. For purposes of defining the location of a nucleotide variant in an oligonucleotide, the center nucleotide of an oligonucleotide with an odd number of nucleotides is considered to be the center. For an oligonucleotide with an even number of nucleotides, the bond between the two center nucleotides is considered to be the center.

In other embodiments of the present invention, isolated nucleic acids are provided which encode a contiguous span of at least 6, 7, 8, 9, 10, 11, 12, 13, 14, 15, 16 or 17 amino acids of an TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein wherein said contiguous span contains at least one amino acid variant in Tables 1-35 according to the present invention.

The oligonucleotides of the present invention can have a detectable marker selected from, e.g., radioisotopes, fluorescent compounds, enzymes, or enzyme co-factors operably linked to the oligonucleotide. The oligonucleotides of the present invention can be useful in genotyping as will be apparent from the description below.

In addition, the present invention also provides DNA microchips or microarray incorporating a variant TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR genomic DNA or cDNA or mRNA or an oligonucleotide according to the present invention. The microchip will allow rapid genotyping and/or haplotyping in a large scale.

As is known in the art, in microchips, a large number of different nucleic acid probes are attached or immobilized in an array on a solid support, e.g., a silicon chip or glass slide. Target nucleic acid sequences to be analyzed can be contacted with the immobilized oligonucleotide probes on the microchip. See Lipshutz et al., Biotechniques, 19:442-447 (1995); Chee et al., Science, 274:610-614 (1996); Kozal et al., Nat. Med. 2:753-759 (1996); Hacia et al., Nat. Genet., 14:441-447 (1996); Saiki et al., Proc. Natl. Acad. Sci. USA, 86:6230-6234 (1989); Gingeras et al., Genome Res., 8:435-448 (1998). The microchip technologies combined with computerized analysis tools allow large-scale high throughput screening. See, e.g., U.S. Pat. No. 5,925,525 to Fodor et al; Wilgenbus et al., J. Mol. Med., 77:761-786 (1999); Graber et al., Curr. Opin. Biotechnol., 9:14-18 (1998); Hacia et al., Nat. Genet., 14:441-447 (1996); Shoemaker et al., Nat. Genet., 14:450-456 (1996); DeRisi et al., Nat. Genet., 14:457-460 (1996); Chee et al., Nat. Genet., 14:610-614 (1996); Lockhart et al., Nat. Genet., 14:675-680 (1996); Drobyshev et al., Gene, 188:45-52 (1997).

In a preferred embodiment, a DNA microchip is provided comprising a plurality of the oligonucleotides of the present invention such that the nucleotide identity at each of the nucleotide variant sites disclosed in Tables 1-35 can be determined in one single microarray. In a preferred embodiment, the microchip incorporates a variant TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR nucleic acid or oligonucleotide of the present invention and contains at least two of the variants in Tables 1-35 and 81, preferably at least ten, more preferably at least 20, 30, 40, 50, or 100 of the variants in Table 1-35 and 81.

In one embodiment, the DNA microchip is designed to detect at least one nucleotide variant associated with a high or low expression phenotype of at least two, preferably at least 3, 4, 5, 6, 7, 8, 9, 10, 11, 12, 13, 14 or at least 15 of the genes chosen from the group of TLK1, WARS2, ARTS2, MSR, AKAP9, DNAJD1, RABEP1, TAP2, DDX58, FKBP1a, SRI, XRRA1, and AMFR. Preferably such a microchip is designed to detect at least one nucleotide variant associated with a high expression phenotype and at least one nucleotide variant associated with a low expression phenotype of at least two, preferably at least 3, 4, 5, 6, 7, 8, 9, 10, 11, 12, 13, 14 or at least 15 of the genes chosen from the group of TLK1, WARS2, ARTS2, MSR, AKAP9, DNAJD1, RABEP1, TAP2, DDX58, FKBP1a, SRI, XRRA1, and AMFR. Such microchips can be used in diagnostic or prognostic or pharmacogenetic response assays related to cancer. Such variants are according to the present invention selected from those in Tables 1-35 and 81. The variants can be contained in nucleic acids, particularly oligonucleotides in the DNA microchip.

### 4. Proteins and Peptides

In one aspect, the present invention provides an isolated ARTS1 protein encoded by one of the novel ARTS1 gene variants according to the present invention. Thus, for example, the present invention provides an isolated ARTS1 protein having an amino acid sequence according to SEQ ID NO:31 but containing one or more amino acid variants selected from the group consisting of P127R and Q725R. In another example, the isolated ARTS1 protein of the present invention has an amino acid sequence at least 95%, preferably 97%, more preferably 99% identical to SEQ ID NO:31 wherein the amino acid sequence contains at least one amino acid variant selected from the group consisting of P127R and Q725R.

In addition, the present invention also encompasses isolated peptides having a contiguous span of at least 6, 7, 8, 9, 10, 11, 12, 13, 15, 17, 19 or 21 or more amino acids of an isolated ARTS1 protein of the present invention said contiguous span encompassing one or more amino acid variants selected from the group consisting of P127R and Q725R. In preferred embodiments, the isolated variant ARTS1 peptides contain no greater than 200 or 100 amino acids, and preferably no greater than 50 amino acids. In specific embodiments, the ARTS1 polypeptides in accordance with the present invention contain one or more of the amino acid variants identified in accordance with the present invention. The peptides can be useful in preparing antibodies specific to the mutant ARTS1 proteins provided in accordance with the present invention.

Thus, as an example, an isolated polypeptide of the present invention can have a contiguous span of at least 6, 7, 8, 9, 10, 11, 12, 13, 14 or 15 amino acid residues of SEQ ID NO:31 encompassing the amino acid variant P127R (amino acid residue No. 127 in SEQ ID NO:31), or a contiguous span of at least 6, 7, 8, 9, 10, 11, 12, 13, 14 or 15 amino acid residues of SEQ ID NO:31 encompassing the amino acid variant Q725R (amino acid residue No. 725 in SEQ ID NO:31).

In another aspect, the present invention provides an isolated MSR protein encoded by one of the novel MSR gene variants according to the present invention. Thus, for example, the present invention provides an isolated MSR protein having an amino acid sequence according to SEQ ID NO:67 but containing one or more amino acid variants selected from the group consisting of K350R or H595Y. In another example, the isolated MSR protein of the present invention has an amino acid sequence at least 95%, preferably 97%, more preferably 99% identical to SEQ ID NO:67 wherein the amino acid sequence contains at least one amino acid variant selected from the group consisting of K350R or H595Y.

In addition, the present invention also encompasses isolated peptides having a contiguous span of at least 6, 7, 8, 9, 10, 11, 12, 13, 15, 17, 19 or 21 or more amino acids of an isolated MSR protein of the present invention said contiguous span encompassing one or more amino acid variants selected from the group consisting of K350R or H595Y. In preferred embodiments, the isolated variant MSR peptides contain no greater than 200 or 100 amino acids, and preferably no greater than 50 amino acids. In specific embodiments, the MSR polypeptides in accordance with the present invention contain one or more of the amino acid variants identified in accordance with the present invention. The peptides can be useful in preparing antibodies specific to the mutant MSR proteins provided in accordance with the present invention.

Thus, as an example, an isolated polypeptide of the present invention can have a contiguous span of at least 6, 7, 8, 9, 10, 11, 12, 13, 14 or 15 amino acid residues of SEQ ID NO:67 encompassing the amino acid variant H595Y (amino acid residue No. 595 in SEQ ID NO:67), or encompassing the amino acid variant K350R (amino acid residue No. 350 in SEQ ID NO:67).

The present invention also provides isolated proteins encoded by one of the isolated nucleic acids according to the present invention. In one aspect, the present invention provides an isolated AKAP9 protein encoded by one of the novel AKAP9 gene variants according to the present invention. Thus, for example, the present invention provides an isolated AKAP9 protein having an amino acid sequence according to SEQ ID NO:91 but containing one or more amino acid variants selected from the group consisting of N2792S and M463I. In another example, the isolated AKAP9 protein of the present invention has an amino acid sequence at least 95%, preferably 97%, more preferably 99% identical to SEQ ID NO:91 wherein the amino acid sequence contains at least one amino acid variant selected from the group consisting of N2792S and M463I.

In addition, the present invention also encompasses isolated peptides having a contiguous span of at least 6, 7, 8, 9, 10, 11, 12, 13, 15, 17, 19 or 21 or more amino acids of an isolated AKAP9 protein of the present invention said contiguous span encompassing one or more amino acid variants selected from the group consisting of N2792S and M463I. In preferred embodiments, the isolated variant AKAP9 peptides contain no greater than 200 or 100 amino acids, and preferably no greater than 50 amino acids. In specific embodiments, the AKAP9 polypeptides in accordance with the present invention contain one or more of the amino acid variants identified in accordance with the present invention. The peptides can be useful in preparing antibodies specific to the mutant AKAP9 proteins provided in accordance with the present invention.

Thus, as an example, an isolated polypeptide of the present invention can have a contiguous span of at least 6, 7, 8, 9, 10, 11, 12, 13, 14 or 15 amino acid residues of SEQ ID NO:91 encompassing the amino acid variant M463I (amino acid residue No. 463 in SEQ ID NO:91), or a contiguous span of at least 6, 7, 8, 9, 10, 11, 12, 13, 14 or 15 amino acid residues of SEQ ID NO:91 encompassing the amino acid variant N2792S (amino acid residue No. 2792 in SEQ ID NO:91).

In another aspect, the present invention provides an isolated DNAJD1 protein encoded by one of the novel DNAJD1 gene variants according to the present invention. Thus, for example, the present invention provides an isolated DNAJD1 protein having an amino acid sequence according to SEQ ID NO:150 but containing the amino acid variant R35G. In another example, the isolated DNAJD1 protein of the present invention has an amino acid sequence at least 95%, preferably 97%, more preferably 99% identical to SEQ ID NO:150 wherein the amino acid sequence contains the amino acid variant R35G.

In addition, the present invention also encompasses isolated peptides having a contiguous span of at least 6, 7, 8, 9, 10, 11, 12, 13, 15, 17, 19 or 21 or more amino acids of an isolated DNAJD1 protein of the present invention said contiguous span encompassing the amino acid variant R35G. In preferred embodiments, the isolated variant DNAJD1 peptides contain no greater than 200 or 100 amino acids, and preferably no greater than 50 amino acids. In specific embodiments, the DNAJD1 polypeptides in accordance with the present invention contain the amino acid variant identified in accordance with the present invention. The peptides can be useful in preparing antibodies specific to the mutant DNAJD1 proteins provided in accordance with the present invention.

Thus, as an example, an isolated polypeptide of the present invention can have a contiguous span of at least 6, 7, 8, 9, 10, 11, 12, 13, 14 or 15 amino acid residues of SEQ ID NO:150 encompassing the amino acid variant R35G (amino acid residue No. 35 in SEQ ID NO:150).

In yet another aspect, the present invention provides an isolated TAP2 protein encoded by one of the novel TAP2 gene variants according to the present invention. Thus, for example, the present invention provides an isolated TAP2 protein having an amino acid sequence according to SEQ ID NO:203 but containing one or more amino acid variants selected from the group consisting of A665T and R651C. In another example, the isolated TAP2 protein of the present invention has an amino acid sequence at least 95%, preferably 97%, more preferably 99% identical to SEQ ID NO:203 wherein the amino acid sequence contains at least one amino acid variant selected from the group consisting of R651C and A665T.

In addition, the present invention also encompasses isolated peptides having a contiguous span of at least 6, 7, 8, 9, 10, 11, 12, 13, 15, 17, 19 or 21 or more amino acids of an isolated TAP2 protein of the present invention said contiguous span encompassing one or more amino acid variants selected from the group consisting of R651C and A665T. In preferred embodiments, the isolated variant TAP2 peptides contain no greater than 200 or 100 amino acids, and preferably no greater than 50 amino acids. In specific embodiments, the TAP2 polypeptides in accordance with the present invention contain one or more of the amino acid variants identified in accordance with the present invention. The peptides can be useful in preparing antibodies specific to the mutant TAP2 proteins provided in accordance with the present invention.

Thus, as an example, an isolated polypeptide of the present invention can have a contiguous span of at least 6, 7, 8, 9, 10, 11, 12, 13, 14 or 15 amino acid residues of SEQ ID NO:203 encompassing the amino acid variant R651C (amino acid residue No. 651 in SEQ ID NO:203), or a contiguous span of at least 6, 7, 8, 9, 10, 11, 12, 13, 14 or 15 amino acid residues of SEQ ID NO:203 encompassing the amino acid variant A665T (amino acid residue No. 665 in SEQ ID NO:195).

The present invention also provides isolated proteins encoded by one of the isolated nucleic acids according to the present invention. In one aspect, the present invention provides an isolated XRRA1 protein encoded by one of the novel XRRA1 gene variants according to the present invention. Thus, for example, the present invention provides an isolated XRRA1 protein having an amino acid sequence according to SEQ ID NO:258 but containing one or more amino acid variants selected from the group consisting of P38R and T502R. In another example, the isolated XRRA1 protein of the present invention has an amino acid sequence at least 95%, preferably 97%, more preferably 99% identical to SEQ ID NO:258 wherein the amino acid sequence contains at least one amino acid variant selected from the group consisting of P38R and T502R.

In addition, the present invention also encompasses isolated peptides having a contiguous span of at least 6, 7, 8, 9, 10, 11, 12, 13, 15, 17, 19 or 21 or more amino acids of an isolated XRRA1 protein of the present invention said contiguous span encompassing one or more amino acid variants selected from the group consisting of P38R and T502R. In preferred embodiments, the isolated variant XRRA1 peptides contain no greater than 200 or 100 amino acids, and preferably no greater than 50 amino acids. In specific embodiments, the XRRA1 polypeptides in accordance with the present invention contain one or more of the amino acid variants identified in accordance with the present invention. The peptides can be useful in preparing antibodies specific to the mutant XRRA1 proteins provided in accordance with the present invention.

Thus, as an example, an isolated polypeptide of the present invention can have a contiguous span of at least 6, 7, 8, 9, 10, 11, 12, 13, 14 or 15 amino acid residues of SEQ ID NO:258 encompassing the amino acid variant P38R (amino acid residue No. 38 in SEQ ID NO:258), or a contiguous span of at least 6, 7, 8, 9, 10, 11, 12, 13, 14 or 15 amino acid residues of SEQ ID NO:258 encompassing the amino acid variant T502R (amino acid residue No. 502 in SEQ ID NO:258).

As will be apparent to an ordinarily skilled artisan, the isolated nucleic acids and isolated polypeptides of the present invention can be prepared using techniques generally known in the field of molecular biology. See generally, Sambrook et al., Molecular Cloning: A Laboratory Manual, 2^{nd} ed., Cold Spring Harbor Laboratory, Cold Spring Harbor, N.Y., 1989. The isolated ARTS2, MSR, AKAP9, DNAJD1, TAP2, or XRRA1 gene or cDNA or oligonucleotides of this invention can be operably linked to one or more other DNA fragments. For example, the isolated ARTS2, MSR, AKAP9, DNAJD1, TAP2, or XRRA1 nucleic acid (e.g., cDNA or oligonucleotides) can be ligated to another DNA such that a fusion protein can be encoded by the ligation product. The isolated ARTS2, MSR, AKAP9, DNAJD1, TAP2, or XRRA1 nucleic acid (e.g., cDNA or oligonucleotides) can also be incorporated into a DNA vector for purposes of, e.g., amplifying the nucleic acid or a portion thereof, and/or expressing a mutant ARTS2, MSR, AKAP9, DNAJD1, TAP2, or XRRA1 polypeptide or a fusion protein thereof.

Thus, the present invention also provides a vector construct containing an isolated nucleic acid of the present invention, such as a mutant ARTS2, MSR, AKAP9, DNAJD1, TAP2, or XRRA1 nucleic acid (e.g., cDNA or oligonucleotides) of the present invention. Generally, the vector construct may include a promoter operably linked to a DNA of interest (including a full-length sequence or a fragment thereof in the 5′ to 3′ direction or in the reverse direction for purposes of producing antisense nucleic acids), an origin of DNA replication for the replication of the vector in host cells and a replication origin for the amplification of the vector in, e.g., E. coli, and selection marker(s) for selecting and maintaining only those host cells harboring the vector. Additionally, the vector preferably also contains inducible elements, which function to control the expression of the isolated gene sequence. Other regulatory sequences such as transcriptional termination sequences and translation regulation sequences (e.g., Shine-Dalgarno sequence) can also be included. An epitope tag-coding sequence for detection and/or purification of the encoded polypeptide can also be incorporated into the vector construct. Examples of useful epitope tags include, but are not limited to, influenza virus hemagglutinin (HA), Simian Virus 5 (V5), polyhistidine (6xHis), c-myc, lacZ, GST, and the like. Proteins with polyhistidine tags can be easily detected and/or purified with Ni affinity columns, while specific antibodies to many epitope tags are generally commercially available. The vector construct can be introduced into the host cells or organisms by any techniques known in the art, e.g., by direct DNA transformation, microinjection, electroporation, viral infection, lipofection, gene gun, and the like. The vector construct can be maintained in host cells in an extrachromosomal state, i.e., as self-replicating plasmids or viruses. Alternatively, the vector construct can be integrated into chromosomes of the host cells by conventional techniques such as selection of stable cell lines or site-specific recombination. The vector construct can be designed to be suitable for expression in various host cells, including but not limited to bacteria, yeast cells, plant cells, insect cells, and mammalian and human cells. A skilled artisan will recognize that the designs of the vectors can vary with the host cell used.

### 5. Antibodies

The present invention also provides antibodies selectively immunoreactive with a variant ARTS2, MSR, AKAP9, DNAJD1, TAP2, or XRRA1 protein or peptide provided in accordance with the present invention and methods for making the antibodies. As used herein, the term “antibody” encompasses both monoclonal and polyclonal antibodies that fall within any antibody classes, e.g., IgG, IgM, IgA, etc. The term “antibody” also means antibody fragments including, but not limited to, Fab and F(ab′)₂, conjugates of such fragments, and single-chain antibodies that can be made in accordance with U.S. Pat. No. 4,704,692, which is incorporated herein by reference. Specifically, the phrase “selectively immunoreactive with one or more of the newly discovered variant ARTS2, MSR, AKAP9, DNAJD1, TAP2, or XRRA1 protein variants” as used herein means that the immunoreactivity of an antibody with a protein variant of the present invention is substantially higher than that with the ARTS2, MSR, AKAP9, DNAJD1, TAP2, or XRRA1 protein heretofore known in the art such that the binding of the antibody to the protein variant of the present invention is readily distinguishable, based on the strength of the binding affinities, from the binding of the antibody to the ARTS2, MSR, AKAP9, DNAJD1, TAP2, or XRRA1 protein having a reference amino acid sequence. Preferably, the binding constant differs by a magnitude of at least 2 fold, more preferably at least 5 fold, even more preferably at least 10 fold, and most preferably at least 100 fold.

To make such an antibody, a variant ARTS2, MSR, AKAP9, DNAJD1, TAP2, or XRRA1 protein or a peptide of the present invention having a particular amino acid variant (e.g., substitution or insertion or deletion) is provided and used to immunize an animal. The variant ARTS2, MSR, AKAP9, DNAJD1, TAP2, or XRRA1 protein or peptide variant can be made by any methods known in the art, e.g., by recombinant expression or chemical synthesis. To increase the specificity of the antibody, a shorter peptide containing an amino acid variant is preferably generated and used as antigen. Techniques for immunizing animals for the purpose of making polyclonal antibodies are generally known in the art. See Harlow and Lane, Antibodies: A Laboratory Manual, Cold Spring Harbor Laboratory, Cold Spring Harbor, N.Y., 1988. A carrier may be necessary to increase the immunogenicity of the polypeptide. Suitable carriers known in the art include, but are not limited to, liposome, macromolecular protein or polysaccharide, or combination thereof. Preferably, the carrier has a molecular weight in the range of about 10,000 to 1,000,000. The polypeptide may also be administered along with an adjuvant, e.g., complete Freund's adjuvant.

The antibodies of the present invention preferably are monoclonal. Such monoclonal antibodies may be developed using any conventional techniques known in the art. For example, the popular hybridoma method disclosed in Kohler and Milstein, Nature, 256:495-497 (1975) is now a well-developed technique that can be used in the present invention. See U.S. Pat. No. 4,376,110, which is incorporated herein by reference. Essentially, B-lymphocytes producing a polyclonal antibody against a protein variant of the present invention can be fused with myeloma cells to generate a library of hybridoma clones. The hybridoma population is then screened for antigen binding specificity and also for immunoglobulin class (isotype). In this manner, pure hybridoma clones producing specific homogenous antibodies can be selected. See generally, Harlow and Lane, Antibodies: A Laboratory Manual, Cold Spring Harbor Press, 1988. Alternatively, other techniques known in the art may also be used to prepare monoclonal antibodies, which include but are not limited to the EBV hybridoma technique, the human N-cell hybridoma technique, and the trioma technique.

In addition, antibodies selectively immunoreactive with a protein or peptide variant of the present invention may also be recombinantly produced. For example, cDNAs prepared by PCR amplification from activated B-lymphocytes or hybridomas may be cloned into an expression vector to form a cDNA library, which is then introduced into a host cell for recombinant expression. The cDNA encoding a specific protein may then be isolated from the library. The isolated cDNA can be introduced into a suitable host cell for the expression of the protein. Thus, recombinant techniques can be used to recombinantly produce specific native antibodies, hybrid antibodies capable of simultaneous reaction with more than one antigen, chimeric antibodies (e.g., the constant and variable regions are derived from different sources), univalent antibodies which comprise one heavy and light chain pair coupled with the Fc region of a third (heavy) chain, Fab proteins, and the like. See U.S. Pat. No. 4,816,567; European Patent Publication No. 0088994; Munro, Nature, 312:597 (1984); Morrison, Science, 229:1202 (1985); Oi et al., BioTechniques, 4:214 (1986); and Wood et al., Nature, 314:446-449 (1985), all of which are incorporated herein by reference. Antibody fragments such as Fv fragments, single-chain Fv fragments (scFv), Fab′ fragments, and F(ab′)₂ fragments can also be recombinantly produced by methods disclosed in, e.g., U.S. Pat. No. 4,946,778; Skerra & Plückthun, Science, 240:1038-1041 (1988); Better et al., Science, 240:1041-1043 (1988); and Bird, et al., Science, 242:423-426 (1988), all of which are incorporated herein by reference.

In a preferred embodiment, the antibodies provided in accordance with the present invention are partially or fully humanized antibodies. For this purpose, any methods known in the art may be used. For example, partially humanized chimeric antibodies having V regions derived from the tumor-specific mouse monoclonal antibody, but human C regions are disclosed in Morrison and Oi, Adv. Immunol., 44:65-92 (1989). In addition, fully humanized antibodies can be made using transgenic non-human animals. For example, transgenic non-human animals such as transgenic mice can be produced in which endogenous immunoglobulin genes are suppressed or deleted, while heterologous antibodies are encoded entirely by exogenous immunoglobulin genes, preferably human immunoglobulin genes, recombinantly introduced into the genome. See e.g., U.S. Pat. Nos. 5,530,101; 5,545,806; 6,075,181; PCT Publication No. WO 94/02602; Green et. al., Nat. Genetics, 7: 13-21 (1994); and Lonberg et al., Nature 368: 856-859 (1994), all of which are incorporated herein by reference. The transgenic non-human host animal may be immunized with suitable antigens such as a protein variant of the present invention to illicit a specific immune response thus producing humanized antibodies. In addition, cell lines producing specific humanized antibodies can also be derived from the immunized transgenic non-human animals. For example, mature B-lymphocytes obtained from a transgenic animal producing humanized antibodies can be fused to myeloma cells and the resulting hybridoma clones may be selected for specific humanized antibodies with desired binding specificities. Alternatively, cDNAs may be extracted from mature B-lymphocytes and used in establishing a library which is subsequently screened for clones encoding humanized antibodies with desired binding specificities.

In a specific embodiment, the antibody is selectively immunoreactive with a variant ARTS1 protein or peptide containing the amino acid variant P127R and/or Q725R.

In another specific embodiment, the antibody is selectively immunoreactive with a variant MSR protein or peptide containing the amino acid variant K350R and/or H595Y.

In another specific embodiment, the antibody is selectively immunoreactive with a variant AKAP9 protein or peptide containing the amino acid variant N2792S and/or M463I.

In another specific embodiment, the antibody is selectively immunoreactive with a variant DNAJD1 protein or peptide containing the amino acid variant R35G.

In yet another specific embodiment, the antibody is selectively immunoreactive with a variant TAP2 protein or peptide containing the amino acid variant R651C and/or T665A.

In yet another specific embodiment, the antibody is selectively immunoreactive with a variant XRRA1 protein or peptide containing the amino acid variant P38R and/or T502R.

### 6. Genotyping

The present invention provides methods for genotyping the TLK1, WARS2, ARTS2, MSR, AKAP9, DNAJD1, GOLPH4, RABEP1, TAP2, NARG2, DDX58, CD39, FKBP1a, SRI, XRRA1, IRF5 and AMFR genes by determining whether an individual, or a tissue sample from an individual, has a nucleotide variant or amino acid variant of the present invention.

Similarly, methods for haplotyping the TLK1, WARS2, ARTS2, MSR, AKAP9, DNAJD1, GOLPH4, RABEP1, TAP2, NARG2, DDX58, CD39, FKBP1a, SRI, XRRA1, IRF5 and AMFR genes are also provided. Haplotyping can be done by any methods known in the art. For example, only one copy of the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene can be isolated from an individual, or from a tissue sample from an individual, and the nucleotide at each of the variant positions is determined. Alternatively, an allele specific PCR or a similar method can be used to amplify only one copy of the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene in an individual, or the tissue sample, and the SNPs at the variant positions of the present invention are determined. The Clark method known in the art can also be employed for haplotyping. A high throughput molecular haplotyping method is also disclosed in Tost et al., Nucleic Acids Res., 30(19):e96 (2002), which is incorporated herein by reference.

Thus, additional variant(s) that are in linkage disequilibrium with the variants and/or haplotypes of the present invention can be identified by a haplotyping method known in the art, as will be apparent to a skilled artisan in the field of genetics and haplotying. The additional variants that are in linkage disequilibrium with a variant or haplotype of the present invention can also be useful in the various applications as described below.

For purposes of genotyping and haplotyping, both genomic DNA and mRNA/cDNA can be used, and both are herein referred to generically as “gene.”

Numerous techniques for detecting nucleotide variants are known in the art and can all be used for the method of this invention. The techniques can be protein-based or DNA-based. In either case, the techniques used must be sufficiently sensitive so as to accurately detect the small nucleotide or amino acid variations. Very often, a probe is utilized which is labeled with a detectable marker. Unless otherwise specified in a particular technique described below, any suitable marker known in the art can be used, including but not limited to, radioactive isotopes, fluorescent compounds, biotin which is detectable using strepavidin, enzymes (e.g., alkaline phosphatase), substrates of an enzyme, ligands and antibodies, etc. See Jablonski et al., Nucleic Acids Res., 14:6115-6128 (1986); Nguyen et al., Biotechniques, 13:116-123 (1992); Rigby et al., J. Mol. Biol., 113:237-251 (1977).

In a DNA-based detection method, a target DNA sample, i.e., a sample containing TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR genomic DNA or cDNA or mRNA must be obtained from the individual to be tested, or from a tissue sample from the individual to be tested. Any tissue or cell sample containing the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR genomic DNA, mRNA, or cDNA or a portion thereof can be used. However, in situations where the individual to be tested is thought or known to have cancer, a tissue sample may preferentially be obtained from the cancerous growth or tumor by biopsy. In such situations, it may be important to ensure that the biopsy sample is primarily composed of cancerous cells, since it may be advantageous to specifically genotype the cancerous growth or tumor, instead of, or in addition to, the somatic tissues of the individual being tested. For all genotyping methods, a tissue sample containing cell nucleus and thus genomic DNA can be obtained from the individual or from the cancerous growth or tumor within the individual. For genotyping somatic tissues, blood samples can be useful, except that only white blood cells and other lymphocytes have cell nuclei, while red blood cells are anucleate and contain only mRNA. Nevertheless, mRNA is also useful as it can be analyzed for the presence of nucleotide variants in its sequence or serve as template for cDNA synthesis. The tissue or cell samples, whether from the somatic tissues of an individual, or from a biopsy of a cancerous growth or tumor, can be analyzed directly without much processing. Alternatively, nucleic acids including the target sequence can be extracted, purified, and/or amplified before they are subject to the various detecting procedures discussed below. Other than tissue or cell samples, cDNAs or genomic DNAs from a cDNA or genomic DNA library constructed using a tissue or cell sample obtained from the individual to be tested, or from a biopsy of a cancerous growth or tumor from an individual, are also useful.

To determine the presence or absence of a particular nucleotide variant, one technique is simply sequencing the target genomic DNA or cDNA, particularly the region encompassing the nucleotide variant locus to be detected. Various sequencing techniques are generally known and widely used in the art including the Sanger method and Gilbert chemical method. The newly developed pyrosequencing method monitors DNA synthesis in real time using a luminometric detection system. Pyrosequencing has been shown to be effective in analyzing genetic polymorphisms such as single-nucleotide polymorphisms and thus can also be used in the present invention. See Nordstrom et al., Biotechnol. Appl. Biochem., 31(2):107-112 (2000); Ahmadian et al., Anal. Biochem., 280:103-110 (2000).

Alternatively, the restriction fragment length polymorphism (RFLP) and AFLP method may also prove to be useful techniques. In particular, if a nucleotide variant in the target TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR DNA results in the elimination or creation of a restriction enzyme recognition site, then digestion of the target DNA with that particular restriction enzyme will generate an altered restriction fragment length pattern. Thus, a detected RFLP or AFLP will indicate the presence of a particular nucleotide variant.

Another useful approach is the single-stranded conformation polymorphism assay (SSCA), which is based on the altered mobility of a single-stranded target DNA spanning the nucleotide variant of interest. A single nucleotide change in the target sequence can result in different intramolecular base pairing pattern, and thus different secondary structure of the single-stranded DNA, which can be detected in a non-denaturing gel. See Orita et al., Proc. Natl. Acad. Sci. USA, 86:2776-2770 (1989). Denaturing gel-based techniques such as clamped denaturing gel electrophoresis (CDGE) and denaturing gradient gel electrophoresis (DGGE) detect differences in migration rates of mutant sequences as compared to wild-type sequences in denaturing gel. See Miller et al., Biotechniques, 5:1016-24 (1999); Sheffield et al., Am. J. Hum, Genet., 49:699-706 (1991); Wartell et al., Nucleic Acids Res., 18:2699-2705 (1990); and Sheffield et al., Proc. Natl. Acad. Sci. USA, 86:232-236 (1989). In addition, the double-strand conformation analysis (DSCA) can also be useful in the present invention. See Arguello et al., Nat. Genet., 18:192-194 (1998).

The presence or absence of a nucleotide variant at a particular locus in the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene of an individual can also be detected using the amplification refractory mutation system (ARMS) technique. See e.g., European Patent No. 0,332,435; Newton et al., Nucleic Acids Res., 17:2503-2515 (1989); Fox et al., Br. J. Cancer, 77:1267-1274 (1998); Robertson et al., Eur. Respir. J, 12:477-482 (1998). In the ARMS method, a primer is synthesized matching the nucleotide sequence immediately 5′ upstream from the locus being tested except that the 3′-end nucleotide which corresponds to the nucleotide at the locus is a predetermined nucleotide. For example, the 3′-end nucleotide can be the same as that in the mutated locus. The primer can be of any suitable length so long as it hybridizes to the target DNA under stringent conditions only when its 3′-end nucleotide matches the nucleotide at the locus being tested. Preferably the primer has at least 12 nucleotides, more preferably from about 18 to 50 nucleotides. If the individual tested has a mutation at the locus and the nucleotide therein matches the 3′-end nucleotide of the primer, then the primer can be further extended upon hybridizing to the target DNA template, and the primer can initiate a PCR amplification reaction in conjunction with another suitable PCR primer. In contrast, if the nucleotide at the locus is of wild type, then primer extension cannot be achieved. Various forms of ARMS techniques developed in the past few years can be used. See e.g., Gibson et al., Clin. Chem. 43:1336-1341 (1997).

Similar to the ARMS technique is the mini sequencing or single nucleotide primer extension method, which is based on the incorporation of a single nucleotide. An oligonucleotide primer matching the nucleotide sequence immediately 5′ to the locus being tested is hybridized to the target DNA or mRNA in the presence of labeled dideoxyribonucleotides. A labeled nucleotide is incorporated or linked to the primer only when the dideoxyribonucleotides matches the nucleotide at the variant locus being detected. Thus, the identity of the nucleotide at the variant locus can be revealed based on the detection label attached to the incorporated dideoxyribonucleotides. See Syvanen et al., Genomics, 8:684-692 (1990); Shumaker et al., Hum. Mutat., 7:346-354 (1996); Chen et al., Genome Res., 10:549-547 (2000).

Another set of techniques useful in the present invention is the so-called “oligonucleotide ligation assay” (OLA) in which differentiation between a wild-type locus and a mutation is based on the ability of two oligonucleotides to anneal adjacent to each other on the target DNA molecule allowing the two oligonucleotides joined together by a DNA ligase. See Landergren et al., Science, 241:1077-1080 (1988); Chen et al, Genome Res., 8:549-556 (1998); Iannone et al., Cytometry, 39:131-140 (2000). Thus, for example, to detect a single-nucleotide mutation at a particular locus in the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene, two oligonucleotides can be synthesized, one having the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR sequence just 5′ upstream from the locus with its 3′ end nucleotide being identical to the nucleotide in the variant locus of the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene, the other having a nucleotide sequence matching the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR sequence immediately 3′ downstream from the locus in the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene. The oligonucleotides can be labeled for the purpose of detection. Upon hybridizing to the target TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene under a stringent condition, the two oligonucleotides are subject to ligation in the presence of a suitable ligase. The ligation of the two oligonucleotides would indicate that the target DNA has a nucleotide variant at the locus being detected.

Detection of small genetic variations can also be accomplished by a variety of hybridization-based approaches. Allele-specific oligonucleotides are most useful. See Conner et al., Proc. Natl. Acad. Sci. USA, 80:278-282 (1983); Saiki et al, Proc. Natl. Acad. Sci. USA, 86:6230-6234 (1989). Oligonucleotide probes (allele-specific) hybridizing specifically to a TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene allele having a particular gene variant at a particular locus but not to other alleles can be designed by methods known in the art. The probes can have a length of, e.g., from 10 to about 50 nucleotide bases. The target TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR DNA and the oligonucleotide probe can be contacted with each other under conditions sufficiently stringent such that the nucleotide variant can be distinguished from the wild-type TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene based on the presence or absence of hybridization. The probe can be labeled to provide detection signals. Alternatively, the allele-specific oligonucleotide probe can be used as a PCR amplification primer in an “allele-specific PCR” and the presence or absence of a PCR product of the expected length would indicate the presence or absence of a particular nucleotide variant.

Other useful hybridization-based techniques allow two single-stranded nucleic acids annealed together even in the presence of mismatch due to nucleotide substitution, insertion or deletion. The mismatch can then be detected using various techniques. For example, the annealed duplexes can be subject to electrophoresis. The mismatched duplexes can be detected based on their electrophoretic mobility that is different from the perfectly matched duplexes. See Cariello, Human Genetics, 42:726 (1988). Alternatively, in a RNase protection assay, a RNA probe can be prepared spanning the nucleotide variant site to be detected and having a detection marker. See Giunta et al., Diagn. Mol. Path., 5:265-270 (1996); Finkelstein et al., Genomics, 7:167-172 (1990); Kinszler et al., Science 251:1366-1370 (1991). The RNA probe can be hybridized to the target DNA or mRNA forming a heteroduplex that is then subject to the ribonuclease RNase A digestion. RNase A digests the RNA probe in the heteroduplex only at the site of mismatch. The digestion can be determined on a denaturing electrophoresis gel based on size variations. In addition, mismatches can also be detected by chemical cleavage methods known in the art. See e.g., Roberts et al., Nucleic Acids Res., 25:3377-3378 (1997).

In the mutS assay, a probe can be prepared matching the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene sequence surrounding the locus at which the presence or absence of a mutation is to be detected, except that a predetermined nucleotide is used at the variant locus. Upon annealing the probe to the target DNA to form a duplex, the E. coli mutS protein is contacted with the duplex. Since the mutS protein binds only to heteroduplex sequences containing a nucleotide mismatch, the binding of the mutS protein will be indicative of the presence of a mutation. See Modrich et al., Ann. Rev. Genet., 25:229-253 (1991).

A great variety of improvements and variations have been developed in the art on the basis of the above-described basic techniques, and can all be useful in detecting mutations or nucleotide variants in the present invention. For example, the “sunrise probes” or “molecular beacons” utilize the fluorescence resonance energy transfer (FRET) property and give rise to high sensitivity. See Wolf et al., Proc. Nat. Acad. Sci. USA, 85:8790-8794 (1988). Typically, a probe spanning the nucleotide locus to be detected are designed into a hairpin-shaped structure and labeled with a quenching fluorophore at one end and a reporter fluorophore at the other end. In its natural state, the fluorescence from the reporter fluorophore is quenched by the quenching fluorophore due to the proximity of one fluorophore to the other. Upon hybridization of the probe to the target DNA, the 5′ end is separated apart from the 3′-end and thus fluorescence signal is regenerated. See Nazarenko et al., Nucleic Acids Res., 25:2516-2521 (1997); Rychlik et al., Nucleic Acids Res., 17:8543-8551 (1989); Sharkey et al., Bio/Technology 12:506-509 (1994); Tyagi et al., Nat. Biotechnol., 14:303-308 (1996); Tyagi et al., Nat. Biotechnol., 16:49-53 (1998). The homo-tag assisted non-dimer system (HANDS) can be used in combination with the molecular beacon methods to suppress primer-dimer accumulation. See Brownie et al., Nucleic Acids Res., 25:3235-3241 (1997).

Dye-labeled oligonucleotide ligation assay is a FRET-based method, which combines the OLA assay and PCR. See Chen et al., Genome Res. 8:549-556 (1998). TaqMan is another FRET-based method for detecting nucleotide variants. A TaqMan probe can be oligonucleotides designed to have the nucleotide sequence of the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene spanning the variant locus of interest and to differentially hybridize with different TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR alleles. The two ends of the probe are labeled with a quenching fluorophore and a reporter fluorophore, respectively. The TaqMan probe is incorporated into a PCR reaction for the amplification of a target TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene region containing the locus of interest using Taq polymerase. As Taq polymerase exhibits 5′-3′ exonuclease activity but has no 3′-5′ exonuclease activity, if the TaqMan probe is annealed to the target TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR DNA template, the 5′-end of the TaqMan probe will be degraded by Taq polymerase during the PCR reaction thus separating the reporting fluorophore from the quenching fluorophore and releasing fluorescence signals. See Holland et al., Proc. Natl. Acad. Sci. USA, 88:7276-7280 (1991); Kalinina et al., Nucleic Acids Res., 25:1999-2004 (1997); Whitcombe et al., Clin. Chem., 44:918-923 (1998).

In addition, the detection in the present invention can also employ a chemiluminescence-based technique. For example, an oligonucleotide probe can be designed to hybridize to either the wild-type or a variant TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene locus but not both. The probe is labeled with a highly chemiluminescent acridinium ester. Hydrolysis of the acridinium ester destroys chemiluminescence. The hybridization of the probe to the target DNA prevents the hydrolysis of the acridinium ester. Therefore, the presence or absence of a particular mutation in the target DNA is determined by measuring chemiluminescence changes. See Nelson et al., Nucleic Acids Res., 24:4998-5003 (1996).

The detection of genetic variation in the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene in accordance with the present invention can also be based on the “base excision sequence scanning” (BESS) technique. The BESS method is a PCR-based mutation scanning method. BESS T-Scan and BESS G-Tracker are generated which are analogous to T and G ladders of dideoxy sequencing. Mutations are detected by comparing the sequence of normal and mutant DNA. See, e.g., Hawkins et al., Electrophoresis, 20:1171-1176 (1999).

Another useful technique that is gaining increased popularity is mass spectrometry. See Graber et al., Curr. Opin. Biotechnol., 9:14-18 (1998). For example, in the primer oligo base extension (PROBE™) method, a target nucleic acid is immobilized to a solid-phase support. A primer is annealed to the target immediately 5′ upstream from the locus to be analyzed. Primer extension is carried out in the presence of a selected mixture of deoxyribonucelotides and dideoxyribonucleotides. The resulting mixture of newly extended primers is then analyzed by MALDI-TOF. See e.g., Monforte et al., Nat. Med., 3:360-362 (1997).

In addition, the microchip or microarray technologies are also applicable to the detection method of the present invention. Essentially, in microchips, a large number of different oligonucleotide probes are immobilized in an array on a substrate or carrier, e.g., a silicon chip or glass slide. Target nucleic acid sequences to be analyzed can be contacted with the immobilized oligonucleotide probes on the microchip. See Lipshutz et al., Biotechniques, 19:442-447 (1995); Chee et al., Science, 274:610-614 (1996); Kozal et al., Nat. Med. 2:753-759 (1996); Hacia et al., Nat. Genet., 14:441-447 (1996); Saiki et al., Proc. Natl. Acad. Sci. USA, 86:6230-6234 (1989); Gingeras et al., Genome Res., 8:435-448 (1998). Alternatively, the multiple target nucleic acid sequences to be studied are fixed onto a substrate and an array of probes is contacted with the immobilized target sequences. See Drmanac et al., Nat. Biotechnol., 16:54-58 (1998). Numerous microchip technologies have been developed incorporating one or more of the above described techniques for detecting mutations. The microchip technologies combined with computerized analysis tools allow fast screening in a large scale. The adaptation of the microchip technologies to the present invention will be apparent to a person of skill in the art apprised of the present disclosure. See, e.g., U.S. Pat. No. 5,925,525 to Fodor et al; Wilgenbus et al., J. Mol. Med., 77:761-786 (1999); Graber et al., Curr. Opin. Biotechnol., 9:14-18 (1998); Hacia et al., Nat. Genet., 14:441-447 (1996); Shoemaker et al., Nat. Genet., 14:450-456 (1996); DeRisi et al., Nat. Genet., 14:457-460 (1996); Chee et al., Nat. Genet., 14:610-614 (1996); Lockhart et al., Nat. Genet., 14:675-680 (1996); Drobyshev et al., Gene, 188:45-52 (1997).

As is apparent from the above survey of the suitable detection techniques, it may or may not be necessary to amplify the target DNA, i.e., the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene or cDNA or mRNA to increase the number of target DNA molecule, depending on the detection techniques used. For example, most PCR-based techniques combine the amplification of a portion of the target and the detection of the mutations. PCR amplification is well known in the art and is disclosed in U.S. Pat. Nos. 4,683,195 and 4,800,159, both which are incorporated herein by reference. For non-PCR-based detection techniques, if necessary, the amplification can be achieved by, e.g., in vivo plasmid multiplication, or by purifying the target DNA from a large amount of tissue or cell samples. See generally, Sambrook et al., Molecular Cloning: A Laboratory Manual, 2^{nd} ed., Cold Spring Harbor Laboratory, Cold Spring Harbor, N.Y., 1989. However, even with scarce samples, many sensitive techniques have been developed in which small genetic variations such as single-nucleotide substitutions can be detected without having to amplify the target DNA in the sample. For example, techniques have been developed that amplify the signal as opposed to the target DNA by, e.g., employing branched DNA or dendrimers that can hybridize to the target DNA. The branched or dendrimer DNAs provide multiple hybridization sites for hybridization probes to attach thereto thus amplifying the detection signals. See Detmer et al., J. Clin. Microbiol., 34:901-907 (1996); Collins et al., Nucleic Acids Res., 25:2979-2984 (1997); Horn et al., Nucleic Acids Res., 25:4835-4841 (1997); Horn et al., Nucleic Acids Res., 25:4842-4849 (1997); Nilsen et al., J. Theor. Biol., 187:273-284 (1997).

In yet another technique for detecting single nucleotide variations, the Invader® assay utilizes a novel linear signal amplification technology that improves upon the long turnaround times required of the typical PCR DNA sequenced-based analysis. See Cooksey et al., Antimicrobial Agents and Chemotherapy 44:1296-1301 (2000). This assay is based on cleavage of a unique secondary structure formed between two overlapping oligonucleotides that hybridize to the target sequence of interest to form a “flap.” Each “flap” then generates thousands of signals per hour. Thus, the results of this technique can be easily read, and the methods do not require exponential amplification of the DNA target. The Invader® system utilizes two short DNA probes, which are hybridized to a DNA target. The structure formed by the hybridization event is recognized by a special cleavase enzyme that cuts one of the probes to release a short DNA “flap.” Each released “flap” then binds to a fluorescently-labeled probe to form another cleavage structure. When the cleavase enzyme cuts the labeled probe, the probe emits a detectable fluorescence signal. See e.g. Lyamichev et al., Nat. Biotechnol., 17:292-296 (1999).

The rolling circle method is another method that avoids exponential amplification. Lizardi et al., Nature Genetics, 19:225-232 (1998) (which is incorporated herein by reference). For example, Sniper™, a commercial embodiment of this method, is a sensitive, high-throughput SNP scoring system designed for the accurate fluorescent detection of specific variants. For each nucleotide variant, two linear, allele-specific probes are designed. The two allele-specific probes are identical with the exception of the 3′-base, which is varied to complement the variant site. In the first stage of the assay, target DNA is denatured and then hybridized with a pair of single, allele-specific, open-circle oligonucleotide probes. When the 3′-base exactly complements the target DNA, ligation of the probe will preferentially occur. Subsequent detection of the circularized oligonucleotide probes is by rolling circle amplification, whereupon the amplified probe products are detected by fluorescence. See Clark and Pickering, Life Science News 6, 2000, Amersham Pharmacia Biotech (2000).

A number of other techniques that avoid amplification all together include, e.g., surface-enhanced resonance Raman scattering (SERRS), fluorescence correlation spectroscopy, and single-molecule electrophoresis. In SERRS, a chromophore-nucleic acid conjugate is absorbed onto colloidal silver and is irradiated with laser light at a resonant frequency of the chromophore. See Graham et al., Anal. Chem., 69:4703-4707 (1997). The fluorescence correlation spectroscopy is based on the spatio-temporal correlations among fluctuating light signals and trapping single molecules in an electric field. See Eigen et al., Proc. Natl. Acad. Sci. USA, 91:5740-5747 (1994). In single-molecule electrophoresis, the electrophoretic velocity of a fluorescently tagged nucleic acid is determined by measuring the time required for the molecule to travel a predetermined distance between two laser beams. See Castro et al., Anal. Chem., 67:3181-3186 (1995).

In addition, the allele-specific oligonucleotides (ASO) can also be used in in situ hybridization using tissues or cells as samples. The oligonucleotide probes which can hybridize differentially with the wild-type gene sequence or the gene sequence harboring a mutation may be labeled with radioactive isotopes, fluorescence, or other detectable markers. In situ hybridization techniques are well known in the art and their adaptation to the present invention for detecting the presence or absence of a nucleotide variant in the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene of a particular individual should be apparent to a skilled artisan apprised of this disclosure.

Protein-based detection techniques may also prove to be useful, especially when the nucleotide variant causes amino acid substitutions or deletions or insertions or frameshift that affect the protein primary, secondary or tertiary structure. To detect the amino acid variations, protein sequencing techniques may be used. For example, an TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein or fragment thereof can be synthesized by recombinant expression using an TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR DNA fragment isolated from an individual to be tested. Preferably, an TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR cDNA fragment of no more than 100 to 150 base pairs encompassing the polymorphic locus to be determined is used. The amino acid sequence of the peptide can then be determined by conventional protein sequencing methods. Alternatively, the recently developed HPLC-microscopy tandem mass spectrometry technique can be used for determining the amino acid sequence variations. In this technique, proteolytic digestion is performed on a protein, and the resulting peptide mixture is separated by reversed-phase chromatographic separation. Tandem mass spectrometry is then performed and the data collected therefrom is analyzed. See Gatlin et al., Anal. Chem., 72:757-763 (2000).

Other useful protein-based detection techniques include immunoaffinity assays based on antibodies selectively immunoreactive with mutant TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR proteins according to the present invention. The method for producing such antibodies is described above in detail. Antibodies can be used to immunoprecipitate specific proteins from solution samples or to immunoblot proteins separated by, e.g., polyacrylamide gels. Immunocytochemical methods can also be used in detecting specific protein polymorphisms in tissues or cells. Other well-known antibody-based techniques can also be used including, e.g., enzyme-linked immunosorbent assay (ELISA), radioimmunoassay (RIA), immunoradiometric assays (IRMA) and immunoenzymatic assays (IEMA), including sandwich assays using monoclonal or polyclonal antibodies. See e.g., U.S. Pat. Nos. 4,376,110 and 4,486,530, both of which are incorporated herein by reference.

Accordingly, the presence or absence of an TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR nucleotide variant or amino acid variant in an individual can be determined using any of the detection methods described above.

Typically, once the presence or absence of an TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR nucleotide variant or an amino acid variant resulting from a nucleotide variant of the present invention is determined, physicians or genetic counselors or patients or other researchers may be informed of the result. Specifically the result can be cast in a transmittable form that can be communicated or transmitted to other researchers or physicians or genetic counselors or patients. Such a form can vary and can be tangible or intangible. The result with regard to the presence or absence of a TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR nucleotide variant of the present invention in the individual tested can be embodied in descriptive statements, diagrams, photographs, charts, images or any other visual forms. For example, images of gel electrophoresis of PCR products can be used in explaining the results. Diagrams showing where a variant occurs in an individual's TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene are also useful in indicating the testing results. The statements and visual forms can be recorded on a tangible media such as papers, computer readable media such as floppy disks, compact disks, etc., or on an intangible media, e.g., an electronic media in the form of email or website on internet or intranet. In addition, the result with regard to the presence or absence of a nucleotide variant or amino acid variant of the present invention in the individual tested can also be recorded in a sound form and transmitted through any suitable media, e.g., analog or digital cable lines, fiber optic cables, etc., via telephone, facsimile, wireless mobile phone, internet phone and the like.

Thus, the information and data on a test result can be produced anywhere in the world and transmitted to a different location. For example, when a genotyping assay is conducted offshore, the information and data on a test result may be generated and cast in a transmittable form as described above. The test result in a transmittable form thus can be imported into the U.S. Accordingly, the present invention also encompasses a method for producing a transmittable form of information on the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR genotype of an individual. The method comprises the steps of (1) determining the presence or absence of a nucleotide variant according to the present invention in the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene of the individual; and (2) embodying the result of the determining step in a transmittable form. The transmittable form is the product of the production method.

The present invention also provides a kit for genotyping TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene, i.e., determining the presence or absence of one or more of the nucleotide or amino acid variants of present invention in a TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene in a sample obtained from a patient. The kit may include a carrier for the various components of the kit. The carrier can be a container or support, in the form of, e.g., bag, box, tube, rack, and is optionally compartmentalized. The carrier may define an enclosed confinement for safety purposes during shipment and storage. The kit also includes various components useful in detecting nucleotide or amino acid variants discovered in accordance with the present invention using the above-discussed detection techniques.

In one embodiment, the detection kit includes one or more oligonucleotides useful in detecting one or more of the nucleotide variants in TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene. Preferably, the oligonucleotides are allele-specific, i.e., are designed such that they hybridize only to a mutant TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene containing a particular nucleotide variant discovered in accordance with the present invention, under stringent conditions. Thus, the oligonucleotides can be used in mutation-detecting techniques such as allele-specific oligonucleotides (ASO), allele-specific PCR, TaqMan, chemiluminescence-based techniques, molecular beacons, and improvements or derivatives thereof, e.g., microchip technologies. The oligonucleotides in this embodiment preferably have a nucleotide sequence that matches a nucleotide sequence of a variant TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene allele containing a nucleotide variant to be detected. The length of the oligonucleotides in accordance with this embodiment of the invention can vary depending on its nucleotide sequence and the hybridization conditions employed in the detection procedure. Preferably, the oligonucleotides contain from about 10 nucleotides to about 100 nucleotides, more preferably from about 15 to about 75 nucleotides, e.g., contiguous span of 18, 19, 20, 21, 22, 23, 24 or 25 to 21, 22, 23, 24, 26, 27, 28, 29 or 30 nucleotide residues of a TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR nucleic acid one or more of the residues being a nucleotide variant of the present invention, i.e., selected from Table 1. Under most conditions, a length of 18 to 30 may be optimum. In any event, the oligonucleotides should be designed such that it can be used in distinguishing one nucleotide variant from another at a particular locus under predetermined stringent hybridization conditions. Preferably, a nucleotide variant is located at the center or within one (1) nucleotide of the center of the oligonucleotides, or at the 3′ or 5′ end of the oligonucleotides. The hybridization of an oligonucleotide with a nucleic acid and the optimization of the length and hybridization conditions should be apparent to a person of skill in the art. See generally, Sambrook et al., Molecular Cloning: A Laboratory Manual, 2^{nd} ed., Cold Spring Harbor Laboratory, Cold Spring Harbor, N.Y., 1989. Notably, the oligonucleotides in accordance with this embodiment are also useful in mismatch-based detection techniques described above, such as electrophoretic mobility shift assay, RNase protection assay, mutS assay, etc.

In another embodiment of this invention, the kit includes one or more oligonucleotides suitable for use in detecting techniques such as ARMS, oligonucleotide ligation assay (OLA), and the like. The oligonucleotides in this embodiment include a TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene sequence of about 10 to about 100 nucleotides, preferably from about 15 to about 75 nucleotides, e.g., contiguous span of 18, 19, 20, 21, 22, 23, 24 or 25 to 21, 22, 23, 24, 26, 27, 28, 29 or 30 nucleotide residues immediately 5′ upstream from the nucleotide variant to be analyzed. The 3′ end nucleotide in such oligonucleotides is a nucleotide variant in accordance with this invention.

The oligonucleotides in the detection kit can be labeled with any suitable detection marker including but not limited to, radioactive isotopes, fluorephores, biotin, enzymes (e.g., alkaline phosphatase), enzyme substrates, ligands and antibodies, etc. See Jablonski et al., Nucleic Acids Res., 14:6115-6128 (1986); Nguyen et al., Biotechniques, 13:116-123 (1992); Rigby et al., J. Mol. Biol., 113:237-251 (1977). Alternatively, the oligonucleotides included in the kit are not labeled, and instead, one or more markers are provided in the kit so that users may label the oligonucleotides at the time of use.

In another embodiment of the invention, the detection kit contains one or more antibodies selectively immunoreactive with certain TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR proteins or polypeptides containing specific amino acid variants discovered in the present invention. Methods for producing and using such antibodies have been described above in detail.

Various other components useful in the detection techniques may also be included in the detection kit of this invention. Examples of such components include, but are not limited to, Taq polymerase, deoxyribonucleotides, dideoxyribonucleotides other primers suitable for the amplification of a target DNA sequence, RNase A, mutS protein, and the like. In addition, the detection kit preferably includes instructions on using the kit for detecting nucleotide variants in TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene sequences.

### 7. Use of Genotyping in Diagnostic Applications

All of the SNPs of the present invention can be used to predict whether or not specific genes exhibit altered levels of expression in individual patients. As such, they all have utility in a variety of applications beyond those specifically described below. In particular, the SNPs can be used to catagorize patient populations into population subgroups based upon the relative level of gene expression associated with a particular SNP. For example, patents can be classified as normal-expressors, when they exhibit average or intermediate levels of gene expression; over-expressors, when they exhibit increased levels of gene expression; or under-expressors, when they exhibit reduced levels of gene expression. These categories or patient population sub-groupings can be useful when evaluating the effect that altered gene expression has on particular physiological or pharmacokinetic parameters, or selecting patients for clinical trials for therapeutic treatments.

In those situations where a protein being expressed directly plays a critical biological role in the human body, assessment of an individual's genotype at a SNP associated with altered levels of expression of the gene encoding this protein may prove more efficient, economical, and more reliably predictive, than assays designed to detect the gene product (e.g., mRNA or protein) itself. One can readily envision large-scale screens of SNPs associated with altered levels of gene expression being conducted for multiple genes on a single microchip designed to simultaneously assess the expression of a large number of genes whose products are known to be involved in increased risk of a particular undesirable phenotype.

For example, where a protein being expressed plays a role in some aspect of drug metabolism, including, for example, detoxification, secretion or excretion, assessment of an individual's genotype at a SNP associated with altered levels of expression of the gene encoding this protein may prove more efficient, economical, and more reliably predictive, than assays designed to detect the effects of the gene product (e.g., enzyme), such as altered pharmacokinetic profiles, rapid excretion, reduced efficacy of the drug, etc.

Additionally, when the relative level of expression of a particular set of genes can be predicted by the presence or absence of a particular SNP, diagnostic assays designed to assess the expression levels of the specific individual genes becomes superflouous, since detection of a single SNP would prove more economical or informative than the quantitiative analysis of expression of the individual genes at the mRNA or protein level. Such a situation would be expected when the single SNP being genotyped is associated with the level of expression of a transcription factor, whose over-expression would result in increased transcription of a multiplicity of genes.

When selecting patients to be included in clinical trails of candidate drug compounds, the SNPs of the instant invention can be used to decide which patients to enroll in the trials, and which patients to exclude. The SNPs could also be used to determine which patients should be included in particular dosage regimes within a clinical trial. For example, if the SNP is associated with expression levels of a gene involved in the metabolism of the candidate drug, patients that are categorized as over-expressors could be intentionally placed in higher dosage regimes, than those identified as under-expressors.

Similarly, the SNPs of the instant invention can provide critically useful information, which can be used to assist health practitioners in making decisions about the method and course of treatment to be given to specific patients. The information provided by these SNPs can, for example, direct the practitioner to choose one particular type of drug over another. In other words, the information provided by these SNPs can be used to direct qualititative decisions for treatment. Additionally, the information provided by these SNPs can be used to direct quantitative decisions for treatment of patients, such as the dosage to be given, the frequency with which it is given, and even the route by which the dosage is to be administered.

The SNPs of the instant invention can also be used to predict physiologic or pharmacokinetic consequences of treatment with a specific drug at a specific dosage.

Specific diagnostic and prognostic applications for the SNPs of the present invention will now be discussed.

TLK1

As indicated in Tables 1, 2 or 3 and Tables 36 and 37, the expression level of the TLK1 gene in human cells is an inheritable “quantitative trait” with genetic determinants. Furthermore, the SNPs and/or haplotypes in accordance with the present invention are associated with the “quantitative trait”, i.e., the TLK1 mRNA level in human cells. Specifically, the SNPs EX7@+63A, EX7@+190C, EX11@51G and EX25@855A of TLK1 are associated with a “low expression phenotype” while the EX7@+63G, EX7@+190T, EX11@51A and EX25@855G of TLK1 are associated with a “high expression phenotype.” Thus, the SNPs and/or haplotypes are particularly useful in predicting the level of TLK1 gene expression in an individual.

Thus, in one aspect, the present invention encompasses a method for predicting or detecting cancer susceptibility in an individual, which comprises the step of genotyping the individual to determine the individual's genotype at one or more of the TLK1 loci identified in the present invention, namely EX7@+63, EX7@+190, EX11@51 and EX25@855; or, at another locus at which the genotype is in linkage disequilibrium with one of the SNPs of the present invention. Thus, if one or more the EX7@+63A, EX7@+190C, EX11@51G and EX25@855A are detected, or a SNP that is in linkage disequilibrium with any one of such SNPs is detected in the individual, then it can be reasonably predicted that there is an increased likelihood that the individual will have an increased level of chromosome missegregation and aneuploidy and, thus, is at an increased risk of developing cancer. In particular, if an individual is homozygous with the TLK1 genotype EX7@+63A/A, EX7@+190C/C, EX11@51G/G and/or EX25@855A/A, then it can be reasonably predicted that the individual has an elevated susceptibility to chromosome missegregation and aneuploidy. In other words, such an individual has an increased likelihood or is at an increased risk of developing cancer. If an individual is heterozygous, then his or her risk of developing cancer is at an intermediate level. On the other hand, if the individual is homozygous with the TLK1 genotype EX7@+63G/G, EX7@+190T/T, EX11@51A/A and/or EX25@855G/G, then it can be reasonably predicted that the individual has a reduced susceptibility to cancer. Alternatively, if the individual is homozygous with a genotype at a TLK1 locus that is in the same haplotype with the SNPs EX7@+63A, EX7@+190C, EX11@51G and/or EX25@855A (in linkage disequilibrium), then it can reasonably be predicted that the individual has an increased susceptibility to cancer.

In another aspect of the present invention, a method is provided for predicting susceptibility to diseases associated with DNA damage including, but not limited to, heart disease and cancer. This method comprises genotyping the individual at one of more of the TLK1 loci identified in the present invention, namely EX7@+63, EX7@+190, EX11@51 and/or EX25@855, or another locus at which the genotype is in linkage disequilibrium with one of the SNPs of the present invention. Thus, if one or more of the SNPs EX7@+63G, EX7@+190T, EX11@51A or EX25@855G are detected, than it can be reasonably predicted that the individual has an increased susceptibility to cancer. Specifically, if the individual is homozygous with the TLK1 genotype EX7@+63G/G, EX7@+190T/T, EX11@51A/A or EX25@855G/G, it can be reasonably predicted that the individual has an elevated susceptibility to DNA damage such as that caused by ionizing radiation. If the individual is heterozygous, it can be predicted the individual will have an intermediate susceptibility to cancer. On the other hand, if the individual is homozygous with the TLK1 genotype EX7@+63G/G, EX7@+190T/T, EX11@51A/A and/or EX25@855G/G, then it can be reasonably predicted that the individual has a reduced susceptibility to cancer, especially those associated with DNA damage.

In yet another aspect, the present invention provides a method of predicting patient and tumor response to cancer treatment. Although some normal cells are affected by radiation, most normal cells appear to recover more fully from the effects of radiation than do cancer cells. In accordance with the present invention, the TLK1 gene of a patient in need of radiation treatment is sequenced to determine the genotype at one or more of the SNPs or haplotypes of the present invention, specifically mainly EX7@+63, EX7@+190, EX11@51 and EX25@855, or another locus at which the genotype is in linkage disequilibrium with any one of the SNPs of the present invention. Expression levels of TLK1 can be utilized to predict the effectiveness of treatment in a patient, and the ability of the patient to recover from the radiation therapy treatment itself. If one or more of the SNPs EX7@+63G, EX7@+190T, EX11@51A and EX25@855G are detected in the patient's genome, or a SNP that is in linkage disequilibrium with any one of such SNPs is detected in an individual, then it can be reasonably predicted that the patient is likely to recover more rapidly from the DNA damaging radiation therapy treatment. In short, the individual will likely have a shorter recovery time from the treatment. On the other hand, where an individual has the TLK1 genotype EX7@+63A/A, EX7@+190C/C, EX11@51A/A and EX25@855G/G, then it can be reasonably predicted that the individual will a slower recovery from cancer treatments involving DNA damage, such as radiation therapy.

While the above discussion relates to the expected recovery of the patient from the DNA damaging radiation therapy, it is necessary to determine the TLK1 genotype of the cancerous growth in order to predict the likely efficacy of radiation treatment in killing the cancer, since increased TLK1 expression by a tumor would be expected to result in increased resistance of the tumor to the DNA damaging treatment. In the event that the cancerous growth has the TLK1 genotype EX7@+63G/G, EX7@+190T/T, EX11@51G/G and EX25@855A/A, it can be reasonably predicted that the cancer will have a decreased response to radiation therapy. If the cancerous growth is heterozygous, it can be predicted that the cancer will have an intermediate response to treatment. On the other hand, where the cancerous growth has the TLK1 genotype EX7@+63A/A, EX7@+190C/C, EX11@51A/A and EX25@855G/G, then it can be reasonably predicted that the cancerous growth will have an increased sensitivity to cancer treatment involving DNA damage, and treatment by ionizing radiation would be expected to be more effective at killing the cancer.

Another aspect of the present invention provides a method of predicting the risk of DNA-damaging therapy in creating new cancer, especially leukemia. This method comprises the step of genotyping the patient to determine the patient's genotype at one or more of the TLK1 loci specified in the present invention; specifically EX7@+63, EX7@+190, EX11@51 and EX25@855, or another locus at which the genotype is in linkage disequilibrium with one of the SNPs or haplotypes of the present invention. Thus, if one or more of the SNPs EX7@+63G, EX7@+190T, EX11@51A and EX25@855G are detected in a patient, it can be predicted that the patient will have an increased DNA-damaging therapy, and would be less likely to develop a new cancer, such as leukemia, as a result of the DNA-damaging therapy used to treat the initial cancer. Alternatively, if one or more of the SNPs EX7@+63A, EX7@+190C, EX11@51G and EX25@855A are present in the patient, it can be predicted that the individual will be more susceptible to developing a secondary cancer as a result of treatment using DNA-damaging therapy.

Particularly, if a patient is homozygous with the TLK1 genotype EX7@+63G/G, EX7@+190T/T, EX11@51A/A and EX25@855G/G, then the patient has a higher resistance to DNA-damaging therapy, such as treatment with ionizing radiation. Conversely, if the patient is homozygous with the TLK1 genotype EX7@+63A/A, EX7@+190C/C, EX11@51G/G and EX25@855A/A, then the patient has an increased susceptibility to secondary cancers caused by the primary cancer treatment with DNA-damaging therapy, such as treatment with ionizing radiation.

In another aspect of the present invention, a method is provided for predicting or detecting susceptibility to metabolic disorders such as diabetes, insulin resistance, Hermansky-Pudlak syndrome and ARC syndrome in an individual, comprising determining the genotype of an individual at one TLK1 loci identified in the present invention, namely EX7@+63, EX7@+190, EX11@51 and EX25@855; or, at another locus which is in linkage disequilibrium with one of the SNPs or haplotypes of the present invention. Thus, if one or more the TLK1 SNPs EX7@+63G, EX7@+190T, EX11@51A and EX25@855G are detected, or a SNP that is in linkage disequilibrium with any one of such SNPs is detected in the individual, then it can be reasonably predicted that the individual is at an increased risk of developing a metabolic disorder such as diabetes or insulin resistance. Particularly, if an individual is homozygous with the TLK1 genotype EX7@+63A/A, EX7@+190C/C, EX11@51G/G and EX25@855A/A, it can be reasonably predicted that the individual will have an increased susceptibility to metabolic disorders such as diabetes and insulin resistance. An individual that is heterozygous will have an intermediate risk of developing metabolic disorders. On the other hand, if an individual is homozygous with the TLK1 genotype EX7@+63G/G, EX7@+190T/T, EX11@51A/A and EX25@855G/G, it can be predicted that an individual will have a decreased susceptibility to metabolic disorders such as diabetes, insulin resistance, Hermansky-Pudlak syndrome and ARC syndrome.

The SNPs listed in Table 3, i.e. those at positions 171,620,667, 171,622,696, 171,622,741 and 171,840,599 of chromosome II, have also been shown to be associated with altered TLK1 mRNA levels. Chromosome II SNPs associated with lower TLK1 mRNA expression levels are 171,620,667G, 171,622,696G, 171,622,741C and 171,840,599G, whereas those associated with higher TLK1 mRNA expression are 171,620,667A, 171,622,696A, 171,622,741T and 171,840,599A. In addition to the SNPs described above, these chromosome II SNPs may be utilized in the applications, as described above.

WARS2

As indicated in Table 4 above and Table 38 and 39 below, the expression level of the WARS2 gene in human cells is an inheritable “quantitative trait” with genetic determinants. Furthermore, the WARS2 gene SNPs in accordance with the present invention are associated with the “quantitative trait”, i.e., the WARS2 mRNA level in human cells. Specifically, the WARS2 SNPs EX1@−963G, EX1@−103T, EX6@780G, EX6@842T and EX6@2152G are associated with a “low expression phenotype” while the EX1@−963A, EX1@−103C, EX6@780A, EX6@842G and EX6@2152A SNPs are associated with a “high expression phenotype.” Thus, the WARS2 SNPs are particularly useful in predicting the level of WARS2 gene expression in an individual.

Thus, in one aspect, the present invention encompasses a method for predicting or detecting susceptibility to neurodegenerative disease in an individual, which comprises the step of genotyping the individual to determine the individual's genotype at one or more of the WARS2 loci identified in the present invention, namely EX1@−963, EX1@−103, EX6@780, EX6@842 or EX6@2152; or, at another locus at which the genotype is in linkage disequilibrium with one of the SNPs of the present invention. Thus, if one or more the SNPs EX1@−963G, EX1@−103T, EX6@780G, EX6@842T or EX6@2152G are detected, or a SNP that is in linkage disequilibrium with any one of such SNPs is detected in the individual, then it can be reasonably predicted that the individual is at an increased risk of developing neurodegenerative disease, particularly Friedreich's ataxia, Huntington's disease, Alzheimer's disease, ALS and Parkinson's disease. In particular, if an individual is homozygous with the WARS2 genotype EX1@−963G/G, EX1@−103T/T, EX6@780G/G, EX6@842T/T or EX6@2152G/G, then it can be reasonably predicted that the individual has an elevated susceptibility to neurodegenerative disease, particularly Friedreich's ataxia, Huntington's disease, Alzheimer's disease, ALS and Parkinson's disease. If an individual is heterozygous, then his or her risk of developing neurodegenerative disease is at an intermediate level. One the other hand, if the individual is homozygous with the WARS2 genotype EX1@−963A/A, EX1@−103C/C, EX6@780A/A, EX6@842G/G or EX6@2152A/A, then it can be reasonably predicted that the individual has a reduced susceptibility to neurodegenerative disease, particularly Friedreich's ataxia, Huntington's disease, Alzheimer's disease, ALS and Parkinson's disease.

In another aspect, the present invention provides a method for predicting or detecting susceptibility to cardiovascular disease in an individual, which comprises the step of genotyping the individual to determine the individual's genotype at one or more of the WARS2 loci identified in the present invention, namely EX1@−963, EX1@−103, EX6@780, EX6@842 or EX6@2152, or another locus at which the genotype is in linkage disequilibrium with one of the SNPs of the present invention. Thus, if one or more the SNPs EX1@−963G, EX1@−103T, EX6@780G, EX6@842T or EX6@2152G are detected, or a SNP that is in linkage disequilibrium with any one of such SNPs is detected in the individual, then it can be reasonably predicted that the individual is at an increased risk of developing cardiovascular disease, such as dilated and hypertrophic cardiomyopathy, cardiac conduction defects, sudden death, ischemic and alcoholic cardiomyopathy, and myocarditis. In particular, if an individual is homozygous with the WARS2 genotype EX1@−963G/G, EX1@−103T/T, EX6@780G/G, EX6@842T/T or EX6@2152G/G, then it can be reasonably predicted that the individual has an elevated susceptibility to cardiovascular disease, such as dilated and hypertrophic cardiomyopathy, cardiac conduction defects, sudden death, ischemic and alcoholic cardiomyopathy, and myocarditis. If an individual is heterozygous, then his or her risk of developing cardiovascular disease is at an intermediate level. One the other hand, if the individual is homozygous with the WARS2 genotype EX1@−963A/A, EX1@−103C/C, EX6@780A/A, EX6@842G/G or EX6@2152A/A, then it can be reasonably predicted that the individual has a reduced susceptibility to cardiovascular disease, such as dilated and hypertrophic cardiomyopathy, cardiac conduction defects, sudden death, ischemic and alcoholic cardiomyopathy, and myocarditis.

In another aspect, the present invention provides a method for predicting or detecting cancer susceptibility in an individual, comprising the step of genotyping the individual to determine the individual's genotype at one or more of the WARS2 loci identified in the present invention, namely EX1@−963, EX1@−103, EX6@780, EX6@842 or EX6@2152, or another locus at which the genotype is in linkage disequilibrium with one of the SNPs of the present invention. Thus, if one or more the SNPs EX1@−963G, EX1@−103T, EX6@780G, EX6@842T or EX6@2152G are detected, or a SNP that is in linkage disequilibrium with any one of such SNPs is detected in the individual, then it can be reasonably predicted that the individual is at an increased risk of developing cancer. In particular, if an individual is homozygous with the WARS2 genotype EX1@−963G/G, EX1@−103T/T, EX6@780G/G, EX6@842T/T or EX6@2152G/G, then it can be reasonably predicted that the individual has an elevated susceptibility to cancer. If an individual is heterozygous, then his or her risk of developing cancer is at an intermediate level. One the other hand, if the individual is homozygous with the WARS2 genotype EX1@−963A/A, EX1@−103C/C, EX6@780A/A, EX6@842G/G or EX6@2152A/A, then it can be reasonably predicted that the individual has a reduced susceptibility to cancer.

In yet another aspect, the present invention provides a method for identifying high-risk patients who have a poor prognosis of cancer, or for the prognosis of cancer, or predicting/determining the invasiveness and metastatic potential of tumor in a patient, particularly a cancer patient. The individual to be tested can be a healthy person or an individual diagnosed with cancer. The method comprises the step of genotyping the individual to determine the individual's genotype at one or more of the WARS2 loci identified in the present invention, namely EX1@−963, EX1@−103, EX6@780, EX6@842 or EX6@2152; or, at another locus at which the genotype is in linkage disequilibrium with one of the SNPs or haplotypes of the present invention. Thus, if one or more the SNPs EX1@−963G, EX1-103T, EX6@780G, EX6@842T or EX6@2152G are detected, or a SNP that is in linkage disequilibrium with any one of such SNPs is detected in the individual, then it can be reasonably predicted that when cancer occurs within that individual, it has high metastatic potential, that the cancer has poor prognosis, and that the tumor cells are likely to be invasive. In other words, the individual has an increased likelihood, or is at an increased risk for cancer metastasis. Particularly, if an individual is homozygous with the WARS2 genotype EX1@−963G/G, EX1@−103T/T, EX6@780G/G, EX6@842T/T or EX6@2152G/G, then the individual has particular poor prognosis because it is likely that the tumor cells are highly invasive. In other words, the individual has a substantially increased likelihood, or is at a substantially increased risk for cancer metastasis. However, if an individual is heterozygous with the genotype EX1@−963G/A, EX1@−103T/C, EX6@780G/A, EX6@842T/G or EX6@2152G/A, then the individual has an intermediate prognosis and that the tumor cells are potentially invasive. Specifically, the individual has an intermediate level of risk for cancer metastasis. That is, the risk is greater than a person having a homozygous WARS2 genotype of EX1@−963A/A, EX1@−103C/C, EX6@780A/A, EX6@842G/G or EX6@2152A/A, but is lower than a person having a homozygous genotype of EX1@−963A/A, EX1@−103C/C, EX6@780A/A, EX6@842G/G or EX6@2152A/A.

Thus, if the individual is homozygous with a WARS2 genotype EX1@−963A/A, EX1@−103C/C, EX6@780A/A, EX6@842G/G or EX6@2152A/A, it can be reasonably predicted that any tumors in that individual have a low metastatic potential, that the cancer has good prognosis, and that the tumor cells are not likely to be invasive. That is, the individual does not have an increased likelihood, or increased risk, of cancer metastasis.

ARTS1

As indicated in Tables 5-11 and Tables 40-44, the expression level of the ARTS1 gene in human cells is an inheritable “quantitative trait” with genetic determinants. Furthermore, the SNPs and/or haplotypes in accordance with the present invention are associated with the “quantitative trait”, i.e., the ARTS1 mRNA level in human cells. Specifically, the SNPs EX1@−1125C, EX2@397C, EX20@1085G, EX6@126G, EX12@44A, EX15@74A, EX6@149C, EX8@−10G, EX9@39C, EX9@+18C, EX11@59G, EX12@−28G, EX12@−7C, EX15@88G, EX19@173A, EX19@328w, EX19@885C, EX20@2105T, EX20@719T and EX20@1038C are associated with a “low expression phenotype” while the EX1@−1125T, EX2@397G, EX20@1085A, EX6@126A, EX12@44G, EX15@74G, EX6@149T, EX8@−10A, EX9@39T, EX9@+18T, EX1@59A, EX12@−28T, EX12@−7A, EX15@88C, EX19@173C, EX19@328m, EX19@885T, EX20@2105C, EX20@719C and EX20@1038A are associated with a “high expression phenotype.” Thus, the SNPs and/or haplotypes are particularly useful in predicting the level of ARTS1 gene expression in an individual.

Thus, in one aspect, the present invention encompasses a method for predicting or detecting cancer susceptibility in an individual, which comprises the step of genotyping the individual to determine the individual's genotype at one or more of the ARTS1 loci identified in the present invention, namely EX1@−1125, EX2@397, EX20@1085, EX6@126, EX12@44, EX15@74, EX6@149, EX8@−10, EX9@39, EX9@+18, EX11@59, EX12@−28, EX12@−7, EX15@88, EX19@173, EX19@328, EX19@885, EX20@2105, EX20@719 or EX20@1038; or, at another locus at which the genotype is in linkage disequilibrium with one of the SNPs or haplotypes of the present invention. Thus, if one or more the ARTS1 SNPs EX1@−1125C, EX2@397C, EX20@1085G, EX6@126G, EX12@44A, EX15@74A, EX6@149C, EX8@−10G, EX9@39C, EX9@+18C, EX11@59G, EX12@−28G, EX12@−7C, EX15@88G, EX19@173A, EX19@328w, EX19@885C, EX20@2105T, EX20@719T or EX20@1038C are detected, or a SNP that is in linkage disequilibrium with any one of such SNPs is detected in the individual, then it can be reasonably predicted that the individual is at an increased risk of developing cancer, particularly skin cancer, lung cancer, ovarian cancer or thyoma. In particular, if an individual is homozygous with the ARTS1 genotype EX1@−1125C/C, EX2@397C/C, EX20@1085G/G, EX6@126G/G, EX12@44A/A, EX15@74A/A, EX6@149C/C, EX8@−10G/G, EX9@39C/C, EX9@+18C/C, EX11@59G/G, EX12@−28G/G, EX12@−7C/C, EX15@88G/G, EX19@173A/A, EX19@328w/w, EX19@885C/C, EX20@2105T/T, EX20@719T/T or EX20@1038C/C, then it can be reasonably predicted that the individual has an elevated susceptibility to cancer, particularly skin cancer (e.g., melanoma), lung cancer (e.g., NSCLCs), ovarian cancer or thyoma. Likewise, if the individual is homozygous with an ARTS1 genotype at a locus that is in the same haplotype with the SNPs EX6@126G, EX12@44A and EX15@74A (in linkage disequilibrium), or in the same haplotype (linkage disequilibrium) with the SNPs EX6@149C and EX8@−10G, or in the same haplotype (linkage disequilibrium) with the SNPs EX9@39C, EX9@+18C, EX11@59G, EX12@−28G, EX12@−7C and EX15@88G, or in the same haplotype (linkage disequilibrium) with the SNPs EX19@173A, EX19@328w, EX19@885C and EX20@2105T, or in the same haplotype (linkage disequilibrium) with the SNPs EX20@719T and EX20@1038C, then it can reasonably be predicted that the individual has an elevated susceptibility to cancer. In other words, such an individual has an increased likelihood or is at an increased risk of developing cancer. If an individual is heterozygous, then his or her risk of developing cancer is at an intermediate level. On the other hand, if the individual is homozygous with the ARTS1 genotype EX1@−1125T/T, EX2@397G/G, EX20@1085A/A, EX6@126A/A, EX12@44G/G, EX15@74G/G, EX6@149T/T, EX8@−10A/A, EX9@39T/T, EX9@+18T/T, EX11@59A/A, EX12@−28T/T, EX12@−7A/A, EX15@88C/C, EX19@173C/C, EX19@328 m/m, EX19@885T/T, EX20@2105C/C, EX20@719C/C or EX20@1038A/A, then it can be reasonably predicted that the individual has a reduced susceptibility to cancer. Similarly, if the individual is homozygous with an ARTS1 genotype at a locus that is in the same haplotype with the SNPs EX6@126A, EX12@44G and EX15@74G (in linkage disequilibrium), or in the same haplotype (linkage disequilibrium) with the SNPs EX6@149T and EX8@−10A, or in the same haplotype (linkage disequilibrium) with the SNPs EX9@39T, EX9@+18T, EX11@59A, EX12@−28T, EX12@−7A and EX15@88C, or in the same haplotype (linkage disequilibrium) with the SNPs EX19@173C, EX19@328m, EX19@885TC and EX20@2105C, or in the same haplotype (linkage disequilibrium) with the SNPs EX20@719C and EX20@1038A, then it can reasonably be predicted that the individual has a reduced susceptibility to cancer.

In another aspect, the present invention provides a method for identifying high-risk patients who have a poor prognosis of cancer, or for the prognosis of cancer, or predicting/determining the invasiveness and metastatic potential of a tumor in a patient, particularly cancer patient. The individual to be tested can be a healthy person or an individual diagnosed of cancer. The method comprises the step of genotyping the individual to determine the individual's genotype at one or more of the ARTS1 loci identified in the present invention, namely EX1@−1125, EX2@397, EX20@1085, EX6@126, EX12@44, EX15@74, EX6@149, EX8@−10, EX9@39, EX9@+18, EX11@59, EX12@−28, EX12@−7, EX15@88, EX19@173, EX19@328, EX19@885, EX20@2105, EX20@719 or EX20@1038; or, at another locus at which the genotype is in linkage disequilibrium with one of the SNPs or haplotypes of the present invention.

Thus, if one or more the ARTS1 SNPs EX1@−1125C, EX2@397C, EX20@1085G, EX6@126G, EX12@44A, EX15@74A, EX6@149C, EX8@−10G, EX9@39C, EX9@+18C, EX11@59G, EX12@−28G, EX12@−7C, EX15@88G, EX19@173A, EX19@328w, EX19@885C, EX20@2105T, EX20@719T or EX20@1038C are detected, or a SNP that is in linkage disequilibrium with any one of such SNPs is detected in the individual, then it can be reasonably predicted that cancers occurring within that individual have high metastatic potential, that the cancer has poor prognosis, and that the tumor cells are likely to be invasive. In other words, the individual has an increased likelihood, or is at an increased risk for cancer metastasis. Particularly, if an individual is homozygous with the ARTS1 genotype EX1@−1125C/C, EX2@397C/C, EX20@1085G/G, EX6@126G/G, EX12@44A/A, EX15@74A/A, EX6@149C/C, EX8@−10G/G, EX9@39C/C, EX9@+18C/C, EX11@59G/G, EX12@−28G/G, EX12@−7C/C, EX15@88G/G, EX19@173A/A, EX19@328w/w, EX19@885C/C, EX20@2105T/T, EX20@719T/T or EX20@1038C/C, then the individual has particularly poor prognosis and the tumor cells are likely highly invasive. In other words, the individual has a substantially increased likelihood, or is at a substantially increased risk from cancer metastasis. However, if an individual is heterozygous with the ARTS1 genotype EX1@−1125T/C, EX2@397C/G, EX20@1085G/A, EX6@126G/A, EX12@44G/A, EX15@74G/A, EX6@149T/C, EX8@−10G/A, EX9@39C/T, EX9@+18T/C, EX11@59G/A, EX12@−28G/T, EX12@−7C/A, EX15@88G/C, EX19@173C/A, EX19@328w/m, EX19@885C/T, EX20@2105T/C, EX20@719T/C or EX20@1038A/C, then the individual has poor prognosis and the tumor cells are likely invasive. Specifically, the individual has an intermediate level of risk of cancer metastasis. That is, the risk is greater than a person having a homozygous ARTS1 genotype of EX1@−1125T/T, EX2@397G/G, EX20@1085A/A, EX6@126A/A, EX12@44G/G, EX15@74G/G, EX6@149T/T, EX8@−10A/A, EX9@39T/T, EX9@+18T/T, EX11@59A/A, EX12@−28T/T, EX12@−7A/A, EX15@88C/C, EX19@173C/C, EX19@328m/m, EX19@885T/T, EX20@2105C/C, EX20@719C/C or EX20@1038A/A, but is lower than a person having a homozygous genotype of EX1@−1125C/C, EX2@397C/C, EX20@1085G/G, EX6@126G/G, EX12@44A/A, EX15@74A/A, EX6@149C/C, EX8@−10G/G, EX9@39C/C, EX9@+18C/C, EX1@59G/G, EX12@−28G/G, EX12@−7C/C, EX15@88G/G, EX19@173A/A, EX19@328w/w, EX19@885C/C, EX20@2105T/T, EX20@719T/T or EX20@1038C/C.

Thus, if the individual is homozygous with the ARTS1 genotype EX1@−1125T/T, EX2@397G/G, EX20@1085A/A, EX6@126A/A, EX12@44G/G, EX15@74G/G, EX6@149T/T, EX8@−10A/A, EX9@39T/T, EX9@+18T/T, EX11@59A/A, EX12@−28T/T, EX12@−7A/A, EX15@88C/C, EX19@173C/C, EX19@328m/m, EX19@885T/T, EX20@2105C/C, EX20@719C/C or EX20@1038A/A, then it can be reasonably predicted that a tumor occurring within this individual has a low metastatic potential, that the cancer has good prognosis, and that the tumor cells are likely not invasive. That is, the individual does not have an increased likelihood or increased risk of cancer metastasis. Similarly, if the individual is homozygous with an ARTS1 genotype at a locus that is in the same haplotype with the SNPs EX6@126A, EX12@44G and EX15@74G (in linkage disequilibrium), or in the same haplotype (linkage disequilibrium) with the SNPs EX6@149T and EX8@−10A, or in the same haplotype (linkage disequilibrium) with the SNPs EX9@39T, EX9@+18T, EX11@59A, EX12@−28T, EX12@−7A and EX15@88C, or in the same haplotype (linkage disequilibrium) with the SNPs EX19@173C, EX19@328m, EX19@885TC and EX20@2105C, or in the same haplotype (linkage disequilibrium) with the SNPs EX20@719C and EX20@1038A, then it can reasonably be predicted that tumors occurring within this individual have a low metastatic potential, that the cancer has good prognosis, and that the tumor cells are likely not invasive. In other words, the individual does not have an increased likelihood, or increased risk for cancer metastasis.

In another aspect, the present invention encompasses a method for predicting or detecting susceptibility in an individual to cardiovascular disease, which comprises the step of genotyping the individual to determine the individual's ARTS1 genotype at one or more of the loci identified in the present invention, namely EX1@−1125, EX2@397, EX20@1085, EX6@126, EX12@44, EX15@74, EX6@149, EX8@−10, EX9@39, EX9@+18, EX11@59, EX12@−28, EX12@−7, EX15@88, EX19@173, EX19@328, EX19@885, EX20@2105, EX20@719 or EX20@1038; or, at another locus at which the genotype is in linkage disequilibrium with one of the SNPs or haplotypes of the present invention. Thus, if one or more the SNPs EX1@−1125C, EX2@397C, EX20@1085G, EX6@126G, EX12@44A, EX15@74A, EX6@149C, EX8@−10G, EX9@39C, EX9@+18C, EX11@59G, EX12@−28G, EX12@−7C, EX15@88G, EX198173A, EX19@328w, EX19@885C, EX20@2105T, EX20719T or EX20@1038C are detected, or a SNP that is in linkage disequilibrium with any one of such SNPs is detected in the individual, then it can be reasonably predicted that the individual is at an increased risk of developing a cardiovascular disease, particularly high blood pressure, hypertension, cardiac hypertrophy, myocardial damage or coronary heart disease. Particularly, if an individual is homozygous with the ARTS1 genotype EX1@−1125C/C, EX2@397C/C, EX20@1085G/G, EX6@126G/G, EX12@44A/A, EX15@74A/A, EX6@149C/C, EX8@−10G/G, EX9@39C/C, EX9@+18C/C, EX11@59G/G, EX12@−28G/G, EX12@−7C/C, EX15@88G/G, EX19@173A/A, EX19@328w/w, EX19@885C/C, EX20@2105T/T, EX20@719T/T or EX20@1038C/C, then it can be reasonably predicted that the individual has an elevated susceptibility to cardiovascular disease, particularly hypertension, cardiac hypertrophy, myocardial damage and coronary heart disease. Likewise, if the individual is homozygous with a genotype at an ARTS1 locus that is in the same haplotype with the SNPs EX6@126G, EX12@44A and EX15@74A (in linkage disequilibrium), or in the same haplotype (linkage disequilibrium) with the SNPs EX6@149C and EX8@−10G, or in the same haplotype (linkage disequilibrium) with the SNPs EX9@39C, EX9@+18C, EX11@59G, EX12@−28G, EX12@−7C and EX15@88G, or in the same haplotype (linkage disequilibrium) with the SNPs EX19@173A, EX19@328w, EX19@885C and EX20@2105T, or in the same haplotype (linkage disequilibrium) with the SNPs EX20@719T and EX20@1038C, then it can reasonably be predicted that the individual has an elevated susceptibility to cardiovascular disease. In other words, such an individual has an increased likelihood, or is at an increased risk for developing cardiovascular disease. If an individual is heterozygous, then his or her risk of developing cardiovascular disease is at an intermediate level. On the other hand, if the individual is homozygous with the ARTS1 genotype EX1@−1125T/T, EX2@397G/G, EX20@1085A/A, EX6@126A/A, EX12@44G/G, EX15@74G/G, EX6@149T/T, EX8@−10A/A, EX9@39T/T, EX9@+18T/T, EX11@59A/A, EX12@−28T/T, EX12@−7A/A, EX15@88C/C, EX19@173C/C, EX19@328m/m, EX19@885T/T, EX20@2105C/C, EX20@719C/C or EX20@1038A/A, then it can be reasonably predicted that the individual has a reduced susceptibility to cardiovascular disease. Similarly, if the individual is homozygous with a genotype at an ARTS1 locus that is in the same haplotype with the SNPs EX6@126A, EX12@44G and EX15@74G (in linkage disequilibrium), or in the same haplotype (linkage disequilibrium) with the SNPs EX6@149T and EX8@−10A, or in the same haplotype (linkage disequilibrium) with the SNPs EX9@39T, EX9@+18T, EX11@59A, EX12@−28T, EX12@−7A and EX15@88C, or in the same haplotype (linkage disequilibrium) with the SNPs EX19@173C, EX19@328m, EX19@885TC and EX20@2105C, or in the same haplotype (linkage disequilibrium) with the SNPs EX20@719C and EX20@1038A, then it can reasonably be predicted that the individual has a reduced susceptibility to cardiovascular disease, particularly high blood pressure, hypertension, cardiac hypertrophy, myocardial damage and coronary heart disease.

In another aspect, the present invention provides a method for predicting/determining immune response and/or resistance to viral infection in an individual. The method comprises the step of genotyping the individual to determine the individual's genotype at one or more of the ARTS1 loci identified in the present invention, namely EX1@−1125, EX2@397, EX20@1085, EX6@126, EX12@44, EX15@74, EX6@149, EX8@−10, EX9@39, EX9@+18, EX11@59, EX12@−28, EX12@−7, EX15@88, EX19@173, EX19@328, EX19@885, EX20@2105, EX20719 or EX20@1038; or, at another locus at which the genotype is in linkage disequilibrium with one of the SNPs or haplotypes of the present invention. Thus, if one or more the SNPs EX1@−1125C, EX2@397C, EX20@1085G, EX6@126G, EX12@44A, EX15@74A, EX6@149C, EX8@−10G, EX9@39C, EX9@+18C, EX11@59G, EX12@−28G, EX12@−7C, EX15@88G, EX19@173A, EX19@328w, EX19@885C, EX20@2105T, EX20@719T or EX20@1038C are detected, or a SNP that is in linkage disequilibrium with any one of such SNPs is detected in the individual, then it can be reasonably predicted that the individual will have a reduced immune response and thus, a decreased resistance to viral infection. In other words, the individual has an increased likelihood of developing viral infection. Particularly, if an individual is homozygous with the ARTS1 genotype EX1@−1125C/C, EX2@397C/C, EX20@1085G/G, EX6@126G/G, EX12@44A/A, EX15@74A/A, EX6@149C/C, EX8@−10G/G, EX9@39C/C, EX9@+18C/C, EX11@59G/G, EX12@−28G/G, EX12@−7C/C, EX15@88G/G, EX19@173A/A, EX19@328w/w, EX19@885C/C, EX20@2105T/T, EX20@719T/T or EX20@1038C/C, then the individual has particularly poor immune response, especially to viral infection. Likewise, if the individual is homozygous with a genotype at an ARTS1 locus that is in the same haplotype with the SNPs EX6@126G, EX12@44A and EX15@74A (in linkage disequilibrium), or in the same haplotype (linkage disequilibrium) with the SNPs EX6@149C and EX8@−10G, or in the same haplotype (linkage disequilibrium) with the SNPs EX9@39C, EX9@+18C, EX11@59G, EX12@−28G, EX12@−7C and EX15@88G, or in the same haplotype (linkage disequilibrium) with the SNPs EX19@173A, EX19@328w, EX19@885C and EX20@2105T, or in the same haplotype (linkage disequilibrium) with the SNPs EX20@719T and EX20@1038C, then it can reasonably be predicted that the individual has a diminished immune response and decrease resistance to viral infection. However, if an individual is heterozygous with the genotype EX1@−1125C/T, EX2@397C/G, EX20@1085G/A, EX6@126G/A, EX12@44G/A, EX15@74G/A, EX6@149T/C, EX8@−10G/A, EX9@39C/T, EX9@+18T/C, EX11@59G/A, EX12@−28G/T, EX12@−7C/A, EX15@88G/C, EX19@173C/A, EX19@328w/m, EX19@885C/T, EX20@2105T/C, EX20@719T/C or EX20@1038A/C, then the individual has intermediate immune response. Specifically, the individual has an intermediate level of risk of viral infection. Alternatively, if the individual is homozygous with the ARTS1 genotype EX1@−1125T/T, EX2@397G/G, EX20@1085A/A, EX6@126A/A, EX12@44G/G, EX15@74G/G, EX6@149T/T, EX8@−10A/A, EX9@39T/T, EX9@+18T/T, EX11@59A/A, EX12@−28T/T, EX12@−7A/A, EX15@88C/C, EX19@173C/C, EX19@328 m/m, EX19@885T/T, EX20@2105C/C, EX20@719C/C or EX20@1038A/A, then it can be reasonably predicted that the individual will have a good immune response. In other word, the individual will have a reduced susceptibility to infection, especially viral infection. Similarly, if the individual is homozygous with a genotype at an ARTS1 locus that is in the same haplotype with the SNPs EX6@126A, EX12@44G and EX15@74G (in linkage disequilibrium), or in the same haplotype (linkage disequilibrium) with the SNPs EX6@149T and EX8@−10A, or in the same haplotype (linkage disequilibrium) with the SNPs EX9@39T, EX9@+18T, EX11@59A, EX12@−28T, EX12@−7A and EX15@88C, or in the same haplotype (linkage disequilibrium) with the SNPs EX19@173C, EX19@328m, EX19@885TC and EX20@2105C, or in the same haplotype (linkage disequilibrium) with the SNPs EX20@719C and EX20@1038A, then it can reasonably be predicted that the individual will have a normal immune response to infection, such as viral infection.

In another aspect, the present invention provides a method for predicting and/or determining susceptibility to inflammatory and autoimmune disease. The method comprises the step of genotyping the individual to determine the individual's genotype at one or more of the ARTS1 loci identified in the present invention, namely EX1@−1125, EX2@397, EX20@1085, EX6@126, EX12@44, EX15@74, EX6@149, EX8@−10, EX9@39, EX9@+18, EX11@59, EX12@−28, EX12@−7, EX15@88, EX19@173, EX19@328, EX19@885, EX20@2105, EX20719 or EX20@1038; or, at another ARTS1 locus which is in linkage disequilibrium with one of the SNPs or haplotypes of the present invention. Thus, if one or more the SNPs EX1@−1125C, EX2@397C, EX20@1085G, EX6@126G, EX12@44A, EX15@74A, EX6@149C, EX8@−10G, EX9@39C, EX9@+18C, EX11@59G, EX12@−28G, EX12@−7C, EX15@88G, EX19@173A, EX19@328w, EX19@885C, EX20@2105T, EX20@719T or EX20@1038C are detected, or a SNP that is in linkage disequilibrium with any one of such SNPs is detected in the individual, then it can be reasonably predicted that the individual will have increased susceptibility to autoimmune disease, particularly endotoxic shock, TNF-dependent arthritis, and encephalomyelitis. Particularly, if an individual is homozygous with the ARTS1 genotype EX1@−1 125C/C, EX2@397C/C, EX20@1085G/G, EX6@126G/G, EX12@44A/A, EX15@74A/A, EX6@149C/C, EX8@−10G/G, EX9@39C/C, EX9@+18C/C, EX11@59G/G, EX12@−28G/G, EX12@−7C/C, EX15@88G/G, EX19@173A/A, EX19@328w/w, EX19@885C/C, EX20@2105T/T, EX20@719T/T or EX20@1038C/C, then the individual has particularly elevated susceptibility to autoimmune disease, especially endotoxic shock, TNF-dependent arthritis, and encephalomyelitis. Likewise, if the individual is homozygous with a genotype at an ARTS1 locus that is in the same haplotype with the SNPs EX6@126G, EX12@44A and EX15@74A (in linkage disequilibrium), or in the same haplotype (linkage disequilibrium) with the SNPs EX6@149C and EX8@−10G, or in the same haplotype (linkage disequilibrium) with the SNPs EX9@39C, EX9@+18C, EX11@59G, EX12@−28G, EX12@−7C and EX15@88G, or in the same haplotype (linkage disequilibrium) with the SNPs EX19@173A, EX19@328w, EX19@885C and EX20@2105T, or in the same haplotype (linkage disequilibrium) with the SNPs EX20@719T and EX20@1038C, then it can reasonably be predicted that the individual has an elevated susceptibility to autoimmune disease, especially endotoxic shock, TNF-dependent arthritis, and encephalomyelitis. However, if an individual is heterozygous with the ARTS1 genotype EX1@−1125C/T, EX2@397C/G, EX20@1085G/A, EX6@126G/A, EX12@44G/A, EX15@74G/A, EX6@149T/C, EX8@−10G/A, EX9@39C/T, EX9@+18T/C, EX1@59G/A, EX12@−28G/T, EX12@−7C/A, EX15@88G/C, EX19@173C/A, EX19@328w/m, EX19@885C/T, EX20@2105T/C, EX20@719T/C or EX20@1038A/C, then the individual has intermediate probability of developing and autoimmune disease. Alternatively, if the individual is homozygous with the ARTS1 genotype EX1@−1125T/T, EX2@397G/G, EX20@1085A/A, EX6@126A/A, EX12@44G/G, EX15@74G/G, EX6@149T/T, EX8@−10A/A, EX9@39T/T, EX9@+18T/T, EX11@59A/A, EX12@−28T/T, EX12@−7A/A, EX15@88C/C, EX19@173C/C, EX19@328m/m, EX19@885T/T, EX20@2105C/C, EX20@719C/C or EX20@1038A/A, then it can be reasonably predicted that the individual will have a decreased likelihood of developing an autoimmune disease, particularly endotoxic shock, TNF-dependent arthritis, and encephalomyelitis. Similarly, if the individual is homozygous with an ARTS1 genotype at a locus that is in the same haplotype with the SNPs EX6@126A, EX12@44G and EX15@74G (in linkage disequilibrium), or in the same haplotype (linkage disequilibrium) with the SNPs EX6@149T and EX8@−10A, or in the same haplotype (linkage disequilibrium) with the SNPs EX9@39T, EX9@+18T, EX11@59A, EX12@−28T, EX12@−7A and EX15@88C, or in the same haplotype (linkage disequilibrium) with the SNPs EX19@173C, EX19@328m, EX19@885TC and EX20@2105C, or in the same haplotype (linkage disequilibrium) with the SNPs EX20@719C and EX20@1038A, then it can reasonably be predicted that the individual has a reduced susceptibility to autoimmune diseases such as endotoxic shock, TNF-dependent arthritis, and encephalomyelitis.

The SNPs listed in Table 11, i.e. those at positions 96,112,196 and 96,134,750 of chromosome 5, have also been shown to be associated with ARTS1 mRNA levels. Chromosome 5 SNPs associated with lower ARTS1 mRNA expression levels are 96,112,196C and 96,134,750C, whereas those associated with lower ARTS1 mRNA expression are 96,112,196T and 96,134,750T. In addition to those mentioned above, these SNPs may be utilized in the applications described above.

MSR

As indicated in Table 12 and 45-47 the expression level of the MSR gene in human cells is an inheritable “quantitative trait” with genetic determinants. Furthermore, the SNPs in accordance with the present invention are associated with the “quantitative trait”, i.e., MSR mRNA levels in human cells. Specifically, the SNPs EX1@−674G, EX1@19C, EX1@+129w, EX5@123T, EX5@136C, EX7@146G, EX10@+83G, EX11@+54C, EX14@14T, EX14@106A, EX14@142G and EX15@686A are associated with a “low expression phenotype” while the EX1@−74T, EX1@119T, EX1@+129m, EX5@123C, EX5@136T, EX7@146A, EX10@+83A, EX11@+54T, EX14@14C, EX14@106G, EX14@142A and EX15@686G are associated with a “high expression phenotype.” Thus, the SNPs are particularly useful in predicting the level of MSR gene expression in an individual.

Thus, in one aspect of the invention a method is provided for predicting or detecting susceptibility to hyperhomocysteinemia, cardiovascular disease, atherosclerosis, recurrent arterial and venous thrombosis, premature coronary artery disease and neural tube defects in an individual, which comprises the steps of genotyping the individual to determine the individual's genotype at one or more loci identified in the present invention wherein one or more of the SNPs are detected in the individual, then it can be predicted whether the individual has an increased risk of developing a metabolic or vascular disease. Thus, if one or more the MSR SNPs EX1@−674G, EX1119C, EX1@+129w, EX5@123T, EX5@136C, EX7@146G, EX10@+83G, EX11@+54C, EX14@14T, EX14@106A, EX14@142G or EX15@686A are detected, or, a SNP that is in linkage disequilibrium with any one of such SNPs is detected in the individual, then it can be reasonably predicted that the individual is at an increased risk of developing disease, particularly hyperhomocysteinemia, cardiovascular disease, atherosclerosis, recurrent arterial and venous thrombosis, premature coronary artery disease and neural tube defects. In particular, if an individual is homozygous with the MSR genotype EX1@−674G/G, EX1@19C/C, EX1@+129w/w, EX5@123T/T, EX5@136C/C, EX7@146G/G, EX10@+83G/G, EX1@+54C/C, EX14@14T/T, EX14@106A/A, EX14@142G/G or EX15@686A/A, or a SNP that is in linkage disequilibrium with any one or more of such SNPs, then it can be reasonably predicted that the individual has an increased susceptibility to hyperhomocysteinemia, cardiovascular disease, atherosclerosis, recurrent arterial and venous thrombosis, premature coronary artery disease and neural tube defects. In other words, such an individual has an increased likelihood or is at an increased risk of developing disease, particularly hyperhomocysteinemia, cardiovascular disease, atherosclerosis, recurrent arterial and venous thrombosis, premature coronary artery disease and neural tube defects. If an individual is heterozygous, then his or her risk of developing the disease is at an intermediate level. On the other hand, if the individual is homozygous with the MSR genotype EX1@−674T/T, EX1@19T/T, EX1@+129m/m, EX5@123C/C, EX5@136T/T, EX7@146A/A, EX10@+83A/A, EX11@+54T/T, EX14@14C/C, EX14@106G/G, EX14@142A/A or EX15@686G/G, or a SNP that is in linkage disequilibrium with any one or more of such SNPs, then it can be reasonably predicted that the individual has a reduced susceptibility to disease, particularly hyperhomocysteinemia, cardiovascular disease, atherosclerosis, recurrent arterial and venous thrombosis, premature coronary artery disease and neural tube defects.

In another aspect, the present invention provides a method for determining the prognosis of a patient having a hyperhomocysteinemia, cardiovascular disease, atherosclerosis, recurrent arterial and venous thrombosis, premature coronary artery disease and neural tube defects. The individual to be tested can be a healthy person or previously diagnosed individual. The method comprises the step of genotyping the individual to determine the individual's genotype at one or more of the loci identified in the present invention, or another locus at which the genotype is in linkage disequilibrium with one of the SNPs or haplotypes of the present invention. Thus, if one or more the MSR SNPs EX1@−674G, EX1@19C, EX1@+129w, EX5@123T, EX5@136C, EX7@146G, EX10@+83G, EX11@+54C, EX14@14T, EX14@106A, EX14@142G or EX15@686A are detected, or a SNP that is in linkage disequilibrium with any one of such SNPs is detected in the individual, then it can be reasonably predicted that the individual has high potential of disease progression and that the prognosis for the individual is poor. In other words, the individual has an increased likelihood or an increased risk of developing the disease and of disease progression. Particularly, if an individual is homozygous with the MSR genotype EX1@−674G/G, EX1@19C/C, EX1@+129w/w, EX5@123T/T, EX5@136C/C, EX7@146G/G, EX10@+83G/G, EX11@+54C/C, EX14@14T/T, EX14@106A/A, EX14@142G/G or EX15@686A/A, or a SNP that is in linkage disequilibrium with any one or more of such SNPs, then the individual has particularly poor prognosis and the disease will likely progress at an increased rate. In other words, the individual has a substantially increased likelihood or a substantially increased risk of disease progression. However, if an individual is heterozygous with the MSR genotype EX1@−674T/G, EX1@19T/C, EX1@+129m/m, EX5@123C/T, EX5@136T/C, EX7@146G/A, EX10@+83G/A, EX11@+54C/T, EX14@14C/T, EX14@106A/G, EX14@142G/A or EX15@686A/G, or is heterozygous with a SNP that is in linkage disequilibrium with any one or more of such SNPs, then the individual has an intermediate prognosis. Specifically, the individual has an intermediate level of disease progression.

Thus, if the individual is homozygous with the MSR genotype EX1@−674T/T, EX1@19T/T, EX1@+129m/m, EX5@123C/C, EX5@136T/T, EX7@146A/A, EX10@+83A/A, EX11@+54T/T, EX14@14C/C, EX14@106G/G, EX14@142A/A or EX15@686G/G is detected, or is homozygous with a SNP that is in linkage disequilibrium with any one or more of such SNPs, then it can be reasonably predicted that disease prognosis is favorable. That is, the individual does not have an increased likelihood or increased risk of hyperhomocysteinemia, cardiovascular disease, atherosclerosis, recurrent arterial and venous thrombosis, premature coronary artery disease and neural tube defects.

In another aspect, the present invention encompasses a method for predicting or detecting cancer susceptibility in an individual, which comprises the step of genotyping the individual to determine the individual's genotype at one or more of the loci identified in the present invention, or another locus at which the genotype is in linkage disequilibrium with one of the SNPs of the present invention. Thus, if one or more the MSR SNPs EX1@−674T, EX1@19T, EX1@+129m, EX5@123C, EX5@136T, EX7@146A, EX10@+83A, EX11@+54T, EX14@14C, EX14@106G, EX14@142A or EX15@686G is detected, or a SNP that is in linkage disequilibrium with any one of such SNPs is detected in the individual, then it can be reasonably predicted that the individual is at an increased risk of developing cancer, particularly colon cancer. In particular, if an individual is homozygous with the MSR genotype EX11@−674T/T, EX1@19T/T, EX1@+129m/m, EX5@123C/C, EX5@136T/T, EX7@146A/A, EX10@+83A/A, EX11@+54T/T, EX14@14C/C, EX14@106G/G, EX14@142A/A or EX15@686G/G is detected, or a SNP that is in linkage disequilibrium with any one or more of such SNPs, then it can be reasonably predicted that the individual has an elevated susceptibility to cancer, particularly colon cancer. In other words, such an individual has an increased likelihood or is at an increased risk of developing cancer, particularly colon cancer. If an individual is heterozygous, then his or her risk of developing cancer is at an intermediate level. On the other hand, if the individual is homozygous with the MSR genotype EX1@−674G/G, EX1@19C/C, EX1@+129w/w, EX5@123T/T, EX5@136C/C, EX7@146G/G, EX10@+83G/G, EX11@+54C/C, EX14@14T/T, EX14@106A/A, EX14@142G/G or EX15@686A/A, then it can be reasonably predicted that the individual has a reduced susceptibility to cancer, particularly colon cancer.

In another aspect, the present invention provides a method for identifying high-risk patients who have cancerous growths with a poor prognosis of recovery, or for predicting/determining the invasiveness and metastatic potential of tumors within a patient, particularly cancer patient, e.g., with non-small cell lung cancers (NSCLCs). The individual to be tested can be a healthy person or an individual diagnosed with cancer. In this aspect of the invention, the methods comprise the step of genotyping the cancerous growth within the individual to determine the tumor's genotype at one or more of the MSR loci identified in the present invention, namely those listed above, or another locus at which the genotype is in linkage disequilibrium with one of the SNPs of the present invention. Thus, if one or more the MSR SNPs EX1@−674T, EX1@19T, EX1@+129m, EX5@123C, EX5@136T, EX7@146A, EX10@+83A, EX11@+54T, EX14@14C, EX14@106G, EX14@142A or EX15@686G are detected, or a SNP that is in linkage disequilibrium with any one of such SNPs is detected in the tumor, then it can be reasonably predicted that the tumor has high metastatic potential, that the individual has poor prognosis, and that the tumor cells are likely invasive and given to robust growth. In other words, the individual has an increased likelihood or an increased risk of cancer metastasis and further growth. Particularly, if the tumor is homozygous with the genotype EX1@−674T/T, EX1@19T/T, EX1@+129m/m, EX5@123C/C, EX5@136T/T, EX7@146A/A, EX10@+83A/A, EX11@+54T/T, EX14@14C/C, EX14@106G/G, EX14@142A/A or EX15@686G/G, then the individual has particular poor prognosis and the tumor cells are likely highly invasive and given to robust growth. In other words, the individual has a substantially increased likelihood or a substantially increased risk of cancer metastasis and aggressive tumor growth. However, if the tumor is heterozygous with the genotype EX1@−674T/G, EX1@19T/C, EX1@+129m/w, EX5@123C/T, EX5@136T/C, EX7@146G/A, EX10@+83G/A, EX11@+54C/T, EX14@14C/T, EX14@106A/G, EX14@142G/A or EX15@686A/G, then the individual has an intermediate prognosis and the tumor cells are potentially invasive. Specifically, the individual has an intermediate level of risk of cancer metastasis and further tumor growth. On the other hand, if the tumor is homozygous with the genotype EX1@−674G/G, EX1@19C/C, EX1@+129w/w, EX5@123T/T, EX5@136C/C, EX7@146G/G, EX10@+83G/G, EX11@+54C/C, EX14@14T/T, EX14@106A/A, EX14@142G/G or EX15@686A/A, then it can be reasonably predicted that the tumor in the individual has low metastatic potential, that the patient has good prognosis, and that the tumor cells are likely not invasive or robust. That is, the individual does not have an increased likelihood or increased risk of cancer metastasis and rapid tumor growth.

The SNP on Chromosome V at position 7,952,909 has also been shown to be associated with MSR mRNA levels. The Chromosome V SNP associated with lower MSR mRNA expression levels is 7,952,909G, whereas that associated with higher mRNA expression levels is 7,952,909C. In addition to those mentioned above, these SNPs may be utilized in the applications described above.

AKAP9

As indicated in Tables 13-14 and 48-55, the expression level of the AKAP9 gene in human cells is an inheritable “quantitative trait” with genetic determinants. Furthermore, the SNPs in accordance with the present invention are associated with the “quantitative trait”, i.e., mRNA level of the AKAP9 gene in human cells. Specifically, the SNPs EX1@−63C, EX1@99G, EX2@+3C, EX9@459T, EX10@186G, EX15@53m, EX16@−59G, EX18@−41G, EX23@−20A, EX26@14T, EX32@+8T, EX34@−45G, EX35@215G, EX35@+8T, EX39@121T, EX44@28C, EX45@−38A, EX50@+58A, EX19@1011C, EX19@1020G, EX19@1033G, EX36@19T, EX40@470A, EX40@910T and EX40@1055G are associated with a “low expression phenotype” while the EX1@−63T, EX1@99C, EX2@+3A, EX9@459G, EX10@186A, EX15@53w, EX16@−59C, EX18@−41T, EX23@−20G, EX26@14C, EX32@+8C, EX34@−45A, EX35@215A, EX35@+8A, EX39@121C, EX44@28A, EX45@−38G, EX50@+58T, EX19@1011T, EX19@1020A, EX19@1033A, EX36@19C, EX40@470G, EX40@910C and EX40@1055A are associated with a “high expression phenotype.” Thus, the SNPs are particularly useful in predicting the level of AKAP9 gene expression in an individual.

Thus, in one aspect, the present invention encompasses a method for predicting or detecting cancer susceptibility in an individual, which comprises the step of genotyping the individual to determine the individual's genotype at one or more of the AKAP9 loci identified in the present invention, namely EX1@63, EX1@99, EX2@+3, EX9@459, EX10@186, EX15@53, EX16@−59, EX18@−41, EX23-20, EX26@14, EX32@+8, EX34@−45, EX35@215, EX35@+8, EX39@121, EX44@28, EX45@−38, EX50@+58, EX19@1011, EX19@1020, EX19@1033, EX36@19, EX40@470, EX40@910 or EX40@1055, or another locus at which the genotype is in linkage disequilibrium with one of the SNPs of the present invention. Thus, if one or more of the AKAP9 SNPs EX1@163T, EX1@99C, EX2@+3A, EX9@459G, EX10@186A, EX15@53w, EX16@−59C, EX18@−41T, EX23@−20G, EX26@14C, EX32@+8C, EX34@−45A, EX35@2215A, EX35@+8A, EX39@121C, EX44@28A, EX45@−38G, EX50@+58T, EX19@1011T, EX19@1020A, EX19@1033A, EX36@19C, EX40@470G, EX40@910C or EX40@1055A are detected, or a SNP that is in linkage disequilibrium with any one of such SNPs is detected in the individual, then it can be reasonably predicted that the individual is at an increased risk of developing cancer. In particular, if an individual is homozygous with the AKAP9 genotype EX1@63T/T, EX1@99C/C, EX2@+3A/A, EX9@459G/G, EX10@186A/A, EX15@53w/w, EX16@−59C/C, EX18@−41T/T, EX23@−20G/G, EX26@14C/C, EX32@+8C/C, EX34@−45A/A, EX35@215A/A, EX35@+8A/A, EX39@121C/C, EX44@28A/A, EX45@−38G/G, EX50@+58T/T, EX19@1011T/T, EX19@1020A/A, EX19@1033A/A, EX36@19C/C, EX40@470G/G, EX40@910C/C or EX40@1055A/A, then it can be reasonably predicted that the individual has an elevated susceptibility to cancer. In other words, such an individual has an increased likelihood or is at an increased risk of developing cancer, particularly skin cancer. If an individual is heterozygous, then his or her risk of developing cancer is at an intermediate level. One the other hand, if the individual is homozygous with the AKAP9 genotype EX1@63C/C, EX1@99G/G, EX2@+3C/C, EX9@459T/T, EX10@186G/G, EX15@53m/m, EX16@−59G/G, EX18@−41G/G, EX23@−20A/A, EX26@14T/T, EX32@+8T/T, EX34@−45G/G, EX@215G/G, EX@+8T/T, EX39@121T/T, EX44@28C/C, EX45@−38A/A, EX50@+58A/A, EX19@1011C/C, EX19@1020G/G, EX19@1033G/G, EX36@19T/T, EX40@470A/A, EX40@910T/T or EX40@1055G/G, then it can be reasonably predicted that the individual has a reduced susceptibility of developing cancer.

In another aspect, the present invention encompasses a method for predicting or detecting susceptibility to neurological disorders in an individual, which comprises the step of genotyping the individual to determine the individual's genotype at one or more of the AKAP9 loci identified in the present invention, namely EX1@63, EX1@99, EX2@+3, EX9@459, EX1@186, EX15@53, EX16@−59, EX18@−41, EX23@−20, EX26@14, EX32@+8, EX34@−45, EX35@215, EX35@+8, EX39@121, EX44@28, EX45@−38, EX50@+58, EX19@1011, EX19@1020, EX19@1033, EX36@19, EX40@470, EX40@910 or EX40@1055, or another locus at which the genotype is in linkage disequilibrium with one of the SNPs of the present invention. Thus, if one or more of the AKAP9 SNPs EX1@−63C, EX1@99G, EX2@+3C, EX9@459T, EX10@186G, EX15@53m, EX16@−59G, EX18@−41G, EX23@−20A, EX26@14T, EX32@+8T, EX34@−45G, EX@215G, EX@+8T, EX39@121T, EX44@28C, EX45@−38A, EX50@+58A, EX19@1011C, EX19@1020G, EX19@1033G, EX36@19T, EX40@470A, EX40@910T or EX40@1055G are detected, or a SNP that is in linkage disequilibrium with any one of such SNPs is detected in the individual, then it can be reasonably predicted that the individual is at an increased risk of developing neurological disorders, particularly AD, ALS, dementia, Creutzfeldt-Jakob disease, Pick's disease and neurodegeneration caused by aging. In particular, if an individual is homozygous with the AKAP9 genotype EX1@63C/C, EX1@99G/G, EX2@+3C/C, EX9@459T/T, EX10@186G/G, EX15@53m/m, EX16@−59G/G, EX18@−41G/G, EX23@−20A/A, EX26@14T/T, EX32@+8T/T, EX34@−45G/G, EX@215G/G, EX@+8T/T, EX39@121T/T, EX44@28C/C, EX45@−38A/A, EX50@+58A/A, EX19@1011C/C, EX19@1020G/G, EX19@1033G/G, EX36@19T/T, EX40@470A/A, EX40@910T/T or EX40@1055G/G, then it can be reasonably predicted that the individual has an elevated susceptibility to neurological disorders. In other words, such an individual has an increased likelihood or is at an increased risk of developing neurological disorders, particularly AD, ALS, dementia, Creutzfeldt-Jakob disease, Pick's disease and neurodegeneration caused by aging. If an individual is heterozygous, then his or her risk of developing neurological disorders is at an intermediate level. One the other hand, if the individual is homozygous with the AKAP9 genotype EX1@63T/T, EX1@99C/C, EX2@+3A/A, EX9@459G/G, EX10@186A/A, EX15@53w/w, EX16@−59C/C, EX18@−41T/T, EX23@−20G/G, EX26@14C/C, EX32@+8C/C, EX34@−45A/A, EX35@215A/A, EX35@+8A/A, EX39@121C/C, EX44@28A/A, EX45@−38G/G, EX50@+58T/T, EX19@1011T/T, EX19@1020A/A, EX19@1033A/A, EX36@19C/C, EX40@470G/G, EX40@910C/C or EX40@1055A/A, then it can be reasonably predicted that the individual has a reduced susceptibility to neurological disorders, particularly AD, ALS, dementia, Creutzfeldt-Jakob disease, Pick's disease and neurodegeneration caused by aging.

In yet another aspect, the present invention encompasses a method for predicting or detecting an individual's susceptibility to heart disease, which comprises the step of genotyping the individual to determine the individual's genotype at one or more of the AKAP9 loci identified in the present invention, namely EX1@63, EX1@99, EX2@+3, EX9@459, EX10@186, EX15@53, EX16@−59, EX18@−41, EX23@−20, EX26@14, EX32@+8, EX34@−45, EX35@215, EX35@+8, EX39@121, EX44@28, EX45@−38, EX50@+58, EX19@1011, EX19@1020, EX19@1033, EX36@19, EX40@470, EX40@910 or EX40@1055, or another locus at which the genotype is in linkage disequilibrium with one of the SNPs of the present invention. Thus, if one or more of the AKAP9 SNPs EX1@63C, EX1@99G, EX2@+3C, EX9@459T, EX10@186G, EX15@53m, EX16@−59G, EX18@−41G, EX23@−20A, EX26@14T, EX32@+8T, EX34@−45G, EX@215G, EX@+8T, EX39@121T, EX44@28C, EX45@−38A, EX50@+58A, EX19@1011C, EX19@1020G, EX19@1033G, EX36@19T, EX40@470A, EX40@910T or EX40@1055G are detected, or a SNP that is in linkage disequilibrium with any one of such SNPs is detected in the individual, then it can be reasonably predicted that the individual is at an increased risk of heart disease, especially arrhythmia, long QT syndrome, ventricular fibrillation, cardiac arrest and sudden death. In particular, if an individual is homozygous with the AKAP9 genotype EX1163C/C, EX1@99G/G, EX2@+3C/C, EX9@459T/T, EX10@186G/G, EX15@53m/m, EX16@−59G/G, EX18@−41G/G, EX23@−20A/A, EX26@14T/T, EX32@+8T/T, EX34@−45G/G, EX@215G/G, EX@+8T/T, EX39@121T/T, EX44@28C/C, EX45@−38A/A, EX50@+58A/A, EX19@1011C/C, EX19@1020G/G, EX19@1033G/G, EX36@19T/T, EX40@470A/A, EX40@910T/T or EX40@1055G/G, then it can be reasonably predicted that the individual has an elevated susceptibility to heart disease, especially arrhythmia, long QT syndrome, ventricular fibrillation, cardiac arrest and sudden death. If an individual is heterozygous, then his or her risk of developing heart disease is at an intermediate level. One the other hand, if the individual is homozygous with the AKAP9 genotype EX1@63T/T, EX1@99C/C, EX2@+3A/A, EX9@459G/G, EX10@186A/A, EX15@53w/w, EX16@−59C/C, EX18@−41T/T, EX23@−20G/G, EX26@14C/C, EX32@+8C/C, EX34@−45A/A, EX35@215A/A, EX35@+8A/A, EX39@121C/C, EX44@28A/A, EX45@−38G/G, EX50@+58T/T, EX19@1011T/T, EX19@1020A/A, EX19@1033A/A, EX36@19C/C, EX40@470G/G, EX40@910C/C or EX40@1055A/A, then it can be reasonably predicted that the individual has a reduced susceptibility to heart disease such as arrhythmia, long QT syndrome, ventricular fibrillation, cardiac arrest and sudden death.

A further aspect of the present invention provides a method for predicting or detecting susceptibility to depression in an individual, comprising the step of genotyping the individual to determine the individual's genotype at one or more of the AKAP9 loci identified in the present invention, namely EX1@63, EX1@99, EX2@+3, EX9@459, EX10@186, EX15@53, EX16@−59, EX18@−41, EX23@−20, EX26@14, EX32@+8, EX34@−45, EX35@215, EX35@+8, EX39@121, EX44@28, EX45@−38, EX50@+58, EX19@1011, EX19@1020, EX19@1033, EX36@19, EX40@470, EX40@910 or EX40@1055, or another locus at which the genotype is in linkage disequilibrium with one of the SNPs of the present invention. Thus, if one or more of the AKAP9 SNPs EX1@63T, EX1@99C, EX2@+3A, EX9@459G, EX10@186A, EX15@53w, EX16@−59C, EX18@−41T, EX23@−20G, EX26@14C, EX32@+8C, EX34@−45A, EX35@215A, EX35@+8A, EX39@121C, EX44@28A, EX45@−38G, EX50@+58T, EX19@1011T, EX19@1020A, EX19@1033A, EX36@19C, EX40@470G, EX40@910C or EX40@1055A are detected, or a SNP that is in linkage disequilibrium with any one of such SNPs is detected in the individual, then it can be reasonably predicted that the individual has an increased susceptibility to depression. In particular, if an individual is homozygous with the AKAP9 genotype EX1@63T/T, EX1@99C/C, EX2@+3A/A, EX9@459G/G, EX10@186A/A, EX15@53w/w, EX16@−59C/C, EX18@−41T/T, EX23@−20G/G, EX26@14C/C, EX32@+8C/C, EX34@−45A/A, EX35@215A/A, EX35@+8A/A, EX39@121C/C, EX44@28A/A, EX45@−38G/G, EX50@+58T/T, EX19@1011T/T, EX19@1020A/A, EX19@1033A/A, EX36@19C/C, EX40@470G/G, EX40@910C/C or EX40@1055A/A, then it can be reasonably predicted that the individual has an elevated susceptibility to depression. If an individual is heterozygous, then his or her risk of developing depression is at an intermediate level. One the other hand, if the individual is homozygous with the AKAP9 genotype EX1@63C/C, EX1@99G/G, EX2@+3C/C, EX9@459T/T, EX10@186G/G, EX15@53m/m, EX16@−59G/G, EX18@−41G/G, EX23@−20A/A, EX26@14T/T, EX32@+8T/T, EX34@−45G/G, EX@215G/G, EX@+8T/T, EX39@121T/T, EX44@28C/C, EX45@−38A/A, EX50@+58A/A, EX19@1011C/C, EX19@1020G/G, EX19@1033G/G, EX36@19T/T, EX40@470A/A, EX40@910T/T or EX40@1055G/G, then it can be reasonably predicted that the individual has a reduced susceptibility to depression.

DNAJD1

As indicated in Tables 15, 16, 57 and 58, the expression level of the DNAJD1 gene in human cells is an inheritable “quantitative trait” with genetic determinants. Furthermore, the SNPs and/or haplotypes in accordance with the present invention are associated with the “quantitative trait”, i.e., DNAJD1 mRNA levels in human cells. Specifically, the SNPs EX1@368T, EX1@527G and EX5@+72m are associated with a “low expression phenotype” while the EX1@368C, EX1@527A and EX5@+72w are associated with a “high expression phenotype.” Thus, the SNPs and/or haplotypes are particularly useful in predicting the level of DNAJD1 gene expression in an individual.

Thus, in one aspect, the present invention encompasses a method for predicting or detecting cancer susceptibility in a patient, which comprises the step of genotyping the individual to determine the individual's genotype at one or more of the DNAJD1 loci identified in the present invention, namely EX1@368, EX1527 or EX5@+72, or another locus at which the genotype is in linkage disequilibrium with one of these SNPs. Thus, if one or more the DNAJD1 SNPs EX1@368T, EX1527G or EX5@+72m are detected, or a SNP that is in linkage disequilibrium with any one of such SNPs is detected in the individual, then it can be reasonably predicted that the individual is at an increased risk of developing cancer, particular ovarian cancer. In particular, if an individual is homozygous with the DNAJD1 genotype SNPs EX1@368T/T, EX1@527G/G or EX5@+72m/m, then it can be reasonably predicted that the individual has an elevated susceptibility to cancer, particularly ovarian cancer. In other words, such an individual has an increased likelihood or is at an increased risk of developing cancer. If an individual is heterozygous, then his or her risk of developing cancer is at an intermediate level. One the other hand, if the individual is homozygous with the DNAJD1 genotype SNPs EX1@368C/C, EX1@527A/A or EX5@+72w/w, then it can be reasonably predicted that the individual has a reduced susceptibility to cancer, particularly ovarian cancer.

In another aspect, the present invention provides a method for identifying high-risk patients who have cancer with a poor prognosis, or for the prognosis of a specific cancer, or predicting/determining the invasiveness and metastatic potential of a tumor in a patient, particularly cancer patient, e.g., an ovarian cancer patient. The individual to be tested can be a healthy person or an individual diagnosed with cancer. The method comprises the step of genotyping the individual to determine the individual's genotype at one or more of the DNAJD1 loci identified in the present invention, namely SNPs EX1@368, EX1@527 or EX5@+72, or another locus at which the genotype is in linkage disequilibrium with one of the SNPs of the present invention. Thus, if one or more the DNAJD1 SNPs EX1@368T, EX1@527G or EX5@+72m are detected, or a SNP that is in linkage disequilibrium with any one of such SNPs is detected in the a cancer patient, then it can be reasonably predicted that the patient's tumor has a high metastastic potential, that the patient has a poor prognosis, and that the tumor cells are likely to be invasive. In other words, the individual has an increased likelihood, or has an increased risk, of cancer metastasis. Particularly, if an individual is homozygous with the DNAJD1 genotype EX1@368T/T, EX1@527G/G or EX5@+72m/m, then the individual has particularly poor prognosis, and the tumor cells are likely to be highly invasive. In other words, the individual has a substantially increased likelihood, or a substantially increased risk for cancer metastasis. However, if an individual is heterozygous with the genotype EX1@368T/C, EX1@527G/A or EX5@+72m/w, then the patient has an intermediate prognosis, and their tumor cells are potentially invasive. Specifically, the individual has an intermediate level of risk of cancer metastasis. That is, the risk is greater than a person having a homozygous DNAJD1 genotype of EX1@368C/C, EX1@527A/A or EX5@+72w/w, but is lower than a person having a homozygous genotype of EX1@368T/T, EX1@527G/G or EX5@+72m/m.

Thus, if the individual is homozygous with the DNAJD1 genotype EX1@368C/C, EX1@527A/A or EX5@+72w/w, then it can be reasonably predicted that the tumor in the individual has low metastatic potential, that the patient has a good prognosis and that the tumor cells are likely not invasive. That is, the individual does not have an increased likelihood or increased risk of cancer metastasis.

In yet another aspect of the present invention, a method is provided for predicting drug response in a patient to treatment with one or more anti-tumor agents. Examples of such drugs are chemotherapeutics including, but not limited to pacilitaxel, topotecan and cisplatin. Thus, in accordance with the present invention, the DNAJD1 gene of a patient, in need of treatment with an anti-cancer agent, is genotyped to determine the genotype at one or more of the DNAJD1 loci identified in the present invention, namely EX1@368, EX1@1527 or EX5@+72, or another locus at which a genotype is in linkage disequilibrium with one of these SNPs. Thus, if one or more the SNPs EX1@368C, EX1@527A or EX5@+72w are detected, or a SNP that is in linkage disequilibrium with any one of such SNPs is detected in the individual patient, then it can be reasonably predicted that the individual's cancer is likely to respond to treatment with an anti-tumor agent. In other words, once an anti-tumor agent is administered, there is an increased likelihood that the inhibitor will cause a positive effect in the individual, including, e.g., shrinkage or elimination of tumor, increased death of tumor cells, etc.

Particularly, if an individual is homozygous with the DNAJD1 genotype EX1@368C/C, EX1@527A/A or EX5@+72w/w, then the individual has a substantially increased likelihood of being responsive to treatment with an anti-tumor agent, e.g., paclitaxel, topotecan or cisplatin. If an individual is heterozygous with the genotype EX1@368C/T, EX1@527A/G or EX5@+72m/w, then the individual is still likely to respond to an anti-tumor agent. Specifically, the individual has an intermediate level of responsiveness to anti-tumor agents. That is, the degree of responsiveness is likely to be greater than that in a person having a homozygous genotype of EX1@368T/T, EX1@527G/G or EX5@+72m/m, but is lower than a person having a homozygous genotype of EX1@368C/C, EX1@527A/A or EX5@+72w/w. Thus, if the individual is homozygous with the DNAJD1 genotype EX1@368T/T, EX1@527G/G or EX5@+72m/m, then it can be reasonably predicted that there is an increased likelihood that the individual exhibits a low responsiveness to treatment with a anti-tumor agent.

In specific embodiments, the individual in need of an anti-tumor agent is diagnosed as having cancer, e.g., ovarian cancer. Also, in certain embodiments, the anti-tumor agent is a chemotherapeutic. In certain examples, such a chemotherapeutic agents are selected from pacilitaxel, topotecan or cisplatin.

Once the prognosis of a patient's response to anti-tumor agent is made, suitable treatment regimens (e.g., dosage and frequency of administration, and the like) can be decided based on the predicted responsiveness of the patient. For example, if the DNAJD1 gene genotyping result suggests a low responsiveness by the patient to anti-tumor agent, then a higher dosage of anti-tumor agent would be desirably to the patient, or it may be simply decided that another class of drugs would be more suitable for the patient. Thus, in another aspect of the invention, a method is provided for determining a dosage of a anti-tumor agent to be administered to a patient, comprising determining the individual's genotype at one or more of the DNAJD1 loci identified in the present invention, namely EX1@368, EX1@527 or EX5@+72, or another locus at which the genotype is in linkage disequilibrium with one of these SNPs, to determine the likely responsiveness of the patient, and determining accordingly the dosage of a anti-tumor agent to be administered to the patient, wherein the presence of one or more of the SNPs EX1@368C, EX1@527A or EX5@+72w, or a SNP that is in linkage disequilibrium with any one of such SNPs would indicate that the patient is likely to respond to said anti-tumor agent at a lower dosage than another patient without the nucleotide variants. In one embodiment, the method is used in treating ovarian cancer. In other embodiments, the method is used in treating breast cancer, melanoma, lung cancer, brain cancer, neuroblastoma, uterine cancer, leukemia, lymphoma, head and neck cancer, thyroid cancer, gastrointestinal cancer, pancreatic cancer, liver cancer, etc.

In another aspect of the invention, a method is provided for selecting an anti-cancer treatment for a particular patient's tumor(s), which comprises determining, in a DNAJD1 gene from a tumor sample isolated from the patient, the presence or absence of a nucleotide variant that is selected from the group consisting of EX1@368C, EX1@527A and EX5@+72w, or a SNP that is in linkage disequilibrium with any one of such SNPs, wherein the presence of said nucleotide variant would indicate that the patient is likely to respond to an anti-tumor agent. Thus, if the DNAJD1 gene of the patient's tumor contains one or more of the nucleotide variants of the present invention, then physicians may decide, based on the tumor genotyping result, whether it would be desirable to treat the patient with anti-tumor agents, particularly chemotherapeutics, such as pacilitaxel, topotecan or cisplatin. In one embodiment, the selection of treatment with an anti-tumor agent is based on the presence of a homozygous genotype of one or more of the above SNPs.

In yet another aspect of the present invention, a method is provided for selecting candidate human subjects for participation in a clinical trial involving a DNAJD1 inhibitor, which comprises (1) determining, in the DNAJD1 gene of a tumor sample from an individual patient, the presence or absence of a nucleotide variant that is selected from the group consisting of EX1@368C, EX1@527A and EX5@+72w, or a SNP that is in linkage disequilibrium with any one of such SNPs, wherein the presence of said nucleotide variant would indicate that the patient's tumor is likely to respond to a anti-tumor agent, such as pacilitaxel, topotecan or cisplatin; and (2) deciding whether to include said individual patient in the clinical trial. For example, if the patient's tumor has one or more of the nucleotide variants, then clinical trial for an anti-tumor agent may include that patient, particularly when the patient's tumor is homozygous in one or more of the SNPs.

In another aspect, the present invention encompasses a method for predicting or detecting susceptibility to neurodegenerative disease in a patient, which comprises the step of genotyping the individual to determine the individual's genotype at one or more of the DNAJD1 loci identified in the present invention, namely EX1@368, EX1@527 and EX5@+72, or another locus at which the genotype is in linkage disequilibrium with any one of these SNPs. Thus, if one or more the SNPs EX1@368T, EX1@527G or EX5@+72m are detected, or a SNP that is in linkage disequilibrium with any one of such SNPs is detected in the individual, then it can be reasonably predicted that the individual has increased risk of neurodegenerative disease, especially Alzheimer's disease, Parkinson's disease, Huntington's disease, prion disorders, CJD, DRPLA, SCA1, SCA3, schizophrenia and depression. In particular, if an individual is homozygous with the DNAJD1 genotype SNPs EX1@368T/T, EX1@527G/G or EX5@+72m/m, then it can be reasonably predicted that the individual has an elevated susceptibility to neurodegenerative disease. In other words, such an individual has an increased likelihood or is at an increased risk of developing a neurodegenerative disease. If an individual is heterozygous, then his or her risk of developing a neurodegenerative disease, particularly, Alzheimer's disease, Parkinson's disease, Huntington's disease, prion disorders, CJD, DRPLA, SCA1, SCA3, schizophrenia or depression is at an intermediate level. One the other hand, if the individual is homozygous with the DNAJD1 genotype SNPs EX1@368C/C, EX1@527A/A or EX5@+72w/w, then it can be reasonably predicted that the individual has a reduced susceptibility to neurodegenerative disease, such as Alzheimer's disease, Parkinson's disease, Huntington's disease, prion disorders, CJD, DRPLA, SCA1, SCA3, schizophrenia and depression.

In another aspect, the present invention provides a method for identifying high-risk patients who have a poor prognosis of a neurodegenerative or neurological disease, or for the prognosis of neurodegenerative or neurological disease, or predicting/determining the ability to recover from neuronal damage resulting from brain trauma. The individual to be tested can be a healthy person or an individual diagnosed with a neurodegenerative disease, or suffering from Alzheimer's disease, Parkinson's disease, Huntington's disease, prion disorders, CJD, DRPLA, SCA1, SCA3, schizophrenia and depression. The method comprises the step of genotyping the individual to determine the individual's genotype at one or more of the DNAJD1 loci identified in the present invention, namely SNPs EX1@368C, EX1@527A or EX5@+72w, or another locus at which the genotype is in linkage disequilibrium with one of the SNPs of the present invention. Thus, if one or more of the DNAJD1 SNPs EX1@368C, EX1@527A or EX5@+72w are detected, or a SNP that is in linkage disequilibrium with any one of such SNPs is detected in the individual, then it can be reasonably predicted that the individual has a decreased likelihood of neurological or neurodegenerative disease, especially Alzheimer's disease, Parkinson's disease, Huntington's disease, prion disorders, CJD, DRPLA, SCA1, SCA3, schizophrenia and depression, or the individual will have a reasonable recovery from neuronal damage resulting from brain trauma. Alternatively, if an individual is homozygous with the DNAJD1 genotype EX1@368T/T, EX1@527G/G or EX5@+72m/m, then the individual can reasonably be assumed to have a particular poor prognosis of neurodegenerative disease, or a protracted or incomplete recovery from neuronal damage resulting from brain trauma. In other words, the individual has a substantially increased likelihood of or is at a substantially increased risk of progression of the neurodegenerative disease or neurological damage. However, if an individual is heterozygous with the DNAJD1 genotype EX1@368T/C, EX1@527G/A or EX5@+72m/w, an intermediate level of risk of neurological disease, especially neurodegeneration, Alzheimer's disease, Parkinson's disease, Huntington's disease, prion disorders, CJD, DRPLA, SCA1, SCA3, schizophrenia and depression. That is, the risk is higher than a person having a homozygous DNAJD1 genotype of EX1@368C/C, EX1@527A/A or EX5@+72w/w, but is lower than a person having a homozygous genotype of EX1@368T/T, EX1@527G/G or EX5@+72m/m. Alternatively, if the individual is homozygous with the DNAJD1 genotype EX1@368C/C, EX1@527A/A or EX5@+72w/w, then it can be reasonably predicted that the individual does not have an increased likelihood or increased risk of neurodegenerative disease or neurological disease.

In yet another aspect, the present invention encompasses a method for predicting or detecting ischemic or ischemic-type injury in an individual, which comprises the step of genotyping the individual to determine the individual's genotype at one or more of the DNAJD1 loci identified in the present invention, namely EX1@368, EX1@527 or EX5@+72, or another locus at which the genotype is in linkage disequilibrium with one of these SNPs. Thus, if one or more the DNAJD1 SNPs EX1@368T, EX1@527G or EX5@+72m are detected, or a SNP that is in linkage disequilibrium with any one of such SNPs is detected in the individual, it can be reasonably predicted that the individual is at an increased risk of ischemic or ischemic-type injury, particularly cardiomyopathy, coronary disease, coronary artery disease, heart attack, stroke, and intestinal ischemia. In particular, if an individual is homozygous with the DNAJD1 genotype EX1@368T/T, EX1@527G/G or EX5@+72m/m, then it can be reasonably predicted that the individual has an elevated susceptibility to ischemia or ischemic-type injury, particularly cardiomyopathy, coronary disease, coronary artery disease, heart attack, stroke, and intestinal ischemia. If an individual is heterozygous, then his or her risk of developing ischemia or ischemic-type injury is at an intermediate level. On the other hand, if the individual is homozygous with the DNAJD1 genotype EX1@368C/C, EX1@527A/A or EX5@+72w/w, then it can be reasonably predicted that the individual has a reduced susceptibility to ischemia or ischemic-type injury, particularly cardiomyopathy, coronary disease, coronary artery disease, heart attack, stroke, and intestinal ischemia.

The SNPs on Chromosome XIII at positions 42,536,771, 42,554,443, 42,554,646, and 42,554,817 have also been shown to be associated with DNAJD1 mRNA levels. The Chromosome XIII SNPs associated with lower DNAJD1 mRNA expression levels are 42,536,771T, 42,554,443A, 42,554,646A, and 42,554,817T, whereas that associated with higher mRNA expression levels are 42,536,771C, 42,554,443C, 42,554,646G, and 42,554,817C. In addition to those mentioned above, these SNPs may be utilized in the applications described above.

GOLPH4

As indicated in Tables 17, 18, 58 and 59, the expression level of the GOLPH4 gene in human cells is an inheritable “quantitative trait” with genetic determinants. Furthermore, the SNPs in accordance with the present invention are associated with the “quantitative trait”, i.e., GOLPH4 mRNA levels in human cells. Specifically, the SNPs EX12@−78C, EX15@−85C, EX15@+86C, EX16@323G, EX16@737A, EX16@771G are associated with a “low expression phenotype” while the EX12@−78T, EX51-85G, EX15@+86G, EX16@323A, EX16@737G, EX16@771A are associated with a “high expression phenotype.” Thus, the SNPs are particularly useful in predicting the level of GOLPH4 gene expression in an individual.

Thus, in one aspect, the present invention encompasses a method for predicting or detecting an individual's susceptibility to bacterial toxins, which comprises the step of genotyping the individual to determine the individual's genotype at one or more of the GOLPH4 loci identified in the present invention, namely, EX12@−78, EX15@−85, EX15@+86, EX16@323, EX16@737, EX16@771, or another locus at which the genotype is in linkage disequilibrium with one of these SNPs. Thus, if one or more EX12@−78T, EX15@−85G, EX15@+86G, EX16@323A, EX16@737G, EX16@771A, or a SNP that is in linkage disequilibrium with any one of such SNPs, is detected in the individual, then it can be reasonably predicted that the individual is at an increased risk of developing an adverse condition caused by bacterial toxins, particularly the ricin, cholera or Shiga toxins.

In particular, if an individual is homozygous with the GOLPH4 genotype EX 12@−78T/T, EX15@−85G/G, EX15@+86G/G, EX16@323A/A, EX16@737G/G, EX16@771A/A, or heterozygous with the GOLPH4 genotype EX12@−78C/T, EX15@−85C/G, EX15@+86C/G, EX16@323G/A, EX16@737A/G or EX16@771G/A, then it can be reasonably predicted that the individual has an elevated susceptibility to bacterial toxins. In other words, such an individual has an increased likelihood or is at an increased risk of developing an adverse condition caused by bacterial toxins, particularly ricin, cholera or Shiga toxins. If an individual is homozygous with the GOLPH4 genotype EX12@−78C/C, EX15@−85C/C, EX15@+86C/C, EX16@323G/G, EX16@737A/A or EX16@771G/G, then it can be reasonably predicted that the individual has a reduced susceptibility to bacterial toxins, particularly ricin, cholera or Shiga toxins.

In another aspect, the present invention provides a method for identifying high-risk patients who have a poor prognosis of adverse conditions caused by bacterial toxins, or for the prognosis of a condition associated with bacterial toxins, or predicting/determining the invasiveness of bacterial toxins in a patient, particularly a patient with an adverse condition caused by ricin, cholera, or Shiga toxin. The individual to be tested can be a healthy person or an individual diagnosed with a condition caused by a bacterial toxin. The method comprises the step of genotyping the individual to determine the individual's genotype at one or more of the GOLPH4 loci identified in the present invention, namely EX12@−78, EX15@−85, EX15@+86, EX16@323, EX16@737, EX16@771, or another locus at which the genotype is in linkage disequilibrium with one of these SNPs. Thus, if one or more the SNPs EX12@−78T, EX15@−85G, EX15@+86G, EX16@323A, EX16@737G, EX16@771A are detected, or a SNP that is in linkage disequilibrium with any one of such SNPs is detected in the individual, then it can be reasonably predicted that the individual has high invasive potential and that the condition caused by bacterial toxins has poor prognosis. If the individual is homozygous with the GOLPH4 genotype EX12@−78T/T, EX15@−85G/G, EX15@+86G/G, EX16@323A/A, EX16@737G/G, EX16@771A/A, it can be reasonably predicted that the individual will have a particularly poor prognosis for bacterial infection. On the other hand, if the individual is homozygous with the GOLPH4 genotype EX12@−78C/C, EX15@−85C/C, EX15@+86C/C, EX16@323G/G, EX16@737A/A, EX16@771G/G, it can be reasonably predicted that the individual has an good prognosis for bacterial infection, especially those associated with ricin, cholera, or Shiga toxin expression.

The SNPs listed in Table 18, i.e. those at positions 169,127,554, 169,140,725, 168,943,494 and 169,109,449 of chromosome III, have also been shown to be associated with GOLPH4 mRNA levels. Chromosome SNPs associated with lower GOLPH4 mRNA expression levels are, whereas those associated with lower GOLPH4 mRNA expression are. In addition to those mentioned above, these SNPs may be utilized in the applications described above.

RABEP1

As indicated in Tables 19-23 and 60-65, the expression level of the RABEP1 gene in human cells is an inheritable “quantitative trait” with genetic determinants. Furthermore, the SNPs and/or haplotypes in accordance with the present invention are associated with the “quantitative trait”, i.e., the RABEP1 mRNA level in human cells. Specifically, the SNPs EX1@−551C, EX1@73T, EX18@276T, EX14@30T, EX18@1782A, EX18@646m, EX18@690w, EX16@−42A, EX17@15A, EX17@36T, EX17@87G, EX18@903w, EX18@1621G, EX18@1676A, EX18@1689w, EX@1806C, EX18@2363A, EX18@2373w, EX18@2397G, EX18@2586T and EX18@2631G are associated with a “low expression phenotype” while the EX1@−551T, EX1@73C, EX18@276C, EX14@30C, EX18@1782T, EX18@646w, EX18@690m, EX16@−42T, EX17@15G, EX17@36C, EX17@87A, EX18@903m, EX18@1621A, EX18@1676G, EX18@1689m, EX@1806T, EX18@2363G, EX18@2373m, EX18@2397C, EX18@2586C and EX18@2631A are associated with a “high expression phenotype.” Thus, the SNPs and/or haplotypes are particularly useful in predicting the level of RABEP1 gene expression in an individual.

Thus, in one aspect, the present invention encompasses a method for predicting or detecting cancer susceptibility in an individual, which comprises the step of genotyping the individual to determine the individual's genotype at one or more of the RABEP1 loci identified in the present invention, namely EX1@−551, EX1@73, EX18@276, EX14@30, EX18@1782, EX18@646, EX18@690, EX16@−42, EX17@15, EX17@36, EX17@87, EX18@903, EX18@1621, EX18@1676, EX18@1689, EX@1806, EX18@2363, EX18@2373, EX18@2397, EX18@2586T and EX18@2631, or another locus at which the genotype is in linkage disequilibrium with one of the SNPs or haplotypes of the present invention. Thus, if one or more the SNPs EX1@−551T, EX1@73C, EX18@276C, EX14@30C, EX18@1782T, EX18@646w, EX18@690m, EX16@−42T, EX17@15G, EX17@36C, EX17@87A, EX18@903m, EX18@1621A, EX18@1676G, EX18@1689m, EX@1806T, EX18@2363G, EX18@2373m, EX18@2397C, EX18@2586C and EX18@2631A are detected, or a SNP that is in linkage disequilibrium with any one of such SNPs is detected in the individual, then it can be reasonably predicted that the individual is at an increased risk of developing cancer. In particularly, if an individual is homozygous with the RABEP1 genotype EX1@−551T/T, EX1@73C/C, EX18@276C/C, EX14@30C/C, EX18@1782T/T, EX18@646w/w, EX18@690m/m, EX16@−42T/T, EX17@15G/G, EX17@36C/C, EX17@87A/A, EX18@903m/m, EX18@1621A/A, EX18@1676G/G, EX18@1689m/m, EX@1806T/T, EX18@2363G/G, EX18@2373m/m, EX18@2397C/C, EX18@2586C/C and EX18@2631A/A, then it can be reasonably predicted that the individual has an elevated susceptibility to cancer. Likewise, if the individual is homozygous with a genotype at a locus that is in the same haplotype with the RABEP1 SNPs EX14@30T, EX18@1782A (in linkage disequilibrium), or in the same haplotype (linkage disequilibrium) with the RABEP1 SNPs EX16@−42A, EX17@15A, EX17@36T, EX17@87G, EX18@903w, EX18@1621G, EX18@1676A, EX18@1689w, EX@1806C, EX18@2363A, EX18@2373w, EX18@2397G, EX18@2586T and EX18@2631G, or in the same haplotype (linkage disequilibrium) with the SNPs EX18@646m, EX18@690w, then it can reasonably be predicted that the individual has an elevated susceptibility to cancer. In other words, such an individual has an increased likelihood or is at an increased risk of developing cancer. If an individual is heterozygous, then his or her risk of developing cancer is at an intermediate level. On the other hand, if the individual is homozygous with the RABEP1 genotype EX1@−551C/C, EX1@73T/T, EX18@276T/T, EX14@30T/T, EX18@1782A/A, EX18@646m/m, EX18@690w/w, EX16@−42A/A, EX17@15A/A, EX17@36T/T, EX17@87G/G, EX18@903w/w, EX18@1621G/G, EX18@1676A/A, EX18@1689w/w, EX@1806C/C, EX18@2363A/A, EX18@2373w/w, EX18@2397G/G, EX18@2586T/T and EX18@2631G/G, then it can be reasonably predicted that the individual has a reduced susceptibility to cancer. Similarly, if the individual is homozygous with a genotype at a locus that is in the same haplotype with the SNPs EX14@30C, EX18@1782T (in linkage disequilibrium), or in the same haplotype (linkage disequilibrium) with the SNPs EX16@−42T, EX17@15G, EX17@36c, EX17@87A, EX18@903m, EX18@1621A, EX18@1676G, EX18@1689m, EX@1806T, EX18@2363G, EX18@2373m, EX18@2397C, EX18@2586C and EX18@2631A or in the same haplotype (linkage disequilibrium) with the SNPs EX18@646w, EX18@690m, then it can reasonably be predicted that the individual has a reduced susceptibility to cancer.

In another aspect, the present invention provides a method for identifying high-risk patients who have a poor prognosis of cancer, or for the prognosis of cancer, or predicting/determining the invasiveness and metastatic potential of a tumor in a patient. The individual to be tested can be a healthy person or an individual diagnosed of cancer. The method comprises the step of genotyping the individual to determine the individual's genotype at one or more of the loci identified in the present invention, namely RABEP1 SNPs EX1@−551, EX1@73, EX18@276, EX14@30, EX18@1782, EX18@646, EX18@690, EX16@−42, EX17@15, EX17@36, EX17@87, EX18@903, EX18@1621, EX18@1676, EX18@1689, EX@1806, EX18@2363, EX18@2373, EX18@2397, EX18@2586T and EX18@2631, or another locus at which the genotype is in linkage disequilibrium with one of these SNPs. Thus, if one or more the RABEP1 SNPs EX1@−551T, EX1@73C, EX18@276C, EX14@30C, EX18@1782T, EX18@646w, EX18@690m, EX16@−42T, EX17@15G, EX17@36C, EX17@87A, EX18@903m, EX18@1621A, EX18@1676G, EX18@1689m, EX@1806T, EX18@2363G, EX18@2373m, EX18@2397C, EX18@2586C and EX18@2631A are detected, or a SNP that is in linkage disequilibrium with any one of such SNPs is detected in the individual, then it can be reasonably predicted that the tumor in such an individual has high metastatic potential, that the cancer has poor prognosis, and that the tumor cells are likely invasive. In other words, the individual has an increased likelihood, or is at an increased risk for cancer metastasis. Particularly, if an individual is homozygous with the RABEP1 genotype EX1@−551T/T, EX1@73C/C, EX18@276C/C, EX14@30C/C, EX18@1782T/T, EX18@646w/w, EX18@690m/m, EX16@−42T/T, EX17@15G/G, EX17@36C/C, EX17@87A/A, EX18@903m/m, EX18@1621A/A, EX18@1676G/G, EX18@1689m/m, EX@1806T/T, EX18@2363G/G, EX18@2373m/m, EX18@2397C/C, EX18@2586C/C and EX18@2631A/A, then the individual has particularly poor prognosis, and cells of their tumor are likely highly invasive. In other words, the individual has a substantially increased likelihood, or is at a substantially increased risk for cancer metastasis. However, if an individual is heterozygous with the RABEP1 genotype EX1@−551C/T, EX1@73T/C, EX18@276T/C, EX14@30T/C, EX18@1782A/T, EX18@646m/w, EX18@690w/m, EX16@−42A/T, EX17@15A/G, EX17@36T/C, EX17@87G/A, EX18@903w/m, EX18@1621G/A, EX18@1676A/G, EX18@1689w/m, EX@1806C/T, EX18@2363A/G, EX18@2373w/m, EX18@2397G/C, EX18@2586T/C and EX18@2631G/A, then the individual has an intermediate prognosis, and the cells of their tumor are potentially invasive. Specifically, the individual has an intermediate level of risk for cancer metastasis. That is, the risk is higher than a person having a homozygous RABEP1 genotype of EX1@−551C/C, EX1@73T/T, EX18@276T/T, EX14@30T/T, EX18@1782A/A, EX18@646m/m, EX18@690w/w, EX16@−42A/A, EX7@15A/A, EX17@36T/T, EX17@87G/G, EX18@903w/w, EX18@1621G/G, EX18@1676A/A, EX18@1689w/w, EX18@1806C/C, EX18@2363A/A, EX18@2373w/w, EX18@2397G/G, EX18@2586T/T and EX18@2631G/G, but is lower than a person having a homozygous RABEP1 genotype of EX1@−551T/T, EX1@73C/C, EX18@276C/C, EX14@30C/C, EX18@1782T/T, EX18@646w/w, EX18@690m/m, EX16@−42T/T, EX17@15G/G, EX17@36C/C, EX17@87A/A, EX18@903m/m, EX18@1621A/A, EX18@1676G/G, EX18@1689m/m, EX18@1806T/T, EX18@2363G/G, EX18@2373m/m, EX18@2397C/C, EX18@2586C/C and EX18@2631A/A.

Further, if the individual is homozygous with the RABEP1 genotype EX1@−551C/C, EX1@73T/T, EX18@276T/T, EX14@30T/T, EX18@1782A/A, EX18@646m/m, EX18@690w/w, EX16@−42A/A, EX17@15A/A, EX17@36T/T, EX17@87G/G, EX18@903w/w, EX18@1621G/G, EX18@1676A/A, EX18@1689w/w, EX18@1806C/C, EX18@2363A/A, EX18@2373w/w, EX18@2397G/G, EX18@2586T/T and EX18@2631G/G, then it can be reasonably predicted that the tumor in the individual has low metastatic potential, that patient with the cancer has a good prognosis, and that the tumor cells are likely not invasive. That is, the individual does not have an increased likelihood, or increased risk, for cancer metastasis.

Another aspect of the present invention encompasses a method for predicting or detecting susceptibility to neurodegenerative disease in a patient, which comprises the step of genotyping the individual to determine the individual's genotype at one or more of the RABEP1 loci identified in the present invention, namely EX1@−551, EX1@73, EX18@276, EX14@30, EX18@1782, EX18@646, EX18@690, EX16@−42, EX17@15, EX17@36, EX17@87, EX18@903, EX18@1621, EX18@1676, EX18@1689, EX@1806, EX18@2363, EX18@2373, EX18@2397, EX18@2586T and EX18@2631, or another locus at which the genotype is in linkage disequilibrium with one of the SNPs of the present invention. Thus, if one or more the RABEP1 SNPs EX1@−551 T, EX1@73C, EX18@276C, EX14@30C, EX18@1782T, EX18@646w, EX18@690m, EX16@−42T, EX17@15G, EX17@36C, EX17@87A, EX18@903m, EX18@1621A, EX18@1676G, EX18@1689m, EX@1806T, EX18@2363G, EX18@2373m, EX18@2397C, EX18@2586C and EX18@2631A are detected, or a SNP that is in linkage disequilibrium with any one of such SNPs is detected in the individual, then it can be reasonably predicted that the individual has increased risk of neurodegenerative disease, especially Parkinson's disease, Alzheimer's disease, Niemann-Pick type C disease and age-related neurodegeneration. In particular, if an individual is homozygous with the genotype EX1@−551T/T, EX1@73C/C, EX18@276C/C, EX14@30C/C, EX18@1782T/T, EX18@646w/w, EX18@690m/m, EX16@−42T/T, EX17@15G/G, EX17@36C/C, EX17@87A/A, EX18@903m/m, EX18@1621A/A, EX18@1676G/G, EX18@1689m/m, EX18@1806T/T, EX18@2363G/G, EX18@2373m/m, EX18@2397C/C, EX18@2586C/C and EX18@2631A/A, then it can be reasonably predicted that the individual has an elevated susceptibility to neurodegenerative disease. In other words, such an individual has an increased likelihood or is at an increased risk of developing a neurodegenerative disease. If an individual is heterozygous, then his or her risk of developing Parkinson's disease, Alzheimer's disease, Niemann-Pick type C disease and age-related neurodegeneration, is at an intermediate level. On the other hand, if the individual is homozygous with the genotype SNPs EX1@−551C/C, EX1@73T/T, EX18@276T/T, EX14@30T/T, EX18@1782A/A, EX18@646m/m, EX18@690w/w, EX16@−42A/A, EX17@15A/A, EX17@36T/T, EX17@87G/G, EX18@903w/w, EX18@1621G/G, EX18@1676A/A, EX18@1689w/w, EX18@1806C/C, EX18@2363A/A, EX18@2373w/w, EX18@2397G/G, EX18@2586T/T and EX18@2631G/G, then it can be reasonably predicted that the individual has a reduced susceptibility to neurodegenerative disease, especially Parkinson's disease, Alzheimer's disease, Niemann-Pick type C disease and age-related neurodegeneration.

In another aspect, the present invention provides a method for identifying high-risk patients who have a poor prognosis of a neurodegenerative or neurological disease, or for the prognosis of neurodegenerative or neurological disease, or predicting/determining the ability to recover from neuronal damage resulting from brain trauma. The individual to be tested can be a healthy person or an individual diagnosed with or neurodegenerative disease. Thus, if one or more of the SNPs EX1@−551T, EX1@73C, EX18@276C, EX14@30C, EX18@1782T, EX18@646w, EX18@690m, EX16@−42T, EX17@15G, EX17@36C, EX17@87A, EX18@903m, EX18@1621A, EX18@1676G, EX18@1689m, EX@1806T, EX18@2363G, EX18@2373m, EX18@2397C, EX18@2586C and EX18@2631A are detected, or a SNP that is in linkage disequilibrium with any one of such SNPs is detected in the individual, then it can be reasonably predicted that the individual has an increased likelihood of neurodegenerative disease, especially Parkinson's disease, Alzheimer's disease, Niemann-Pick type C disease and age-related neurodegeneration. Particularly, if an individual is homozygous with the genotype EX1@−551T/T, EX1@73C/C, EX18@276C/C, EX14@30C/C, EX18@1782T/T, EX18@646w/w, EX18@690m/m, EX16@−42T/T, EX17@15G/G, EX17@36C/C, EX17@87A/A, EX18@903m/m, EX18@1621A/A, EX18@1676G/G, EX18@1689m/m, EX18@1806T/T, EX18@2363G/G, EX18@2373m/m, EX18@2397C/C, EX18@2586C/C and EX18@2631A/A, then the individual has particular poor prognosis neurodegenerative disease or damage will have greater effects. In other words, the individual has a substantially increased likelihood or at a substantially increased risk of progression of the neurodegenerative disease or damage. However, if an individual is heterozygous with the genotype EX1@−551C/T, EX1@73T/C, EX18@276T/C, EX14@30T/C, EX18@1782A/T, EX18@646m/w, EX18@690w/m, EX16@−42A/T, EX17@15A/G, EX17@36T/C, EX17@87G/A, EX18@903w/m, EX18@1621G/A, EX18@1676A/G, EX18@1689w/m, EX@1806C/T, EX18@2363A/G, EX18@2373w/m, EX18@2397G/C, EX18@2586T/C and EX18@2631G/A, an intermediate level of risk of neurological disease, especially Parkinson's disease, Alzheimer's disease, Niemann-Pick type C disease and age-related neurodegeneration. That is, the risk is higher than a person having a homozygous genotype of EX1@−551C/C, EX1@73T/T, EX18@276T/T, EX14@30T/T, EX18@1782A/A, EX18@646m/m, EX1 8@690w/w, EX16@−42A/A, EX17@15A/A, EX17@36T/T, EX17@87G/G, EX18@903w/w, EX18@1621G/G, EX18@1676A/A, EX18@1689w/w, EX@1806C/C, EX18@2363A/A, EX18@2373w/w, EX18@2397G/G, EX18@2586T/T and EX18@2631G/G, but is lower than a person having a homozygous genotype of EX1@−551 T/T, EX1@73C/C, EX18@276C/C, EX14@30C/C, EX18@1782T/T, EX18@646w/w, EX18@690m/m, EX16@−42T/T, EX17@15G/G, EX17@36C/C, EX17@87A/A, EX18@903m/m, EX18@1621A/A, EX18@1676G/G, EX18@1689m/m, EX18@1806T/T, EX18@2363G/G, EX18@2373m/m, EX18@2397C/C, EX18@2586C/C and EX18@2631A/A.

The SNPs listed in Table 23, i.e. those at positions 5,238,870, 5,264,880, 5,265,310, 5,251,617, 5,250,885 and 5,255,563 of chromosome 17, have also been shown to be associated with RABEP1 mRNA levels. In addition to those mentioned above, these SNPs may be utilized in the applications described above.

TAP2

As indicated in Tables 24, 25 and 66-70 below, the expression level of the TAP2 gene in human cells is an inheritable “quantitative trait” with genetic determinants. Furthermore, the SNPs and/or haplotypes in accordance with the present invention are associated with the “quantitative trait”, i.e., mRNA level of the TAP2 gene in human cells. Specifically, the SNPs EX8@36C, EX10@+23T, EX12@19C, EX12@61A, EX12@127T, EX12@332A, EX12@356G, EX11@17A, EX11@+9C, EX12@159G, EX12@291G, EX12@358w, EX12@466A, EX12@586C, EX12@668T, EX12@754G, EX12@755C, EX12@793G, EX12@847C and EX12@1132w are associated with a “low expression phenotype” while the EX8@36T, EX10@+23C, EX12@19T, EX12@61G, EX12@127C, EX12@332G, EX12@356T, EX11@17G, EX11@+9T, EX12@159T, EX12@291A, EX12@358m, EX12@466G, EX12@586T, EX12@668C, EX12@754A, EX12@755T, EX12@793T, EX12@847T and EX12@1132m are associated with a “high expression phenotype.” Thus, the SNPs and/or haplotypes are particularly useful in predicting the level of TAP2 gene expression in an individual.

Thus, in one aspect, the present invention encompasses a method for predicting or detecting cancer susceptibility in an individual, which comprises the step of genotyping the individual to determine the individual's genotype at one or more of the TAP2 loci identified in the present invention, namely EX8@36, EX10@+23, EX12@19, EX12@61, EX12@127, EX12@332, EX12@356, EX11@17, EX1@1B+9, EX12@159G, EX12@291, EX12@358, EX12@466, EX12@586, EX12@668, EX12@754, EX12@755, EX12@793, EX12@847 or EX12@1132, or another locus at which the genotype is in linkage disequilibrium with one of the SNPs or haplotypes of the present invention. Thus, if one or more the EX8@36C, EX10@+23T, EX12@19C, EX12@61A, EX12@127T, EX12@332A, EX12@356G, EX11@17A, EX11@+9C, EX12@159G, EX12@291G, EX12@358w, EX12@466A, EX12@586C, EX12@668T, EX12@754G, EX12@755C, EX12@793G, EX12@847C or EX12@1132w are detected, or a SNP that is in linkage disequilibrium with any one of such SNPs is detected in the individual, then it can be reasonably predicted that the individual is at an increased risk of developing cancer, particularly skin cancer, breast cancer or small cell lung cancer. In particularly, if an individual is homozygous with the TAP2 genotype EX8@36C/C, EX10@+23T/T, EX12@19C/C, EX12@61A/A, EX12@127T/T, EX12@332A/A, EX12@356G/G, EX11@17A/A, EX11@+9C/C, EX12@159G/G, EX12@291G/G, EX12@358w/w, EX12@466A/A, EX12@586C/C, EX12@668T/T, EX12@754G/G, EX12@755C/C, EX12@793G/G, EX12@847C/C or EX12@1132w/w, then it can be reasonably predicted that the individual has an elevated susceptibility to cancer, particularly skin cancer, breast cancer or small cell lung cancer. Likewise, if the individual is homozygous with a genotype at a TAP2 locus that is in the same haplotype with the SNPs EX12@61A, EX12@127T, EX12@332A, EX12@356G, EX11@17A, EX11@+9C, EX12@159G, EX12@291G, EX12@358w, EX12@466A, EX12@586C, EX12@668T, EX12@754G, EX12@755C, EX12@793G, EX12@847C or EX12@1132w (in linkage disequilibrium), then it can reasonably be predicted that the individual has an elevated susceptibility to cancer, particularly skin cancer, breast cancer or small cell lung cancer. In other words, such an individual has an increased likelihood or is at an increased risk of developing cancer, particularly skin cancer, breast cancer or small cell lung cancer. If an individual is heterozygous, then his or her risk of developing cancer is at an intermediate level. On the other hand, if the individual is homozygous with the TAP2 genotype EX8@36T/T, EX10@+23C/C, EX12@19T/T, EX12@61G/G, EX12@127C/C, EX12@332G/G, EX12@356T/T, EX11@17G/G, EX1 1@+9T/T, EX12@159T/T, EX12@291A/A, EX12@358m/m, EX12@466G/G, EX12@586T/T, EX12@668C/C, EX12@754A/A, EX12@755T/T, EX12@793T/T, EX12@847T/T or EX12@1132m/m, then it can be reasonably predicted that the individual has a reduced susceptibility to cancer, particularly skin cancer, breast cancer or small cell lung cancer. Similarly, if the individual is homozygous with a genotype at a TAP2 locus that is in the same haplotype with the SNPs EX12@61G, EX12@127C, EX12@332G, EX12@356T, EX11@17G, EX11@+9T, EX12@159T, EX12@291A, EX12@358m, EX12@466G, EX12@586T, EX12@668C, EX12@754A, EX12@755T, EX12@793T, EX12@847T and EX12@1132m (in linkage disequilibrium), then it can reasonably be predicted that the individual has a reduced susceptibility to cancer, particularly skin cancer, breast cancer or small cell lung cancer.

In another aspect, the present invention provides a method for identifying patients who's cancer has a poor prognosis, or for predicting or determining the potential invasiveness and metastatic potential of tumor in a patient, particularly cancer patient, e.g., with cancer such as melanoma, breast cancer or small cell lung cancer. The patient to be tested can be a patient diagnosed with cancer, particularly melanoma, breast cancer or small cell lung cancer. The method comprises the steps of genotyping the cancerous growth, or tumor, to determine the genotype of the cancer itself, by determining the nucleotide present at one or more of the TAP2 loci identified in the present invention, namely EX8@36, EX10@+23, EX12@19, EX12@61, EX12@127, EX12@332, EX12@356, EX11@17, EX11@+9, EX12@159G, EX12@291, EX12@358, EX12@466, EX12@586, EX12@668, EX12@754, EX12@755, EX12@793, EX12@847 or EX12@1132, or another locus at which the genotype is in linkage disequilibrium with one of the SNPs or haplotypes of the present invention. Thus, if one or more the TAP2 SNPs EX8@36C, EX10@+23T, EX12@19C, EX12@61A, EX12@127T, EX12@332A, EX12@356G, EX11@17A, EX11@+9C, EX12@159G, EX12@291G, EX12@358w, EX12@466A, EX12@586C, EX12@668T, EX12@754G, EX12@755C, EX12@793G, EX12@847C or EX12@1132w are detected, or a SNP that is in linkage disequilibrium with any one of such SNPs is detected in the cancerous growth or tumor, then it can be reasonably predicted that the cancerous growth or tumor has high metastatic potential, that the cancer has poor prognosis, and that the cells of the cancerous growth or tumor are likely invasive. In other words, the patient with a cancerous growth or tumor with such a genotype has an increased likelihood, or increased risk, of metastatic cancer. Particularly, if the cancerous growth or tumor is homozygous with the TAP2 genotype EX8@36C/C, EX10@+23T/T, EX12@19C/C, EX12@61A/A, EX12@127T/T, EX12@332A/A, EX12@356G/G, EX11@17A/A, EX11@+9C/C, EX12@159G/G, EX12@291G/G, EX12@358w/w, EX12@466A/A, EX12@586C/C, EX12@668T/T, EX12@754G/G, EX12@755C/C, EX12@793G/G, EX12@847C/C or EX12@1132w/w, then the patient has particularly poor prognosis and their tumor cells are likely highly invasive capable of rapid growth. In other words, the individual has a substantially increased likelihood or at a substantially increased risk of cancer metastasis and rapid cancer growth. However, if the cancerous growth or tumor is heterozygous with the TAP2 genotype EX8@36C/T, EX10@+23T/C, EX12@19C/T, EX12@61A/G, EX12@127T/C, EX12@332A/G, EX12@356G/T, EX11@17A/G, EX11@+9C/T, EX12@159G/T, EX12@291G/A, EX12@358w/m, EX12@466A/G, EX12@586C/T, EX12@668T/C, EX12@754G/A, EX12@755C/T, EX12@793G/T, EX12@847C/T or EX12@1132w/m, then the patient has a somewhat less poor prognosis and their tumor cells are likely only moderately invasive. Specifically, the patient has an intermediate level of risk of cancer metastasis. That is, the risk is greater than a patient having a cancerous growth or tumor that has a homozygous TAP2 genotype of EX8@36C/C, EX10@+23T/T, EX12@19C/C, EX12@61A/A, EX12@127T/T, EX12@332A/A, EX12@356G/G, EX11@17A/A, EX11@+9C/C, EX12@159G/G, EX12@291G/G, EX12@358w/w, EX12@466A/A, EX12@586C/C, EX12@668T/T, EX12@754G/G, EX12@755C/C, EX12@793G/G, EX12@847C/C or EX12@1132w/w, but is lower than a patient having a cancerous growth or tumor that has a homozygous TAP2 genotype of EX8@36T/T, EX10@+23C/C, EX12@19T/T, EX12@61G/G, EX12@127C/C, EX12@332G/G, EX12@356T/T, EX11@17G/G, EX11@+9T/T, EX12@159T/T, EX12@291A/A, EX12@358m/m, EX12@466G/G, EX12@586T/T, EX12@668C/C, EX12@754A/A, EX12@755T/T, EX12@793T/T, EX12@847T/T or EX12@1132m/m.

Thus, if the patient has a cancerous growth or tumor that has a homozygous TAP2 genotype of EX8@36T/T, EX10@+23C/C, EX12@19T/T, EX12@61G/G, EX12@127C/C, EX12@332G/G, EX12@356T/T, EX11@17G/G, EX11@+9T/T, EX12@159T/T, EX12@291A/A, EX12@358m/m, EX12@466G/G, EX12@586T/T, EX12@668C/C, EX12@754A/A, EX12@755T/T, EX12@793T/T, EX12@847T/T or EX12@1132m/m, then it can be reasonably predicted that the tumor in the individual has low metastic potential, that the cancer has a better prognosis, and that the tumor cells are likely less invasive and give to rapid growth. That is, the patient does not have an increased likelihood or increased risk of cancer metastasis. Similarly, if the cancerous growth or tumor is homozygous with a genotype at a TAP2 locus that is in the same haplotype with the SNPs EX12@61G/G, EX12@127C/C, EX12@332G/G, EX12@356T/T, EX11@17G/G, EX11@+9T/T, EX12@159T/T, EX12@291A/A, EX12@358m/m, EX12@466G/G, EX12@586T/T, EX12@668C/C, EX12@754A/A, EX12@755T/T, EX12@793T/T, EX12@847T/T or EX12@1132m/m (in linkage disequilibrium), then it can reasonably be predicted that the cancer has a low metastatic potential, has good prognosis, and the tumor cells are likely less invasive. In other words, the patient does not have an increased likelihood or increased risk of cancer metastasis.

In another aspect, the present invention encompasses a method for predicting or detecting susceptibility to autoimmune disease in an individual, which comprises the step of genotyping the individual to determine the individual's genotype at one or more of the TAP2 loci identified in the present invention, namely EX8@36, EX10@+23, EX12@19, EX12@61, EX12@127, EX12@332, EX12@356, EX11@17, EX11@+9, EX12@159G, EX12@291, EX12@358, EX12@466, EX12@586, EX12@668, EX12@754, EX12@755, EX12@793, EX12@847 or EX12@1132, or another locus at which the genotype is in linkage disequilibrium with one of the SNPs or haplotypes of the present invention. Thus, if one or more the TAP2 SNPs EX8@36C, EX10@+23T, EX12@19C, EX12@61A, EX12@127T, EX12@332A, EX12@356G, EX11@17A, EX1@+9C, EX12@159G, EX12@291G, EX12@358w, EX12@466A, EX12@586C, EX12@668T, EX12@754G, EX12@755C, EX12@793G, EX12@847C or EX12@1132w are detected, or a SNP that is in linkage disequilibrium with any one of such SNPs is detected in the individual, then it can be reasonably predicted that the individual is at an increased risk of developing an autoimmune disease, particularly Wegener's granulomatosis, multiple sclerosis, type 1 diabetes mellitus, lupus, and rheumatoid arthritis. In particularly, if an individual is homozygous with the TAP2 genotype EX8@36C/C, EX10@+23T/T, EX12@19C/C, EX12@61A/A, EX12@127T/T, EX12@332A/A, EX12@356G/G, EX11@17A/A, EX11@+9C/C, EX12@159G/G, EX12@291G/G, EX12@358w/w, EX12@466A/A, EX12@586C/C, EX12@668T/T, EX12@754G/G, EX12@755C/C, EX12@793G/G, EX12@847C/C or EX12@1132w/w, then it can be reasonably predicted that the individual has an elevated susceptibility to autoimmune disease, particularly Wegener's granulomatosis, multiple sclerosis, type 1 diabetes mellitus, lupus, and rheumatoid arthritis. Likewise, if the individual is homozygous with a TAP2 genotype at a locus that is in the same haplotype with the SNPs EX12@61A, EX12@127T, EX12@332A, EX12@356G, EX11@17A, EX11@+9C, EX12@159G, EX12@291G, EX12@358w, EX12@466A, EX12@586C, EX12@668T, EX12@754G, EX12@755C, EX12@793G, EX12@847C or EX12@1132w (in linkage disequilibrium), then it can reasonably be predicted that the individual has an elevated susceptibility to autoimmune disease, particularly Wegener's granulomatosis, multiple sclerosis, type 1 diabetes mellitus, lupus, and rheumatoid arthritis. In other words, such an individual has an increased likelihood or is at an increased risk of developing autoimmune disease, particularly Wegener's granulomatosis, multiple sclerosis, type 1 diabetes mellitus, lupus, and rheumatoid arthritis. If an individual is heterozygous, then his or her risk of developing autoimmune disease is at an intermediate level. One the other hand, if the individual is homozygous with the TAP2 genotype EX8@36T/T, EX10@+23C/C, EX12@19T/T, EX12@61G/G, EX12@127C/C, EX12@332G/G, EX12@356T/T, EX11@17G/G, EX11@+9T/T, EX12@159T/T, EX12@291A/A, EX12@358m/m, EX12@466G/G, EX12@586T/T, EX12@668C/C, EX12@754A/A, EX12@755T/T, EX12@793T/T, EX12@847T/T or EX12@1132m/m, then it can be reasonably predicted that the individual has a reduced susceptibility to autoimmune disease, particularly Wegener's granulomatosis, multiple sclerosis, type 1 diabetes mellitus, lupus, and rheumatoid arthritis. Similarly, if the individual is homozygous with a genotype at a locus that is in the same haplotype with the TAP2 SNPs EX12@61G, EX12@127C, EX12@332G, EX12@356T, EX11@17G, EX11@+9T, EX12@159T, EX12@291A, EX12@358m, EX12@466G, EX12@586T, EX12@668C, EX12@754A, EX12@755T, EX12@793T, EX12@847T and EX12@1132m (in linkage disequilibrium), then it can reasonably be predicted that the individual has a reduced susceptibility to autoimmune disease, particularly Wegener's granulomatosis, multiple sclerosis, type 1 diabetes, lupus, mellitus and rheumatoid arthritis.

In yet another aspect, the present invention encompasses a method for predicting or detecting susceptibility to viral infection in an individual, which comprises the step of genotyping the individual to determine the individual's genotype at one or more of the TAP2 loci identified in the present invention, namely EX8@36, EX10@+23, EX12@19, EX12@61, EX12@127, EX12@332, EX12@356, EX11@17, EX11@+9, EX12@159G, EX12@291, EX12@358, EX12@466, EX12@586, EX12@668, EX12@754, EX12@755, EX12@793, EX12@847 or EX12@1132, or another locus at which the genotype is in linkage disequilibrium with one of the SNPs or haplotypes of the present invention. Thus, if one or more the TAP2 SNPs EX8@36C, EX10@+23T, EX12@19C, EX12@61A, EX12@127T, EX12@332A, EX12@356G, EX11@17A, EX11@+9C, EX12@159G, EX12@291G, EX12@358w, EX12@466A, EX12@586C, EX12@668T, EX12@754G, EX12@755C, EX12@793G, EX12@847C or EX12@1132w are detected, or a SNP that is in linkage disequilibrium with any one of such SNPs is detected in the individual, then it can be reasonably predicted that the individual is at an increased risk of developing viral infection. In particularly, if an individual is homozygous with the TAP2 genotype EX8@36C/C, EX10@+23T/T, EX12@19C/C, EX12@61A/A, EX12@127T/T, EX12@332A/A, EX12@3356G/G, EX11@17A/A, EX11@+9C/C, EX12@159G/G, EX12@291G/G, EX12@358w/w, EX12@466A/A, EX12@586C/C, EX12@668T/T, EX12@754G/G, EX12@755C/C, EX12@793G/G, EX12@847C/C or EX12@1132w/w, then it can be reasonably predicted that the individual has an elevated susceptibility to viral infection. Likewise, if the individual is homozygous with a genotype at a TAP2 locus that is in the same haplotype with the SNPs EX12@61A, EX12@127T, EX12@332A, EX12@356G, EX11@17A, EX11@+9C, EX12@159G, EX12@291G, EX12@358w, EX12@466A, EX12@586C, EX12@668T, EX12@754G, EX12@755C, EX12@793G, EX12@847C or EX12@1132w (in linkage disequilibrium), then it can reasonably be predicted that the individual has an elevated susceptibility to viral infection. In other words, such an individual has an increased likelihood or is at an increased risk of developing viral infection. If an individual is heterozygous, then his or her risk of developing viral infection is at an intermediate level. One the other hand, if the individual is homozygous with the TAP2 genotype EX8@36T/T, EX10@+23C/C, EX12@19T/T, EX12@61G/G, EX12@127C/C, EX12@332G/G, EX12@356T/T, EX11@17G/G, EX11@+9T/T, EX12@159T/T, EX12@291A/A, EX12@358m/m, EX12@466G/G, EX12@586T/T, EX12@668C/C, EX12@754A/A, EX12@755T/T, EX12@793T/T, EX12@847T/T or EX12@1132m/m, then it can be reasonably predicted that the individual has a reduced susceptibility to viral infection. Similarly, if the individual is homozygous with a genotype at a TAP2 locus that is in the same haplotype with the SNPs EX12@61G, EX12@127C, EX12@332G, EX12@356T, EX11@17G, EX11@+9T, EX12@159T, EX12@291A, EX12@358m, EX12@466G, EX12@586T, EX12@668C, EX12@754A, EX12@755T, EX12@793T, EX12@847T and EX12@1132m (in linkage disequilibrium), then it can reasonably be predicted that the individual has a reduced susceptibility to viral infection.

In still another aspect, the present invention provides a method for identifying high-risk patients who have a poor viral infection prognosis, or for the prognosis of viral infection, or predicting/determining the invasiveness and potential for viral replication in a patient. The individual to be tested can be a healthy person or an individual diagnosed with viral infection. The method comprises the step of genotyping the individual to determine the individual's genotype at one or more of the TAP2 loci identified in the present invention, namely EX8@36, EX10@+23, EX12@19, EX12@61, EX12@127, EX12@332, EX12@356, EX11@17, EX11@+9, EX12@159G, EX12@291, EX12@358, EX12@466, EX12@586, EX12@668, EX12@754, EX12@755, EX12@793, EX12@847 or EX12@1132, or another locus at which the genotype is in linkage disequilibrium with one of the SNPs or haplotypes of the present invention. Thus, if one or more the TAP2 SNPs EX8@36C, EX10@+23T, EX12@19C, EX12@61A, EX12@127T, EX12@332A, EX12@356G, EX11@17A, EX11@+9C, EX12@159G, EX12@291G, EX12@358w, EX12@466A, EX12@586C, EX12@668T, EX12@754G, EX12@755C, EX12@793G, EX12@847C or EX12@1132w are detected, or a SNP that is in linkage disequilibrium with any one of such SNPs is detected in the individual, then it can be reasonably predicted that the individual has an increase likelihood or increased potential of viral replication. Particularly, if an individual is homozygous with the TAP2 genotype EX8@36C/C, EX1O@+23T/T, EX12@19C/C, EX12@61A/A, EX12@127T/T, EX12@332A/A, EX12@356G/G, EX11@17A/A, EX11@+9C/C, EX12@159G/G, EX12@291G/G, EX12@358w/w, EX12@466A/A, EX12@586C/C, EX12@668T/T, EX12@754G/G, EX12@755C/C, EX12@793G/G, EX12@847C/C or EX12@1132w/w, then the individual has particularly poor viral infection prognosis. In other words, the individual has a substantially increased likelihood or at a substantially increased risk of viral replication. However, if an individual is heterozygous with the TAP2 genotype EX8@36C/T, EX1O@+23T/C, EX12@19C/T, EX12@61A/G, EX12@127T/C, EX12@332A/G, EX12@356G/T, EX11@17A/G, EX11@+9C/T, EX12@159G/T, EX12@291G/A, EX12@358w/m, EX12@466A/G, EX12@586C/T, EX12@668T/C, EX12@754G/A, EX12@755C/T, EX12@793G/T, EX12@847C/T or EX12@1132w/m, then the individual has poor prognosis. Specifically, the individual has an intermediate level of risk of progression of viral infection and/or viral replication. That is, the risk is greater than a person having a homozygous TAP2 genotype of EX8@36C/C, EX10@+23T/T, EX12@19C/C, EX12@61A/A, EX12@127T/T, EX12@332A/A, EX12@356G/G, EX11@17A/A, EX11@+9C/C, EX12@159G/G, EX12@291G/G, EX12@358w/w, EX12@466A/A, EX12@586C/C, EX12@668T/T, EX12@754G/G, EX12@755C/C, EX12@793G/G, EX12@847C/C or EX12@1132w/w, but is lower than a person having a homozygous TAP2 genotype of EX8@36T/T, EX10@+23C/C, EX12@19T/T, EX12@61G/G, EX12@127C/C, EX12@332G/G, EX12@356T/T, EX11@17G/G, EX1 1@+9T/T, EX12@159T/T, EX12@291A/A, EX12@358m/m, EX12@466G/G, EX12@586T/T, EX12@668C/C, EX12@754A/A, EX12@755T/T, EX12@793T/T, EX12@847T/T or EX12@1132m/m.

Thus, if the individual is homozygous with the TAP2 genotype EX8@36T/T, EX10@+23C/C, EX12@19T/T, EX12@61G/G, EX12@127C/C, EX12@332G/G, EX12@356T/T, EX11@17G/G, EX11@+9T/T, EX12@159T/T, EX12@291A/A, EX12@358m/m, EX12@466G/G, EX12@586T/T, EX12@668C/C, EX12@754A/A, EX12@755T/T, EX12@793T/T, EX12@847T/T or EX12@1132m/m, then it can be reasonably predicted that the viral infection in the individual has low replication potential, that the individual has a good prognosis. That is, the individual does not have an increased likelihood or increased risk of viral replication and/or viral infection progression. Similarly, if the individual is homozygous with a genotype at a TAP2 locus that is in the same haplotype with the SNPs EX12@61G/G, EX12@127C/C, EX12@332G/G, EX12@356T/T, EX11@17G/G, EX11@+9T/T, EX12@159T/T, EX12@291A/A, EX12@358m/m, EX12@466G/G, EX12@586T/T, EX12@668C/C, EX12@754A/A, EX12@755T/T, EX12@793T/T, EX12@847T/T or EX12@1132m/m (in linkage disequilibrium), then it can reasonably be predicted that the individual has a low replication potential, that the individual has a good prognosis. In other words, the individual does not have an increased likelihood or increased risk of viral infection progression.

The SNPs listed in Table 25, i.e. those at positions 32,511,862 and 32,512,605 of chromosome 6, have also been shown to be associated with TAP2 mRNA levels. Chromosome 6 SNPs associated with lower TAP2 mRNA expression levels are 32,511,862T and 32,512,605A, whereas those associated with lower TAP2 mRNA expression are 32,511,862C and 32,512,605C. In addition to those mentioned above, these SNPs may be utilized in the applications described above.

NARG2

As indicated in Tables 26, 71 and 72, the expression level of the NARG2 gene in human cells is an inheritable “quantitative trait” with genetic determinants. Furthermore, the SNPs in accordance with the present invention are associated with the “quantitative trait”, i.e., mRNA level of the NARG2 gene in human cells. Specifically, the SNPs EX10@−23A, EX12@48C, EX14@+15C, EX16@1757m, EX16@2306C, EX16@2547T and EX16@4025w are associated with the “low expression phenotype” while the EX10@−23C, EX12@48T, EX14@+15G, EX16@1757w, EX16@2306G, EX16@2547G and EX16@4025m are associated with “high expression phenotype.” Thus, the SNPs are particularly useful in predicting the NARG2 gene expression and also NMDA receptor gene expression in an individual.

Thus, in one aspect, the present invention encompasses a method for predicting in an individual NARG2 gene expression (mRNA and/or protein) level or NMDA receptor (NMDAR1) expression level, and the biological, pharmacological or pharmacokinetic consequences thereof.

In one embodiment, the present invention encompasses a method for predicting the pharmacokinetic consequences of NMDA receptor expression, i.e., the responsiveness of an individual to an NMDA receptor antagonist, or the dose of an NMDA receptor antagonist to be used in an individual, or potential toxicity of an NMDA receptor antagonist on an individual, which can all correlate with the NMDA receptor expression level. In specific embodiments, the individual is diagnosed of a disease, e.g., neurodegenerative disease (particularly ALS, Parkinson's disease, Alzheimer's disease), epilepsy, stroke, and other types of brain and spinal cord injury. Specifically, if one or more the SNPs EX10@−23A, EX12@48C, EX14@+15C, EX16@1757m, EX16@2306C, EX16@2547T and EX16@4025w are detected, or a SNP that is in linkage disequilibrium with any one of such SNPs is detected in the individual, then low expression of NARG2 and high expression of NMDAR1 are predicted, and the pharmacokinetic consequences are then predicted. More specifically, the individual's responsiveness to NMDA receptor antagonists is predicted. Selection of patients for inclusion in clinical trials involving an NMDA receptor antagonist can be made based on the predicted NMDA receptor expression. In addition, whether or not to treat the individual with an NMDA receptor antagonist and the dosage or other treatment regimen to be used can also be decided based on the SNP profile in NARG2 gene and the predicted NARG2 and NMDA receptor expression. For example, if a SNP associated with low NARG2 expression and thus high NMDAR1 expression is detected in an individual, then a higher dosage or more frequent treatment may be administered to the individual.

In another aspect, the present invention encompasses a method for predicting or detecting susceptibility to neurodegenerative disease in an individual, which comprises the step of genotyping the individual to determine the individual's genotype at one or more of the loci identified in the present invention, namely EX10@−23, EX12@48, EX14@+15, EX16@1757, EX16@2306, EX16@2547 and EX16@4025, or another locus at which the genotype is in linkage disequilibrium with one of the SNPs or haplotypes of the present invention. Thus, if one or more the SNPs EX10@−23A, EX12@48C, EX14@+15C, EX16@1757m, EX16@2306C, EX16@2547T and EX16@4025w are detected, or a SNP that is in linkage disequilibrium with any one of such SNPs is detected in the individual, then it can be reasonably predicted that the individual is at an increased risk of developing neurodegenerative disease, particularly ALS, Parkinson's disease, Alzheimer's disease and other types of brain and spinal cord injury. In particular, if an individual is homozygous with the genotype EX10@−23A/A, EX12@48C/C, EX14@+15C/C, EX16@1757m/m, EX16@2306C/C, EX16@2547T/T and EX16@4025w/w, then it can be reasonably predicted that the individual has an elevated susceptibility to neurodegenerative disease, particularly ALS, Parkinson's disease, Alzheimer's disease and other types of brain and spinal cord injury. If an individual is heterozygous, then his or her risk of developing neurodegenerative disease is at an intermediate level. On the other hand, if the individual is homozygous with the genotype EX10@−23C/C, EX12@48T/T, EX14@+15G/G, EX16@1757w/w, EX16@2306G/G, EX16@2547G/G and EX16@4025m/m, then it can be reasonably predicted that the individual has a reduced susceptibility to neurodegenerative disease, particularly ALS, Parkinson's disease, Alzheimer's disease and other types of brain and spinal cord injury.

In another aspect, the present invention provides a method for identifying high-risk patients who have a poor prognosis of neurodegenerative disease, or for the prognosis of a neurodegenerative disease, or predicting/determining the potential progression of a neurodegenerative disease. The individual to be tested can be a healthy person or an individual diagnosed with a neurodegenerative disease. The method comprises the step of genotyping the individual to determine the individual's genotype at one or more of the loci identified in the present invention, namely EX10@−23, EX12@48, EX14@+15, EX16@1757, EX16@2306, EX16@2547 and EX16@4025, or another locus at which the genotype is in linkage disequilibrium with one of the SNPs of the present invention. Thus, if one or more the SNPs EX10@−23A, EX12@48C, EX14@+15C, EX16@1757m, EX16@2306C, EX16@2547T and EX16@4025w are detected, or a SNP that is in linkage disequilibrium with any one of such SNPs is detected in the individual, then it can be reasonably predicted that the individual has poor prognosis. In other words the individual has a high potential of neurodegenerative disease progression. Particularly, if an individual is homozygous with the genotype EX10@−23A/A, EX12@48C/C, EX14@+15C/C, EX16@1757m/m, EX16@2306C/C, EX16@2547T/T and EX16@4025w/w, then the individual has particularly poor prognosis. However, if an individual is heterozygous with the genotype EX10@−23A/C, EX12@48C/T, EX14@+15C/G, EX16@1757m/w, EX16@2306C/G, EX16@2547T/G and EX16@4025w/m, then the individual has an intermediate level of risk of neurodegenerative disease progression, especially associated with ALS, Parkinson's disease, Alzheimer's disease and other types of brain and spinal cord injury. That is, the risk is greater than a person having a homozygous genotype of EX10@−23C/C, EX12@48T/T, EX14@+15G/G, EX16@1757w/w, EX16@2306G/G, EX16@2547G/G and EX16@4025m/m, but is lower than a person having a homozygous genotype of EX10@−23A/A, EX12@48C/C, EX14@+15C/C, EX16@1757m/m, EX16@2306C/C, EX16@2547T/T and EX16@4025w/w.

Thus, if the individual is homozygous with the genotype EX10@−23C/C, EX12@48T/T, EX14@+15G/G, EX16@1757w/w, EX16@2306G/G, EX16@2547G/G and EX16@4025m/m, then it can be reasonably predicted that the neurodegenerative disease in the individual will progress and that the individual has a good prognosis. That is, the individual does not have an increased risk or likelihood of neurodegenerative disease progression.

In yet another aspect, the present invention provides a method for predicting or detecting the ability to recover from brain and spinal cord injury in an individual, which comprises the step of genotyping the individual to determine the individual's genotype at one or more of the loci identified in the present invention, namely EX10@−23, EX12@48, EX14@+15, EX16@1757, EX16@2306, EX16@2547 and EX16@4025, or another locus at which the genotype is in linkage disequilibrium with one of the SNPs or haplotypes of the present invention. Thus, if one or more the SNPs EX10@−23C, EX12@48T, EX14@+15G, EX16@1757w, EX16@2306G, EX16@2547G and EX16@4025m are detected, or a SNP that is in linkage disequilibrium with any one of such SNPs is detected in the individual, then it can be reasonably predicted that the individual has a higher expression of NARG2 and is less prone to neuronal differentiation, and has worse prognosis of recovering from brain and spinal cord injury. In particular, if an individual is homozygous with the genotype EX10@−23C/C, EX12@48T/T, EX14@+15G/G, EX16@1757w/w, EX16@2306G/G, EX16@2547G/G and EX16@4025m/m, then it can be reasonably predicted that the individual has a high probability or likelihood of slow recovering from brain and spinal cord injury. If an individual is heterozygous, then his or her likelihood of recovery is at an intermediate level. On the other hand, if the individual is homozygous with the genotype EX10@−23A/A, EX12@48C/C, EX14@+15C/C, EX16@1757m/m, EX16@2306C/C, EX16@2547T/T and EX16@4025w/w, then it can be reasonably predicted that the NARG2 expression in the individual is lower and the individual has high probability of recovering from brain and spinal cord injury.

DDX58

As indicated in Tables 27 and 73 below, the expression level of the DDX58 gene in human cells is an inheritable “quantitative trait” with genetic determinants. Furthermore, the SNPs and/or haplotypes in accordance with the present invention are associated with the “quantitative trait”, i.e., mRNA level of the DDX58 gene in human cells. Specifically, the SNPs EX14@+78C and EX17@63A are associated with the “low expression phenotype” while the SNPs EX14@+78T, EX17@63C are associated with “high expression phenotype.” Thus, the SNPs are particularly useful in predicting the DDX58 gene expression in an individual. Furthermore, other SNPs that are in linkage disequilibrium with the SNPs can also have similar predictive value.

Thus, in one aspect, the present invention encompasses a method for predicting in an individual DDX58 (RIG-1) gene expression (mRNA and/or protein) level, and the biological, pharmacological or pharmacokinetic consequences thereof.

Thus, in one aspect, the present invention encompasses a method for predicting or detecting cancer susceptibility in an individual, which comprises the step of genotyping the individual to determine the individual's genotype at one or more of the loci identified in the present invention, namely EX14@+78 and EX17@63, or another locus at which the genotype is in linkage disequilibrium with one of the SNPs of the present invention. Thus, if one or more the SNPs EX14@+78T, EX17@63C are detected, or a SNP that is in linkage disequilibrium with any one of such SNPs is detected in the individual, then it can be reasonably predicted that the individual is at an increased risk of developing cancer, particularly skin cancer, lung cancer, ovarian cancer or thyoma. In particularly, if an individual is homozygous with the genotype EX14@+78T/T, EX17@63C/C, then it can be reasonably predicted that the individual has an elevated susceptibility to cancer. If an individual is heterozygous, then his or her risk of developing cancer is at an intermediate level. One the other hand, if the individual is homozygous with the genotype EX14@+78C, EX17@63A, then it can be reasonably predicted that the individual has a reduced susceptibility to cancer.

In another aspect, the present invention provides a method for identifying high-risk patients who have a poor prognosis of cancer, or for the prognosis of cancer, or predicting/determining the invasiveness and metastatic potential of tumor in a patient, particularly cancer patient, e.g., with cancer such as melanoma, colon cancer, lung cancer, ovarian cancer, non-small cell lung cancers (NSCLCs), and thyoma. The individual to be tested can be a healthy person or an individual diagnosed of cancer. In this aspect, in patients diagnosed with cancer, either normal tissue or cells, or tumor tissue or cells can be used in genotyping for germline genotype or somatic genotype in tumor samples. The method comprises the step of genotyping the individual to determine the individual's genotype in the sample at one or more of the loci identified in the present invention, namely EX14@+78 and EX17@63, or another locus at which the genotype is in linkage disequilibrium with one of the SNPs of the present invention. Thus, if one or more the SNPs EX14@+78T or EX17@63C are detected, or a SNP that is in linkage disequilibrium with any one of such SNPs is detected in the individual, then it can be reasonably predicted that the individual has high metastasis potential, that the cancer has poor prognosis and that the tumor cells are invasive. In other words, the individual has an increased likelihood or at an increased risk of cancer metastasis. Particularly, if an individual is homozygous with the genotype EX14@+78T/T or EX17@63C/C, then the individual has particular poor prognosis and that the tumor cells are highly invasive. In other words, the individual has a substantially increased likelihood or at a substantially increased risk of cancer metastasis. However, if an individual is heterozygous with the genotype EX14@+78T/C or EX17@63C/A, then the individual has poor prognosis and that the tumor cells are invasive. Specifically, the individual has an intermediate level of risk of cancer metastasis. That is, the risk is greater than a person having a homozygous genotype of EX14@+78C/C or EX17@863A/A, but is lower than a person having a homozygous genotype of EX14@+78T/T or EX17@63C/C.

Thus, if the individual is homozygous with the genotype EX14@+78C/C or EX17@63A/A, then it can be reasonably predicted that the tumor in the individual has low metastasis potential, that the cancer has good prognosis and that the tumor cells are not invasive. That is, the individual does not have an increased likelihood or increased risk of cancer metastasis.

In another aspect, the present invention provides a method for predicting in an individual immune response to viral infection, particularly infection of RNA viruses, and more particularly double-stranded RNA viruses. The method comprises the step of genotyping the individual to determine the individual's genotype at one or more of the loci identified in the present invention, namely EX14@+78 and EX17@63, or another locus at which the genotype is in linkage disequilibrium with one of the SNPs or haplotypes of the present invention. Thus, if one or more of the SNPs associated with high expression of DDX58 (EX14@+78T or EX17@63C, or a SNP that is in linkage disequilibrium with any one of such SNPs) is present in the individual, then it will be reasonable to predict that the individual has an increased likelihood of having a stronger immune response to viral infection, i.e., strong host antiviral response, particularly to double-stranded RNA viruses, e.g., paramyxoviruses, influenza virus and Japanese encephalitis virus. Such individuals will also have an increased resistance to viral infection, particularly RNA viruses, e.g., paramyxoviruses, HIV, HCV, influenza virus and Japanese encephalitis virus. In addition, individuals with such genotypes will also have an increased inflammatory response to viral infection, particularly double-stranded RNA viruses, e.g., paramyxoviruses, influenza virus and Japanese encephalitis virus.

Thus, if one or more the SNPs EX14@+78C or EX17@63A are detected, or a SNP that is in linkage disequilibrium with any one of such SNPs is detected in the individual, then it can be reasonably predicted that the individual will have a decreased resistance to viral infection, more attenuated host antiviral response, decreased immune response to viral infection, and relatively lower inflammatory response to viral infection, particularly double-stranded RNA viruses, e.g., paramyxoviruses, influenza virus and Japanese encephalitis virus. In other words, the individual has an increased likelihood of developing viral infection. Particularly, if an individual is homozygous with the genotype EX14@+78C/C or EX17@63A/A, then the individual has particularly poor immune response and low inflammatory response to viral infection. However, if an individual is heterozygous with the genotype EX14@+78T/C or EX17@63C/A, then the individual has intermediate resistance to viral infection and inflammatory response. Alternatively, if the individual is homozygous with the genotype EX14@+78T/T or EX17@63C/C, then it can be reasonably predicted that the individual will have an increased resistance to viral infection. In other word, the individual will have a reduced susceptibility viral infection, especially infection of double-stranded RNA viruses.

In yet another aspect, the present invention encompasses a method for predicting the pharmacokinetic consequences of DDX58 expression, e.g., the responsiveness of an individual to an anticancer agent or an antiviral agent, or the dose of an anticancer agent or an antiviral agent to be used in an individual, or potential toxicity of an anticancer agent or an antiviral agent on an individual, which can all correlate with the DDX58 or COX-2 expression level. In specific embodiments, the individual is diagnosed of a disease, e.g., cancer or viral infection (particularly infection of a RNA virus, especially double-stranded RNA virus). Specifically, if one or more of the SNPs EX14@+78C and EX17@63A are detected, or an LD SNP that is in linkage disequilibrium with any one of such SNPs is detected in the individual, then low expression of DDX58 is predicted, and the pharmacokinetic consequences are then predicted. More specifically, the individual's responsiveness to an anticancer or antiviral agent is predicted. Selection of patients for inclusion in clinical trials involving an anticancer (particularly COX-2 inhibitor or anti-DDX58) or antiviral agent (particularly RNA virus-specific antiviral agent) can be made based on the predicted DDX58 expression. In addition, the dosage or other treatment regimen to be used can also be decided based on the SNP profile in DDX58 gene and the predicted DDX58 expression. For example, if a SNP associated with low DDX58 expression is detected in a cancer patient, then a lower dosage of, or less frequent treatment with, a COX-2 inhibitor may be administered to the individual in the cancer patient, but the cancer patient may be less responsive to a COX-2 or DDX58 inhibitor. If a SNP associated with high DDX58 expression is detected, especially a homozygosity thereof, then a higher dosage of, or more frequent administration may be required, but the patient may be more responsive to a COX-2 or DDX58 inhibitor.

In another example, if a SNP associated with low DDX58 expression is detected in a patient, especially a homozygosity thereof, then a higher dosage of, or more frequent treatment with, an antiviral agent may be required for treating infection of a RNA virus. Particularly, a higher dosage of interferon may be required. If a SNP associated with high DDX58 expression is detected, especially a homozygosity thereof, then the patient may require a lower dosage of an antiviral agent, or less frequent administration thereof.

CD39

As indicated in Table 28 and 74, the expression level of the CD39 gene in human cells is an inheritable “quantitative trait” with genetic determinants. Furthermore, the SNPs in accordance with the present invention are associated with the “quantitative trait”, i.e., expression level of the CD39 gene in human cells. Specifically, the SNPs EX4@−10T and EX10@3061A are associated with the “low expression phenotype” while the SNPs EX4@−10C and EX10@3061G are associated with “high expression phenotype.” Thus, the SNPs are particularly useful in predicting the CD39 gene expression in an individual. Furthermore, other SNPs that are in linkage disequilibrium with the SNPs can also have similar predictive value.

Thus, in one aspect of the invention a method is provided for predicting or detecting susceptibility to vascular injury in an individual, which comprises the steps of genotyping the individual to determine the individual's genotype at one or more loci identified in the present invention wherein one or more of the SNPs associated with low expression phenotype of CD39 are detected in the individual, then it can be predicted that the individual has an increased risk of developing a metabolic or vascular disease. Thus, if one or more the SNPs EX4@−10T and EX10@3061A in CD39 are detected, or an LD SNP that is in linkage disequilibrium with any one of such SNPs is detected in the individual, then it can be reasonably predicted that the individual is at an increased risk of vascular injury. In particular, if an individual is homozygous with the genotype EX4@−10T/T or EX10@3061A/A, or homozygous with an LD SNP that is in linkage disequilibrium with any one or more of such SNPs, then it can be reasonably predicted that the individual has a increased susceptibility to vascular injury. In other words, such an individual has an increased likelihood or is at an increased risk of developing vascular disease or vascular injury. If an individual is heterozygous, then his or her risk of developing the disease is at an intermediate level. On the other hand, if the individual is homozygous with the genotype EX4@−10C/C and EX10@3061G/G, or a SNP that is in linkage disequilibrium with any one or more of such SNPs, then it can be reasonably predicted that the individual has a reduced susceptibility to vascular injury.

In another aspect, the present invention provides a method for determining the prognosis of an individual with vascular injury. The individual to be tested can be a healthy person or previously diagnosed individual. The method comprises the step of genotyping the individual to determine the individual's genotype at one or more of the loci identified in the present invention, or another locus at which the genotype is in linkage disequilibrium with one of the SNPs of the present invention. Thus, if one or more the SNPs EX4@−10T or EX10@3061A are detected, or an LD SNP that is in linkage disequilibrium with any one of such SNPs is detected in the individual, then it can be reasonably predicted that the individual has a high potential of disease progression and that the prognosis for the individual is poor, or recovery is difficult or slow. In other words, the individual has an increased likelihood or an increased risk of disease progression. Particularly, if an individual is homozygous with the genotype EX4@−10T/T or EX10@3061A/A, or a SNP that is in linkage disequilibrium with any one or more of such SNPs, then the individual has particular poor prognosis and that the disease will progress at an increased rate. In other words, the individual has a substantially increased likelihood or at a substantially increased risk of disease progression. However, if an individual is heterozygous with the genotype EX4@−10T/C or EX10@3061A/G, or is heterozygous with a SNP that is in linkage disequilibrium with any one or more of such SNPs, then the individual has a poor prognosis. Specifically, the individual has an intermediate level of disease progression.

In another aspect of the invention a method is provided for predicting or detecting susceptibility coronary disease in an individual, which comprises the steps of genotyping the individual to determine the individual's genotype at one or more loci identified in the present invention wherein one or more of the SNPs are detected in the individual, then it can be predicted that the individual has an increased risk of developing a metabolic or vascular disease. Thus, if one or more the SNPs EX4@−10C and EX10@3061G are detected, or a SNP that is in linkage disequilibrium with any one of such SNPs is detected in the individual, then it can be reasonably predicted that the individual is at a decreased risk of coronary disease. In particular, if an individual is homozygous with the genotype EX4@−10C/C or EX10@3061G/G, or a SNP that is in linkage disequilibrium with any one or more of such SNPs, then it can be reasonably predicted that the individual has a diminished risk of coronary disease. In other words, such an individual has an decreased likelihood or is at an decreased risk of developing coronary disease. If an individual is heterozygous, then his or her risk of developing the disease is at an intermediate level. On the other hand, if the individual is homozygous with the genotype EX4@−10T/T or EX10@3061A/A, or a SNP that is in linkage disequilibrium with any one or more of such SNPs, then it can be reasonably predicted that the individual has an elevated susceptibility to coronary disease.

In another aspect, the present invention provides a method for identifying patients with a high risk of transplant rejection. The individual to be tested can be a healthy individual or an individual in need of a transplant. The method comprises the step of genotyping the individual to determine the individual's genotype at one or more of the loci identified in the present invention, namely EX4@−10 and EX10@3061 in CD39, or another locus at which the genotype is in linkage disequilibrium with one of the SNPs of the present invention. Thus, if one or more the SNPs EX4@−10C and EX10@3061G are detected, or an LD SNP that is in linkage disequilibrium with any one of such SNPs is detected in the individual, then it can be reasonably predicted that the individual has low transplant rejection risk. Particularly, if an individual is homozygous with the genotype EX4@−10C/C or EX10@3061G/G, then the individual has particular low risk of transplant rejection. However, if an individual is heterozygous with the genotype EX4@−10T/C or EX10@3061A/G, then the individual has an intermediate risk of transplant rejection. Thus, if the individual is homozygous with the genotype EX4@−10T/T or EX10@3061A/A, or a homozygous LD SNP thereof then it can be reasonably predicted that the individual has a particularly increased risk of transplant rejection.

For example, in one embodiment, selection of a donor of transplant organ or tissue is aided by genotyping a donor candidate and determining the determine the individual's genotype at one or more of the loci identified in the present invention, namely EX4@−10 and EX10@3061 in CD39, or another locus at which the genotype is in linkage disequilibrium with one of the SNPs of the present invention. Specifically preferred donors should have a homozygous EX4169 −10C/C or EX10@3061G/G, or a homozygous LD SNP thereof. Individuals with a homozygous EX4@−10T/T or EX10@3061A/A, or a homozygous LD SNP thereof are preferably excluded as organ or tissue transplant donors.

In another aspect, the present invention provides a method for predicting and/or determining inflammatory response, particularly to irritants and immunogens. The method comprises the step of genotyping the individual to determine the individual's genotype at one or more of the loci identified in the present invention, namely EX4@−10 and EX10@3061 in CD39, or another locus at which the genotype is in linkage disequilibrium with one of the SNPs of the present invention. Thus, if the SNP EX4@−10T or EX10@3061A is detected, or an LD SNP that is in linkage disequilibrium with either of the SNPs is detected in the individual, then it can be reasonably predicted that the individual will have an increased inflammatory response, particularly in response to irritants and immunogens. Particularly, if an individual is homozygous with the genotype EX4@−10T/T or EX10@3061A/A, then the individual has particularly high inflammatory response. However, if an individual is heterozygous with the genotype EX4@−10T/C or EX10@3061A/G, then the individual has intermediate level of inflammatory response. Alternatively, if the individual is homozygous with the genotype EX4@−10C/C or EX10@3061G/G, then it can be reasonably predicted that the individual will have a decreased inflammatory response, especially in response to irritants and immunogens. Such prediction can be used in, e.g., determining the degree of harm of irritants and immunogens to a particular individual, and deciding on whether to administer an immunogen or vaccine to an individual and whether to include an individual to a clinical trial particularly a clinical trial involving an immunogen or vaccine.

In yet another aspect, the present invention encompasses a method for predicting the pharmacokinetic consequences of CD39 expression, e.g., the responsiveness of an individual to a drug, or the dose of a drug to be used in an individual, or potential toxicity of a drug on an individual, which can all correlate with the CD39 expression level.

In another aspect, the present invention relates a method of selecting individuals for inclusion in clinical trials. Such clinical trials can be on any drugs or medical or surgical procedures in which platelet aggregation, vascular injury, organ or tissue transplantation, or inflammatory response is a relevant factor for safety or efficacy concerns. Thus, the method generally comprises genotyping an individual to determine the genotype at one or more of the loci identified in the present invention, namely EX4@−10 and EX10@3061 in CD39, or another locus at which the genotype is in linkage disequilibrium with one of the CD39 SNPs of the present invention, and considering the genotype in making a decision as to whether or not to include the individual in a clinical trial.

The SNPs on Chromosome X at position 97,374,982 and position 97,536,166 have also been shown to be associated with CD39 mRNA levels. The Chromosome X SNPs associated with lower CD39 mRNA expression levels are 97,374,982G and 97,536,166G, whereas that associated with higher mRNA expression levels are 97,374,982A and 97,536,166C. In addition to those mentioned above, these SNPs may be utilized in the applications described above.

FKABP1a

As indicated in Table 30 and 75, the expression level of the FKBP1a gene in human cells is an inheritable “quantitative trait” with genetic determinants. Furthermore, the SNPs in accordance with the present invention are associated with the “quantitative trait”, i.e., FKBP1a mRNA levels in human cells. Specifically, the SNP EX5@8G is associated with a “low expression phenotype” while the SNP EX5@8A is associated with a “high expression phenotype.” Thus, the SNPs are particularly useful in predicting the level of FKBP1a gene expression in an individual. Furthermore, other SNPs that are in linkage disequilibrium with these SNPs can also have similar predictive value.

In one aspect, the present invention provides a method for determining the prognosis of an individual with nerve damage, which comprises the steps of genotyping and determining the FKBP1a genotype of the individual at EX5@8. The individual to be tested can be a healthy person or previously diagnosed individual. Thus, if the FKBP1a SNP EX5@8G is detected, or an LD SNP that is in linkage disequilibrium with such SNP is detected in the individual, then it can be reasonably predicted that the individual has low potential of recovery from nerve damage and/or decreased nerve regeneration and that the prognosis for the individual is poor. Particularly, if an individual is homozygous with the FKBP1a genotype EX5@8G/G, or a SNP that is in linkage disequilibrium with such SNP, then the individual has particularly poor prognosis and that the regeneration will progress at a decreased rate. If an individual is heterozygous with the FKBP1a genotype EX5@A/G, or is heterozygous with a SNP that is in linkage disequilibrium with SNP, then the individual has a moderately poor prognosis. Specifically, the individual has an intermediate rate of nerve regeneration. However, an individual homozygous with the FKBP1a genotype EX5@A/A will have improved recovery from nerve damage and increased rate of nerve regeneration following damage, and thus a better prognosis.

In another aspect of the invention, a method is provided for predicting or detecting response to treatment with macrolide immunosuppressant drugs such as rapamycin and FK506, which comprises the steps of genotyping the individual to determine the individual's FKBP1a genotype at the SNP identified in the present invention, wherein detection of the SNP in the individual is useful in predicting that the individual has an increased or decreased response to macrolide immunosuppressant drugs such as rapamycin and FK506 treatment, particularly in an individual diagnosed with cancer. Thus, if the FKBP1a SNP EX5@8A is detected, or a SNP that is in linkage disequilibrium with such SNP is detected in the individual, then it can be reasonably predicted that the individual will have an increased response to macrolide immunosuppressant drugs such as rapamycin and FK506 treatment. In particular, if an individual is homozygous with the FKBP1a genotype EX5@8A/A, or an LD SNP that is in linkage disequilibrium with this SNP, then it can be reasonably predicted that the macrolide immunosuppressant drug treatment will have an increased effectiveness in the individual. In other words, such an individual has an increased likelihood of successful treatment with macrolide immunosuppressant drugs such as rapamycin and FK506 treatment. If an individual is heterozygous, then his or her response to macrolide immunosuppressant drug treatment is predicted to be intermediate. On the other hand, if the individual is homozygous with the FKBP1a genotype EX5@8G/G, or a SNP that is in linkage disequilibrium with such SNP, then it can be reasonably predicted that treatment with macrolide immunosuppressant drugs such as rapamycin and FK506 will have decreased effectiveness and the individual will have a diminished response to treatment. The predicted response can be used as a criterion in deciding whether or how much to use a macrolide immunosuppressant drug on a particular individual, and in deciding whether to include an individual in a clinical trial in which FKBP1a gene expression is a factor affecting safety or efficacy, or in which a macrolide immunosuppressant drug is involved.

Thus, the present invention also provides a method of treating a patient with cancer comprising genotyping the patient in the FKBP1a gene to predict the patient's response to treatment with macrolide immunosuppressant drugs such as rapamycin and FK506, and deciding on whether to administer a macrolide immunosuppressant drug to the patient.

SRI

As indicated in Table 31 and 76, the expression level of the SRI gene in human cells is an inheritable “quantitative trait” with genetic determinants. Furthermore, the SNP in accordance with the present invention is associated with the “quantitative trait”, i.e., SRI mRNA levels in human cells. Specifically, the SNP EX9@351C is associated with a “low expression phenotype” while the SNP EX9@351T is associated with a “high expression phenotype.” Thus, the SNPs are particularly useful in predicting the level of SRI gene expression in an individual. Furthermore, other SNPs that are in linkage disequilibrium with the SNP can also have similar predictive value.

In another aspect, the present invention provides a method for identifying high-risk patients who have a poor prognosis of cancer, or for determining the prognosis of cancer, particularly in a cancer patient, e.g., with cancer such as acute myeloid leukemia (AML). The individual to be tested can be a healthy person or an individual diagnosed of cancer. The genotyping can be performed on a healthy tissue sample or a tumor sample to determine germline or somatic genotype. For example, the method can comprise the steps of genotyping the somatic tissues of an individual, and genotyping the cancer cells of that individual, to determine the genotype the SRI loci identified in the present invention, namely EX9@351, or another locus at which the genotype is in linkage disequilibrium with the SNP of the present invention. Thus, if the SRI SNP EX9@351T is detected, or a SNP that is in linkage disequilibrium with EX9@351T is detected in the individual, particularly within the cancer cells, then it can be reasonably predicted that the cancerous growth or tumor has high metastatic potential, that the patient has poor prognosis, and that the tumor cells of the cancer are likely invasive. In other words, the individual has an increased likelihood or is at an increased risk of cancer metastasis. Particularly, if an individual or their cancer is homozygous with the SRI genotype EX9@351T/T, then the individual has particularly poor prognosis and the cancer cells are likely highly invasive and give to rapid growth. However, if an individual, or their cancer, is heterozygous with the SRI genotype EX9@351T/C, then the individual has a moderately poor prognosis and the tumor cells are moderately invasive. Specifically, the individual has an intermediate level of risk of cancer metastasis, especially that associated with AML. That is, the clinical outcome is worse than a person having a homozygous SRI genotype of EX9@351C/C, but is better than a person having a homozygous SRI genotype of EX9@351T/T.

In another aspect, the present invention provides a method for predicting cancer remission rates in an individual diagnosed with cancer, especially acute myeloid leukemia (AML). The method comprises the steps of genotyping the healthy tissues of an individual, or the cancer cells of that individual, to determine the genotype the SRI loci identified in the present invention, namely EX9@351, or another locus at which the genotype is in linkage disequilibrium with the SNP of the present invention. Thus, if the SRI SNP EX9@351T is detected, or a SNP that is in linkage disequilibrium with EX9@351T is detected in the individual or their cancer cells, then it can be reasonably predicted that the individual has a low rate of remission. Particularly, if an individual, or their cancer, is homozygous with the SRI genotype EX9@351T/T, then the individual has particularly low rate of remission. However, if an individual, or their cancer, is heterozygous with the genotype EX9@351T/C, then the individual has an intermediate cancer remission rate, especially that associated with AML. That is, cancer remission rate is increased in a individual having a homozygous SRI genotype of EX9@351C/C in their somatic tissues or in their cancer, but is decreased in an individual having a homozygous SRI genotype of EX9@351T/T.

In yet another aspect, the present invention provides a method of predicting patient response to cancer treatment, especially to treatment with chemotherapeutics that inhibit DNA synthesis (such as doxorubicin and etoposide), inhibit protein synthesis (such as homoharringtonine) and antimicrotubule agents (such as vincristine). In accordance with the present invention, the SRI gene of a patient in need of chemotherapeutic treatment is genotyped in their healthy tissues or in a biopsy sample of their cancer cells, to determine the genotype at the EX9@351 locus of the present invention, specifically EX9@351, or another locus at which the genotype is in linkage disequilibrium with EX9@351 of the present invention. Expression levels of SRI can be utilized to predict the effectiveness of treatment in a patient. Further, the amount of resistance will be indicative successful recovery of a patient undergoing radiation therapy. If the SNP EX9@351T are detected, or a SNP that is in linkage disequilibrium with any one of such SNP is detected in an individual or their cancer, then it can be reasonably predicted that the cancer is more likely to be resistant cancer treatment using chemotherapeutics. In short, the individual will have a worse response, longer recovery time and poor prognosis. In the event that the individual has the SRI genotype EX9@351T/T, it can be reasonably predicted that the individual will have a decreased response to cancer treatment using chemotherapeutics. If the individual is heterozygous, it can be predicted that the individual will have an intermediate response to treatment. On the other hand, where an individual has the SRI genotype EX9@351C/C, then it can be reasonably predicted that the individual will have decreased resistance to cancer treatment involving chemotherapeutics.

In yet another aspect, the present invention encompasses a method for predicting or detecting an individual's ability to recover from heart disease, especially cardiomyopathy, which comprises the step of genotyping the individual to determine the individual's genotype the SRI loci identified in the present invention, namely EX9@351, or another locus at which the genotype is in linkage disequilibrium with the SNP of the present invention. Thus, if the SRI SNP EX9@351C is detected, or a SNP that is in linkage disequilibrium with the SNP is detected in the individual, then it can be reasonably predicted that the individual will have a decreased recovery rate for cardiovascular disease, especially for cardiomyopathy. In particular, if an individual is homozygous with the SRI genotype EX9@351C/C, then it can be reasonably predicted that the individual has a low ability to recover from cardiovascular disease. On the other hand, if the individual is homozygous with the SRI genotype EX9@351T/T, then it can be reasonably predicted that the individual has an increased ability to recover from cardiovascular disease.

The predicted response can be used as a criterion in deciding whether or how much to use a drug on a particular individual, and in deciding whether to include an individual in a clinical trial in which SRI gene expression is a factor affecting safety or efficacy.

XRRA1

As indicated in Table 32 and 77, the expression level of the XRRA1 gene in human cells is an inheritable “quantitative trait” with genetic determinants. Furthermore, the SNPs and/or haplotypes in accordance with the present invention are associated with the “quantitative trait”, i.e., XRRA1 mRNA level in human cells. Specifically, the SNPs EX2@26C, EX2@+40C, EX11@51C, EX13@62C and EX17@665G are associated with a “low expression phenotype” while the SNPs EX2@26G, EX2@+40T, EX11@51T, EX13@62G and EX17@665A are associated with a “high expression phenotype.” Thus, the SNPs are particularly useful in predicting the level of XRRA1 gene expression in an individual. Furthermore, other SNPs that are in linkage disequilibrium with the SNPs can also have similar predictive value.

In one aspect, the present invention encompasses a method for predicting or detecting sensitivity to ionizing radiation in an individual, which comprises the step of genotyping the individual to determine the individual's genotype at one or more of the XRRA1 loci identified in the present invention, namely EX2@26, EX2@+40, EX15@51, EX13@62 and EX17@665, or another locus at which the genotype is in linkage disequilibrium with one of the SNPs of the present invention. The genotyping can be performed on a healthy tissue sample or a disease tissue sample (e.g., tumor sample) to determine germline or somatic genotype. Thus, if one or more the XRRA1 SNPs EX2@26G, EX2@+40T, EX11@51T, EX13@62G and EX17@665A are detected, or a SNP that is in linkage disequilibrium with any one of such SNPs is detected in the individual, then it can be reasonably predicted that the individual has increased sensitivity to ionizing radiation. In particularly, if an individual is homozygous with the XRRA1 genotype EX2@26G/G, EX2@+40T/T, EX11@51T/T, EX13@62G/G and EX17@665A/A, then it can be reasonably predicted that the individual has an increased sensitivity to ionizing radiation. If an individual is heterozygous with the XRRA1 genotype EX2@26G/C, EX2@+40T/C, EX11@51T/C, EX13@62G/C and EX17@665A/G, then his or her sensitivity to radiation therapy is at an intermediate level. One the other hand, if the individual is homozygous with the XRRA1 genotype EX2@26C/C, EX2@+40C/C, EX11@51C/C, EX13@62C/C and EX17@665G/G, then it can be reasonably predicted that the individual has a decreased sensitivity to ionizing radiation.

The predicted XRRA1 expression level and sensitivity to ionizing radiation can be used as a criterion in deciding whether or how much to use ionizing radiation on a particular individual, and in deciding whether to include an individual in a clinical trial in which XRRA1 gene expression is a factor affecting safety or efficacy, or involving ionizing radiation.

Thus, a treatment method is provided comprising genotyping the patient in the XRRA1 gene to predict the patient's sensitivity to treatment with ionizing radiation, and deciding on whether to treat the patient with ionizing radiation or the dose thereof.

IRF5

As indicated in Tables 33, 78 and 79, the expression level of the IRF5 gene in human cells is an inheritable “quantitative trait” with genetic determinants. Furthermore, the SNPs in accordance with the present invention are associated with the “quantitative trait”, i.e., IRF5 mRNA levels in human cells. Specifically, the SNPs EX1@−709G, EX1@−396C, EX1@−82m, EX6@91m, EX9@801A and EX9@862A are associated with a “low expression phenotype” while the EX1@−709T, EX1@−396T, EX1@−82w, EX6@91w, EX9@801G and EX9@862G are associated with a “high expression phenotype.” Thus, the SNPs are particularly useful in predicting the level of IRF5 gene expression in an individual.

Thus, in one aspect, the present invention provides a method for predicting or determining immune response to viral infection in an individual. The method comprises the step of genotyping the individual to determine the individual's genotype at one or more of the IRF5 loci identified in the present invention, namely EX1@−709, EX1@−39C, EX1@−82, EX6@91, EX9@801 or EX9@862, or another locus at which the genotype is in linkage disequilibrium with one of the SNPs or haplotypes of the present invention. Thus, if one or more the SNPs EX1@−709G, EX1-396C, EX1@−82m, EX6@91m, EX9@801A or EX9@862A are detected, or a SNP that is in linkage disequilibrium with any one of such SNPs is detected in the individual, then it can be reasonably predicted that the individual will have a diminished immune response. In other words, the individual has an increased likelihood of developing viral infection. Particularly, if an individual is homozygous with the IRF5 genotype EX1@−709G/G, EX1@−396C/C, EX1@−82m/m, EX6@91m/m, EX9@801A/A or EX9@862A/A, then the individual has particularly poor immune response, especially to viral infection. However, if an individual is heterozygous with the IRF5 genotype EX1@−709G/T, EX1@−396C/T, EX1@−82m/w, EX6@91m/w, EX9@801A/G or EX9@862A/G, then the individual has intermediate immune response. Specifically, the individual has an intermediate level of risk of viral infection. Alternatively, if the individual is homozygous with the IRF5 genotype EX1@−709T/T, EX1@−396T/T, EX1@−82w/w, EX6@91w/w, EX9@801G/G or EX9@862G/G, then it can be reasonably predicted that the individual will have a good immune response. In other word, the individual will have a reduced susceptibility to infection, especially viral infection.

In another aspect, the present invention provides a method for identifying high-risk patients who have a poor prognosis of viral infection, or predicting/determining the invasiveness and viral progression in an individual. The individual to be tested can be a healthy person or an individual diagnosed with viral infection. The method comprises the step of genotyping the individual to determine the individual's genotype at one or more of the IRF5 loci identified in the present invention, namely EX1@−709, EX1@−39C, EX1@−82, EX6@91, EX9@801 or EX9@862, or another locus at which the genotype is in linkage disequilibrium with one of the SNPs of the present invention. Thus, if one or more the IRF5 SNPs EX1@−709G, EX1@−396C, EX1@−82m, EX6@91m, EX9@801A or EX9@862A are detected, or a SNP that is in linkage disequilibrium with any one of such SNPs is detected in the individual, then it can be reasonably predicted that the individual has a poor prognosis for viral infection, or a poor prognosis for viral invasiveness and progression. In other words, the individual has an increased likelihood or at an increased risk of viral infection. Particularly, if an individual is homozygous with the IRF5 genotype EX1@−709G/G, EX1-396C/C, EX1@−82m/m, EX6@91m/m, EX9@801A/A or EX9@862A/A, then the individual has particular poor prognosis. In other words, the individual has a substantially increased likelihood or at a substantially increased risk of viral progression after infection. However, if an individual is heterozygous with the IRF5 genotype EX1@−709G/T, EX1@−396C/T, EX1@−82m/w, EX6@91m/w, EX9@801A/G or EX9@862A/G, then the individual has an intermediate risk of viral progression. Thus, if the individual is homozygous with the IRF5 genotype EX1@−709T/T, EX1@−3 96T/T, EX1@−82w/w, EX6@91w/w, EX9@801 G/G or EX9@862G/G, then it can be reasonably predicted that individual will have a good prognosis. That is, the individual does not have an increased likelihood or increased risk of viral progression after infection.

In another aspect, the present invention encompasses a method for predicting or detecting susceptibility to autoimmune disease in an individual, which comprises the step of genotyping the individual to determine the individual's genotype at one or more of the IRF5 loci identified in the present invention, namely EX1@−709, EX1@−39C, EX1@−82, EX6@91, EX9@801 or EX9@862, or another locus at which the genotype is in linkage disequilibrium with one of the SNPs or haplotypes of the present invention. Thus, if one or more the IRF5 SNPs EX1@−709T, EX1@−396T, EX1@−82w, EX6@91w, EX9@801G and EX9@862G are detected, or a SNP that is in linkage disequilibrium with any one of such SNPs is detected in the individual, then it can be reasonably predicted that the individual is at an increased risk of developing an autoimmune disease, particularly Wegener's granulomatosis, multiple sclerosis, type 1 diabetes mellitus, lupus, and rheumatoid arthritis. In particularly, if an individual is homozygous with the IRF5 genotype EX1@−709T/T, EX1@−396T/T, EX1@−82w/w, EX6@91w/w, EX9@801G/G or EX9@862G/G, then it can be reasonably predicted that the individual has an elevated susceptibility to autoimmune disease, particularly Wegener's granulomatosis, multiple sclerosis, type 1 diabetes mellitus, lupus, and rheumatoid arthritis. Likewise, if the individual is homozygous with a IRF5 genotype at a locus that is in the same haplotype with the SNPs EX1@−709T/T, EX1@−396T/T, EX1@−82w/w, EX6@91w/w, EX9@801G/G or EX9@862G/G (in linkage disequilibrium), then it can reasonably be predicted that the individual has an elevated susceptibility to autoimmune disease, particularly Wegener's granulomatosis, multiple sclerosis, type 1 diabetes mellitus, lupus, and rheumatoid arthritis. In other words, such an individual has an increased likelihood or is at an increased risk of developing autoimmune disease, particularly Wegener's granulomatosis, multiple sclerosis, type 1 diabetes mellitus, lupus, and rheumatoid arthritis. If an individual is heterozygous, then his or her risk of developing autoimmune disease is at an intermediate level. One the other hand, if the individual is homozygous with the IRF5 genotype EX1@−709G/G, EX1@−396C/C, EX1@−82m/m, EX6@91m/m, EX9@801A/A or EX9@862A/A, then it can be reasonably predicted that the individual has a reduced susceptibility to autoimmune disease, particularly Wegener's granulomatosis, multiple sclerosis, type 1 diabetes mellitus, lupus, and rheumatoid arthritis. Similarly, if the individual is homozygous with a genotype at a locus that is in the same haplotype with the IRF5 SNPs EX1@−709G, EX1@−396C, EX1@−82m, EX6@91m, EX9@801A or EX9@862A (in linkage disequilibrium), then it can reasonably be predicted that the individual has a reduced susceptibility to autoimmune disease, particularly Wegener's granulomatosis, multiple sclerosis, type 1 diabetes mellitus, lupus, and rheumatoid arthritis.

The SNP at position 128,208,314 of chromosome 7, has also shows association with IRF5 mRNA expression levels. In addition to those mentioned above, these SNPs may be utilized in the applications described above.

AMFR

As indicated in Table 34, 35 and 80, the expression level of the AMFR gene in human cells is an inheritable “quantitative trait” with genetic determinants. Furthermore, the SNPs and/or haplotypes in accordance with the present invention are associated with the “quantitative trait”, i.e., AMFR mRNA levels in human cells. Specifically, the SNPs in Haplotype I (e.g., EX4@+14T, EX12@+62A, EX14@1359C) and the SNP EX14@483G are associated with a “low expression phenotype” while the SNPs in Haplotypes II (e.g., EX4@+14C, EX12@+62G, EX14@1359T) and the SNP EX14@483A are associated with a “high expression phenotype.” Thus, the SNPs and/or haplotypes are particularly useful in predicting the level of AMFR gene expression in an individual. Furthermore, other SNPs that are in linkage disequilibrium with the SNPs and/or haploytes can also have similar predictive value.

Thus, in one aspect, the present invention encompasses a method for predicting or detecting cancer susceptibility in an individual, which comprises the step of genotyping the individual to determine the individual's genotype at one or more of the AMFR loci identified in the present invention, namely EX4@+14, EX12@+62, EX14@1359 and EX14@483, or another locus at which the genotype is in linkage disequilibrium with one of the SNPs or haplotypes of the present invention. Thus, if one or more the AMFR SNPs EX4@+14C, EX12@+62G, EX14@1359T and SNP EX14@483A are detected, or a SNP that is in linkage disequilibrium with any one of such SNPs is detected in the individual, then it can be reasonably predicted that the individual is at an increased risk of developing cancer, particularly skin cancer, lung cancer, ovarian cancer or thyoma. In particularly, if an individual is homozygous with the AMFR genotype EX4@+14C/C, EX12@+62G/G, EX14@1359T/T or EX14@483A/A, then it can be reasonably predicted that the individual has an elevated susceptibility to cancer, particularly skin cancer (e.g., melanoma), lung cancer (e.g., NSCLCs), ovarian cancer or thyoma. Likewise, if the individual is homozygous with an AMFR genotype at a locus that is in the same haplotype with the SNPs EX4@+14C, EX12@+62G and EX14@1359T (in linkage disequilibrium), or in linkage disequilibrium with the SNP EX14@483A, then it can reasonably be predicted that the individual has an elevated susceptibility to cancer, particularly skin cancer (e.g., melanoma), lung cancer (e.g., NSCLCs) or thyoma. In other words, such an individual has an increased likelihood or is at an increased risk of developing cancer, particularly skin cancer (e.g., melanoma), lung cancer (e.g., NSCLCs) or thyoma. If an individual is heterozygous, then his or her risk of developing cancer is at an intermediate level. One the other hand, if the individual is homozygous with the AMFR genotype EX4@+14T/T or EX12@+62A/A or EX14@1359C/C or EX14@483G/G, then it can be reasonably predicted that the individual has a reduced susceptibility to cancer, particularly skin cancer, lung cancer, ovarian cancer or thyoma. Similarly, if the individual is homozygous with a genotype at an AMFR locus that is in the same haplotype with the SNPs EX4@+14T, EX12@+62A and EX14@1359C (in linkage disequilibrium), or in the same haplotype (linkage disequilibrium) with the SNP EX14@483G, then it can reasonably be predicted that the individual has a reduced susceptibility to cancer, particularly skin cancer, lung cancer or thyoma.

In another aspect, the present invention provides a method for identifying high-risk patients who have a poor prognosis of cancer, or for the prognosis of cancer, or predicting/determining the invasiveness and metastatic potential of tumor in a patient, particularly cancer patient, e.g., with cancer such as melanoma, lung cancer, ovarian cancer, non-small cell lung cancers (NSCLCs), and thyoma. The individual to be tested can be a healthy person or an individual diagnosed of cancer. The method comprises the step of genotyping the individual to determine the individual's genotype at one or more of the AMFR loci identified in the present invention, namely EX4@+14, EX12@+62, EX14@1359 and EX14@483, or another locus at which the genotype is in linkage disequilibrium with one of the SNPs or haplotypes of the present invention. Thus, if one or more the AMFR SNPs EX4@+14C, EX12@+62G, EX14@1359T and SNP EX14@483A are detected, or a SNP that is in linkage disequilibrium with any one of such SNPs is detected in the individual, then it can be reasonably predicted that the individual has high metastasis potential, that the cancer has poor prognosis and that the tumor cells are invasive. In other words, the individual has an increased likelihood or at an increased risk of cancer metastasis. Particularly, if an individual is homozygous with the AMFR genotype EX4@+14C/C, EX12@+62G/G, EX14@1359T/T or SNP EX14@483A/A, then the individual has particular poor prognosis and that the tumor cells are highly invasive. In other words, the individual has a substantially increased likelihood or at a substantially increased risk of cancer metastasis. However, if an individual is heterozygous with the AMFR genotype EX4@+14T/C, EX12@+62A/G, EX14@1359C/T or SNP EX14@483G/A, then the individual has poor prognosis and that the tumor cells are invasive. Specifically, the individual has an intermediate level of risk of cancer metastasis. That is, the risk is greater than a person having a homozygous AMFR genotype of EX4@+14T/T or EX12@+62A/A or EX14@1359C/C or EX14@483G/G, but is lower than a person having a homozygous genotype of EX4@+14C/C, EX12@+62G/G, EX14@1359T/T or SNP EX14@483A/A.

Thus, if the individual is homozygous with the AMFR genotype EX4@+14T/T or EX12@+62A/A or EX14@1359C/C or EX14@483G/G, then it can be reasonably predicted that the tumor in the individual has low metastasis potential, that the cancer has good prognosis and that the tumor cells are not invasive. That is, the individual does not have an increased likelihood or increased risk of cancer metastasis. Similarly, if the individual is homozygous with a genotype at a locus that is in the same haplotype with the SNPs EX4@+14T, EX12@+62A and EX14@1359C (in linkage disequilibrium), or in the same haplotype (linkage disequilibrium) with the SNP EX14@483G, then it can reasonably be predicted that the individual has a low metastasis potential, that the cancer has good prognosis and that the tumor cells are not invasive. In other words, the individual does not have an increased likelihood or increased risk of cancer metastasis.

In another aspect, the present invention provides a method for identifying high-risk patients who have cancerous growths or tumors, and who have a poor prognosis of recovery, or for predicting/determining the invasiveness and metastatic potential of the cancerous growth or tumor within a patient, particularly cancer patient, e.g., with melanoma, lung cancer, ovarian cancer, non-small cell lung cancers (NSCLCs), or thyoma. In this aspect of the invention, the methods comprise the step of genotyping the cancerous growth or tumor within the individual to determine the cancerous growth or tumor's genotype at one or more of the AMFR loci identified in the present invention, namely those listed above, or another locus at which the genotype is in linkage disequilibrium with one of the SNPs of the present invention.

Particularly, if the tumor or cancerous growth turns out to be homozygous with the AMFR genotype EX4@+14C/C, EX12@+62G/G, EX14@1359T/T or SNP EX14@483A/A, then the tumor or cancerous growth is likely highly invasive, and the patient has a particularly poor prognosis. In other words, the patient has a substantially increased likelihood or at a substantially increased risk of cancer metastasis because the tumor has a genotype expected to overexpress AMFR. However, the tumor or cancerous growth turns out to be heterozygous with the AMFR genotype EX4@+14T/C, EX12@+62A/G, EX14@1359C/T or SNP EX14@483G/A, then the tumor or cancerous growth is likely moderately invasive, and the patient has an intermediate prognosis. Specifically, the patient has an intermediate level of risk of cancer metastasis. That is, the risk is greater than a patient having a tumor or cancerous growth that has a homozygous AMFR genotype of EX4@+14T/T or EX12@+62A/A or EX14@1359C/C or EX14@483G/G, but is lower than a patient having a tumor or cancerous growth that homozygous genotype of EX4@+14C/C, EX12@+62G/G, EX14@1359T/T or SNP EX14@483A/A.

Thus, if the tumor or cancerous growth is homozygous with the AMFR genotype EX4@+14T/T or EX12@+62A/A or EX14@1359C/C or EX14@483G/G, then it can be reasonably predicted that the tumor or cancerous growth within the patient has a low metastatic potential, that the patient has a good prognosis, and that their tumor's cells are not likely to be highly invasive. That is, the patient does not have an increased likelihood or increased risk of cancer metastasis. Similarly, if the tumor or cancerous growth is homozygous with a genotype at an AMFR locus that is in the same haplotype with the SNPs EX4@+14T, EX12@+62A and EX14@1359C (in linkage disequilibrium), or in linkage disequilibrium with the SNP EX14@483G, then it can reasonably be predicted that the tumor or cancerous growth within the patient has a low metastatic potential, that the patient has a good prognosis, and that their tumor's cells are not likely to be highly invasive. In other words, the patient does not have an increased likelihood or increased risk of cancer metastasis.

In yet another aspect of the present invention, a method is provided for predicting drug response in a patient to treatment with inhibitors of VEGFRs. There are many inhibitors of VEGFR known in the art. For example, bevacizumab (Avastin® from Genentech, Inc.) is a recombinant humanized anti-VEGF antibody that inhibits the VEGFR functions. Avastino has approved in the US by the FDA for treating colon cancer. Other inhibitors of VEGFR include tyrosine kinase inhibitors such as SU5416, SU11248 and PTK787. Thus, in accordance with the present invention, the AMFR gene of a patient in need of treatment with a VEGFR inhibitor is genotyped to determine the genotype at one or more of the AMFR loci identified in the present invention, namely EX4@+14, EX12@+62, EX14@1359 and EX14@483, or another locus at which a genotype is in linkage disequilibrium with one of the SNPs or haplotypes of the present invention. Thus, if one or more the AMFR SNPs EX4@+14C, EX12@+62G, EX14@1359T and SNP EX14@483A are detected, or a SNP that is in linkage disequilibrium with any one of such SNPs is detected in the individual, then it can be reasonably predicted that the individual is likely to respond to treatment with an inhibitor of VEGFR. In other words, once an inhibitor of VEGFR is administered, there is an increased likelihood that the inhibitor will cause positive effect in the individual, including, e.g., shrinkage or elimination of tumor, increased death of tumor cells, etc.

Particularly, if an individual is homozygous with the AMFR genotype EX4@+14C/C, EX12@+62G/G, EX14@1359T/T or SNP EX14@483A/A, then the individual has a substantially increased likelihood of being responsive to treatment with a VEGFR inhibitor. If an individual is heterozygous with the AMFR genotype EX4@+14T/C, EX12@+62A/G, EX14@1359C/T or SNP EX14@483G/A, then the individual is still likely to respond to a VEGFR inhibitor. Specifically, the individual has an intermediate level of responsiveness to VEGFR inhibitors. That is, the degree of responsiveness is likely to be greater than that in a person having a homozygous AMFR genotype of EX4@+14T/T or EX12@+62A/A or EX14@1359C/C or EX14@483G/G, but is lower than a person having a homozygous genotype of EX4@+14C/C, EX12@+62G/G, EX14@1359T/T or SNP EX14@483A/A.

Thus, if the individual is homozygous with the AMFR genotype EX4@+14T/T or EX12@+62A/A or EX14@1359C/C or EX14@483G/G, then it can be reasonably predicted that there is an increased likelihood that the individual exhibits a low responsiveness to treatment with a VEGFR inhibitor. Similarly, if an individual is homozygous with a genotype at a locus that is in the same haplotype with the SNPs EX4@+14T, EX12@+62A and EX14@1359C (in linkage disequilibrium), or in the same haplotype (linkage disequilibrium) with the SNP EX14@483G, then it can reasonably be predicted that there is an increased likelihood that the individual has a low responsiveness to treatment with a VEGFR.

In specific embodiments, the individual in need of VEGFR inhibitor treatment is diagnosed as having cancer, e.g., colorectal cancer or ovarian cancer. Also, in certain embodiments, the VEGFR inhibitor is an antibody specifically immunoreactive with VEGF or VEGFR. In one example, such an antibody is bevacizumab (e.g., Avastin® from Genentech, Inc.).

Once the prognosis of a patient's response to VEGFR inhibitors is made, suitable treatment regimens (e.g., dosage and frequency of administration, and the like) can be decided based on the predicted responsiveness of the patient. For example, if the AMFR genotyping result suggests a low responsiveness by the patient to VEGFR inhibitors, then a higher dosage of VEGFR inhibitors would be desirably to the patient, or it may be simply decided that another class of drugs would be more suitable for the patient. Thus, in another aspect of the invention, a method is provided for determining a dosage of a VEGFR inhibitor to be administered to a patient, comprising determining the individual's genotype in the AMFR gene at one or more of the loci identified in the present invention, namely EX4@+14, EX12@+62, EX14@1359 and EX14@483, or another locus at which the genotype is in linkage disequilibrium with one of the SNPs or haplotypes of the present invention to determine the likely responsiveness of the patient, and determining accordingly the dosage of a VEGFR inhibitor to be administered to the patient, wherein the presence of one or more of the AMFR SNPs EX4@+14C, EX12@+62G, EX14@1359T and EX14@483A, or a SNP that is in linkage disequilibrium with any one of such SNPs would indicate that the patient is likely to respond to said VEGFR inhibitor at a lower dosage than another patient without the nucleotide variants. In one embodiment, the method is used in treating colon cancer. In other embodiments, the method is used in treating breast cancer, melanoma, ovarian cancer, brain cancer, neuroblastoma, uterine cancer, leukemia, lymphoma, head and neck cancer, thyroid cancer, gastrointestinal cancer, pancreatic cancer, liver cancer, etc. In preferred embodiments, the VEGFR inhibitor is an antibody specific to VEGF or VEGFR.

In another aspect of the invention, a method is provided for selecting an anti-cancer treatment for a patient, which comprises determining, in an AMFR gene in a sample isolated from the patient, the presence or absence of a nucleotide variant that is selected from the group consisting of EX4@+14C, EX12@+62G, EX14@1359T and EX14@483A, or a SNP that is in linkage disequilibrium with any one of such SNPs, wherein the presence of said nucleotide variant would indicate that the patient is likely to respond to a VEGFR inhibitor. Thus, if the AMFR genes in a patient contain one or more of the nucleotide variants of the present invention, then physicians or other decision makers may decide based on the genotyping result that it would be desirable to treat the patient with VEGFR inhibitors, particularly antibodies specifically immunoreactive with VEGF or VEGFR, e.g., bevacizumab (e.g., Avastin® from Genentech, Inc.). In one embodiment, the selection of treatment with a VEGFR inhibitor is based on the presence of a homozygous genotype of one or more of the above SNPs. In one embodiment, the method is used in selecting a treatment of colon cancer. In other embodiments, the method is used in selecting a treatment of NSCLCs, breast cancer, melanoma, ovarian cancer, thyoma, brain cancer, neuroblastoma, uterine cancer, leukemia, lymphoma, head and neck cancer, thyroid cancer, gastrointestinal cancer, pancreatic cancer, liver cancer, etc.

In yet another aspect of the present invention, a method is provided for selecting candidate human subjects for participation in a clinical trial involving a VEGFR inhibitor, which comprises (1) determining, in the AMFR gene of an individual, the presence or absence of a nucleotide variant that is selected from the group consisting of EX4@+14C, EX12@+62G, EX14@1359T and EX14@483A, or a SNP that is in linkage disequilibrium with any one of such SNPs, wherein the presence of said nucleotide variant would indicate that the patient is likely to respond to a VEGFR inhibitor; and (2) deciding whether to include said individual in the clinical trial. For example, if the patient has one or more of the nucleotide variants, then clinical trial for a VEGFR inhibitor may include that patient, particularly when the patient is homozygous in one or more of the SNPs. In one embodiment, the method is used in a clinical trial for testing a VEGFR inhibitor in colon cancer. In other embodiments, the method is used in selecting patients for inclusion in clinical trials for testing a VEGFR inhibitor in breast cancer, melanoma, ovarian cancer, brain cancer, neuroblastoma, uterine cancer, leukemia, lymphoma, head and neck cancer, thyroid cancer, gastrointestinal cancer, pancreatic cancer, liver cancer, etc. In preferred embodiments, the VEGFR inhibitor is an antibody specifically immunoreactive with VEGF or VEGFR.

### 8. Screening Assays

The present invention further provides a method for identifying compounds for treating or preventing symptoms amendable to treatment by alteration of TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein activities. For this purpose, variant TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein or fragment thereof containing a particular amino acid variant in accordance with the present invention can be used in any of a variety of drug screening techniques. Drug screening can be performed as described herein or using well known techniques, such as those described in U.S. Pat. Nos. 5,800,998 and 5,891,628, both of which are incorporated herein by reference. The candidate therapeutic compounds may include, but are not limited to proteins, small peptides, nucleic acids, and analogs thereof. Preferably, the compounds are small organic molecules having a molecular weight of no greater than 10,000 dalton, more preferably less than 5,000 dalton.

In one embodiment of the present invention, the method is primarily based on binding affinities to screen for compounds capable of interacting with or binding to a TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein containing one or more amino acid variants. Compounds to be screened may be peptides or derivatives or mimetics thereof, or non-peptide small molecules. Conveniently, commercially available combinatorial libraries of compounds or phage display libraries displaying random peptides are used.

Various screening techniques known in the art may be used in the present invention. The TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein variants (drug target) can be prepared by any suitable methods, e.g., by recombinant expression and purification. The polypeptide or fragment thereof may be free in solution but preferably is immobilized on a solid support, e.g., in a protein microchip, or on a cell surface. Various techniques for immobilizing proteins on a solid support are known in the art. For example, PCT Publication WO 84/03564 discloses synthesizing a large numbers of small peptide test compounds on a solid substrate, such as plastic pins or other surfaces. Alternatively, purified mutant TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein or fragment thereof can be coated directly onto plates such as multi-well plates. Non-neutralizing antibodies, i.e., antibodies capable binding to the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein or fragment thereof but do not substantially affect its biological activities may also be used for immobilizing the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein or fragment thereof on a solid support.

To effect the screening, test compounds can be contacted with the immobilized TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein or fragment thereof to allow binding to occur to form complexes under standard binding assays. Either the drug target or test compounds are labeled with a detectable marker using well known labeling techniques. To identify binding compounds, one may measure the formation of the drug target-test compound complexes or kinetics for the formation thereof.

Alternatively, a known ligand capable of binding to the drug target can be used in competitive binding assays. Complexes between the known ligand and the drug target can be formed and then contacted with test compounds. The ability of a test compound to interfere with the interaction between the drug target and the known ligand is measured using known techniques. One exemplary ligand is an antibody capable of specifically binding the drug target. Particularly, such an antibody is especially useful for identifying peptides that share one or more antigenic determinants of the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein or fragment thereof.

In another embodiment, a yeast two-hybrid system may be employed to screen for proteins or small peptides capable of interacting with a TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein variant. For example, a battery of fusion proteins each contains a random small peptide fused to e.g., Gal 4 activation domain, can be co-expressed in yeast cells with a fusion protein having the Gal 4 binding domain fused to a TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein variant. In this manner, small peptides capable of interacting with the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein variant can be identified. Alternatively, compounds can also be tested in a yeast two-hybrid system to determine their ability to inhibit the interaction between the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein variant and a known protein capable of interacting with the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein or polypeptide or fragment thereof. Again, one example of such proteins is an antibody specifically against the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein variant. Yeast two-hybrid systems and use thereof are generally known in the art and are disclosed in, e.g., Bartel et al., in: Cellular Interactions in Development: A Practical Approach, Oxford University Press, pp. 153-179 (1993); Fields and Song, Nature, 340:245-246 (1989); Chevray and Nathans, Proc. Natl. Acad. Sci. USA, 89:5789-5793 (1992); Lee et al., Science, 268:836-844 (1995); and U.S. Pat. Nos. 6,057,101, 6,051,381, and 5,525,490, all of which are incorporated herein by reference.

The compounds thus identified can be further tested for activities, e.g., in stimulating the niological activities of the variant TLK1, WARS2, ARTS2, MSR, AKAP9, DNAJD1, GOLPH4, RABEP1, TAP2, NARG2, DDX58, CD39, FKBP1a, SRI or XRRA1, e.g., in DNA mismatch repair.

Once an effective compound is identified, structural analogs or mimetics thereof can be produced based on rational drug design with the aim of improving drug efficacy and stability, and reducing side effects. Methods known in the art for rational drug design can be used in the present invention. See, e.g., Hodgson et al., Bio/Technology, 9:19-21 (1991); U.S. Pat. Nos. 5,800,998 and 5,891,628, all of which are incorporated herein by reference. An example of rational drug design is the development of HIV protease inhibitors. See Erickson et al., Science, 249:527-533 (1990). Preferably, rational drug design is based on one or more compounds selectively binding to a variant TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein or a fragment thereof.

In one embodiment, the three-dimensional structure of, e.g., a TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein variant, is determined by biophysics techniques such as X-ray crystallography, computer modeling, or both. Desirably, the structure of the complex between an effective compound and the variant TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein is determined, and the structural relationship between the compound and the protein is elucidated. In this manner, the moieties and the three-dimensional structure of the selected compound, i.e., lead compound, critical to the its binding to the variant TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein are revealed. Medicinal chemists can then design analog compounds having similar moieties and structures. In addition, the three-dimensional structure of wild-type TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein is also desirably deciphered and compared to that of a variant TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein. This will aid in designing compounds selectively interacting with the variant TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein.

In another approach, a selected peptide compound capable of binding the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein variant can be analyzed by an alanine scan. See Wells, et al., Methods Enzymol., 202:301-306 (1991). In this technique, an amino acid residue of the peptide is replaced by Alanine, and its effect on the peptide's binding affinity to the variant TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein is tested. Amino acid residues of the selected peptide are analyzed in this manner to determine the domains or residues of the peptide important to its binding to variant TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR protein. These residues or domains constituting the active region of the compound are known as its “pharmacophore.” This information can be very helpful in rationally designing improved compounds.

Once the pharmacophore has been elucidated, a structural model can be established by a modeling process which may include analyzing the physical properties of the pharmacophore such as stereochemistry, charge, bonding, and size using data from a range of sources, e.g., NMR analysis, x-ray diffraction data, alanine scanning, and spectroscopic techniques and the like. Various techniques including computational analysis, similarity mapping and the like can all be used in this modeling process. See e.g., Perry et al., in OSAR: Quantitative Structure-Activity Relationships in Drug Design, pp. 189-193, Alan R. Liss, Inc., 1989; Rotivinen et al., Acta Pharmaceutical Fennica, 97:159-166 (1988); Lewis et al., Proc. R. Soc. Lond., 236:125-140 (1989); McKinaly et al., Annu. Rev. Pharmacol. Toxiciol., 29:111-122 (1989). Commercial molecular modeling systems available from Polygen Corporation, Waltham, Mass., include the CHARMm program, which performs the energy minimization and molecular dynamics functions, and QUANTA program which performs the construction, graphic modeling and analysis of molecular structure. Such programs allow interactive construction, visualization and modification of molecules. Other computer modeling programs are also available from BioDesign, Inc. (Pasadena, Calif.), Hypercube, Inc. (Cambridge, Ontario), and Allelix, Inc. (Mississauga, Ontario, Canada).

A template can be formed based on the established model. Various compounds can then be designed by linking various chemical groups or moieties to the template. Various moieties of the template can also be replaced. In addition, in case of a peptide lead compound, the peptide or mimetics thereof can be cyclized, e.g., by linking the N-terminus and C-terminus together, to increase its stability. These rationally designed compounds are further tested. In this manner, pharmacologically acceptable and stable compounds with improved efficacy and reduced side effect can be developed.

### EXAMPLE 1

### Generation of Expression and SNP Genotyping Data

Human lymphoblastoid cell lines were purchased from Coriell (Camden, N.J.). Cell lines were grown in RPMI1640 media containing 15% heat inactivated FBS, 2 mM L-glutamine and 1× antibiotic antimycotic to a density of 5×10⁵. Then fresh media was added and the cells were harvested 24 hours later. Total number of cells harvested for RNA isolation is approximately 10×10⁶, and for DNA is approximately 5×10⁶. RNA was prepared using the Ribopure kit provided by Ambion, Inc. DNA was isolated using a DNeasy Tissue kit from Qiagen.

RNA was converted to labeled cRNA using the protocol recommended by Affymetrix, Inc. High density Hu133A expression chips from Affymetrix were hybridized to the cRNA, washed, stained and scanned using the recommended protocols.

DNA template for the Centurion SNP chip was generated using the protocol recommended by Affymetrix Inc. Centurion SNP chips from Affymetrix were hybridized to the DNA template, washed, stained and scanned using the recommended protocols.

### EXAMPLE 2

### Linkage and Association Analysis of Expression and SNP Genotyping Data

mRNA expression data was extracted using RMA (robust multi-array average) as the summary measure for Affymetrix oligonucleotide array data. See Irizarry et al., Biostatistics, 4(2):249-264 (2003). RMA values were normalized by subtracting means for each sex. Association analysis between Affy SNP genotypes and mRNA levels was done by a standard statistical test. A subset of 10,000 SNPs from Affy 120K SNP chip was used for linkage analysis. See Thomas et al., Statistics and Computing, 10:259-269 (2000). MCLINK program was used to define inheritance at the subset of 10,000 SNPs. A blocked Gibbs sampler was used to fit finite normal mixtures to sex-corrected RMA data. These normal mixtures were used to create a linkage model where mRNA levels were treated as QTL phenotypes. See Ishwaran and James, J. Comp. Graph. Statist., 11(3):1-26 ((2002). Robust multipoint Lod scores for each mRNA level were calculated at all 10,000 SNP locations. See Abkevich et al., Genetic Epidemiology, 21 (Suppl 1):S492-497 (2001). Heritability estimation is done by MERLIN software. See Abecasis et al., Nat. Genet., 30:97-101 (2002).

The TLK1 mRNA expression level was identified to be inheritable at a LOD score of greater than 5.6. SNP probes in the TLK1 gene region were also associated with the mRNA expression levels at a p value of 1.5×10⁻¹³ or less. SNP probes in the WARS2 gene region were also associated with the mRNA expression levels at a p value of 2.5×10⁻⁸ or less. The ARTS1 mRNA expression level was identified to be inheritable at a LOD score of greater than 9.7. The MSR mRNA expression level was identified to be inheritable at a LOD score of greater than 4.3. SNP probes in the MSR gene region were also associated with the mRNA expression levels at a p value of 0.00634 or less. SNP probes in the AKAP9 gene region were also associated with the mRNA expression levels at a p value of 1.35e-08 or less. SNP probes in the DNAJD1 gene region were associated with the mRNA expression levels at a p value of 3.76×10⁻⁷ or less. The GOLPH4 mRNA expression level was identified to be inheritable at a LOD score of greater than 7.9. The RABEP1 mRNA expression level was identified to be inheritable at a LOD score of greater than 9.2. SNP probes in the RABEP1 gene region were also associated with the mRNA expression levels at a p value of 6.6×10⁻⁹ or less. The TAP2 mRNA expression level was identified to be inheritable at a LOD score of greater than 4.2. SNP probes in the NARG2 gene region were also associated with the mRNA expression levels at a p value of 3.6×10⁻⁷ or less. The TAP2 mRNA expression level was identified to be inheritable at a LOD score of greater than 4.2. SNP probes in the NARG2 gene region were also associated with the mRNA expression levels at a p value of 3.6×10⁻⁷ or less. The DDX58 mRNA expression level was identified to be inheritable at a LOD score of greater than 5.8. The CD39 mRNA expression level was identified to be inheritable at a LOD score of greater than 1.39. SNP probes in the CD39 gene region were also associated with the mRNA expression levels at a p value of 2.4×10⁻⁵ or less. The FKBP1a mRNA expression level was identified to be inheritable at a LOD score of greater than 5.3. SNP probes in the FKBP1a gene region were also associated with the mRNA expression levels at a p value of 31.9×10⁻⁵ or less. The SRI mRNA expression level was identified to be inheritable at a LOD score of greater than 2.86. SNP probes in the SRI gene region were also associated with the mRNA expression levels at a p value of 5.7×10⁻⁸ or less. The XRRA1 mRNA expression level was identified to be inheritable at a LOD score of greater than 3.8. SNP probes in the XRRA1 gene region were also associated with the mRNA expression levels at a p value of 4.0×10⁻⁶ or less.

### EXAMPLE 3

### Variant Discovery

To identify sequence variants that serve as diagnostics for predicting RNA levels, variant discovery was carried out on all exons of the TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene and 1 kb of upstream regulatory sequence. The 30 parent individuals of the 15 families used in RNA profiling represent the genomic variability of the entire sample set and were therefore selected for variant detection.

For each exon and 1000 bases upstream of exon 1 two pairs of nested primers were designed using proprietary software. The nested primer pair was tailed with universal M13 primers. Primers were positioned to include a minimum of 30 bases of intronic sequence at either end of an exon in the final PCR product. This allows for examination of exon/intron boundaries. Large exons and continuous promoter sequence was amplified with overlapping primers sets. All amplicons were amplified using a robotic system and standard PCR conditions. PCR products were treated with shrimp alkaline phosphatase to remove free nucleotides and submitted to dye-primer sequencing using forward and reverse M13 sequencing primers. Products were separated on capillary sequencing machines (MegaBACE) and base-called using proprietary software. Detection of variants was performed by computer software that compares individual base-called sequence traces to a reference sequence.

Tables 36-80 show the association of the different SNPs and haplotypes with TLK1, WARS2, ARTS2, MSR, AKAP9, DNAJD1, GOLPH4, RABEP1, TAP2, NARG2, DDX58, CD39, FKBP1a, SRI, XRRA1, IRF5 or AMFR gene expression levels. For T-statistic calculation, a standard two-sample t-test assuming equal variances was performed to compare the mean expression values for individuals who carry a certain genotype and those who do not. The table gives the t-statistic and the p-value for a one-sided hypothesis test.

### EXAMPLE 4

### Discovery of Additional SNPs in Linkage Disequilibrium with the SNPs Associated with mRNA Levels

After the initial identification of the association of an mRNA level-associated SNP, a search for additional SNPs (or nucleotide variants) in linkage disequilibrium (LD) with the mRNA level-associated SNP (or nucleotide variant) is undertaken. For this purpose, the available LD data for the relevant chromosome of the CEU population of HapMap phase II can be downloaded and queried.

Parameters for the query are set to reveal other SNPs in LD with the mRNA level-associated SNP (or nucleotide variant) with r² values ≧0.8. The query identifies additional LD SNPs.

HapMap SNPs with r² values ≧0.8 are culled from the query if their distance from the seed SNP is too great, i.e., >100 kbp, or if the LD appears to have arisen by chance.

### EXAMPLE 5

### Identification of Alleles in Linkage Disequilibrium with the mRNA Level-Associated Allele

To determine which alleles at LD SNPs are in LD with the mRNA level-associated nucleotide variant, genotype calls for mRNA level-associated SNP and the identified LD SNPs from 90 individuals within the CEU population can be downloaded from HapMap and used to construct haplotypes. Frequencies of each haplotype are calculated. LD nucleotide variants are therefore identified based on such frequencies.

LD SNPs can be extrapolated using the “Single Nucleotide Polymorphism dbSNP search” tool, which is available from the National Center for Biotechnology Information, U.S. National Library of Medicine (Bethesda, Md., U.S.A.). as of the priority date or filing date. This can be done in accordance with the methods describes in this Example and in Example 4. Representative LD SNPs are shown in Table 81.

It is noted that the nucleotide sequences surrounding each of the SNPs are provided in Sequence Listing and as indicated in Tables 1-35. While there may be alternatively spliced variants of gene transcripts and the chromosome locations may change over time, the exon and intron numbering and the SNP positions of the present invention would be clearly understood by a skilled artisan by reference to the sequences in the sequence listing together with GenBank Accession Numbers or a variant or modification of this GenBank sequence. However, it is noted that the SNPs or nucleotide variants of the present invention are by no means limited to be only in the context of the sequences in the sequence listings or the particular GenBank entry referred to herein. Rather, it is recognized that GenBank sequences may contain unrecognized sequence errors only to be corrected at a later date, and additional gene variants may be discovered in the future. The present invention encompasses SNPs or nucleotide variants as referred to in Tables 1-35 irrespective of such sequence contexts. Indeed, even if the GenBank entries or chromosome locations referred to herein are changed based on either error corrections or additional variants discovered, skilled artisans apprised of the present disclosure would still be able to determine or analyze the SNPs or haplotypes of the present invention in the new sequence contexts.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. An isolated nucleic acid or the complement thereof, said isolated nucleic acid comprising a contiguous span of at least 18 nucleotide residues of:
(a) an ARTS nucleic acid, at least one of said residues being a nucleotide variant of EX1@−1125 or EX12@44;
(b) a DNAJD1 nucleic acid, at least one of said residues being a nucleotide variant of EX5@+72; and
(c) a RABEP1 nucleic acid, at least one of said residues being a nucleotide variant of EX1@−551, EX18@646 or EX18@690.

2. A DNA microchip comprising one or more said isolated nucleic acid according to claim 1.

3. A method of genotyping a human individual, comprising detecting, or determining the presence or absence of, a nucleotide variant of claim 1 in said individual.

4. A method of predicting the expression of a gene in a human subject, comprising determining the presence or absence of a nucleotide variant associated with high gene expression phenotype, wherein:
(a) said gene is TLK1 and said nucleotide variant is chosen from the group of EX7@+63G, EX7@+190T, EX11@51A, EX25@855G and an LD SNP thereof;
(b) said gene is WARS2 and said nucleotide variant is chosen from the group of EX1@−963A, EX1@−103C, EX6@780A, EX6@842G and EX6@2152A, and LD SNPs thereof;
(c) said gene is ARTS1 and said nucleotide variant is chosen from the group of EX1@−1125T, EX2@397G, EX20@1085A, EX6@126A, EX12@44G, EX15@74G, EX6@149T, EX8@−10A, EX9@39T, EX9@+18T, EX11@59A, EX12@−28T, EX12@−7A, EX15@88C, EX19@173C, EX19@328m, EX19@885T, EX20@2105C, EX20@719C and EX20@1038A;
(d) said gene is MSR and said nucleotide variant is chosen from the group of EX1@−674T, EX1@19T, EX1@+129m, EX5@123C, EX5@136T, EX7@146A, EX10@+83A, EX11@+54T, EX14@14C, EX14@106G, EX14@142A and EX15@686G, and LD SNPs thereof.

5. A method of predicting the pathological, pharmacological or pharmacokinetic characteristic of a human subject, comprising:
determining the genotype at a loci of in Tables 1-35 and Table 81, said genotype is associated with a specific gene expression phenotype; and
predicting a pathological, pharmacological or pharmacokinetic characteristic of a human subject according to said specific gene expression phenotype.

6. The method of claim 5, wherein the TLK1 gene is genotyped, and where the presence of one or more of the SNPs EX7@+63G, EX7@+190T, EX11@51A, EX25@855G and LD SNPs thereof would indicate that the individual has an increased susceptibility to diseases associated with DNA damage including cancer.

7. The method of claim 5, wherein WARS2 gene is genotyped and wherein the presence of one or more of EX1@−963A, EX1@−103C, EX6@780A, EX6@842G and EX6@2152A, and LD SNPs thereof would indicate that the human subject has an increased likelihood of developing cardiovascular disease, cancer or neurodegenerative disease, or has a poor prognosis of such a disease.
